(19) United States
(12) Patent Application Publication
(10) Pub. No.: US 2022/0411777 A1
(43) Pub. Date: Dec. 29, 2022

(54) C-to-G Transversion DNA Base Editors

(71) Applicant: The General Hospital Corporation, Boston, MA (US)

(72) Inventors: J. Keith Joung, Winchester, MA (US); Ibrahim Cagri Kurt, Chelsea, MA (US); Ronghao Zhou, Charlestown, MA (US); Julian Grunewald, Charlestown, MA (US)

(21) Appl. No.: 17/638,157

(22) PCT Filed: Aug. 31, 2020
(86) PCT No.: PCT/US2020/048777

§ 371 Date: Feb. 24, 2022

Related U.S. Application Data
(60) Provisional application No. 63/023,208, filed on May 11, 2020.
(60) Provisional application No. 62/916,654, filed on Oct. 17, 2019.
(60) Provisional application No. 62/910,912, filed on Oct. 4, 2019.
(60) Provisional application No. 62/894,628, filed on Aug. 30, 2019.

(51) Int. Cl. C12N 9/78; C12N 15/10

(52) U.S. Cl. CPC C12N 9/78; C12N 15/102; C12Y 305/04005

(57) ABSTRACT

Engineered transversion base editors that enable expanded amino acid modifications and methods of using the same. Described herein, for example, are fusion proteins containing cytidine deaminases (e.g. human or rat APOBECs, pmCDA1 or AID) or adenosine deaminases (e.g. E. coli TadAs) or a combination thereof, catalytically impaired CRISPR-Cas proteins (e.g. Cas9, CasX or Cas12 nucleases), linkers, nuclear localization signals (NLSs) and a human or E. coli uracil-n-glycosylase (UNG) and/or REV1 protein that enable the CRISPR-guided programmable introduction of C-to-G and G-to-C transversions in DNA. The UNG may be fused to the deaminase-Cas fusion or not, in which case endogenous UNG may be recruited using molecular machinery that is integrated into the deaminase-Cas fusion architecture, e.g. using peptide or RNA aptamers or scFVs, sdABs or Fabs.

### CLAIM OF PRIORITY

This application claims the benefit of U.S. Patent Application Ser. No. 62/894,628 filed on Aug. 30, 2019; 62/910,912 filed on Oct. 4, 2019; 62/916,654 filed on Oct. 17, 2019; and 63/023,208, filed on May 11, 2020. The entire contents of the foregoing are hereby incorporated by reference.

### FEDERALLY SPONSORED RESEARCH OR DEVELOPMENT

This invention was made with Government support under Grant No. HG009490 awarded by the National Institutes of Health and contract HR0011-17-2-0042 awarded by the Defense Advanced Research Projects Agency of the Department of Defense. The Government has certain rights in the invention.

### TECHNICAL FIELD

Described herein are fusion proteins containing cytidine deaminases (e.g. human or rat APOBECs, pmCDA1 or AID) or adenosine deaminases (e.g. E. coli TadAs) or a combination thereof, catalytically impaired CRISPR-Cas proteins (e.g. Cas9, CasX or Cas12 nucleases), linkers, nuclear localization signals (NLSs) and a human or E. coli uracil-n-glycosylase (UNG) and/or REV1 protein that enable the CRISPR-guided programmable introduction of C-to-G and G-to-C transversions in DNA. The UNG may be fused to the deaminase-Cas fusion or not, in which case endogenous UNG may be recruited using molecular machinery that is integrated into the deaminase-Cas fusion architecture, e.g. using peptide or RNA aptamers or scFVs, sdABs or Fabs.

### BACKGROUND

DNA base editors represent a new class of genome editing tools that enable the programmable installation of single or multiple base substitutions. Current generations of cytosine base editors (CBE) and adenine base editors (ABE) allow for the targeted deamination of cytosines and adenines that get exposed on ssDNA by RNA-guided CRISPR-Cas proteins¹⁻⁴. The majority of disease-associated genetic perturbations known to date are point mutations, also known as single nucleotide variants (SNVs). Current iterations of CBEs and ABEs can target disease-relevant transition mutations and revert them to the original genotype, e.g. correcting G-to-A (C-to-T) mutations using ABE. However, a relevant fraction of disease-associated SNVs represent C-to-G and G-to-C substitutions that cannot be targeted using current BEs.

### SUMMARY

Described herein are CRISPR-guided C-to-G transversion base editors (CGBE) that enable the installation of cytosine-to-guanine and guanine-to-cytosine base edits in the ssDNA bubble generated by RNA-guided fusion proteins that contain adenine (e.g. E. coli TadA) and/or cytosine (e.g. rat APOBEC1) deaminases as well as CRISPR-Cas proteins (e.g. S. pyogenes Cas9) and/or REV1 or UNG proteins that are directly fused and/or recruited to the deaminase-Cas fusion protein. CGBE comprises a programmable DNA-binding domain (e.g. catalytically impaired dead or nicking Cas9) fused to a cytosine and/or adenosine deaminase. The adenosine deaminase can be a wild type (WT) or mutant E. coli TadA or previously described engineered TadA variants in the form of monomers, homodimers or heterodimers thereof, to decrease RNA editing activity while still preserving DNA editing activity (SECURE or RRE variants, Grünewald et al, NBT 2019—in press). The cytidine deaminase can be, e.g. rat APOBEC1, A3A, AID or pmCDA1, or previously described engineered variants of these deaminases (e.g. rAPOBEC1 with mutations from SECURE-BE3) with reduced RNA editing activity and preserved DNA editing capabilities⁵⁻⁹. In some embodiments, CGBE comprises one or more uracil-N-glycosylases (UNGs) fused to the N and/or C-terminus of the CBE or ABE fusion protein without uracil-N-glycosylase inhibitors (UGIs) and potentially with fused REV1 proteins. In some embodiments, CGBE comprise a linker between the adenosine or cytidine deaminase and the programmable DNA binding domain as well as between the deaminase domain and the UNG or the DNA binding domain and the UNG. In some embodiments the TadA domain can be monomeric, homodimeric or heterodimeric and contain all combinations of wild type (WT) E. coli TadA, or mutant variants of TadA).

Thus, provided herein are C-to-G transversion base editors (CGBEs) comprising a cytidine deaminase, a programmable DNA binding domain, and further comprising one or more nuclear localization sequences (NLS), and optionally one or more human or E. coli or other uracil-n-glycosylases (UNGs) or SMUG1, preferably wherein the CGBE does not comprise a uracil-N-glycosylase inhibitors (UGI).

In some embodiments, the cytidine deaminase comprises an active cytidine deaminase domain, preferably a monomeric domain, from a wild type and/or engineered rat APOBEC1 (rAPOBEC1), human APOBEC3A, human APOBEC3G, human AID, pmCDA1 (e.g., shown in Tables A and B) or variations thereof bearing mutations that reduce RNA or DNA off-target editing while retaining efficient DNA base editing.

In some embodiments, the cytidine deaminase comprises one or more mutations corresponding to mutations in rAPOBEC1, human APOBEC3A, human APOBEC3G, human AID or pmCDA1 or in any homologue or orthologue thereof (optionally those in Tables A and B).

In some embodiments, the cytidine deaminase is a rAPOBEC1 or any one of its ortho- or paralogues listed in Tables A or B, comprises one or more mutations that decrease RNA editing activity while preserving DNA editing activity, wherein the mutations are at amino acid positions that correspond to residues R33, P29, K34, E181, and/or L182 of rAPOBEC1 (SEQ ID NO:67) or to W90Y, R126E, R132E, W90Y+R126E (double mutant), R126E+R132E (double mutant), W90Y+R132E (double mutant), W90Y+R126E+R132E (triple mutant) (see, e.g., Ref. 16).

In some embodiments, the one or more mutations comprises a mutation at amino acid position that correspond to: (1) residue R33 of WT rAPOBEC1 or evoAPOBEC1; or (2) residue R13 in evoFERNY-APOBEC1; or (3) residue R12 in FERNY-APOBEC1.

In some embodiments, the mutation at amino acid position that correspond to residue R33 is a R33A substitution mutation.

In some embodiments, the CGBE comprises N- or C-terminal fusions of one or more human or E. coli UNG or SMUG1 or other orthologues of UNG or SMUG1 (e.g. as shown in Table J).

In some embodiments, the one or more UNGs are E. coli UNGs.

In some embodiments, the UNG(s) is absent, e.g., to minimize indel formation and reduce the size/length of the editor (e.g. miniCGBE1).

In some embodiments, the cytidine deaminase is a wildtype or engineered rAPOBEC1 (or any one of its ortho- or paralogues listed in Tables A or B) and the cytidine deaminase bears one or more mutations at positions: P29F, P29T, R33A, K34A, R33A+K34A (double mutant), E181Q and/or L182A of rAPOBEC1 (SEQ ID NO:67).

In some embodiments, the CGBE further includes one or more mutations at its cytidine deaminase rAPOBEC1 (or any one of its ortho- or paralogues listed in Tables A or B) residues corresponding to E24, V25; R118, Y120, H121, R126; W224-K229; P168-1186; L173+L180; R15, R16, R17, to K15-17 &A15-17; Deletion E181-L210; P190+P191; Deletion L210-K229 (C-terminal); and/or Deletion S2-L14 (N-terminal) of SEQ ID NO:67.

In some embodiments, the CGBE does not comprise one or more UNGs and/or the CGBE further comprises translesion polymerase REV1 (SEQ ID NO: 200) on either the N- or C-terminus or on both. In some embodiments, the CGBE comprises one or more UNGs and the tvBE further comprises a translesion polymerase REV1 (SEQ ID NO: 200). In some embodiments, the translesion polymerase REV1 (SEQ ID NO: 200) is fused to either the N- or C-terminus or both.

In some embodiments, the CGBE includes a linker between the cytosine deaminase monomer and/or between the cytosine deaminase monomer or single-chain dimers and the programmable DNA binding domain.

Exemplary Constructs Include:

1. CGBE1:

bpNLS-E.coliUNG-LINKER-rAPOBEC1(R33A)-LINKER-SpCas9(D10A)-bpNLS

2. miniCGBE1:

bpNLS-rAPOBEC1(R33A)-LINKER-SpCas9(D10A)-bpNLS

In some embodiments, the programmable DNA binding domain is selected from the group consisting of an engineered C2H2 zinc-finger, a transcription activator effector-like effector (TALE), and a Clustered Regularly Interspaced Short Palindromic Repeats (CRISPR) Cas RNA-guided nuclease (RGNs) and variants thereof.

The CGBE of any one of claims 1-15, wherein the CRISPR RGN is a ssDNA nickase or a catalytically inactive CRISPR Cas RNA-guided nuclease (e.g., a Cas9 or Cas12a that has ssDNA nickase activity or is catalytically inactive); in some embodiments, the Cas RGN is from SpCas9-NG or VRQR-Cas9.

Also provided herein are base editing systems comprising:

(i) a CGBE as described herein, wherein the programmable DNA binding domain is a CRISPR Cas RGN or a variant thereof; and
(ii) at least one guide RNA compatible with the base editor comprising a spacer sequence that directs the base editor to a target sequence, preferably wherein the target sequence comprises a cytosine at position 4-8, 5-7, or position 6 (with 1 being the most PAM-distal position).

Also provided herein are isolated nucleic acids encoding a CGBE as described herein, vectors comprising the isolated nucleic acids, and isolated host cells, preferably mammalian host cells (but also plant, bacterial, etc), comprising the nucleic acids or the vectors described herein. In some embodiments, the isolated host cell expresses the CGBE of any one of claims 1-17.

Additionally provided herein are methods for generating a cytosine-to-guanine and guanine-to-cytosine alteration in a nucleic acid, the method comprising contacting the nucleic acid with the CGBE of any one of claims 1-17, or the base editing system of claim 18.

In some embodiments, the CGBE achieves at least 15%, at least 20%, at least 25%, at least 30%, at least 35%, at least 40%, at least 45%, least 50%, at least 55%, at least 60%, or at least 63% C-to-G conversions in a target sequence.

In some embodiments, the target sequence is a sequence within or adjacent to one of the genes in Table E1 or Table E2.

Also provided herein are methods for generating a cytosine-to-guanine and guanine-to-cytosine alteration in a selected nucleotide of a target region of a nucleic acid. The methods include contacting the nucleic acid with:

(i) a C-to-G transversion base editor (CGBE) comprising an adenosine deaminase, e.g., a wild type and/or engineered (e.g. ABEs 0.1, 0.2, 1.1, 1.2, 2.1, 2.2, 2.3, 2.4, 2.5, 2.6, 2.7, 2.8, 2.9, 2.10, 2.11, 2.12, 3.1, 3.2, 3.3, 3.4, 3.5, 3.6, 3.7, 3.8, 4.1, 4.2, 4.3, 5.1, 5.2, 5.3, 5.4, 5.5, 5.6, 5.7, 5.8, 5.9, 5.10, 5.11, 5.12, 5.13, 5.14, 6.1, 6.2, 6.3, 6.4, 6.5, 6.6, 7.1, 7.2, 7.3, 7.4, 7.5, 7.6, 7.7, 7.8, 7.9, 7.10, ABEmax) E. coli TadA monomer, or variations of homo- or heterodimers thereof, bearing one or more mutations in either or both monomers that decrease RNA editing activity while preserving DNA editing activity, wherein the mutations are at amino acid positions that correspond to residues of E. coli TadA as listed in Table H, a programmable DNA binding domain comprising a ssDNA nickase or a catalytically inactive CRISPR Cas RNA-guided nuclease; and

(ii) at least one guide RNA compatible with the base editor and comprising a spacer that directs the base editor to the target sequence, preferably wherein the target sequence comprises a cytosine at position 4-8, 5-7, or position 6 (with 1 being the most PAM-distal position).

In some embodiments, the cytosine-to-guanine or guanine-to-cytosine alteration is listed in Table D.

Also provided herein are compositions comprising a CGBE or base editing systems as described herein, optionally including one or more ribonucleoprotein (RNP) complexes.

Additionally provided herein are the CGBE or base editing systems described herein, for use in generating a cytosine-to-guanine and guanine-to-cytosine alteration in a cell, wherein the alteration corrects a specific disease-related mutation provided in Tables E1 and E2.

In some embodiments, the CGBE does not comprise a UNG, and the CGBE recruits endogenous UNG with the help of a peptide aptamer fused to the CGBE.

In some embodiments, the CGBE does not comprise a UNG, and CGBE recruits endogenous UNG with the help of RNA aptamers fused to the gRNA.

In some embodiments, the CGBE does not comprise a UNG, and the CGBE recruits endogenous UNG with the help of a Fab, scFV or sdAb elements fused to the CGBE.

In some embodiments, the CGBE does not comprise a UNG, and wherein the CGBE recruits endogenous REV1 translesion polymerase.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. C-to-G transversion at position C6 in the FANCF site 1 spacer as an on-target byproduct of ABEmax and miniABEmax treatment in human HEK293T cells. FIG. 1A. Efficient DNA on-target A-to-G editing of the adenine in position 4 of the spacer (with 1 being the most PAM-distal position) by ABEmax and two miniABEmax variants compared to a nCas9-only negative control. 1B. C-to-G editing of the DNA cytosine in position 6 of the FANCF site 1 spacer in all ABE variants tested in the same experiment as shown in FIG. 1A. 1C. C-to-T editing of the DNA cytosine in position 6 of the FANCF site 1 spacer in all ABE variants tested in the same experiment as shown in FIG. 1A. 1D. C-to-A editing of the DNA cytosine in position 6 of the FANCF site 1 spacer in all ABE variants tested in the same experiment as shown in a. All data generated from independent quadruplicate experiments (n=4).

FIGS. 2A-2C. C-to-G transversion at position C6 is the predominant on-target byproduct on three genomic sites in human HEK293T cells treated with ABEmax and miniABEmax. 2A. C-to-G editing of the DNA cytosine in position 6 of the HEK site 2, ABE site 7, and FANCF site 1 spacer in all ABE variants tested with FANCF site 1 exhibiting the highest editing efficiencies as shown in FIGS. 1A-D. 2B. C-to-T editing of C6 was seen only at FANCF site 1. 2C. C-to-A editing in position 6 was only seen at consistently high levels at around 1-5% at FANCF site 1.

FIG. 3. Potential mechanism of action explaining C-to-G editing byproducts induced by ABE treatment in human HEK293T cells—part I. Schematic of an ABEmax protein inducing parallel targeted A-to-I deamination in the target ssDNA bubble as well as potentially inducing byproduct C-to-U deamination on position 6 of the spacer.

FIG. 4. Potential mechanism of action explaining C-to-G editing byproducts induced by ABE treatment in human HEK293T cells—part II. Schematic of uracil excision by UNG after the byproduct C-to-U deamination on position 6 was induced by ABE, leading to an abasic site at position 6 of the spacer. Downstream activity of mismatch repair (MMR) pathways and of the translesion polymerase REV1 as well as secondary deamination of adenines in C-to-A byproducts could potentially explain the higher proportion of C-to-G outcomes in position 6.

FIG. 5. Schematic drawing of approach to increase C-to-G product. Leveraging downstream processing of abasic sites by e.g. MMR and REV1, we propose using a CBE fusion protein containing a cytidine deaminase to enhance C-to-U deamination compared to ABE. In contrast to conventional CBE architectures, we propose to exchange the UGIs for a single or multiple UNG proteins to further increase the creation of abasic sites, thereby increasing the input for potential MMR and REV1 processing that may eventually lead to improved C-to-G editing yield.

FIG. 6. Schematic drawing of a C-to-G transversion base editor (CGBE) architecture. An N-terminal deaminase domain, e.g. rAPOBEC1, FERNY-APOBEC1, evoFERNY-APOBEC1, evoAPOBEC1, AID, A3A, eA3A, pmCDA1, A3G or an E. coli TadA mutant was fused to a catalytically impaired DNA binding protein, e.g. dCas9 or Cas9 nickase (D10A). An E. coli or human UNG protein was fused to the C-terminus.

FIG. 7. Schematic drawing of a C-to-G transversion base editor (CGBE) architecture that can show reduced indel byproduct frequency by fusing bacteriophage Mu Gam protein. The depicted fusion proteins showed a highly similar composition as the construct in FIG. 6 with the exception of the N-terminal (or C-terminal) fusion of the bacteriophage Mu Gam protein to reduce indel fractions, i.e. also in combination with the use of catalytically inactive Cas9 (dCas9).

FIG. 8. Schematic drawing of a C-to-G transversion base editor (CGBE) architecture with a fusion of the translesion polymerase REV1. In this construct, the anatomy of the initial CGBE (FIG. 6) was altered by exchanging UNG for REV1 on the C- or N-terminus.

FIG. 9. Schematic drawing of a C-to-G transversion base editor (CGBE) architecture with a fusion of both UNG and the translesion polymerase REV1. In this construct, the anatomy of the initial CGBE (FIG. 6) was altered by adding REV1 on the C- or N-terminus, leading to a CGBE variant that contains both UNG and REV1 as a direct fusion.

FIG. 10. Schematic drawing showing a construct where the anatomy of the initial CGBE (FIG. 6) was altered by fusing a peptide aptamer to the C- or N-terminus in order to recruit endogenous UNG instead of directly fusing UNG to the deaminase-Cas9 fusion protein.

FIG. 11. Schematic drawing showing a construct where the anatomy of the initial CGBE (FIG. 6) was altered by fusing a scFV, Fab or sdAb to the C- or N-terminus in order to recruit endogenous UNG instead of directly fusing UNG to the deaminase-Cas9 fusion protein.

FIG. 12. Schematic drawing showing a construct where the anatomy of the initial CGBE (FIG. 6) was altered by encoding an RNA aptamer directly in the gRNA in order to recruit endogenous UNG instead of directly fusing UNG to the deaminase-Cas9 fusion protein.

FIG. 13. Engineering of a C-to-G base editor. Bar plots showing on-target DNA base editing frequencies with various base editor architectures using seven gRNAs targeting genomic sites in HEK293T cells. N and C indicate amino-terminal and carboxy-terminal ends, respectively, of the various base editors. Gray overlay bars at top represent deletions at each editing window. Target cytosines are highlighted. Editing frequencies of three independent replicates (n=3) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits. Arrows point at examples of C-to-G edits.

FIG. 14. On-target activities of nCas9 controls, ABE variants, and more CBE variants tested for C-to-G editing in HEK293T cells. Bar plots showing the on-target DNA base editing frequencies induced by nCas9 negative controls, ABE and ABE variants, and other CBE variants with seven gRNAs in HEK293T cells. N and C indicate amino-terminal and carboxy-terminal ends, respectively, of the various base editors. Gray overlay bars at top represent deletions at each editing window. Editing frequencies of three independent replicates (n=3) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits.

FIGS. 15A-B. Indel frequencies of nCas9 controls, ABE variants, and CBE variants tested for C-to-G editing in HEK293T cells. a,b, Dot plots representing percentage of alleles that contain an insertion or deletion across the entire protospacer from experiments with various base editor architectures reported in FIG. 14 (15a) or FIGS. 13 and 14 (15b). Single dots represent individual replicates.

FIG. 16. On-target activities of non-APOBEC1 CBE variants tested for C-to-G editing in HEK293T cells. Bar plots showing the on-target DNA base editing frequencies induced by non-APOBEC1 CBEs and their variants with h/eUNG with seven gRNAs in HEK293T cells. N and C indicate amino-terminal and carboxy-terminal ends, respectively, of the various base editors. Gray overlay bars at top represent deletions at each editing window. Editing frequencies of three independent replicates (n=3) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits.

FIG. 17. Indel frequencies of non-APOBEC1 CBE variants tested for C-to-G editing in HEK293T cells. Dot plots representing percentage of alleles that contain an insertion or deletion across the entire protospacer from experiments with non-APOBEC1 CBE variants reported in FIG. 16. Single dots represent individual replicates.

FIGS. 18A-B. Additional characterization of CGBE1 on-target editing activities in HEK293T cells. A,B, Bar plots showing the on-target DNA base editing frequencies induced by BE4max(R33A) and CGBE1 using 12 gRNAs with a C at position 6 (C6-sites; 18A) and 6 gRNAs with a C at position 4, 5, 7, or 8 (non-C6-sites; 18B) in HEK293T cells. N and C indicate amino-terminal and carboxy-terminal ends, respectively, of the various base editors. Gray overlay bars at top represent deletions at each editing window. Editing frequencies of three independent replicates (n=3) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits.

FIG. 19. Aggregated distribution of editing and indel frequencies across protospacer of BE4max(R33A) and CGBE1 in HEK293T cells. Dot and box plots representing the combined distribution of C-to-G, C-to-T, C-to-A, and indel frequencies (labeled) across the entire protospacer from experiments performed with BE4max(R33A) and CGBE1 using 25 guides. Boxes span the interquartile range (IQR; first to third quartiles), horizontal lines indicate the median (second quartile), and whiskers extend to ±1.5×IQR. Single dots represent individual replicates. The graphs were derived from the data shown in FIGS. 13 and 18A-B.

FIGS. 20A-B. On-target activities of nCas9 controls and CGBE1-related variants with more gRNAs in HEK293T cells. A,B, Bar plots showing the on-target DNA base editing frequencies of nCas9 controls and CGBE1-related variants using 12 gRNAs with a C at position 6 (C6-sites; 20A) and 6 gRNAs with a C at position 4, 5, 7, or 8 (non-C6-sites; 20B) in HEK293T cells. N and C indicate amino-terminal and carboxy-terminal ends, respectively, of the various base editors. Gray overlay bars at top represent deletions at each editing window. Editing frequencies of three independent replicates (n=3) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits.

FIG. 21. Indel frequencies of CGBE1 and CGBE1-related variants with more gRNAs in HEK293T cells. Dot plots representing percentage of alleles that contain an insertion or deletion across the entire protospacer from experiments with CGBE1-related variants reported in FIGS. 18A-B and 20A-B. Single dots represent individual replicates.

FIGS. 22A-B. Comparison of CGBE1 and miniCGBE1 on-target editing activities with 25 gRNAs in HEK293T cells. A,B, Bar plots showing the on-target DNA base editing frequencies of CGBE1 and miniCGBE1 using 19 gRNAs with a C at position 6 (C6-sites; 22A) and 6 gRNAs with a C at position 4, 5, 7, or 8 (non-C6-sites; 22B) in HEK293T cells. N and C indicate amino-terminal and carboxy-terminal ends, respectively, of the various base editors. Gray overlay bars at top represent deletions at each editing window. Editing frequencies of three independent replicates (n=4) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits.

FIGS. 23A-B. On-target activities of nCas9 control with 25 gRNAs in HEK293T cells. A,B, Bar plots showing the on-target DNA base editing frequencies observed with expression of a nCas9 negative control using 19 gRNAs with a C at position 6 (C6-sites; 23A) and 6 gRNAs with a C at position 4, 5, 7, or 8 (non-C6-sites; 23B) in HEK293T cells. N and C indicate amino-terminal and carboxy-terminal ends, respectively, of the various base editors. Gray overlay bars at top represent deletions at each editing window. Editing frequencies of three independent replicates (n=4) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits in respective CGBE experiments.

FIG. 24. Indel frequencies of CGBE1 and miniCGBE1 variants with 25 gRNAs in HEK293T cells. Dot plots representing percentage of alleles that contain an insertion or deletion across the entire protospacer from experiments with CGBE1 and miniCGBE1 reported in FIG. 22 and control experiments reported in FIG. 23. Single dots represent individual replicates.

FIG. 25. Additional comparison of CGBE1 and miniCGBE1 on-target editing activities with 23 non-C6 gRNAs in HEK293T cells. Bar plots showing the on-target DNA base editing frequencies induced by nCas9 control, BE4max, BE4max(R33A), CGBE1, and miniCGBE1 with 23 gRNAs for sites with a C at position 4, 5, 7, or 8 (non-C6 sites) in HEK293T cells. N and C indicate amino-terminal and carboxy-terminal ends, respectively, of the various base editors. Gray overlay bars at top represent deletions at each editing window. Editing frequencies of three independent replicates (n=3) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits.

FIG. 26. Indel frequencies of CGBE1 and miniCGBE1 variants with 23 non-C6 gRNAs in HEK293T cells. Dot plots representing percentage of alleles that contain an insertion or deletion across the entire protospacer from experiments with BE4max, BE4max(R33A), CGBE1 and miniCGBE1 reported in FIG. 25. Single dots represent individual replicates.

FIGS. 27A-B. Aggregated distribution of C-to-G editing frequencies across protospacer of CGBE1 and miniCGBE1 in HEK293T cells. A,B, Dot and box plots representing the aggregate distribution of C-to-G (yellow) editing frequencies across the entire protospacer from experiments performed with CGBE1 (27A) and miniCGBE1 (27B) with all 48 tested gRNAs. Boxes span the interquartile range (IQR; first to third quartiles), horizontal lines indicate the median (second quartile), and whiskers extend to ±1.5×IQR. Single dots represent individual replicates. The graphs were derived from the data shown in FIGS. 22A-B and 25.

FIG. 28. Off-target DNA editing activities of CGBE1 and miniCGBE1 in HEK293T cells. Bar plots showing the off-target DNA base editing frequencies induced by nCas9 control, BE4max, BE4max(R33A), CGBE1, and miniCGBE1 using HEK site 2, HEK site 3, HEK site 4, EMX1 site 1, and FANCF site 1 gRNAs in HEK293T cells. N and C indicate amino-terminal and carboxy-terminal ends, respectively, of the various base editors. Gray overlay bars at top represent deletions at each editing window. Editing frequencies of three independent replicates (n=3) at each base are displayed side-by-side. Percentage values below specific cytosine bases indicate the average C-to-D (D=A/T/G) editing observed (values below 1% not reported). Numbering on the bottom indicates position of the base in the protospacer with 1 being the most PAM-distal base. Arrowheads indicate cytosines showing C-to-G edits.

FIG. 29. Indel frequencies of CGBE1 and miniCGBE1 variants for DNA off-targets in HEK293T cells. Dot plots representing percentage of alleles that contain an insertion or deletion across the entire protospacer from experiments with BE4max, BE4max(R33A), CGBE1 and miniCGBE1 reported in FIG. 28. Single dots represent individual replicates.

FIG. 30. On-target DNA editing activities of NG and VRQR variants of CGBE1 and miniCGBE1 in HEK293T cells. Bar plots showing the on-target DNA base editing frequencies induced by NG and VRQR variants of nCas9, CGBE1, and miniCGBE1 using 6 gRNAs that target AT-rich genomic loci with PAMs that are compatible with SpCas9-NG (NGT) and SpCas9-VRQR (NGAG) variants in HEK293T cells. N and C indicate amino-terminal and carboxy-terminal ends, respectively, of the various base editors. Gray overlay bars at top represent deletions at each editing window. Editing frequencies of three independent replicates (n=4) at each base are displayed side-by-side. Percentage values below specific cytosine bases indicate the average C-to-G editing observed (values below 3% not reported). Numbering on the bottom indicates position of the base in the protospacer with 1 being the most PAM-distal base. Arrowheads indicate cytosines showing C-to-G edits.

FIG. 31. Indel frequencies of NG and VRQR variants of CGBE1 and miniCGBE1 variants in HEK293T cells. Dot plots representing percentage of alleles that contain an insertion or deletion across the entire protospacer from experiments with NG and VRQR variants of CGBE1 and miniCGBE1 reported in FIG. 30. Single dots represent individual replicates.

FIG. 32. Potential mechanism of prime editing system. Schematic of prime editing (PE) used to install a C-to-G substitution. PE fusion protein consists of an SpCas9-H840A nickase fused to an engineered Moloney murine leukemia virus reverse transcriptase (MMLV-RT). The prime editing guide RNA (pegRNA) consists of a standard targetable SpCas9 gRNA that also harbors a 3′ extension containing a primer binding site (PBS) and a reverse transcription template (RTT) that encodes the desired edit. PE2 system encompasses the prime editor fusion protein and a pegRNA. PE3 system additionally includes a nicking gRNA (ngRNA).

FIGS. 33A-B. Testing PE2 and PE3 in multiple human cell lines. A,B, Bar and dot plots representing the on-target DNA prime editing and indel frequencies of PE2 and PE3 targeting FANCF site 1 for G-to-T prime editing (33A) and HEK site 3 for PE-induced CTT insertion (33B) in 4 cell lines. Single dots represent individual replicates. Error bars represent standard deviation.

FIG. 34. Comparing the editing activities of CGBEs and PEs in multiple human cell lines. Bar plots showing the average on-target DNA C-to-G base or prime editing frequencies induced by CGBE1, miniCGBE1, PE2, or PE3 on four genomic target loci. Each site in each cell line was tested with four independent replicates in HEK293T cells and three independent replicates in K562, U205, and HeLa cells. Single dots represent individual replicates. A two-tailed Student's t-test with p-values adjusted for multiple testing was used to calculate the shown p-values. Error bars represent standard deviations.

FIG. 35. Testing pegRNAs and nicking gRNAs with wild-type SpCas9 in HEK293T cells. Bar and dot plots representing the frequency of alleles with indels (%) induced by pegRNAs and nicking gRNAs used in the experiments in FIGS. 33 and 34 (and FANCF site 1+21 ngRNA control) with wild-type SpCas9 in HEK293T. pegRNAs/ngRNAs designed by Anzalone et al. and by us are separated by the dashed line. Single dots represent individual replicates. Error bars represent standard deviations. ND, not done.

FIG. 36. Additional comparisons of CGBE1, miniCGBE1, PE2, and PE3 on-target editing activities in HEK293T, K562, U2OS, and HeLa cells. Bar plots showing the on-target DNA editing frequencies induced by nCas9 controls, CGBE1, miniCGBE1, PE2, and PE3 with four gRNAs (CGBEs), four pegRNAs (PE2), or 4 pegRNA/nicking gRNA combinations (PE3), designed to install a C-to-G substitution at the same cytosine at four genomic loci in four cell lines. Gray overlay bars at top represent deletions at each site. Editing frequencies of four independent replicates (n=4) for HEK293T cells or three independent replicates (n=3) for K562, U2OS, and HeLa cells at each base are displayed side-by-side. Percentage values below cytosine bases reflect the average C-to-G editing observed (values below 3% not reported). Numbering on the bottom indicates position of the base with 1 being the most PAM-distal base for base editors, or the first nucleotide 3′ of the pegRNA/Cas9-induced nick for prime editors. Arrowheads indicate cytosines showing C-to-G edits.

FIG. 37. Indel frequencies of CGBE1, miniCGBE1, PE2, and PE3 in HEK293T, K562, U2OS, and HeLa cells. Dot plots representing percentage of alleles that contain an insertion or deletion across the entire protospacer from experiments with CGBE1, miniCGBE1, PE2, and PE3 reported in FIGS. 34 and 36. Single dots represent individual replicates.

### DETAILED DESCRIPTION

ABEs install A-to-G substitutions in DNA while CBEs allow for the introduction of C-to-T mutations. However, both these types of mutations represent transitions and the extensive subset of disease-associated transversion mutations—e.g. C-to-G mutations-cannot be directly targeted with neither CBEs nor ABEs.

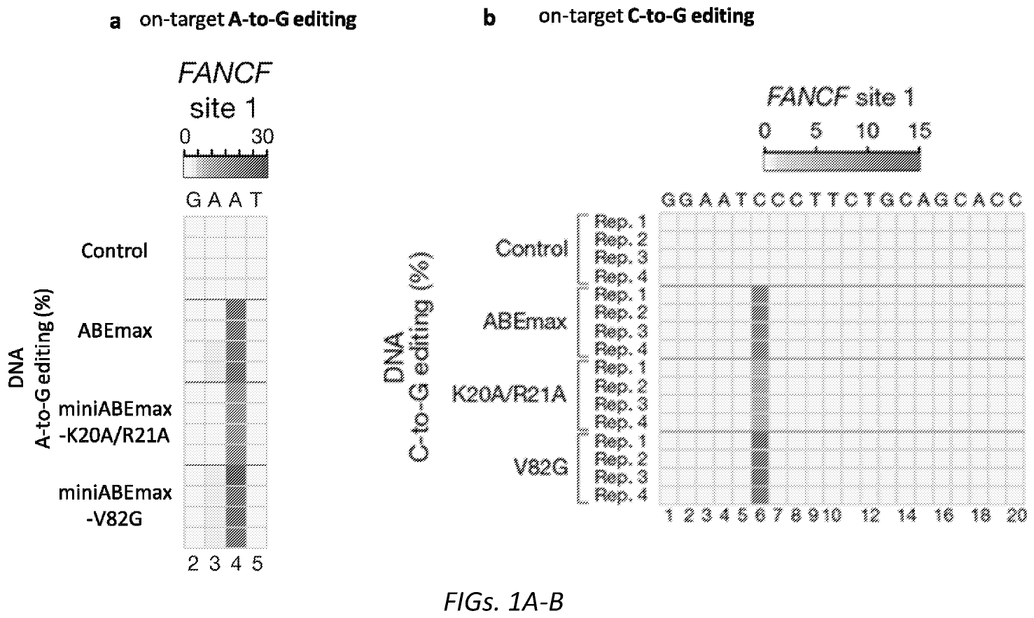

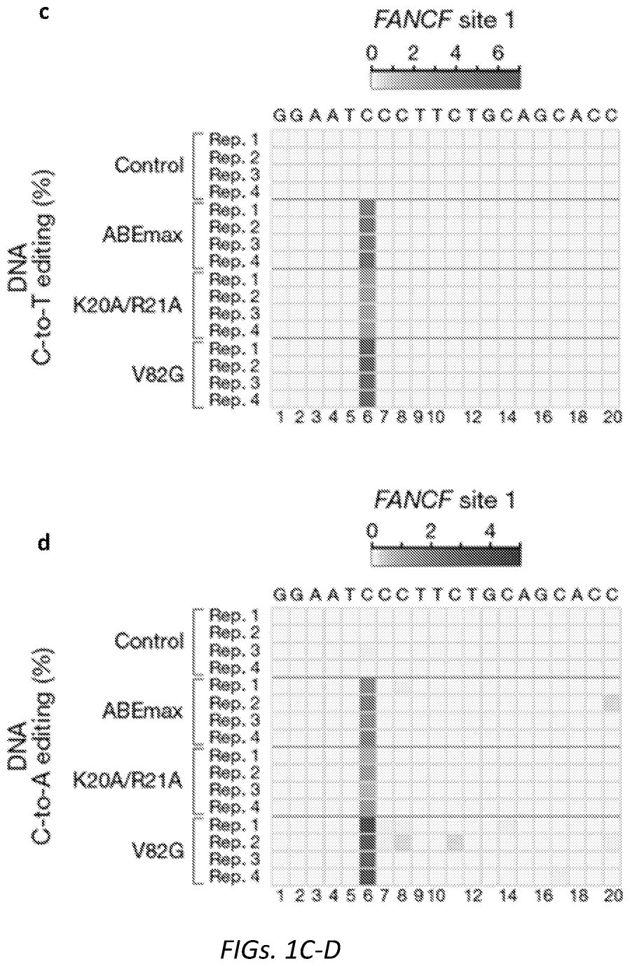

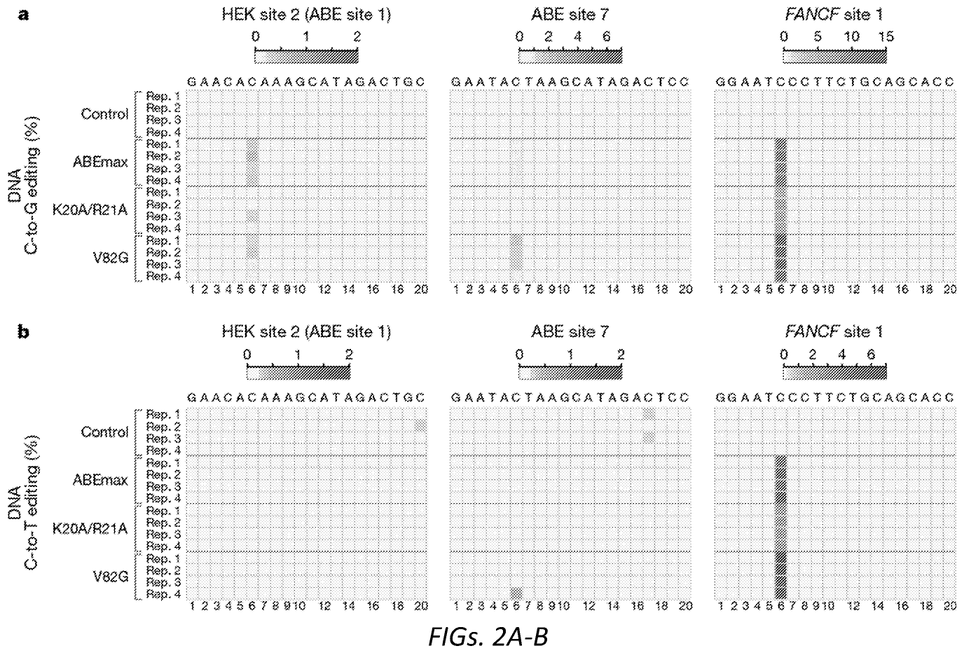

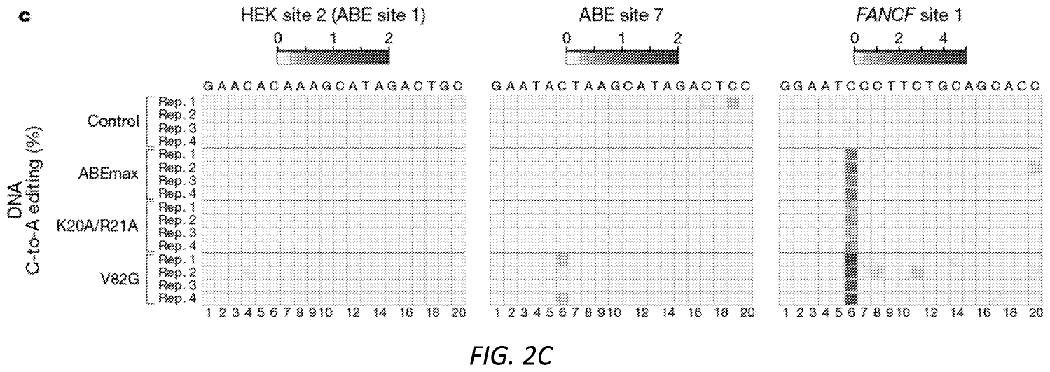

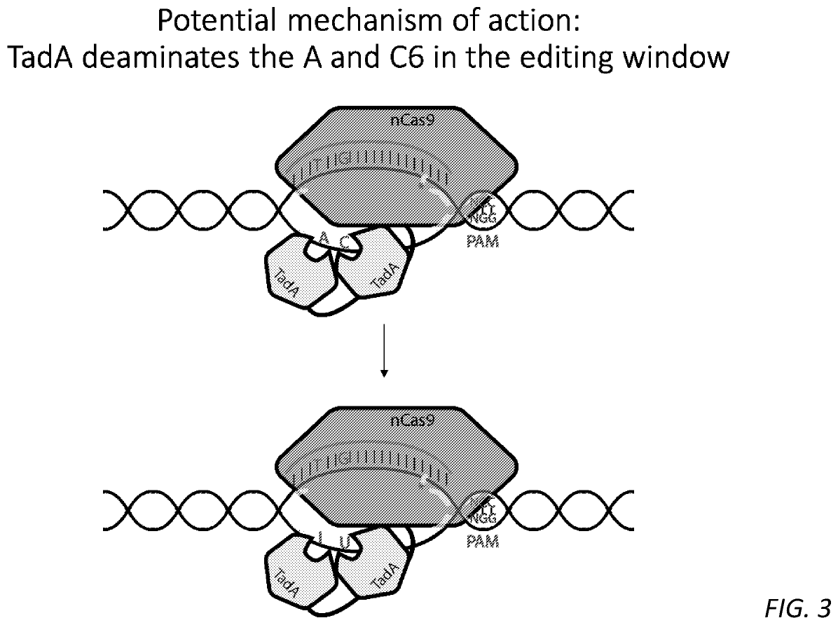

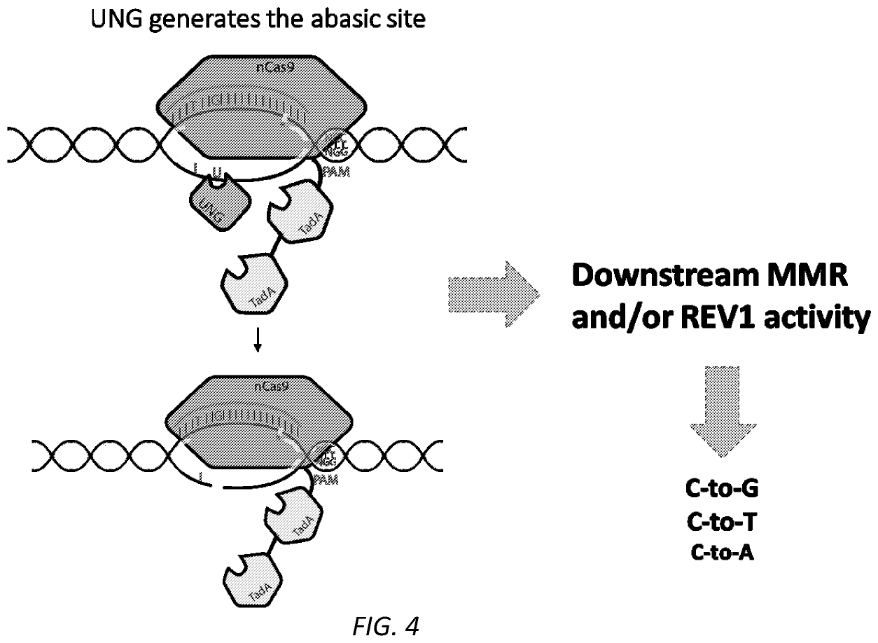

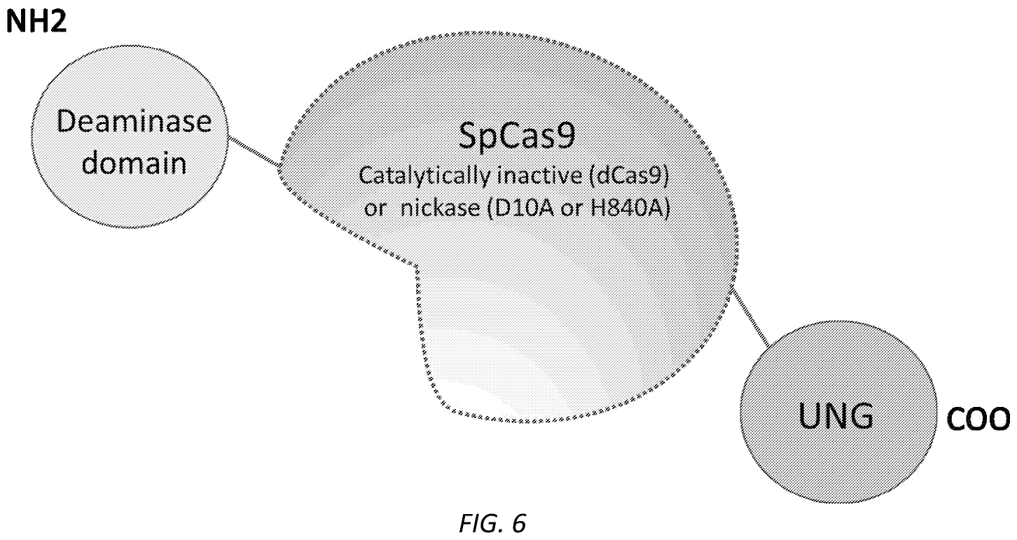

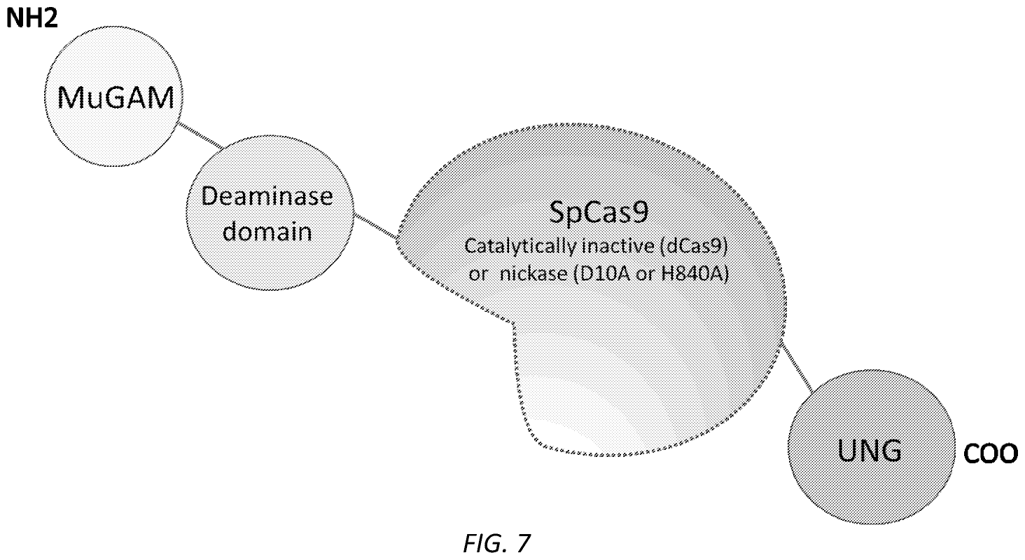

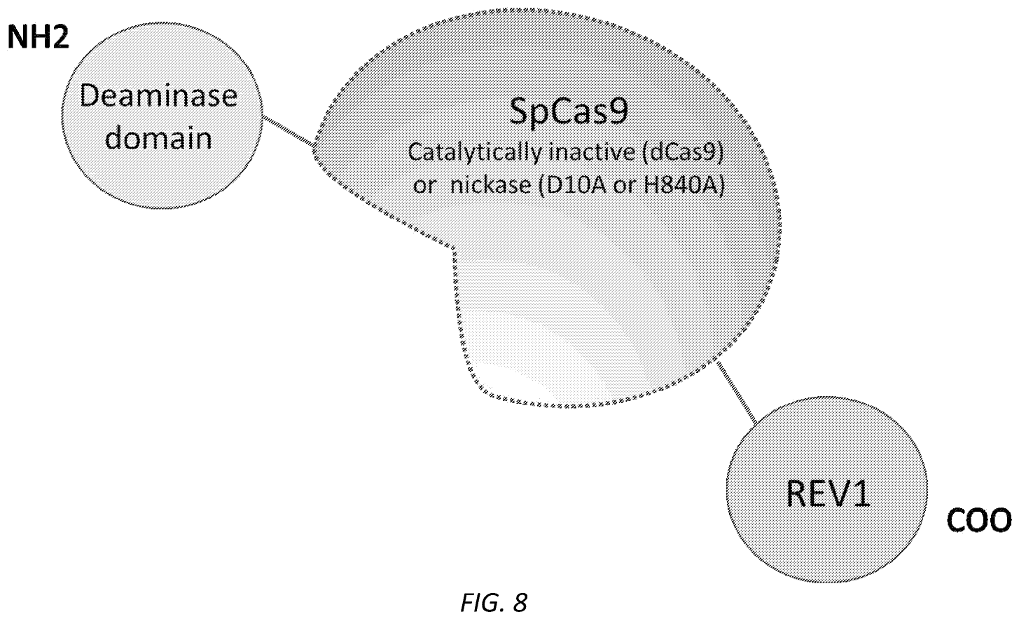

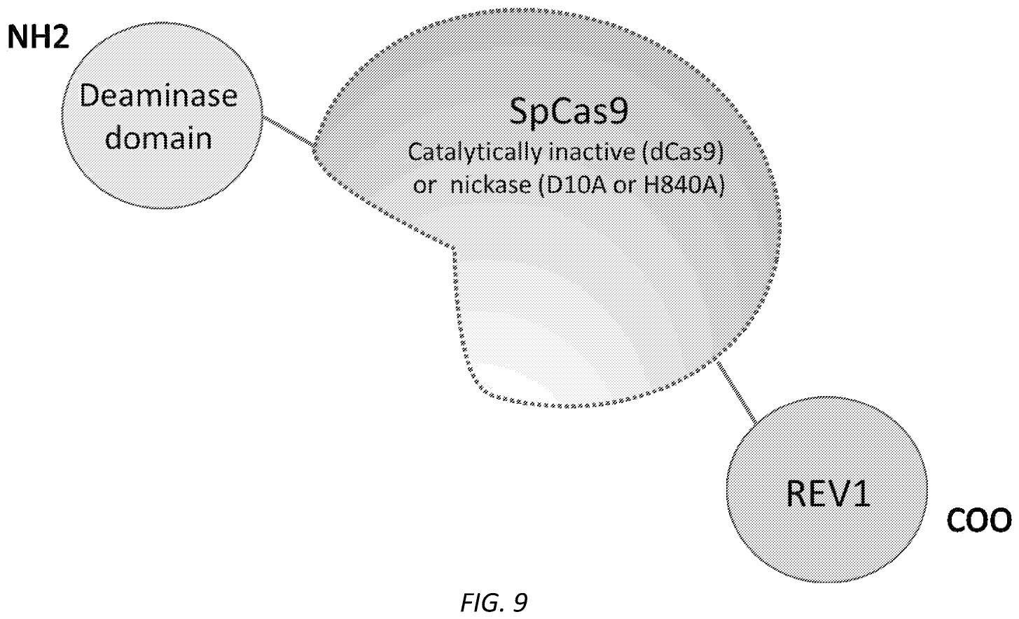

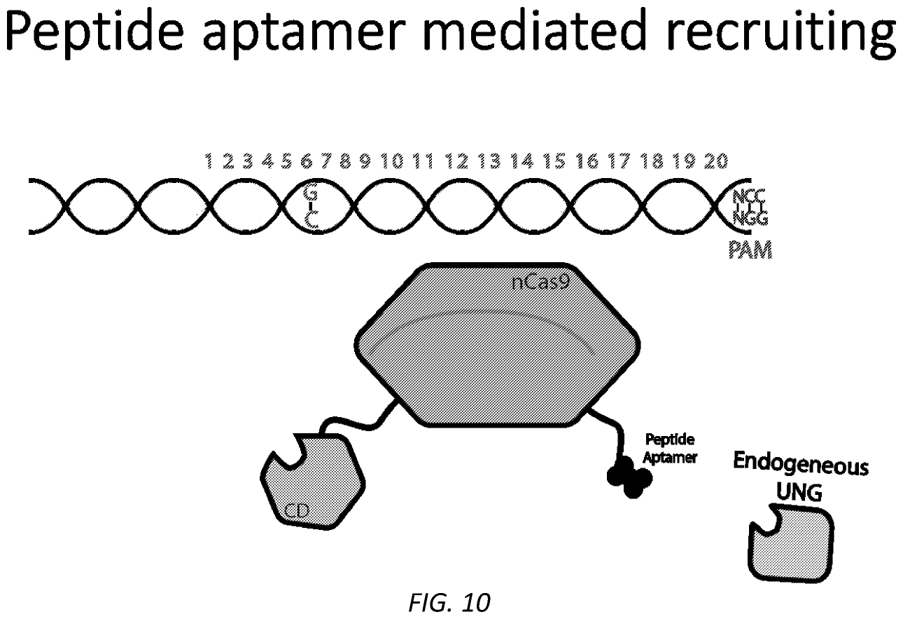

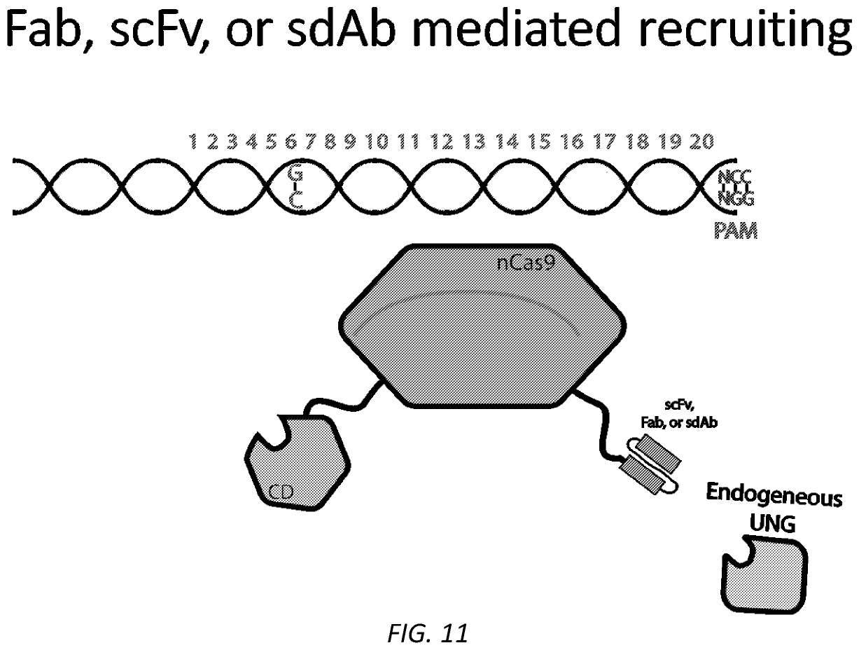

We sought to engineer a C-to-G transversion base editor (CGBE) that enables the programmable installation of C-to-G and G-to-C mutations. Based on our finding that ABE proteins that do not comprise UGIs can reproducibly induce C-to-G editing at position 6 of the spacer (with 1 being the most PAM-distal position) at mutliple genomic sites (FIGS. 1 and 2; Grunewald et al, Nature Biotechnology 2019), we hypothesized that we could engineer a base editing construct that might allow for higher C-to-G yield. We engineered CGBEs comprised of cytidine deaminases or adenosine deaminases or both (e.g. as in dual-deaminase architecture of bifunctional adenine and cytosine base editors, BACE) fused to DNA binding proteins (e.g. dCas9 or nickase Cas9) as well as to UNG or REV1 proteins or a combination thereof. We hypothesized that using a cytidine deaminase will increase C-to-U deamination rates at C6 or neighboring cytosines at the target ssDNA bubble, and fusing base excision repair (BER) protein UNG or translesion polymerase REV1 (without fusing a UGI) might enable increased formation of an abasic site at position 6 of the genomic target site. Downstream processing of the abasic site via MMR or translesion synthesis could subsequently yield higher C-to-G product (FIG. 3-5). Described herein are a number of different fusion protein architectures involving the abovementioned domains and proteins. Some embodiments use dCas9 and/or bacteriophage Mu Gam (FIG. 6-9; Komor et al, Sci Adv 2017) to reduce insertion/deletion (indel) byproducts, thereby further increasing relative C-to-G product yield and purity. In some embodiments, the methods include recruiting endogenous UNG to the programmable base editing target site with the use of peptide aptamers fused to CGBEs (delta UNG), RNA aptamers integrated into the gRNA or CGBE (delta UNG) fusion proteins harboring scFVs, sdABs or Fabs to recruit endogenous UNG (FIG. 10-12).

Thus, described herein are variants of base editor fusion proteins that enable the programmable introduction of transversion base edits, specifically C-to-G and G-to-C. A table of potentially actionable codon and amino acid changes are shown in Table D and a list of potential disease targets (using Cas proteins compatible with NGG, NG, and NGA-PAMs) is shown in Tables E1-E3.

### Exemplary Cytidine Deaminase Domains Used for CGBE

In some embodiments, the cytidine deaminase is pmCDA1 (sea lamprey) or APOBEC1 from rat, or from a different species (Table A), e.g., a different mammalian species such as H. sapiens. The APOBEC, AICDA (AID) and CDA1 family members have high sequence homology and represent potential candidates for CGBE architectures (Table B)^{2,15-18}.

Specifically, reduced RNA editing variants of rAPOBEC1, enhanced human A3A, and human AID are candidates for inclusion into CGBE architectures.

In some embodiments, CGBE described herein can be a wild-type BE4max or SECURE-BE4max-R33A as well as eA3A variants with truncated UGIs and additional N- or C-terminal fusion of a human or E. coli UNG.

In some embodiments, the cytidine deaminases in Anc-BE4max, evoAPOBEC1-BE4max (SEQ ID 205), FERNY-BE4max, evoFERNY-BE4max (SEQ ID 204), CDA1-BE4max, and evoCDA1-BE4max may be used in a BE4max architecture with truncated UGIs and optionally also have UNGs (human or E. coli, N- or C-terminal) added. In other embodiments, the SECURE-CBE R33 and/or K34 residue changes may be introduced in evoAPOBEC1.

In some embodiments, R13 and/or K14 residue changes are introduced in FERNY and evoFERNY-APOBEC1 (these residue changes are embedded in the same amino acid sequence motif as R33 and K34 in WT rat APOBEC1 that was used in BE3, BE4, and BE4max). These modifications (single or double residue change) can greatly reduce RNA off-target editing and enhance on-target C-to-G editing. All of the APOBEC1-based CBEs described herein can used with or without the proposed mutations in the context of a C-to-G transversion base editor.

The cytidine deaminase domain need not include an entire full protein, but can be a variant as described herein that has changes or truncations that do not abolish the cytidine deaminase activity.

### Exemplary Adenosine Deaminase Domains Used for CGBE

In some embodiments, the adenosine deaminase is TadA from E. coli, or an orthologue from a different prokaryote, e.g. S. aureus, or a homologue from the eukaryotic domain, such as yeast TAD1/2 or a mammalian species such as human (e.g. ADAT2; Table C). The tRNA-specific adenosine deaminase family members have high sequence homology and many of these orthologues may be compatible with one or more of the amino acid substitutions in E. coli TadA expected to cause an RRE phenotype and would be desirable in a CGBE architecture.

The wild type sequence of wild type E. coli TadA, available in uniprot at P68398, is as follows:

(SEQ ID NO: 1)
MSEVEFSHEYWMRHALTLAKRAWDEREVPVGAVLVHNNRVIGEGWNRPI
GRHDPTAHAEIMALRQGGLVMQNYRLIDATLYVTLEPCVMCAGAMIHSR
IGRVVFGARDAKTGAAGSLMDVLHHPGMNHRVEITEGILADECAALLSD
FFRMRRQEIKAQKKAQSSTD.

The engineered E. coli TadA sequence present in ABE7.10 and ABEmax is as follows:

SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMPRQVFNAQKKAQSSTD.

In the most commonly used ABEs (ABE7.10 and ABEmax), these two proteins were fused using a 32 amino acid linker (bolded in sequence below), forming a heterodimer, the sequence of which is as follows:

MSEVEFSHEYWMRHALTLAKRAWDEREVPVGAVLVHNNRVIGEGWNRPI
GRHDPTAHAEIMALRQGGLVMQNYRLIDATLYVTLEPCVMCAGAMIHSR
IGRVVFGARDAKTGAAGSLMDVLHHPGMNHRVEITEGILADECAALLSD
FFRMRRQEIKAQKKAQSSTDSGGSSGGSSGSETPGTSESATPESSGGSS
GGSSEVEFSHEYWMRHALTLAKRARDEREVPVGAVLVLNNRVIGEGWNR
AIGLHDPTAHAEIMALRQGGLVMQNYRLIDATLYVTFEPCVMCAGAMIH
SRIGRVVFGVRNAKTGAAGSLMDVLHYPGMNHRVEITEGILADECAALL
CYFFRMPRQVFNAQKKAQSSTD.

Other exemplary sequences are shown in Table C. These tRNA-specific adenosine deaminase orthologues and homologues also represent candidates for inclusion of the mutations previously described at analogous positions in these proteins.

In some embodiments, the base editors included catalytically dead adenine deaminase variants, e.g. E59A. (Gaudelli et al, 2017, PMID: 29160308) as part of a heterodimer.

The adenine deaminase domain need not include an entire full protein, but can be a variant as described herein that has changes or truncations that do not abolish the adenine deaminase activity.

### Uracil DNA Dlycosylase (UNG)

Cellular molecular pathways are in complete homeostasis within healthy cells. Especially, DNA repair pathways are balanced in ways that potentially mutagenic lesions are repaired at the optimal level. In mammalian cells, there is continuous generation of deamination mutations and repair of deamination reactions occurring in the background. Impairments in this process can lead to disruption of this homeostasis. On the deamination side, aberrant overexpression of deaminases that can induce spontaneous deamination at DNA and RNA levels has been shown to be responsible for inducing different cancers.^{10,11} On the other hand, expression levels of DNA glycosylases—a family of enzymes responsible for repairing the deaminated bases via the base excision repair (BER) pathway—are also crucial. DNA glycosylases carry out their activity by removing the lesions and creating abasic sites. Overexpression of uracil DNA glycosylase (UNG) has been shown to confer chemotherapy resistance in certain cancers.¹² Moreover, overexpression of uracil glycosylase inhibitor (UGI), a component of CBEs, is potentially responsible for the observed levels of toxicity and genome-wide Cas9-independent DNA off-target effects that can be induced by CBEs. In the light of these aforementioned independent observations, it is clear that one needs to control and optimize the expression levels of the exogeneous base editor constructs in order to minimize the potential unwanted side-effects to the target cells and preserve the homeostasis.

In some embodiments of the C-to-G transversion base editors (CGBEs) described herein, Uracil-DNA glycosylase (UNG) is a critical component that carries out the generation of abasic sites after cytosines are deaminated to uracil.

Exemplary UNG/SMUG Sequences for Inclusion in CGBE

In some embodiments, the CGBE fusion proteins described herein include a functional UNG or Single-Strand-Selective Monofunctional Uracil-DNA Glycosylase 1 (SMUG1) domain. Table J provides a list of UNG and SMUG1 orthologues.

Recruiting Endogenous UNG to Target and Edit Genetic Loci

While overexpression of engineered constructs is the first and main strategy to edit genomic loci, it has been well established that overexpression of exogeneous proteins can have unwanted and fatal consequences. In the context of base editors specifically, it has been demonstrated that overexpression of base editors can induce hundreds to thousands of off-target single nucleotide variations (SNVs) on DNA and RNA.^{6,7,13,14} All in all, there is great need to temporally and spatially control the expression levels of base editors in target cells. To this end, recruiting the endogenous cellular machinery to carry out the enzymatic reactions of interest, instead of exogenously providing a protein in excess, is a prominent bypass to minimize exogeneous components that need to be overexpressed.

It is possible that exogeneous overexpression of human or bacterial UNG may alter the repair pathway balance towards more efficient abasic site generation genome-wide. While more research is warranted to elucidate the impact of such UNG overexpression in mammalian cells, bypassing the need for overexpression of an immunogenic (in the case of E. coli UNG) protein and preserving the natural endogenous expression levels of UNG would be advantageous. To this end, we are proposing to utilize three alternative methods/constructs with the aim of recruiting the endogenous UNG to the target site of deamination.

### Section 1: Peptide Aptamer Mediated Recruiting of UNG to the Target Site

Peptide aptamers are small amino acid sequences that can be designed and selected against virtually any given protein of interest. Peptide aptamers can have dissociation constants similar to naturally found antibodies. Owing to their small size, ease of production, high specificity, higher stability and solubility, peptide aptamers represent a significant alternative to the antibodies. Starting from an initial randomized library of peptides, peptide aptamers can be selected and further optimized via various methods in vitro and in vivo.

Fusing an engineered peptide aptamer against human UNG into our CGBE constructs would allow us to recruit endogenous UNG bypassing the need to overexpress the protein exogenously. (FIG. 10)

Also, various peptide aptamers can be engineered from scratch against human UNG by methods including but not limited to yeast-two-hybrid systems in vivo, and phage-display in vitro systems. Candidate peptide aptamers displaying strong affinity against human UNG will be sequenced and the identified DNA and amino acid sequences will be employed as fusion partners in our next generation CGBE constructs. Optimal conformation of the peptide aptamer fusion will be determined empirically by cloning it into different sites in our constructs with different linkers.

### Section 2: RNA Aptamer Mediated Recruiting of UNG to the Target Site

RNA aptamers are short stretches (80-120 nucleotides) of RNA molecules with strong and selective affinity against the target proteins of interest. Candidate RNA aptamers can be chemically synthesized as randomized libraries and several rounds of in vitro and in vivo selections can be applied. Employing the method called Systematic Evolution of Ligands by EXponential enrichment (SELEX), a number of candidate RNA aptamer molecules can be identified against one's target protein of interest.

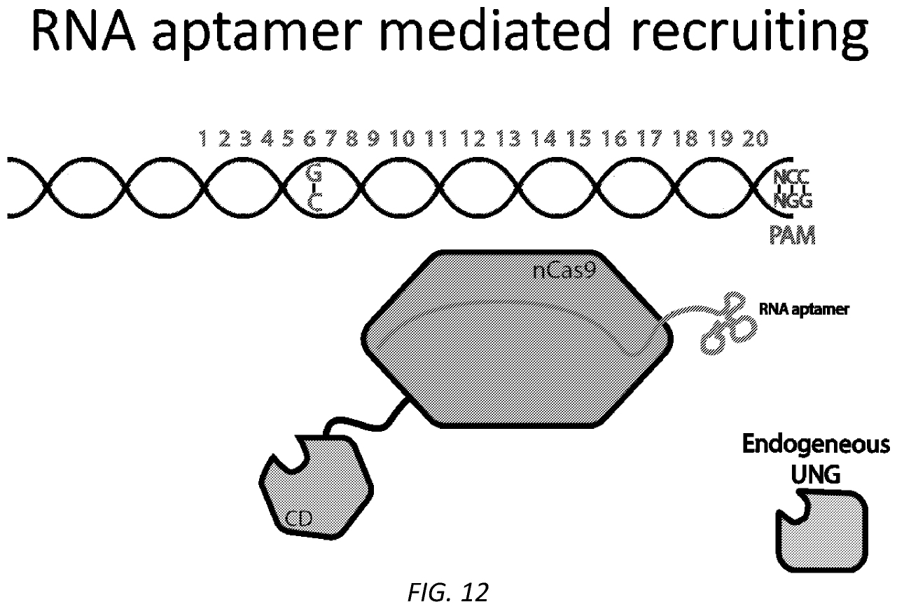

As an example, the fusion of MS2 aptamers to CRISPR gRNAs is a widely used and well-known example of such a strategy. In this strategy, MS2 RNA aptamers are fused to the ends of gRNA constructs, thereby enabling specific recruitment of MS2 bacteriophage coat protein fused target proteins. Therefore, we propose that fusing an already engineered RNA aptamer against human UNG, if any exists, into the gRNA component of our CGBE constructs would allow us to recruit endogenous UNG bypassing the need to overexpress exogenously. (FIG. 12)

Also, various RNA aptamers against human UNG can be engineered by strategies including but not limited to the available in vitro and in vivo SELEX strategies in the literature. Candidate RNA aptamers displaying strong affinity against human UNG will be sequenced and identified RNA sequences will be employed as gRNA fusion partners in our next generation CGBE constructs. Optimal conformation of the RNA aptamer fusion will be determined empirically by cloning it into different sites in our gRNA constructs with different linkers.

Section 3: Fab, scFV, or sdAb Mediated Recruiting of UNG to the Target Site

Antibodies are naturally expressed immunological proteins comprised of two light and two heavy chain proteins expressed from different genes. They are selected against specific parts (epitopes) of specific target proteins (antigens) in immune cells. Therefore, they can selectively bind to target antigens with high affinities. Antibodies are large molecules (˜150 kDa) consisting of a constant region (Fc) and antigen binding regions (Fab) with number of disulfide bonds in between chains. Therefore, it is not practical to generate a single peptide fusion protein fused with a large intact multimeric antibody and one's protein of interest.

However, getting rid of the Fc portion and using a single Fab portion of an antibody is a smaller (˜50 kDa) and more viable option to have than having a UNG fusion partner. Important to note is that the Fab portion still has constant regions of heavy and light chains that can be further resected while retaining the antigen specific binding affinity. This approach produces a shorter fragment (˜25 kdA) called single-chain variable fragment (scFv) that is linked with each other via short peptide linker. scFv consists of variable domains of heavy and light chains. Taking one step further and separating variable domains of heavy and light chains and producing a single chain (thus single variable domain) antibody fragment is called single-domain antibodies (sdAb) or nanobodies. This is the smallest of all antibody fragments (˜12-15 kDa) around 110 amino acids in length.

Given these premises, fusing an Fab, scFv or sdAb raised against human UNG target protein to our CGBE constructs in different conformations would be a viable option to recruit the endogenous human UNG to the target loci.

Also, various new Fabs, scFvs and sdAbs against human UNG can be generated by methods including but not limited to generating a mouse hybridoma clone, then converting full IgG (or IgM) into a scFv, Fab or sdAb; generating an immunized phage display scFv, Fab or sdAb mouse library, then using human UNG to screen the library; screening a premade scFv, Fab or sdAb antibody phage display library; generating synthetic libraries by altering the variable domains of antibodies via introducing random oligonucleotides, then screening against human UNG.

Candidate Fabs, scFvs or sdAbs displaying strong affinity against human UNG will be sequenced and the identified DNA and amino acid sequences will be employed as fusion partners in our next generation CGBE constructs. Optimal conformation of the fusion partners will be determined empirically by cloning it into different sites in our constructs with different linkers.

### Programmable DNA Binding Domain

In some embodiments, the base editors include programmable DNA binding domains such as engineered C2H2 zinc-fingers, transcription activator effector-like effectors (TALEs), and Clustered Regularly Interspaced Short Palindromic Repeats (CRISPR) Cas RNA-guided nucleases (RGNs) and their variants, including ssDNA nickases (nCas9) or their analogs and catalytically inactive dead Cas9 (dCas9) and its analogs (e.g., as shown in Table F), and any engineered protospacer-adjacent motif (PAM) or high-fidelity variants (e.g., as shown in Table G). A programmable DNA binding domain is one that can be engineered to bind to a selected target sequence.

### CRISPR-Cas Nucleases

Although herein we refer to Cas9, in general any Cas9-like nickase could be used (including the related Cpf1/Cas12a enzyme classes), unless specifically indicated. These orthologs, and mutants and variants thereof as known in the art, can be used in any of the fusion proteins described herein. See, e.g., WO 2017/040348 (which describes variants of SaCas9 and SpCas 9 with increased specificity) and WO 2016/141224 (which describes variants of SaCas9 and SpCas 9 with altered PAM specificity).

The Cas9 nuclease from S. pyogenes (hereafter simply Cas9) can be guided via simple base pair complementarity between 17-20 nucleotides of an engineered guide RNA (gRNA), e.g., a single guide RNA or crRNA/tracrRNA pair, and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). The engineered CRISPR from Prevotella and Francisella 1 (Cpf1, also known as Cas12a) nuclease can also be used, e.g., as described in Zetsche et al., Cell 163, 759-771 (2015); Schunder et al., Int J Med Microbiol 303, 51-60 (2013); Makarova et al., Nat Rev Microbiol 13, 722-736 (2015); Fagerlund et al., Genome Biol 16, 251 (2015). Unlike SpCas9, Cpf1/Cas12a requires only a single 42-nt crRNA, which has 23 nt at its 3′ end that are complementary to the protospacer of the target DNA sequence (Zetsche et al., 2015). Furthermore, whereas SpCas9 recognizes an NGG PAM sequence that is 3′ of the protospacer, AsCpf1 and LbCp1 recognize TTTN PAMs that are found 5′ of the protospacer (Id.).

In some embodiments, the present system utilizes a wild type or variant Cas9 protein from S. pyogenes or Staphylococcus aureus, or a wild type or variant Cpf1 protein from Acidaminococcus sp. BV3L6 or Lachnospiraceae bacterium ND2006 either as encoded in bacteria or codon-optimized for expression in mammalian cells and/or modified in its PAM recognition specificity and/or its genome-wide specificity. A number of variants have been described; see, e.g., WO 2016/141224, PCT/US2016/049147, Kleinstiver et al., Nat Biotechnol. 2016 August; 34(8):869-74; Tsai and Joung, Nat Rev Genet. 2016 May; 17(5):300-12; Kleinstiver et al., Nature. 2016 Jan. 28; 529(7587):490-5; Shmakov et al., Mol Cell. 2015 Nov. 5; 60(3):385-97; Kleinstiver et al., Nat Biotechnol. 2015 December; 33(12):1293-1298; Dahlman et al., Nat Biotechnol. 2015 November; 33(11):1159-61; Kleinstiver et al., Nature. 2015 Jul. 23; 523(7561):481-5; Wyvekens et al., Hum Gene Ther. 2015 July; 26(7):425-31; Hwang et al., Methods Mol Biol. 2015; 1311:317-34; Osborn et al., Hum Gene Ther. 2015 February; 26(2):114-26; Konermann et al., Nature. 2015 Jan. 29; 517(7536):583-8; Fu et al., Methods Enzymol. 2014; 546:21-45; and Tsai et al., Nat Biotechnol. 2014 June; 32(6):569-76, inter alia. Concerning rAPOBEC1 itself, a number of variants have been described, e.g. Chen et al, RNA. 2010 May; 16(5):1040-52; Chester et al, EMBO J. 2003 Aug. 1; 22(15):3971-82.: Teng et al, J Lipid Res. 1999 April; 40(4):623-35.; Navaratnam et al, Cell. 1995 Apr. 21; 81(2):187-95.; MacGinnitie et al, J Biol Chem. 1995 Jun. 16; 270(24):14768-75.; Yamanaka et al, J Biol Chem. 1994 Aug. 26; 269(34):21725-34. The guide RNA is expressed or present in the cell together with the Cas9 or Cpf1. Either the guide RNA or the nuclease, or both, can be expressed transiently or stably in the cell or introduced as a purified protein or nucleic acid.

In some embodiments, the Cas9 also includes one of the following mutations, which reduce nuclease activity of the Cas9; e.g., for SpCas9, mutations at D10A or H840A (which creates a single-strand nickase).

In some embodiments, the SpCas9 variants also include mutations at one of each of the two sets of the following amino acid positions, which together destroy the nuclease activity of the Cas9: D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432).

In some embodiments, the Cas9 is fused to one or more SV40 or bipartite (bp) nuclear localization sequences (NLSs) protein sequences; an exemplary (bp)NLS sequence is as follows: (KRTADGSEFES)PKKKRKV (SEQ ID NO: 204). Typically, the NLSs are at the N- and C-termini of an ABEmax fusion protein, but can also be positioned at the N- or C-terminus in other ABEs, or between the DNA binding domain and the deaminase domain. Linkers as known in the art can be used to separate domains.

### TAL Effector Repeat Arrays

Transcription activator like effectors (TALEs) of plant pathogenic bacteria in the genus Xanthomonas play important roles in disease, or trigger defense, by binding host DNA and activating effector-specific host genes. Specificity depends on an effector-variable number of imperfect, typically ˜33-35 amino acid repeats. Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. In some embodiments, the polymorphic region that grants nucleotide specificity may be expressed as a triresidue or triplet.

Each DNA binding repeat can include a RVD that determines recognition of a base pair in the target DNA sequence, wherein each DNA binding repeat is responsible for recognizing one base pair in the target DNA sequence. In some embodiments, the RVD can comprise one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

TALE proteins may be useful in research and biotechnology as targeted chimeric nucleases that can facilitate homologous recombination in genome engineering (e.g., to add or enhance traits useful for biofuels or biorenewables in plants). These proteins also may be useful as, for example, transcription factors, and especially for therapeutic applications requiring a very high level of specificity such as therapeutics against pathogens (e.g., viruses) as non-limiting examples.

Methods for generating engineered TALE arrays are known in the art, see, e.g., the fast ligation-based automatable solid-phase high-throughput (FLASH) system described in U.S. Ser. No. 61/610,212, and Reyon et al., Nature Biotechnology 30,460-465 (2012); as well as the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci USA 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci USA 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeye et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and Zhang et al., Nat Biotechnol 29, 149-153 (2011); all of which are incorporated herein by reference in their entirety.

### Zinc Fingers

Zinc finger (ZF) proteins are DNA-binding proteins that contain one or more zinc fingers, independently folded zinc-containing mini-domains, the structure of which is well known in the art and defined in, for example, Miller et al., 1985, EMBO J., 4:1609; Berg, 1988, Proc. Natl. Acad. Sci. USA, 85:99; Lee et al., 1989, Science. 245:635; and Klug, 1993, Gene, 135:83. Crystal structures of the zinc finger protein Zif268 and its variants bound to DNA show a semi-conserved pattern of interactions, in which typically three amino acids from the alpha-helix of the zinc finger contact three adjacent base pairs or a “subsite” in the DNA (Pavletich et al., 1991, Science, 252:809; Elrod-Erickson et al., 1998, Structure, 6:451). Thus, the crystal structure of Zif268 suggested that zinc finger DNA-binding domains might function in a modular manner with a one-to-one interaction between a zinc finger and a three-base-pair “subsite” in the DNA sequence. In naturally occurring zinc finger transcription factors, multiple zinc fingers are typically linked together in a tandem array to achieve sequence-specific recognition of a contiguous DNA sequence (Klug, 1993, Gene 135:83).

Multiple studies have shown that it is possible to artificially engineer the DNA binding characteristics of individual zinc fingers by randomizing the amino acids at the alpha-helical positions involved in DNA binding and using selection methodologies such as phage display to identify desired variants capable of binding to DNA target sites of interest (Rebar et al., 1994, Science, 263:671; Choo et al., 1994 Proc. Natl. Acad. Sci. USA, 91:11163; Jamieson et al., 1994, Biochemistry 33:5689; Wu et al., 1995 Proc. Natl. Acad. Sci. USA, 92: 344). Such recombinant zinc finger proteins can be fused to functional domains, such as transcriptional activators, transcriptional repressors, methylation domains, and nucleases to regulate gene expression, alter DNA methylation, and introduce targeted alterations into genomes of model organisms, plants, and human cells (Carroll, 2008, Gene Ther., 15:1463-68; Cathomen, 2008, Mol. Ther., 16:1200-07; Wu et al., 2007, Cell. Mol. Life Sci., 64:2933-44).

One existing method for engineering zinc finger arrays, known as “modular assembly,” advocates the simple joining together of pre-selected zinc finger modules into arrays (Segal et al., 2003, Biochemistry, 42:2137-48; Beerli et al., 2002, Nat. Biotechnol., 20:135-141; Mandell et al., 2006, Nucleic Acids Res., 34:W516-523; Carroll et al., 2006, Nat. Protoc. 1:1329-41; Liu et al., 2002, J. Biol. Chem., 277:3850-56; Bae et al., 2003, Nat. Biotechnol., 21:275-280; Wright et al., 2006, Nat. Protoc., 1:1637-52). Although straightforward enough to be practiced by any researcher, recent reports have demonstrated a high failure rate for this method, particularly in the context of zinc finger nucleases (Ramirez et al., 2008, Nat. Methods, 5:374-375; Kim et al., 2009, Genome Res. 19:1279-88), a limitation that typically necessitates the construction and cell-based testing of very large numbers of zinc finger proteins for any given target gene (Kim et al., 2009, Genome Res. 19:1279-88).

Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92; Isalan et al., 2001, Nat. Biotechnol., 19:656-660). In preferred embodiments, the zinc finger arrays are described in, or are generated as described in, WO 2011/017293 and WO 2004/099366. Additional suitable zinc finger DBDs are described in U.S. Pat. Nos. 6,511,808, 6,013,453, 6,007,988, and 6,503,717 and U.S. patent application 2002/0160940.

### Variants

In some embodiments, the components of the fusion proteins are at least 80%, e.g., at least 85%, 90%, 95%, 97%, or 99% identical to the amino acid sequence of a exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. Optionally the differences can include truncations or deletions. In preferred embodiments, the variant retains a desired activity of the parent, e.g., deaminase activity, and/or the ability to interact with a guide RNA and/or target DNA, optionally with improved specificity or altered substrate specificity.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present disclosure, the comparison of sequences and determination of percent identity between two sequences can be accomplished using a Blosum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Also provided herein are isolated nucleic acids encoding the base editor fusion proteins, vectors comprising the isolated nucleic acids, optionally operably linked to one or more regulatory domains for expressing the variant proteins, and host cells, e.g., mammalian host cells, comprising the nucleic acids, and optionally expressing the variant proteins. In some embodiments, the host cells are stem cells, e.g., hematopoietic stem cells.

In some embodiments, the fusion proteins include a linker between the DNA binding domain (e.g., ZFN, TALE, or nCas9) and the BE domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:135) or GGGGS (SEQ ID NO:136), e.g., two, three, four, or more repeats of the GGGS (SEQ ID NO:137) or GGGGS (SEQ ID NO:138) unit. Other linker sequences can also be used.

In some embodiments, the CGBE fusion protein includes a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); E1-Andaloussi et al., (2005) Curr Pharm Des. 11(28):3597-611; and Deshayes et al., (2005) Cell Mol Life Sci. 62(16):1839-49.

Cell penetrating peptides (CPPs) are short peptides that facilitate the movement of a wide range of biomolecules across the cell membrane into the cytoplasm or other organelles, e.g. the mitochondria and the nucleus. Examples of molecules that can be delivered by CPPs include therapeutic drugs, plasmid DNA, oligonucleotides, siRNA, peptide-nucleic acid (PNA), proteins, peptides, nanoparticles, and liposomes. CPPs are generally 30 amino acids or less, are derived from naturally or non-naturally occurring protein or chimeric sequences, and contain either a high relative abundance of positively charged amino acids, e.g. lysine or arginine, or an alternating pattern of polar and non-polar amino acids. CPPs that are commonly used in the art include Tat (Frankel et al., (1988) Cell. 55:1189-1193, Vives et al., (1997) J. Biol. Chem. 272:16010-16017), penetratin (Derossi et al., (1994) J. Biol. Chem. 269:10444-10450), polyarginine peptide sequences (Wender et al., (2000) Proc. Natl. Acad. Sci. USA 97:13003-13008, Futaki et al., (2001) J. Biol. Chem. 276:5836-5840), and transportan (Pooga et al., (1998) Nat. Biotechnol. 16:857-861).

CPPs can be linked with their cargo through covalent or non-covalent strategies. Methods for covalently joining a CPP and its cargo are known in the art, e.g. chemical cross-linking (Stetsenko et al., (2000) J. Org. Chem. 65:4900-4909, Gait et al. (2003) Cell. Mol. Life. Sci. 60:844-853) or cloning a fusion protein (Nagahara et al., (1998) Nat. Med. 4:1449-1453). Non-covalent coupling between the cargo and short amphipathic CPPs comprising polar and non-polar domains is established through electrostatic and hydrophobic interactions.

CPPs have been utilized in the art to deliver potentially therapeutic biomolecules into cells. Examples include cyclosporine linked to polyarginine for immunosuppression (Rothbard et al., (2000) Nature Medicine 6(11):1253-1257), siRNA against cyclin B1 linked to a CPP called MPG for inhibiting tumorigenesis (Crombez et al., (2007) Biochem Soc. Trans. 35:44-46), tumor suppressor p53 peptides linked to CPPs to reduce cancer cell growth (Takenobu et al., (2002) Mol. Cancer Ther. 1(12):1043-1049, Snyder et al., (2004) PLoS Biol. 2:E36), and dominant negative forms of Ras or phosphoinositol 3 kinase (PI3K) fused to Tat to treat asthma (Myou et al., (2003) J. Immunol. 171:4399-4405).

CPPs have been utilized in the art to transport contrast agents into cells for imaging and biosensing applications. For example, green fluorescent protein (GFP) attached to Tat has been used to label cancer cells (Shokolenko et al., (2005) DNA Repair 4(4):511-518). Tat conjugated to quantum dots have been used to successfully cross the blood-brain barrier for visualization of the rat brain (Santra et al., (2005) Chem. Commun. 3144-3146). CPPs have also been combined with magnetic resonance imaging techniques for cell imaging (Liu et al., (2006) Biochem. and Biophys. Res. Comm. 347(1):133-140). See also Ramsey and Flynn, Pharmacol Ther. 2015 Jul. 22. pii: S0163-7258(15)00141-2.

Alternatively or in addition, the CGBE fusion proteins can include a nuclear localization sequence, e.g., SV40 large T antigen NLS (PKKKRRV (SEQ ID NO:348)) and nucleoplasmin NLS (KRPAATKKAGQAKKKK (SEQ ID NO:349)). Other NLSs are known in the art; see, e.g., Cokol et al., EMBO Rep. 2000 Nov. 15; 1(5): 411-415; Freitas and Cunha, Curr Genomics. 2009 December; 10(8): 550-557.

In some embodiments, the CGBE fusion proteins include a moiety that has a high affinity for a ligand, for example GST, FLAG or hexahistidine sequences. Such affinity tags can facilitate the purification of recombinant CGBE fusion proteins.

The CGBE fusion proteins described herein can be used for altering the genome of a cell. The methods generally include expressing or contacting the CGBE fusion proteins in the cells; in versions using one or two Cas9s, the methods include using a guide RNA having a region complementary to a selected portion of the genome of the cell. Methods for selectively altering the genome of a cell are known in the art, see, e.g., U.S. Pat. No. 8,993,233; US 20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US20160024529; US20160024524; US20160024523; US20160024510; US20160017366; US20160017301; US20150376652; US20150356239; US20150315576; US20150291965; US20150252358; US20150247150; US20150232883; US20150232882; US20150203872; US20150191744; US20150184139; US20150176064; US20150167000; US20150166969; US20150159175; US20150159174; US20150093473; US20150079681; US20150067922; US20150056629; US20150044772; US20150024500; US20150024499; US20150020223; US20140356867; US20140295557; US20140273235; US20140273226; US20140273037; US20140189896; US20140113376; US20140093941; US20130330778; US20130288251; US20120088676; US20110300538; US20110236530; US20110217739; US20110002889; US20100076057; US20110189776; US20110223638; US20130130248; US20150050699; US20150071899; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; US20140170753; WO/2008/108989; WO/2010/054108; WO/2012/164565; WO/2013/098244; WO/2013/176772; US 20150071899; Makarova et al., “Evolution and classification of the CRISPR-Cas systems” 9(6) Nature Reviews Microbiology 467-477 (1-23) (June 2011); Wiedenheft et al., “RNA-guided genetic silencing systems in bacteria and archaea” 482 Nature 331-338 (Feb. 16, 2012); Gasiunas et al., “Cas9-crRNA ribonucleoprotein complex mediates specific DNA cleavage for adaptive immunity in bacteria” 109(39) Proceedings of the National Academy of Sciences USA E2579-E2586 (Sep. 4, 2012); Jinek et al., “A Programmable Dual-RNA-Guided DNA Endonuclease in Adaptive Bacterial Immunity” 337 Science 816-821 (Aug. 17, 2012); Carroll, “A CRISPR Approach to Gene Targeting” 20(9) Molecular Therapy 1658-1660 (September 2012); U.S. Appl. No. 61/652,086, filed May 25, 2012; Al-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289; Hale et al., Essential Features and Rational Design of CRISPR RNAs That Function With the Cas RAMP Module Complex to Cleave RNAs, Molecular Cell, (2012) vol. 45, Issue 3, 292-302.

For methods in which the CGBE fusion proteins are delivered to cells, the proteins can be produced using any method known in the art, e.g., by in vitro translation, or expression in a suitable host cell from nucleic acid encoding the CGBE fusion protein; a number of methods are known in the art for producing proteins. For example, the proteins can be produced in and purified from yeast, E. coli, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the CGBE fusion proteins can be linked to a moiety that facilitates transfer into a cell, e.g., a lipid nanoparticle, optionally with a linker that is cleaved once the protein is inside the cell. See, e.g., LaFountaine et al., Int J Pharm. 2015 Aug. 13; 494(1):180-194.

### Expression Systems

To use the CGBE fusion proteins described herein, it may be desirable to express them from a nucleic acid that encodes them. This can be performed in a variety of ways. For example, the nucleic acid encoding the CGBE fusion can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the CGBE fusion for production of the CGBE fusion protein. The nucleic acid encoding the CGBE fusion protein can also be cloned into an expression vector, for administration to a plant cell, animal cell, preferably a mammalian cell or a human cell, fungal cell, bacterial cell, or protozoan cell.

To obtain expression, a sequence encoding a CGBE fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the CGBE fusion protein is to be administered in vivo for gene regulation, either a constitutive or an inducible promoter can be used, depending on the particular use of the CGBE fusion protein. In addition, a preferred promoter for administration of the CGBE fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the CGBE fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the CGBE fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the CGBE fusion protein can include RNA Pol III promoters to drive expression of the guide RNAs, e.g., the H1, U6 or 7SK promoters. These human promoters allow for expression of CGBE fusion protein in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the CGBE fusion protein.

In methods wherein the fusion proteins include a Cas9 domain, the methods also include delivering at least one gRNA that interacts with the Cas9, or a nucleic acid that encodes a gRNA.

Alternatively, the methods can include delivering the CGBE fusion protein and guide RNA together, e.g., as a complex. For example, the CGBE fusion protein and gRNA can be can be overexpressed in a host cell and purified, then complexed with the guide RNA (e.g., in a test tube) to form a ribonucleoprotein (RNP), and delivered to cells. In some embodiments, the CGBE fusion protein can be expressed in and purified from bacteria through the use of bacterial expression plasmids. For example, His-tagged CGBE fusion protein can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you′d get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

The present invention also includes the vectors and cells comprising the vectors, as well as kits comprising the proteins and nucleic acids described herein, e.g., for use in a method described herein.

### Methods of Use

The base editors described herein can be used to generate transversion mutations—i.e., C-to-G mutations—in a nucleic acid sequence, e.g., in a cell, e.g., a cell in an animal (e.g., a mammal such as a human or veterinary subject), or a synthetic nucleic acid substrate. The methods include contacting the nucleic acid with a base editor as described herein. Where the base editor includes a CRISPR Cas9 or Cas12a protein, the methods further include the use of one or more guide RNAs that direct binding of the base editor to a sequence to be deaminated.

For example, the base editors described herein can be used for in vitro, in vivo or in situ directed evolution, e.g., to engineer polypeptides or proteins based on a synthetic selection framework, e.g. antibiotic resistance in E. coli or resistance to anti-cancer therapeutics being assayed in mammalian cells (e.g. CRISPR-X Hess et al, PMID: 27798611 or BE-plus systems Jiang et al, PMID: 29875396).

### Tables

TABLE A
Exemplary APOBEC1 proteins. This table lists (in
alphabetical order) important APOBEC1 homologues.

| | | Uniprot | | Seq |
|---|---|---|---|---|
| APOBEC1 | | accession | | ID |
| orthologue | Species | number | Version number | NO: |
| African elephant | Loxodonta | G3U0R4 | version 30 of the entry | 1 |
| | africana | | and version 1 of the | |
| | | | sequence | |
| African lungfish | Protopterus | A0A0M3N0G8 | version 4 of the entry | 2 |
| | annectens | | and version 1 of the | |
| | | | sequence | |
| American alligator | Alligator | A0A151P6M4 | version 9 of the entry | 3 |
| | mississippiensis | | and version 1 of the | |
| | | | sequence | |
| American | Anolis | F1CGT0 | version 16 of the entry | 4 |
| chameleon | carolinensis | | and version 1 of the | |
| | | | sequence | |
| American crow | Corvus | A0A091EQ78 | version 8 of the entry | 5 |
| | brachyrhynchos | | and version 1 of the | |
| | | | sequence | |
| Anna's | Calypte anna | A0A091IIG0 | version 9 of the entry | 6 |
| hummingbird | | | and version 1 of the | |
| | | | sequence | |
| Atlantic bottle- | Tursiops | A0A2U4ALA1 | version 2 of the entry | 7 |
| nosed dolphin | truncatus | | and version 1 of the | |
| | | | sequence | |
| Barn owl | Tyto alba | A0A093FY71 | version 6 of the entry | 8 |
| | | | and version 1 of the | |
| | | | sequence | |
| Black flying fox | Pteropus alecto | L5KGJ8 | version 13 of the entry | 9 |
| | | | and version 1 of the | |
| | | | sequence | |
| Black snub-nosed | Rhinopithecus | A0A2K6KS69 | version 5 of the entry | 10 |
| monkey | bieti | | and version 1 of the | |
| | | | sequence | |
| Beluga whale | Delphlnapterus | A0A2Y9NGP5 | version 1 of the entry | 11 |
| | leucas | | and version 1 of the | |
| | | | sequence | |
| Bengalese finch | Lonchura striata | A0A218ULD2 | version 3 of the entry | 12 |
| | domestica | | and version 1 of the | |
| | | | sequence | |
| Blue-fronted | Amazona | A0A0Q3WRD0 | version 5 of the entry | 13 |
| Amazon parrot | aestiva | | and version 1 of the | |
| | | | sequence | |
| Bolivian squirrel | Saimiri | A0A2K6U925 | version 5 of the entry | 14 |
| monkey | boliviensis | | and version 1 of the | |
| | boliviensis | | sequence | |
| Bonobo | Pan paniscus | A0A2R9A0R0 | version 2 of the entry | 15 |
| | | | and version 1 of the | |
| | | | sequence | |
| Bornean | Pongo | Q694B3 | version 60 of the entry | 16 |
| orangutan | pygmaeus | | and version 2 of the | |
| | | | sequence | |
| Bovine | Bos taurus | E1BP99 | version 40 of the entry | 17 |
| | | | and version 1 of the | |
| | | | sequence | |
| Brandt's bat | Myotis brandtii | S7PYX0 | version 9 of the entry | 18 |
| | | | and version 1 of the | |
| | | | sequence | |
| Cat | Felis catus | M3WB96 | version 31 of the entry | 19 |
| | | | and version 2 of the | |
| | | | sequence | |
| Cebus capucinus | Cebus capucinus | A0A2K5PZC0 | version 5 of the entry | 20 |
| imitator | imitator | | and version 1 of the | |
| | | | sequence | |
| Chimpanzee | Pan troglodytes | H2Q5C6 | version 32 of the entry | 21 |
| | | | and version 1 of the | |
| | | | sequence | |
| Chinese alligator | Alligator | A0A1U7S7K7 | version 5 of the entry | 22 |
| | sinensis | | and version 1 of the | |
| | | | sequence | |
| Chinese hamster | Cricetulus | G3I1S7 | version 15 of the entry | 23 |
| | griseus | | and version 1 of the | |
| | | | sequence | |
| Chuck-will's- | Antrostomus | A0A094MFH1 | version 10 of the entry | 24 |
| widow | carolinensis | | and version 1 of the | |
| | | | sequence | |
| Coquerel's sifaka | Propithecus | A0A2K6EVT9 | version 5 of the entry | 25 |
| | coquereli | | and version 1 of the | |
| | | | sequence | |
| Crab-eating | Macaca | G8F4P7 | version 11 of the entry | 26 |
| macaque | fascicularis | | and version 1 of the | |
| | | | sequence | |
| Crested ibis | Nipponia | A0A091V7F8 | version 9 of the entry | 27 |
| | nippon | | and version 1 of the | |
| | | | sequence | |
| Dalmatian pelican | Pelecanus | A0A091SSF0 | version 8 of the entry | 28 |
| | crispus | | and version 1 of the | |
| | | | sequence | |
| Damaraland mole | Fukomys | A0A091CVE5 | version 9 of the entry | 29 |
| rat | damarensis | | and version 1 of the | |
| | | | sequence | |
| David's myotis | Myotis davidii | L5LUG3 | version 11 of the entry | 30 |
| | | | and version 1 of the | |
| | | | sequence | |
| Dog | Canis lupus | F1PUJ5 | version 41 of the entry | 31 |
| | familiaris | | and version 2 of the | |
| | | | sequence | |
| Downy | Dryobates | A0A093GVH6 | version 9 of the entry | 32 |
| woodpecker | pubescens | | and version 1 of the | |
| | | | sequence | |
| Drill | Mandrillus | A0A2K5Z8Y4 | version 4 of the entry | 33 |
| | leucophaeus | | and version 1 of the | |
| | | | sequence | |
| East African grey | Balearica | A0A087VMP5 | version 8 of the entry | 34 |
| crowned-crane | regulorum | | and version 1 of the | |
| | gibbericeps | | sequence | |
| Emperor penguin | Aptenodytes | A0A087QNJ5 | version 8 of the entry | 35 |
| | forsteri | | and version 1 of the | |
| | | | sequence | |
| Enhydra lutris | Enhydra lutris | A0A2Y9IYV0 | version 1 of the entry | 36 |
| kenyoni | kenyoni | | and version 1 of the | |
| | | | sequence | |
| European | Mustela | B2NIW5 | version 34 of the entry | 37 |
| domestic ferret | putorius furo | | and version 1 of the | |
| | | | sequence | |
| Florida manatee | Trichechus | A0A2Y9E587 | version 1 of the entry | 38 |
| | manatus | | and version 1 of the | |
| | latirostris | | sequence | |
| Giant panda | Ailuropoda | G1LKL4 | version 27 of the entry | 39 |
| | melanoleuca | | and version 1 of the | |
| | | | sequence | |
| Golden-collared | Manacus | A0A093PWR2 | version 8 of the entry | 40 |
| manakin | vitellinus | | and version 1 of the | |
| | | | sequence | |
| Golden hamster | Mesocricetus | Q9EQP0 | version 73 of the entry | 41 |
| | auratus | | and version 1 of the | |
| | | | sequence | |
| Golden snub- | Rhinopithecus | A0A2K6PRF3 | version 4 of the entry | 42 |
| nosed monkey | roxellana | | and version 1 of the | |
| | | | sequence | |
| Green monkey | Chlorocebus | A0A0D9RBS4 | version 11 of the entry | 43 |
| | sabaeus | | and version 1 of the | |
| | | | sequence | |
| Guinea pig | Cavia porcellus | A0A286XNR2 | version 5 of the entry | 44 |
| | | | and version 1 of the | |
| | | | sequence | |
| Hawaiian monk | Neomonachus | A0A2Y9HAT6 | version 1 of the entry | 45 |
| seal | schauinslandi | | and version 1 of the | |
| | | | sequence | |
| Hoatzin | Opisthocomus | A0A091XJL0 | version 8 of the entry | 46 |
| | hoazin | | and version 1 of the | |
| | | | sequence | |
| Horse | Equus ferus | F6WR88 | version 28 of the entry | 47 |
| | caballus | | and version 1 of the | |
| | | | sequence | |
| Human | Homo sapiens | P41238 | version 166 of the entry | 48 |
| | | | and version 3 of the | |
| | | | sequence | |
| Kea | Nestor notabilis | A0A091RU17 | version 8 of the entry | 49 |
| | | | and version 1 of the | |
| | | | sequence | |
| Little egret | Egretta garzetta | A0A091IWL9 | version 10 of the entry | 50 |
| | | | and version 1 of the | |
| | | | sequence | |
| Ma's night | Aotus | A0A2K5DG70 | version 6 of the entry | 51 |
| monkey | nancymaae | | and version 1 of the | |
| | | | sequence | |
| Mouse | Mus musculus | P51908 | version 150 of the entry | 52 |
| | | | and version 1 of the | |
| | | | sequence | |
| Naked mole rat | Heterocephalus | G5BPM8 | version 16 of the entry | 53 |
| | glaber | | and version 1 of the | |
| | | | sequence | |
| Northern carmine | Merops nubicus | A0A091QEK6 | version 8 of the entry | 54 |
| bee-eater | | | and version 1 of the | |
| | | | sequence | |
| Northern fulmar | Fulmarus | A0A093LP85 | version 9 of the entry | 55 |
| | glacialis | | and version 1 of the | |
| | | | sequence | |
| Northern white- | Nomascus | G1QZV0 | version 31 of the entry | 56 |
| cheeked gibbon | leucogenys | | and version 1 of the | |
| | | | sequence | |
| Olive baboon | Papio anubis | A0A096MWB4 | version 19 of the entry | 57 |
| | | | and version 2 of the | |
| | | | sequence | |
| Gray short-tailed | Monodelphis | Q9TUI7 | version 101 of the entry | 58 |
| Opossum | domestica | | and version 1 of the | |
| | | | sequence | |
| Ord's kangaroo | Dipodomys ordii | A0A1S3FTE2 | version 3 of the entry | 59 |
| rat | | | and version 1 of the | |
| | | | sequence | |
| Pacific walrus | Odobenus | A0A2U3WPA5 | version 2 of the entry | 60 |
| | rosmarus | | and version 1 of the | |
| | divergens | | sequence | |
| Patagioenas | Patagioenas | A0A1V4JAP2 | version 3 of the entry | 61 |
| fasciata monilis | fasciata monilis | | and version 1 of the | |
| | | | sequence | |
| Peters' Angolan | Colobus | A0A2K5JKV4 | version 4 of the entry | 62 |
| colobus | angolensis | | and version 1 of the | |
| | palliatus | | sequence | |
| Philippine tarsier | Tarsius syrichta | A0A1U7U8J6 | version 3 of the entry | 63 |
| | | | and version 1 of the | |
| | | | sequence | |
| Pig | Sus scrofa | F1SLW4 | version 37 of the entry | 64 |
| | | | and version 2 of the | |
| | | | sequence | |
| Pig-tailed | Macaca | A0A2K6BGI5 | version 4 of the entry | 65 |
| macaque | nemestrina | | and version 1 of the | |
| | | | sequence | |
| Rabbit | Oryctolagus | P47855 | version 96 of the entry | 66 |
| | cuniculus | | and version 1 of the | |
| | | | sequence | |
| Rat | Rattus | P38483 | version 137 of the entry | 67 |
| | norvegicus | | and version 1 of the | |
| | | | sequence | |
| Red-legged | Cariama | A0A091M4D7 | version 10 of the entry | 68 |
| seriema | cristata | | and version 1 of the | |
| | | | sequence | |
| Red throated | Gavia stellata | A0A093F3R4 | version 8 of the entry | 69 |
| diver | | | and version 1 of the | |
| | | | sequence | |
| Rhesus macaque | Macaca mulatta | G7N5W0 | version 19 of the entry | 70 |
| | | | and version 1 of the | |
| | | | sequence | |
| Rifleman | Acanthisitta | A0A091MEP8 | version 8 of the entry | 71 |
| (Acanthisitta | chloris | | and version 1 of the | |
| chloris) | | | sequence | |
| Rock dove | Columba livia | A0A2I0LXZ8 | version 3 of the entry | 72 |
| | | | and version 1 of the | |
| | | | sequence | |
| Sheep | Ovis aries | W5NVH9 | version 19 of the entry | 73 |
| | | | and version 1 of the | |
| | | | sequence | |
| Small-eared | Otolemur | H0XVG8 | version 27 of the entry | 74 |
| galago | gamettii | | and version 1 of the | |
| (Garnett's greater | | | sequence | |
| bushbaby) | | | | |
| Smooth | Stylophora | A0A2B4RXQ3 | version 4 of the entry | 75 |
| cauliflower coral | pistillata | | and version 1 of the | |
| | | | sequence | |
| Sooty mangabey | Cercocebus | A0A2K5L2J6 | version 5 of the entry | 76 |
| | atys | | and version 1 of the | |
| | | | sequence | |
| Sperm whale | Physeter | A0A2Y9T649 | version 1 of the entry | 77 |
| | macrocephalus | | and version 1 of the | |
| | | | sequence | |
| Sumatran | Pongo abelii | H2NGD0 | version 24 of the entry | 78 |
| orangutan | | | and version 1 of the | |
| | | | sequence. | |
| Sunbittern | Eurypyga helias | A0A093JI54 | version 8 of the entry | 79 |
| | | | and version 1 of the | |
| | | | sequence | |
| Tasmanian devil | Sarcophilus | G3W4I1 | version 32 of the entry | 80 |
| | harrisii | | and version 1 of the | |
| | | | sequence | |
| Weddell seal | Leptonychotes | A0A2U3Y3M5 | version 2 of the entry | 81 |
| | weddellii | | and version 1 of the | |
| | | | sequence | |
| Western | Erinaceus | A0A1S3AN78 | version 3 of the entry | 82 |
| European | europaeus | | and version 1 of the | |
| hedgehog | | | sequence | |
| White-tailed sea- | Haliaeetus | A0A091PSV3 | version 8 of the entry | 83 |
| eagle | albicilla | | and version 1 of the | |
| | | | sequence | |
| White tufted ear | Callithrix | F7F6M6 | version 31 of the entry | 84 |
| marmoset | jacchus | | and version 2 of the | |
| | | | sequence | |
| Wild yak | Bos mutus | L8IDZ0 | version 15 of the entry | 85 |
| | | | and version 1 of the | |
| | | | sequence | |
| Yellow-throated | Pterocles | A0A093CIQ8 | version 5 of the entry | 86 |
| sandgrouse | gutturalis | | and version 1 of the | |
| | | | sequence | |

TABLE B
Exemplary APOBEC/AID family proteins. The following table lists
(in alphabetical order) exemplary APOBEC family homologues.

| APOBEC/AID | Uniprot | | |
|---|---|---|---|
| family | accession | | Seq. |
| homologue | number | Version number | ID |
| Rat APOBEC1 | P38483 | version 137 of the entry | 67 |
| | | and version 1 of the | |
| | | sequence | |
| Human AID | Q9GZX7 | version 155 of the entry | 87 |
| (AICDA) | | and version 1 of the | |
| | | sequence | |
| Human | P41238 | version 166 of the entry | 48 |
| APOBEC1 | | and version 3 of the | |
| | | sequence | |
| Human | Q9Y235 | version 132 of the entry | 88 |
| APOBEC2 | | and version 1 of the | |
| | | sequence | |
| Human | P31941 | version 160 of the entry | 89 |
| APOBEC3A | | and version 3 of the | |
| | | sequence | |
| Human | Q9UH17 | version 150 of the entry | 90 |
| APOBEC3B | | and version 1 of the | |
| | | sequence | |
| Human | Q9NRW3 | version 147 of the entry | 91 |
| APOBEC3C | | and version 2 of the | |
| | | sequence | |
| Human | Q96AK3 | version 127 of the entry | 92 |
| APOBEC3D | | and version 1 of the | |
| | | sequence | |
| Human | Q8IUX4 | version 143 of the entry | 93 |
| APOBEC3F | | and version 3 of the | |
| | | sequence | |
| Human | Q9HC16 | version 168 of the entry | 94 |
| APOBEC3G | | and version 1 of the | |
| | | sequence | |
| Human | Q6NTF7 | version 115 of the entry | 95 |
| APOBEC3H | | and version 4 of the | |
| | | sequence | |
| Petromyzon | NCBI Genbank: | Version 1 of the entry, | 96 |
| marinus cytosine | ABO15149.1 | accession EF094822.1 | |
| deaminase | Uniprot: A5H718 | | |
| (pmCDA1) | | | |
| Petromyzon | Same sequence | — | 97 |
| marinus cytosine | as ID 96, but | | |
| deaminase | with R187W | | |
| (pmCDA1) | mutation | | |

TABLE C
Exemplary TadA proteins. Some or all residues listed
in Table A as well as combinations thereof might
also be introduced in any of these TadA orthologues
or tRNA adenosine deaminase homologues (see FIG.
5 for alignments of these TadA proteins).

| tRNA-specific | Uniprot | | |
|---|---|---|---|
| adenosine | accession | Sequence | Seq. |
| deaminase | number | version # | ID |
| E. coli TadA | P68398 | 2 | 98 |
| S. aureus TadA | Q99W51 | 1 | 99 |
| S. pyogenes TadA | Q5XE14 | 2 | 100 |
| S. typhi TadA | Q8XGY4 | 2 | 101 |
| A. aeolicus TadA | O67050 | 1 | 102 |
| S. pombe TAD2 | O94642 | 2 | 103 |
| S. cerevisiae TAD1 | P53065 | 1 | 104 |
| S. cerevisiae TAD2 | P47058 | 1 | 105 |
| A. thaliana TAD2 | Q6IDB6 | 1 | 106 |
| X. laevis ADAT2 | Q4V7V8 | 1 | 107 |
| X. tropicalis ADAT2 | Q0P4H0 | 1 | 108 |
| D. rerio ADAT2 | Q5RIV4 | 2 | 109 |
| B. taurus ADAT2 | Q5E9J7 | 1 | 110 |
| M. musculus ADAT2 | Q6P6J0 | 1 | 111 |
| H. sapiens ADAT2 | Q7Z6V5 | 1 | 112 |

TABLE D
Specific codons and amino acid modifications that are actionable with CGBE. Listing
potential codon changes, as well as amino acid modifications that can be induced by
CGBE. WT = wild type; AA = amino acid; = same AA also included in potential outcome.

| wt | wt | codon mutations, | AA mutations, | codon mutations, | AA mutations, |
|---|---|---|---|---|---|
| codon | AA | C-to-G | C-to-G | G-to-C | G-to-C |
| AAA | K | AAA | = | AAA | = |
| AAC | N | AAG | N > K | AAC | = |
| AAG | K | AAG | = | AAC | K > N |
| AAT | N | AAT | = | AAT | = |
| ACA | T | AGA | T > R | ACA | = |
| ACC | T | AGG, AGC, ACG | T > R, T > S, = | ACC | = |
| ACG | T | AGG | T > R | ACC | = |
| ACT | T | AGT | T > S | ACT | = |
| AGA | R | AGA | = | ACA | R > T |
| AGC | S | AGG | S > R | ACC | S > T |
| AGG | R | AGG | = | ACC, ACG, AGC | R > T, R > S |
| AGT | S | AGT | = | ACT | S > T |
| ATA | I | ATA | = | ATA | = |
| ATC | I | ATG | I > M | ATC | = |
| ATG | M | ATG | = | ATC | M > I |
| ATT | I | ATT | = | ATT | = |
| CAA | Q | GAA | Q > E | CAA | = |
| CAC | H | GAG, GAC, CAG | H > E, H > D, H > Q | CAC | = |
| CAG | Q | GAG | Q > E | CAC | Q > H |
| CAT | H | GAT | H > D | CAT | = |
| CCA | P | GGA, GCA, CGA | P > G, P > A, P > R | CCA | = |
| CCC | P | GGG, GCC, CGC, | P > G, P > A, | CCC | = |
| | | CCG, GGC, CGG, | P > R, = | | |
| | | GCG | | | |
| CCG | P | GGG, GCG, CGG | P > G, P > A, P > R | CCC | = |
| CCT | P | GGT, GCT, CGT | P > G, P > A, P > R | CCT | = |
| CGA | R | GGA | R > G | CCA | R > P |
| CGC | R | GGG, GGC, CGG | R > G, = | CCC | R > P |
| CGG | R | GGG | R > G | CCC, CCG, CGC | R > P, = |
| CGT | R | GGT | R > G | CCT | R > P |
| CTA | L | GTA | L > V | CTA | = |
| CTC | L | GTG, GTC, CTG | L > V, = | CTC | = |
| CTG | L | GTG | L > V | CTC | = |
| CTT | L | GTT | L > V | CTT | = |
| GAA | E | GAA | = | CAA | E > Q |
| GAC | D | GAG | D > E | CAC | D > H |
| GAG | E | GAG | = | CAC, CAG, GAC | E > H, E > Q, E > D |
| GAT | D | GAT | = | CAT | D > H |
| GCA | A | GGA | A > G | CCA | A > P |
| GCC | A | GGG, GGC, GCG | A > G, = | CCC | A > P |
| GCG | A | GGG | A > G | CCC, CCG, GCC | A > P, = |
| GCT | A | GGT | A > G | CCT | A > P |
| GGA | G | GGA | = | CCA, CGA, GCA | G > P, G > R, G > A |
| GGC | G | GGG | = | CCC, CGC, GCC | G > P, G > R, G > A |
| GGG | G | GGG | = | CCC, CGG, GCG, | G > P, G > R, |
| | | | | GGC, CCG, GCC, | G > A, = |
| | | | | CGC | |
| GGT | G | GGT | = | CCT, CGT, GCT | G > P, G > R, G > A |
| GTA | V | GTA | = | CTA | V > L |
| GTC | V | GTG | = | CTC | V > L |
| GTG | V | GTG | = | CTC, CTG, GTC | V > L, = |
| GTT | V | GTT | = | CTT | V > L |
| TAA | * | TAA | = | TAA | = |
| TAC | Y | TAG | Y > * | TAC | = |
| TAG | * | TAG | = | TAC | * > Y |
| TAT | Y | TAT | = | TAT | = |
| TCA | S | TGA | S > * | TCA | = |
| TCC | S | TGG, TGC, TCG | S > W, S > C, = | TCC | = |
| TCG | S | TGG | S > W | TCC | = |
| TCT | S | TGT | S > C | TCT | = |
| TGA | * | TGA | = | TCA | * > S |
| TGC | C | TGG | C > W | TCC | C > S |
| TGG | W | TGG | = | TCC, TCG, TGC | W > S, W > C |
| TGT | C | TGT | = | TCT | C > S |
| TTA | L | TTA | = | TTA | = |
| TTC | F | TTG | F > L | TTC | = |
| TTG | L | TTG | = | TTC | L > F |
| TTT | F | TTT | = | TTT | = |

TABLE E1
Specific targetable mutations from the ClinVar database that can be
corrected with CGBE using Cas9 proteins with NGG-PAM recognition.

| snpId | name | geneId | phenotypeList |
|---|---|---|---|
| 121908088 | C > G | 7173\|TPO | Deficiency of iodide peroxidase, not provided |
| 143367518 | C > G | 1161\|ERCC8 | Cockayne syndrome type A, not provided |
| 74953290 | C > G | 324\|APC | Hereditary cancer-predisposing syndrome, not |
| | | | provided, not specified |
| 201732356 | C > G | 5428\|POLG | not provided |
| 587783598 | C > G | 1785\|DNM2 | Myopathy, centronuclear, not provided |
| 879254375 | C > G | 3949\|LDLR | Familial hypercholesterolemia |
| 752596535 | C > G | 3949\|LDLR | Familial hypercholesterolemia |
| 121908725 | C > G | 100\|ADA | Severe combined immunodeficiency due to ADA |
| | | | deficiency, not provided |
| 587777526 | C > G | 23394\|ADNP | Helsmoortel-van der aa syndrome, Inborn genetic |
| | | | diseases, not provided |
| 398123527 | G > C | 2629\|GBA | Gaucher disease, Gaucher's disease, type 1 |
| 794728589 | G > C | 4000\|LMNA | Primary dilated cardiomyopathy, not provided |
| 267607570 | G > C | 4000\|LMNA | Cardiovascular phenotype, Charcot-Marie-Tooth |
| | | | disease, type 2, Dilated cardiomyopathy 1A, not |
| | | | provided |
| 1167218743 | G > C | 3030\|HADHA | Long-chain 3-hydroxyacyl-CoA dehydrogenase |
| | | | deficiency, Long-chain 3-hydroxyacyl-CoA |
| | | | dehydrogenase deficiency, Mitochondrial trifunctional |
| | | | protein deficiency |
| 727504799 | G > C | 7273\|TTN | Cardiomyopathy, Primary dilated cardiomyopathy |
| 767978961 | G > C | 729920\|CRPPA | Congenital muscular dystrophy-dystroglycanopathy |
| | | | with brain and eye anomalies, type A7, Muscular |
| | | | dystrophy-dystroglycanopathy (limb-girdle), type |
| | | | c, 7, not provided |
| 1325951163 | G > C | 673\|BRAF | Global developmental delay, not provided |
| 398123181 | G > C | 2592\|GALT | Deficiency of UDPglucose-hexose-1-phosphate |
| | | | uridylyltransferase, not provided |
| 137853150 | G > C | 10312\|TCIRG1 | Osteopetrosis autosomal recessive 1 |
| 759520465 | G > C | 472\|ATM | Ataxia-telangiectasia syndrome, Hereditary cancer- |
| | | | predisposing syndrome, not provided |
| 539407162 | G > C | 89910\|UBE3B | Inborn genetic diseases, Kaufman oculocerebrofacial |
| | | | syndrome |
| 63750473 | G > C | 368\|ABCC6 | Pseudoxanthoma elasticum, not provided |
| 397516354 | G > C | 7137\|TNNI3 | Hypertrophic cardiomyopathy, Primary familial |
| | | | hypertrophic cardiomyopathy |

TABLE E2
Specific targetable mutations from the ClinVar database that can be
corrected with CGBE using Cas9 proteins with NGA-PAM recognition.

| snpId | name | geneId | phenotypeList |
|---|---|---|---|
| 536746349 | C > G | 1716\|DGUOK | Progressive external ophthalmoplegia with mitochondrial |
| | | | DNA deletions, autosomal recessive 4, not provided |
| | C > G | 4703\|NEB | Nemaline myopathy, Nemaline myopathy 2 |
| 398123350 | C > G | 272O\|GLB1 | GM1 gangliosidosis type 2, GM1 gangliosidosis type |
| | | | 2, Gangliosidosis GM1 type 3, Gangliosidosis GM1 type |
| | | | 3, Infantile GM1 gangliosidosis, Infantile GM1 |
| | | | gangliosidosis, Mucopolysaccharidosis, MPS-IV- |
| | | | B, Mucopolysaccharidosis, MPS-IV-B, not provided |
| 121913286 | C > G | 5290\|PIK3CA | Adenocarcinoma of prostate, Adenocarcinoma of |
| | | | stomach, Breast adenocarcinoma, Glioblastoma, Malignant |
| | | | melanoma of skin, Malignant neoplasm of body of |
| | | | uterus, Medulloblastoma, Neoplasm of brain, Neoplasm of |
| | | | the breast, Neoplasm of the large intestine, Squamous cell |
| | | | carcinoma of the head and neck, Transitional cell |
| | | | carcinoma of the bladder, Uterine Carcinosarcoma, Uterine |
| | | | cervical neoplasms |
| | C > G | 5896\|RAG1 | Alpha/beta T-cell lymphopenia with gamma/delta T-cell |
| | | | expansion, severe cytomegalovirus infection, and |
| | | | autoimmunity, Combined cellular and humoral immune |
| | | | defects with granulomas, Combined cellular and humoral |
| | | | immune defects with granulomas, Histiocytic medullary |
| | | | reticulosis, Severe immunodeficiency, autosomal |
| | | | recessive, T-cell negative, B-cell negative, NK cell- |
| | | | positive, Severe immunodeficiency, autosomal recessive, T- |
| | | | cell negative, B-cell negative, NK cell-positive |
| 1057517774 | C > G | 4647\|MYO7A | Deafness, autosomal recessive 2, Usher syndrome, type |
| | | | 1, not provided |
| 749491616 | C > G | 35\|ACADS | Deficiency of butyryl-CoA dehydrogenase, not provided |
| 761649878 | C > G | 5428\|POLG | POLG-Related disorder, Progressive sclerosing |
| | | | poliodystrophy, not provided |
| 104894718 | C > G | 6324\|SCN1B | Atrial fibrillation, familial, 13, Atrial |
| | | | fibrillation, familial, 13, Brugada syndrome 5, Brugada |
| | | | syndrome 5, Epileptic encephalopathy, early |
| | | | infantile, 52, Generalized epilepsy with febrile seizures |
| | | | plus, Generalized epilepsy with febrile seizures plus, type |
| | | | 1, Generalized epilepsy with febrile seizures plus, type |
| | | | 1, Seizures, not provided |
| 876657730 | G > C | 7399\|USH2A | Retinitis pigmentosa 39, Usher syndrome, type 2A, Usher |
| | | | syndrome, type 2A, not provided |
| 869320742 | G > C | 7273\|TTN | Hereditary myopathy with early respiratory failure, not |
| | | | provided |
| 672601366 | G > C | 547\|KIF1A | Mental retardation, autosomal dominant 9 |
| 863224905 | G > C | 64324\|NSD1 | Beckwith-Wiedemann syndrome, Sotos syndrome 1 |
| 756013171 | G > C | 157680\|VPS13B | Cohen syndrome, not provided |
| 120074186 | G > C | 3784\|KCNQ1 | Cardiovascular phenotype, Congenital long QT |
| | | | syndrome, Jervell and Lange-Nielsen syndrome 1, not |
| | | | provided |
| 121908195 | G > C | 1200\|TPP1 | Ceroid lipofuscinosis neuronal 2, not provided |
| 1057517420 | G > C | 6833\|ABCC8 | Familial hyperinsulinism, Persistent hyperinsulinemic |
| | | | hypoglycemia of infancy |
| 81002840 | G > C | 675\|BRCA2 | Familial cancer of breast, Hereditary breast and ovarian |
| | | | cancer syndrome, not provided |
| 730882218 | G > C | 4247\|MGAT2 | Abnormal facial shape, Abnormal glycosylation (CDG |
| | | | IIa), Carbohydrate-deficient glycoprotein syndrome type |
| | | | II, Global developmental delay |
| 1555534596 | G > C | 4763\|NF1 | Hereditary cancer-predisposing syndrome, not provided |
| 80358254 | G > C | 4864\|NPC1 | Niemann-Pick disease type C1, Niemann-Pick disease, type |
| | | | C, not provided |
| | G > C | 6261\|RYR1 | not provided |
| 147484110 | G > C | 1476\|CSTB | Epilepsy, progressive myoclonic 1A (Unverricht and |
| | | | Lundborg), Inborn genetic diseases, Progressive myoclonic |
| | | | epilepsy, Unverricht-Lundborg syndrome, not provided |

TABLE E3
Specific targetable mutations from the ClinVar database that can be
corrected with CGBE using Cas9 proteins with NG-PAM recognition.

| snpId | name | geneId | phenotypeList |
|---|---|---|---|
| 370124822 | C > G | 4595\|MUTYH | Hereditary cancer-predisposing syndrome, MYH-associated |
| | | | polyposis, not provided |
| 121908088 | C > G | 7173\|TPO | Deficiency of iodide peroxidase, not provided |
| 536746349 | C > G | 1716\|DGUOK | Progressive external ophthalmoplegia with mitochondrial DNA |
| | | | deletions, autosomal recessive 4, not provided |
| | C > G | 4703\|NEB | Nemaline myopathy, Nemaline myopathy 2 |
| 557312035 | C > G | 7273\|TTN | Dilated cardiomyopathy 1G, Limb-girdle muscular |
| | | | dystrophy, type 2J, Primary dilated cardiomyopathy |
| 587781707 | C > G | 580\|BARD1 | Breast cancer, susceptibility to, Familial cancer of |
| | | | breast, Hereditary cancer-predisposing syndrome, not provided |
| 398123350 | C > G | 272O\|GLB1 | GM1 gangliosidosis type 2, GM1 gangliosidosis type |
| | | | 2, Gangliosidosis GM1 type 3, Gangliosidosis GM1 type |
| | | | 3, Infantile GM1 gangliosidosis, Infantile GM1 |
| | | | gangliosidosis, Mucopolysaccharidosis, MPS-IV- |
| | | | B, Mucopolysaccharidosis, MPS-IV-B, not provided |
| 121913286 | C > G | 5290\|PIK3CA | Adenocarcinoma of prostate, Adenocarcinoma of |
| | | | stomach, Breast adenocarcinoma, Glioblastoma, Malignant |
| | | | melanoma of skin, Malignant neoplasm of body of |
| | | | uterus, Medulloblastoma, Neoplasm of brain, Neoplasm of the |
| | | | breast, Neoplasm of the large intestine, Squamous cell |
| | | | carcinoma of the head and neck, Transitional cell carcinoma of |
| | | | the bladder, Uterine Carcinosarcoma, Uterine cervical |
| | | | neoplasms |
| 143367518 | C > G | 1161\|ERCC8 | Cockayne syndrome type A, not provided |
| 74953290 | C > G | 324\|APC | Hereditary cancer-predisposing syndrome, not provided, not |
| | | | specified |
| 730881857 | C > G | 4683\|NBN | Hereditary cancer-predisposing |
| | | | syndrome, Microcephaly, normal intelligence and |
| | | | immunodeficiency, not provided |
| 878853697 | C > G | 2705\|GJB1 | Charcot-Marie-Tooth Neuropathy X, not provided |
| 33941377 | C > G | 3043\|HBB | Beta thalassemia intermedia, Beta-plus-thalassemia, beta |
| | | | Thalassemia, not provided |
| | C > G | 5896\|RAG1 | Alpha/beta T-cell lymphopenia with gamma/delta T-cell |
| | | | expansion, severe cytomegalovirus infection, and |
| | | | autoimmunity, Combined cellular and humoral immune defects |
| | | | with granulomas, Combined cellular and humoral immune |
| | | | defects with granulomas, Histiocytic medullary |
| | | | reticulosis, Severe immunodeficiency, autosomal recessive, T- |
| | | | cell negative, B-cell negative, NK cell-positive, Severe |
| | | | immunodeficiency, autosomal recessive, T-cell negative, B-cell |
| | | | negative, NK cell-positive |
| 397515905 | C > G | 4607\|MYBPC3 | Cardiovascular phenotype, Familial hypertrophic |
| | | | cardiomyopathy 1, Hypertrophic cardiomyopathy, Primary |
| | | | familial hypertrophic cardiomyopathy, not provided |
| 1057517774 | C > G | 4647\|MYO7A | Deafness, autosomal recessive 2, Usher syndrome, type 1, not |
| | | | provided |
| 587779833 | C > G | 472\|ATM | Ataxia-telangiectasia syndrome, Ataxia-telangiectasia |
| | | | syndrome, Familial cancer of breast, Hereditary cancer- |
| | | | predisposing syndrome, not provided |
| 137853043 | C > G | 7846\|TUBA1A | Tubulinopathies, not provided |
| 1057520574 | C > G | 7846\|TUBA1A | Tubulinopathies, not provided |
| 749491616 | C > G | 35\|ACADS | Deficiency of butyryl-CoA dehydrogenase, not provided |
| 201732356 | C > G | 5428\|POLG | not provided |
| 761649878 | C > G | 5428\|POLG | POLG-Related disorder, Progressive sclerosing |
| | | | poliodystrophy, not provided |
| 769410130 | C > G | 5428\|POLG | Progressive sclerosing poliodystrophy, not provided |
| 587783598 | C > G | 1785\|DNM2 | Myopathy, centronuclear, not provided |
| 879254375 | C > G | 3949\|LDLR | Familial hypercholesterolemia |
| 752596535 | C > G | 3949\|LDLR | Familial hypercholesterolemia |
| 875989909 | C > G | 3949\|LDLR | Familial hypercholesterolemia, Familial hypercholesterolemias |
| 104894718 | C > G | 6324\|SCN1B | Atrial fibrillation, familial, 13, Atrial |
| | | | fibrillation, familial, 13, Brugada syndrome 5, Brugada syndrome |
| | | | 5, Epileptic encephalopathy, early infantile, 52, Generalized |
| | | | epilepsy with febrile seizures plus, Generalized epilepsy with |
| | | | febrile seizures plus, type 1, Generalized epilepsy with febrile |
| | | | seizures plus, type 1, Seizures, not provided |
| 121908725 | C > G | 100\|ADA | Severe combined immunodeficiency due to ADA |
| | | | deficiency, not provided |
| 587777526 | C > G | 23394\|ADNP | Helsmoortel-van der aa syndrome, Inborn genetic diseases, not |
| | | | provided |
| 869312901 | G > C | 6497\|SKI | Shprintzen-Goldberg syndrome, not provided |
| 397516833 | G > C | 6390\|SDHB | Gastrointestinal stroma tumor, Hereditary Paraganglioma- |
| | | | Pheochromocytoma Syndromes, Hereditary cancer- |
| | | | predisposing syndrome, Paragangliomas 4, Paragangliomas |
| | | | 4, Pheochromocytoma, not provided |
| 398123527 | G > C | 2629\|GBA | Gaucher disease, Gaucher's disease, type 1 |
| 794728589 | G > C | 4000\|LMNA | Primary dilated cardiomyopathy, not provided |
| 267607570 | G > C | 4000\|LMNA | Cardiovascular phenotype, Charcot-Marie-Tooth disease, type |
| | | | 2, Dilated cardiomyopathy 1A, not provided |
| 397517977 | G > C | 7399\|USH2A | Retinitis pigmentosa 39, Usher syndrome, type 2A, Usher |
| | | | syndrome, type 2A |
| 876657730 | G > C | 7399\|USH2A | Retinitis pigmentosa 39, Usher syndrome, type 2A, Usher |
| | | | syndrome, type 2A, not provided |
| 1167218743 | G > C | 3030\|HADHA | Long-chain 3-hydroxyacyl-CoA dehydrogenase |
| | | | deficiency, Long-chain 3-hydroxyacyl-CoA dehydrogenase |
| | | | deficiency, Mitochondrial trifunctional protein deficiency |
| 869320742 | G > C | 7273\|TTN | Hereditary myopathy with early respiratory failure, not |
| | | | provided |
| 727504799 | G > C | 7273\|TTN | Cardiomyopathy, Primary dilated cardiomyopathy |
| 672601366 | G > C | 547\|KIF1A | Mental retardation, autosomal dominant 9 |
| 587784141 | G > C | 64324\|NSD1 | Beckwith-Wiedemann syndrome, Sotos syndrome 1 |
| 863224905 | G > C | 64324\|NSD1 | Beckwith-Wiedemann syndrome, Sotos syndrome 1 |
| 988423880 | G > C | 5395\|PMS2 | Hereditary cancer-predisposing syndrome, Lynch syndrome |
| 767978961 | G > C | 729920\|CRPPA | Congenital muscular dystrophy-dystroglycanopathy with brain |
| | | | and eye anomalies, type A7, Muscular dystrophy- |
| | | | dystroglycanopathy (limb-girdle), type c, 7, not provided |
| 1325951163 | G > C | 673\|BRAF | Global developmental delay, not provided |
| 756013171 | G > C | 157680\|VPS13B | Cohen syndrome, not provided |
| 398123181 | G > C | 2592\|GALT | Deficiency of UDPglucose-hexose-1-phosphate |
| | | | uridylyltransferase, not provided |
| 137853022 | G > C | 8518\|ELP1 | Familial dysautonomia |
| 104894845 | G > C | 2717\|GLA | Fabry disease, not provided |
| 104894229 | G > C | HRAS, LRRC56 | Neoplasm of the large intestine, Neoplasm of the thyroid gland |
| 120074186 | G > C | 3784\|KCNQ1 | Cardiovascular phenotype, Congenital long QT |
| | | | syndrome, Jervell and Lange-Nielsen syndrome 1, not provided |
| 121908195 | G > C | 1200\|TPP1 | Ceroid lipofuscinosis neuronal 2, not provided |
| 1057517420 | G > C | 6833\|ABCC8 | Familial hyperinsulinism, Persistent hyperinsulinemic |
| | | | hypoglycemia of infancy |
| 748523268 | G > C | 582\|BBS1 | Bardet-Biedl syndrome 1 |
| 137853150 | G > C | 10312\|TCIRG1 | Osteopetrosis autosomal recessive 1 |
| 876659710 | G > C | 472\|ATM | Ataxia-telangiectasia syndrome, Hereditary cancer- |
| | | | predisposing syndrome |
| 759520465 | G > C | 472\|ATM | Ataxia-telangiectasia syndrome, Hereditary cancer- |
| | | | predisposing syndrome, not provided |
| 539407162 | G > C | 89910\|UBE3B | Inborn genetic diseases, Kaufman oculocerebrofacial syndrome |
| 199474813 | G > C | 4633\|MYL2 | Familial hypertrophic cardiomyopathy 10, not provided |
| 81002840 | G > C | 675\|BRCA2 | Familial cancer of breast, Hereditary breast and ovarian cancer |
| | | | syndrome, not provided |
| 80358871 | G > C | 675\|BRCA2 | Breast-ovarian cancer, familial 2, Hereditary cancer- |
| | | | predisposing syndrome, not provided |
| 730882218 | G > C | 4247\|MGAT2 | Abnormal facial shape, Abnormal glycosylation (CDG |
| | | | IIa), Carbohydrate-deficient glycoprotein syndrome type |
| | | | II, Global developmental delay |
| 2229311 | G > C | 3712\|IVD | Isovaleryl-CoA dehydrogenase deficiency |
| 778768583 | G > C | 825\|CAPN3 | Limb-girdle muscular dystrophy, type 2A, not provided |
| 63750473 | G > C | 368\|ABCC6 | Pseudoxanthoma elasticum, not provided |
| 912983346 | G > C | 6687\|SPG7 | Hereditary spastic paraplegia, not provided |
| 587778720 | G > C | 7157\|TP53 | Adenocarcinoma of prostate, Adenocarcinoma of |
| | | | stomach, Adenoid cystic carcinoma, Adrenocortical |
| | | | carcinoma, Carcinoma of |
| | | | esophagus, Glioblastoma, Hepatocellular carcinoma, Hereditary |
| | | | cancer-predisposing syndrome, Lung |
| | | | adenocarcinoma, Malignant melanoma of skin, Malignant |
| | | | neoplasm of body of uterus, Nasopharyngeal |
| | | | Neoplasms, Neoplasm of brain, Neoplasm of the |
| | | | breast, Neoplasm of the large intestine, Ovarian Serous |
| | | | Cystadenocarcinoma, Pancreatic adenocarcinoma, Renal cell |
| | | | carcinoma, papillary, 1, Squamous cell carcinoma of the head |
| | | | and neck, Squamous cell carcinoma of the skin, Squamous cell |
| | | | lung carcinoma, Transitional cell carcinoma of the |
| | | | bladder, Uterine Carcinosarcoma, not specified |
| 1555534596 | G > C | 4763\|NF1 | Hereditary cancer-predisposing syndrome, not provided |
| 80358010 | G > C | 672\|BRCA1 | Breast-ovarian cancer, familial 1, Hereditary cancer- |
| | | | predisposing syndrome |
| 80358254 | G > C | 4864\|NPCI | Niemann-Pick disease type C1, Niemann-Pick disease, type |
| | | | C, not provided |
| 200727689 | G > C | 3949\|LDLR | Familial hypercholesterolemia |
| 879254565 | G > C | 3949\|LDLR | Familial hypercholesterolemia |
| 879254729 | G > C | 3949\|LDLR | Familial hypercholesterolemia |
| 121908036 | G > C | 3949\|LDLR | Familial hypercholesterolemia |
| 28942082 | G > C | 3949\|LDLR | Familial hypercholesterolemia |
| 875989926 | G > C | 3949\|LDLR | Familial hypercholesterolemia |
| | G > C | 6261\|RYR1 | not provided |
| 398123508 | G > C | 593\|BCKDHA | Maple syrup urine disease, not provided |
| 397516354 | G > C | 7137\|TNNI3 | Hypertrophic cardiomyopathy, Primary familial hypertrophic |
| | | | cardiomyopathy |
| 147484110 | G > C | 1476\|CSTB | Epilepsy, progressive myoclonic 1A (Unverricht and |
| | | | Lundborg), Inborn genetic diseases, Progressive myoclonic |
| | | | epilepsy, Unverricht-Lundborg syndrome, not provided |

TABLE F
List of Exemplary Cas9 or Cas12a Orthologs

| | UniProt or | |
|---|---|---|
| | GenBank | Nickase |
| | Accession | Mutations/Catalytic |
| Ortholog | Number | residues |
| S. pyogenes Cas9 | Q99ZW2.1 | D10A, E762A, H840A, |
| (SpCas9) | | N854A, N863A, D986A¹⁷ |
| S. aureus Cas9 | J7RUA5.1 | D10A and N580¹⁸ |
| (SaCas9) | | |
| S. thermophilus Cas9 | G3ECR1.2 | D31A and N891A¹⁹ |
| (St1Cas9) | | |
| S. pasteurianus Cas9 | BAK30384.1 | D10, H599* |
| (SpaCas9) | | |
| C. jejuni Cas9 | Q0P897.1 | D8A, H559A²⁰ |
| (CjCas9) | | |
| F. novicida Cas9 | A0Q5Y3.1 | D11, N995²¹ |
| (FnCas9) | | |
| P. lavamentivorans | A7HP89.1 | D8, H601* |
| Cas9 (PlCas9) | | |
| C. lari Cas9 (ClCas9) | G1UFN3.1 | D7, H567* |
| Pasteurella multocida | Q9CLT2.1 | |
| Cas9 | | |
| F. novicida Cpf1 | A0Q7Q2.1 | D917, E1006, D1255²¹ |
| (FnCpf1) | | |
| M. bovoculi Cpf1 | WP 052585281.1 | D986A** |
| (MbCpf1) | | |
| A. sp. BV3L6 Cpf1 | U2UMQ6.1 | D908, 993E, Q1226, D1263²³ |
| (AsCpf1) | | |
| L. bacterium N2006 | A0A182DWE3.1 | D832A²⁴ |
| (LbCpf1) | | |

*predicted based on UniRule annotation on the UniProt database.
**Unpublished but deposited at addgene by Ervin Welker: pTE4565 (Addgene plasmid # 88903)

TABLE G
List of Exemplary High Fidelity and/or PAM-relaxed RGN Orthologs

| Published HF/PAM-RGN | | |
|---|---|---|
| variants | PMID | Mutations* |
| S. pyogenes Cas9 | 26628643 | K810A/K1003A/R1060A (1.0); |
| (SpCas9) eSpCasS | | K848A/K1003A/R1060A(1.1) |
| S. pyogenes Cas9 | 29431739 | M495V/Y515N/K526E/R661Q; |
| (SpCas9) evoCas9 | | (M495V/Y515N/K526E/R661S; |
| | | M495V/Y515N/K526E/R661L) |
| S. pyogenes Cas9 | 26735016 | N497A/R661A/Q695A/Q926A |
| (SpCas9) HF1 | | |
| S. pyogenes Cas9 | 30082871 | R691A |
| (SpCas9) HiFi Cas9 | | |
| S. pyogenes Cas9 | 28931002 | N692A, M694A, Q695A, H698A |
| (SpCas9) HypaCas9 | | |
| S. pyogenes Cas9 | 30082838 | F539S, M763I, K890N |
| (SpCas9) Sniper-Cas9 | | |
| S. pyogenes Cas9 | 29512652 | A262T, R324L, S409I, E480K, E543D, |
| (SpCas9) xCas9 | | M694I, E1219V |
| S. pyogenes Cas9 | 30166441 | R1335V, L1111R, D1135V, G1218R, |
| (SpCas9) SpCas9-NG | | E1219F, A1322R, T1337R |
| S. pyogenes Cas9 | 26098369 | D1135V, R1335Q, T1337R; |
| (SpCas9) VQR/VRER | | D1135V/G1218R/R1335E/T1337R |
| S. aureus Cas9 | 26524662 | E782K/N968K/R1015H |
| (SaCas9)-KKH | | |
| enAsCas12a | U.S. Ser. No. | One or more of: E174R, S170R, S542R, |
| | 15/960,271 | K548R, K548V, N551R, N552R, K607R, |
| | | K607H, e.g., E174R/S542R/K548R, |
| | | E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, |
| | | S170R/S542R/K607R, or |
| | | S170R/S542R/K548V/N552R |
| enAsCas12a-HF | U.S. Ser. No. | One or more of: E174R, S542R, K548R, |
| | 15/960,271 | e.g., E174R/S542R/K548R, |
| | | E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, |
| | | S170R/S542R/K607R, or |
| | | S170R/S542R/K548V/N552R, with the |
| | | addition of one or more of: N282A, |
| | | T315A, N515A and K949A |
| enLbCas12a(HF) | U.S. Ser. No. | One or more of T152R, T152K, D156R, |
| | 15/960,271 | D156K, Q529K, G532R, G532K, G532Q, |
| | | K538R, K538V, D541R, Y542R, M592A, |
| | | K595R, K595H, K595S or K595Q, e.g., |
| | | D156R/G532R/K538R, |
| | | D156R/G532R/K595R, |
| | | D156R/G532R/K538V/Y542R, |
| | | T152R/G532R/K538R, T152R/D156R, |
| | | D156R/G532R, T152R/G532R, |
| | | D156R/G532R/K538R/D541R, |
| | | D156R/G532R/K595H, |
| | | T152R/G532R/K595R, |
| | | T152R/G532R/K538V/Y542R, optionally |
| | | with the addition of one or more of: |
| | | N260A, N256A, K514A, D505A, K881A, |
| | | S286A, K272A, K897A |
| enFnCas12a(HF) | U.S. Ser. No. | One or more of T177A, K180R, K180K, |
| | 15/960,271 | E184R, E184K, T604K, N607R, N607K, |
| | | N607Q, K613R, K613V, D616R, N617R, |
| | | M668A, K671R, K671H, K671S, or K671Q, |
| | | e.g., E184R/N607R/K613R, |
| | | E184R/N607R/K671R, |
| | | E184R/N607R/K613V/N617R, |
| | | K180R/N607R/K613R, K180R/E184R, |
| | | E184R/N607R, K180R/N607R, |
| | | E184R/N607R/K613R/D616R, |
| | | E184R/N607R/K671H, |
| | | K180R/N607R/K671R, |
| | | K180R/N607R/K613V/N617R, optionally |
| | | with the addition of one or more of: |
| | | N305A, N301A, K589A, N580A, K962A, |
| | | S334A, K320A, K978A |
| chimeric Cas9 | 30718489 | S. aureus Cas9 with PAM interaction |
| cCas9 | | domain from SaCas9 orthologues, |
| | | expands recognition and targetability |
| | | of NNVRRN, NNVACT, NNVATG, |
| | | NNVATT, NNVGCT, NNVGTG, and |
| | | NNVGTT PAM sequences |
| Streptococcus | doi: https://doi.org/ | Recognizes 5′-NAA-3′ PAM |
| macacae (Smac) Cas9 | 10.1101/429654 | |
| NCTC 11558 | | |
| Spy-mac Cas9, | doi: https://doi.org/ | Recognizes 5′-NAA-3′ PAM |
| Smac-py Cas9 | 10.1101/429654 | |
| N. meningitidis | 30581144 | Recognizes N₄CC PAM |
| Nme2Cas9 | | |
| SpG Cas9 | 32217751 | SpCas9 variant capable of targeting |
| (SEQ-ID 158) | | NGN PAMs |
| | | D1135L/S1136W/G1218K/E1219Q/ |
| | | R1335Q/T1337R |
| | | Also as SpG-HF1 in combination |
| | | with N497A/R661A/Q695A/Q926A |
| SpRY Cas9 | 32217751 | SpCas9 variant capable of targeting |
| (SEQ-ID 157) | | NRN > NYN PAMs |
| | | SpRY(A61R/L1111R/D1135L/S1136W/ |
| | | G1218K/E1219Q/N1317R/A1322R/ |
| | | R1333P/R1335Q/T1337R); also as |
| | | SpRY-HF1 in combination with |
| | | N497A/R661A/Q695A/Q926A |

*predicted based on UniRule annotation on the UniProt database.

TABLE H
Amino acid substitutions predicted to generate ABE variants
with reduced RNA editing. This table lists the residue changes
in either or both TadA domains of the TadA heterodimer (present
in e.g., ABE7.10) predicted to cause an RRE phenotype, next
to the reasoning behind the proposed changes.

| | |
|---|---|
| Residues to Change | Rationale |

| Wild type | Engineered | Protein | Binding |
|---|---|---|---|
| (WT) TadA | TadA | structure | prediction |
| S7 | S205 | | x |
| H8 | H206 | | x |
| E9 | E207 | | x |
| Y10 | Y208 | | x |
| W11 | W209 | | x |
| M12 | M210 | | x |
| R13 | R211 | x | x |
| H14 | H212 | | x |
| T17 | T215 | | x |
| K20 | K218 | x | x |
| R21 | R219 | x | x |
| W23 | R221 | x | |
| E25 | E223 | x | x |
| R26 | R224 | x | x |
| E27 | E225 | x | |
| V28 | V226 | x | x |
| P29 | P227 | | x |
| V30 | V228 | x | |
| G31 | G229 | | x |
| H36 | L234 | | x |
| N37 | N235 | | x |
| N38 | N236 | | x |
| N46 | N244 | x | |
| R47 | R245 | | x |
| P48 | A246 | x | |
| I49 | I247 | x | |
| G50 | G248 | | x |
| R51 | I249 | | x |
| H52 | H250 | | x |
| D53 | D251 | | x |
| P54 | P252 | | x |
| T55 | T253 | | x |
| A56 | A254 | x | |
| H57 | H255 | x | x |
| A58 | A256 | x | |
| E59 | E257 | x | |
| R64 | R262 | | x |
| Q65 | Q263 | | x |
| G67 | G265 | | x |
| L68 | L266 | | x |
| Q71 | Q269 | | x |
| N72 | N270 | | x |
| R74 | R272 | | x |
| I76 | I274 | | x |
| D77 | D275 | | x |
| Y81 | Y279 | | x |
| V82 | V280 | x | |
| T83 | T281 | | x |
| L84 | F282 | x | |
| E85 | E283 | x | |
| P86 | P284 | x | x |
| C87 | C285 | x | x |
| V88 | V286 | | x |
| M89 | M287 | | x |
| C90 | C288 | x | x |
| R98 | R296 | | x |
| G100 | G298 | | x |
| R101 | R299 | | x |
| A106 | V304 | x | |
| R107 | R305 | x | |
| D108 | N306 | x | |
| A109 | A307 | x | |
| K110 | K308 | x | |
| T111 | T309 | x | |
| D119 | D317 | | x |
| H122 | H320 | | x |
| H123 | Y321 | | x |
| P124 | P322 | | x |
| G125 | G323 | | x |
| M126 | M324 | | x |
| N127 | N325 | | x |
| H128 | H326 | | x |
| R129 | R327 | | x |
| V130 | V328 | | x |
| E131 | E329 | | x |
| I132 | I330 | | x |
| T133 | T331 | | x |
| E134 | E332 | | x |
| G135 | G333 | | x |
| L137 | L335 | | x |
| A138 | A336 | x | x |
| D139 | D337 | | x |
| E140 | E338 | | x |
| C141 | C339 | x | x |
| A142 | A340 | x | x |
| A143 | A341 | x | x |
| L144 | L342 | | x |
| L145 | L343 | x | x |
| S146 | C344 | x | |
| D147 | Y345 | | x |
| F148 | F346 | x | x |
| F149 | F347 | x | x |
| R150 | R348 | x | x |
| M151 | M349 | | x |
| R152 | P350 | x | x |
| R153 | R351 | x | |
| Q154 | Q352 | | x |
| E155 | V353 | x | x |
| I156 | F354 | | x |
| K157 | N355 | | x |
| K160 | K358 | | x |
| K161 | K359 | | x |

TABLE I
Amino acid residues whose mutation may be expected to
yield base editor RRE variants. These positions were
chosen based on a APOBEC1 structural model and RNA/DNA
binding predictions or based on previous description
in the literature as residues whose mutation reduced
the RNA editing or binding activities of isolated APOBEC1.

| Residue Change | Reasoning |
|---|---|
| E24, V25 | model & RNA binding prediction |
| R118, Y120, H121, R126 | model & RNA binding prediction |
| W224-K229 | model & RNA binding prediction |
| P168-I186 | model & RNA binding prediction |
| L173 + L180 | model & RNA binding prediction |
| R15, R16, R17, to K15-17 & A15-17 | Teng et al, J Lipid Research 1999 |
| Deletion E181-L210 | Teng et al, J Lipid Research 1999 |
| P190 + P191 | Teng et al, J Lipid Research 1999 |
| Deletion L210-K229 (C-terminal) | Teng et al, J Lipid Research 1999 |
| Deletion S2-L14 (N-terminal) | Teng et al, J Lipid Research 1999 |
| V64, F66 | Teng et al, J Lipid Research 1999 |
| L180A | Teng et al, J Lipid Research 1999 |
| C192, L193, L196, P201, L203, | Teng et al, J Lipid Research 1999 |
| L210, P219, P220 | |
| P92 | MacGinnitie et al, JBC 1995 |

TABLE J
UNG and SMUG analogues

| | Uniprot | |
|---|---|---|
| | accession number | |
| UNG orthologue | | |
| Mouse | P97931 | SEQ ID NO: 318 |
| Rat | Q5BK44 | SEQ ID NO: 319 |
| Baker's yeast | P12887 | SEQ ID NO: 320 |
| Caenorhabditis | Q9U221 | SEQ ID NO: 321 |
| elegans | | |
| Mouse-ear cress | Q9LIH6 | SEQ ID NO: 322 |
| Zebrafish | Q7ZVD1 | SEQ ID NO: 323 |
| Rabbit | G1SJ42 | SEQ ID NO: 324 |
| Polar bear | A0A452THE0 | SEQ ID NO: 325 |
| Black snub-nosed | A0A2K6MB33 | SEQ ID NO: 326 |
| monkey | | |
| Common wombat | A0A4X2KC02 | SEQ ID NO: 327 |
| Mycobacterium | A0A1X2AUJ0 | SEQ ID NO: 328 |
| riyadhense | | |
| Indian major carp | A0A498LRM7 | SEQ ID NO: 329 |
| Fission yeast | O74834 | SEQ ID NO: 330 |
| Japanese pufferfish | A0A3B5KG53 | SEQ ID NO: 331 |
| Thirteen-lined ground | I3M8Q6 | SEQ ID NO: 332 |
| squirrel | | |
| Japanese rice fish | A0A3P9H4T8 | SEQ ID NO: 333 |
| Electric eel | A0A4W4HK79 | SEQ ID NO: 334 |
| Western clawed frog | A0A5G3K4Q6 | SEQ ID NO: 335 |
| Enterobacter cloacae | A0A0F0TTY1 | SEQ ID NO: 336 |
| subsp, cloacae | | |
| Clostridium oryzae | A0A1V4IJH4 | SEQ ID NO: 337 |
| Lactobacillus apis | A0A1C3ZIJ7 | SEQ ID NO: 338 |
| Flavobacterium sp. | A0A519N079 | SEQ ID NO: 339 |
| Delftia lacustris | A0A1H3TI78 | SEQ ID NO: 340 |
| Lactococcus garvieae | A0A3D4RH89 | SEQ ID NO: 341 |
| Lactobacillus rodentium | A0A2Z6T8A7 | SEQ ID NO: 342 |
| SMUG orthologue | | |
| Human | Q53HV7 | SEQ ID NO: 343 |
| Rat | Q811Q1 | SEQ ID NO: 344 |
| Mouse | Q6P5C5 | SEQ ID NO: 345 |
| African clawed frog | Q9YGN6 | SEQ ID NO: 346 |
| Bovine | Q59I47 | SEQ ID NO: 347 |

### EXAMPLES

The invention is further described in the following examples, which do not limit the scope of the invention described in the claims.

### Methods

### Molecular Cloning

All base editor (BE) and prime editor (PE) constructs were cloned into a mammalian expression plasmid backbone under the control of a pCMV promoter (AgeI and NotI restriction digest of parental plasmid Addgene #112101). The wild-type SpCas9 construct (SQT 817; Addgene #53373) is expressed under the control of a CAG promoter. All BE and PE constructs were encoded as P2A-eGFP fusions for co-translational expression of the base/prime editors and eGFP. Gibson fragments with matching overlaps were PCR-amplified using Phusion High-fidelity polymerase (NEB). Fragments were gel-purified and assembled for 1 hour at 50° C. and transformed into chemically competent E. coli (XL1-Blue, Agilent). The UNGs used in our experiments originated either from E. coli (eUNG; UniProtKB-P12295) or Homo sapiens (hUNG; UniProtKB-P13051), were codon-optimized for expression in human cells and synthesized as gblocks (IDT). All guide RNA (gRNA) constructs were cloned into a BsmBI-digested pUC19-based entry vector (BPK1520, Addgene #65777) with a U6 promoter driving gRNA expression. We designed the pegRNAs to implement the same C-to-G changes that the CGBE constructs would install and followed previously described default design rules for designing pegRNAs and ngRNAs15. PegRNAs were cloned into the BsaI-digested pU6-pegRNA-GG-acceptor entry vector (Addgene #132777) and ngRNAs were cloned into the abovementioned BsmBI-digested entry vector BPK1520. Oligos containing the spacer, the 5′phosphorylated pegRNA scaffold, and the 3′ extension sequences were annealed to form dsDNA fragments with compatible overhangs and ligated using T4 ligase (NEB). All plasmids used for transfection experiments were prepared using Qiagen Midi or Maxi Plus kits.

### Guide RNAs

All gRNAs for base editors were of the form
(SEQ ID NO 145)
5′-NNNNNNNNNNNNNNNNNNNNCGTTTTAGAGCTAGAAATAGCAAGTT
AAAATAAGGCTAGTCCGTTATCAACTTGAAAAAGTGGCACCGAGTCGGT
GCTTTTTTT-3′.

TABLE K
Shown below are the protospacer regions (NNNNNNNNNNNNNNNNNNNN in
SEQ ID NO: 146) for these gRNAs (all written 5′ to 3′).

| target gene/site | protospacer sequence | SEQ ID NO: |
|---|---|---|
| ABE site 7 | GAATACTAAGCATAGACTCC | 216 |
| ABE site 8 | GTAAACAAAGCATAGACTGA | 217 |
| ABE site 9 | GAAGACCAAGGATAGACTGC | 218 |
| ABE site 18 | ACACACACACTTAGAATCTG | 219 |
| ABE site 19 | CACACACACTTAGAATCTGT | 220 |
| ABE site 20 | TTAAGCTGTAGTATTATGAA | 221 |
| ABE site 21 | CCTGGCCTGGGTCAATCCTT | 222 |
| EMX1 site 1 | GAGTCCGAGCAGAAGAAGAA | 223 |
| EMX1 site 2 | GTATTCACCTGAAAGTGTGC | 224 |
| FANCF site 1 | GGAATCCCTTCTGCAGCACC | 225 |
| HEK site 2 (ABE site 1) | GAACACAAAGCATAGACTGC | 226 |
| HEK site 3 | GGCCCAGACTGAGCACGTGA | 227 |
| HEK site 4 | GGCACTGCGGCTGGAGGTGG | 228 |
| HEK site 5 | CTGGCCTGGGTCAATCCTTG | 229 |
| HEK site 6 | CAAAGCAGGATGACAGGCAG | 230 |
| PDCD1 site 2 | ACTTCCACATGAGCGTGGTC | 231 |
| PPP1R12C site 2 | GGCACTCGGGGGCGAGAGGA | 232 |
| PPP1R12C site 3 | GAGCTCACTGAACGCTGGCA | 233 |
| PPP1R12C site 4 | GACCCTCAGCCGTGCTGCTC | 234 |
| PPP1R12C site 5 | GCTGACTCAGAGACCCTGAG | 235 |
| PPP1R12C site 6 | GGGGCTCAACATCGGAAGAG | 236 |
| PPP1R12C site 7 | GCTGGCTCAGGTTCAGGAGA | 237 |
| PPP1R12C site 8 | CTGCTCGGGGTGGGACTCTG | 238 |
| RNF2 site 1 | GTCATCTTAGTCATTACCTG | 239 |
| VEGFA site 4 | GAGGACGTGTGTGTCTGTGT | 240 |

For C5, 7, 8 guides

| | | |
|---|---|---|
| ABE site 23 | TAAGCATAGACTCCAGGATA | 241 |
| ABE site 24 | TACTCTGAGTGTACAAAAGA | 242 |
| ABE site 25 | AGTAAACAAAGCATAGACTG | 243 |
| ABE site 26 | TTTGTGCAAACACAGATTGC | 244 |
| ABE site 27 | CGGGCATCAGAATTCCCTGG | 245 |
| EMX1 site 3 | AAAGTACAAACGGCAGAAGC | 246 |
| EMX1 site 4 | GTACAAACGGCAGAAGCTGG | 247 |
| FANCF site 2 | GCTGCAGAAGGGATTCCATG | 248 |
| FANCF site 3 | CGCCGTCTCCAAGGTGAAAG | 249 |
| FANCF site 4 | AGCGATCCAGGTGCTGCAGA | 250 |
| HEK site 7 | GGAACACAAAGCATAGACTG | 251 |
| HEK site 8 | TGTGTTCCAGTTTCCTTTAC | 252 |
| HEK site 9 | TTGTTTGCAGCTATTCAGGC | 253 |
| PPP1R12C site 9 | AAGTCGAGGGAGGGATGGTA | 254 |
| PPP1R12C site 10 | GACACGTGGATTGTGCTGTC | 255 |
| PPP1R12C site 11 | GTCATACACTGGGCTGGCCA | 256 |
| PPP1R12C site 12 | CAAAGTCCAGGACCGGCTGG | 257 |
| PPP1R12C site 13 | GCATGGCTCTAGTGCTTTCC | 258 |
| PPP1R12C site 14 | GGTCATACACTGGGCTGGCC | 259 |
| PPP1R12C site 15 | AAGGAGACAAAGTCCAGGAC | 260 |
| PPP1R12C site 16 | GATTGTGCTGTCAGGAGCTC | 261 |
| RNF2 site 2 | ATGACTAAGATGACTGCCAA | 262 |
| RNF2 site 3 | TGAGTTACAACGAACACCTC | 263 |

For guides with NGT or NGAG PAM

| | | |
|---|---|---|
| CGBE_NG site 1 | ACCATCTTTTGTACACTCAG | 264 |
| CGBE_NG site 2 | CACTTCTCTTCCTGCCCTCT | 265 |
| CGBE_NG site 3 (EMX1) | AGCTTCTGCCGTTTGTACTT | 266 |
| CGBE_NG site 4 (RNF2) | CGTCTCATATGCCCCTTGGC | 267 |
| CGBE_NG site 5 | ATAGACTCCAGGATAAGGTA | 268 |
| CGBE_NG site 6 | CTCAACATCGGAAGAGGGGA | 269 |
| (PPP1R12C) | | |
| CGBE_VRQR site 1 | TCAATCCTTGGGGCCCAGAC | 270 |
| CGBE_VRQR site 2 | ATGTTCCAATCAGTACGCAG | 271 |
| (FANCF) | | |
| CGBE_VRQR site 3 | GATGACTGCCAAGGGGCATA | 272 |
| (RNF2) | | |
| CGBE_VRQR site 4 | AAGTACAAGCACTCAATGTG | 273 |
| CGBE_VRQR site 5 | ACACACACTTAGAATCTGTG | 274 |
| CGBE_VRQR site 6 | GCGGACAGTGGACGCGGCGG | 275 |
| (VEGFA) | | |

TABLE L
Shown below are the sequence for DNA off-target
sites (all written 5′ to 3′).

| | | SEQ |
|---|---|---|
| | | ID |
| target site | sequence | NO: |
| HEK site 2 off 1 | GAACACAATGCATAGATTGC | 276 |
| HEK site 2 off 2 | AAACATAAAGCATAGACTGC | 277 |
| HEK site 3 off 1 | CACCCAGACTGAGCACGTGC | 278 |
| HEK site 3 off 2 | GACACAGACTGGGCACGTGA | 279 |
| HEK site 3 off 3 | AGCTCAGACTGAGCAAGTGA | 280 |
| HEK site 3 off 4 | AGACCAGACTGAGCAAGAGA | 281 |
| HEK site 3 off 5 | GAGCCAGAATGAGCACGTGA | 282 |
| HEK site 4 off 1 | TGCACTGCGGCCGGAGGAGG | 283 |
| HEK site 4 off 2 | GGCTCTGCGGCTGGAGGGGG | 284 |
| HEK site 4 off 3 | GGCACGACGGCTGGAGGTGG | 285 |
| HEK site 4 off 4 | GGCATCACGGCTGGAGGTGG | 286 |
| HEK site 4 off 5 | GGCGCTGCGGCGGGAGGTGG | 287 |
| EMX1 site 1 off 1 | GAGTCTAAGCAGAAGAAGAA | 288 |
| EMX1 site 1 off 2 | GAGGCCGAGCAGAAGAAAGA | 289 |
| EMX1 site 1 off 3 | GAGTCCTAGCAGGAGAAGAA | 290 |
| EMX1 site 1 off 4 | GAGTCCGGGAAGGAGAAGAA | 291 |
| EMX1 site 1 off 5 | GAGCCGGAGCAGAAGAAGGA | 292 |
| FANCF site 1 off 1 | GGAACCCCGTCTGCAGCACC | 293 |
| FANCF site 1 off 2 | GGAGTCCCTCCTACAGCACC | 294 |
| FANCF site 1 off 3 | AGAGGCCCCTCTGCAGCACC | 295 |
| FANCF site 1 off 4 | ACCATCCCTCCTGCAGCACC | 296 |
| FANCF site 1 off 5 | GGATTGCCATCCGCAGCACC | 297 |
| FANCF site 1 off 6 | TGAATCCCATCTCCAGCACC | 298 |

All pegRNAs for prime editors were of the form
(SEQ ID NO: 299)
5′-NNNNNNNNNNNNNNNNNNNNGTTTTAGAGCTAGAAATAGCAAGTTA
AAATAAGGCTAGTCCGTTATCAACTTGAAAAAGTGGCACCGAGTCGGTG
CNNNNNNNNNNNNNNNNNNNNTTTTTTT-3′.

TABLE M
Shown below are the protospacer and 3′ extension sequences for these pegRNAs
(all written 5′ to 3′).

| target | protospacer | SEQ ID | | SEQ ID |
|---|---|---|---|---|
| gene/site | sequence | NO: | 3′ extension sequence | NO: |
| ABE_site7_ | CTATATTACTT | 300 | GAATAGTAAGCATAGACTC | 301 |
| CtoG | ACCTTATCC | | CAGGATAAGGTAAGTAATAT | |
| ABE_site8_ | ATGAGGAAAG | 302 | GTAAAGAAAGCATAGACTG | 303 |
| CtoG | GGACTAGAGT | | AGGGGTACAATCCTACTCT | |
| | | | AGTCCCTTTCCTC | |
| HEK_site2_ | GCTGGCCCTG | 304 | GCTTTCTGTTCCAGTTTCCT | 305 |
| CtoG | TAAAGGAAAC | | TTACAGGGCCA | |
| RNF2_site1_ | TGAGTTACAA | 306 | GTCATGTTAGTCATTACCTG | 307 |
| CtoG | CGAACACCTC | | AGGTGTTCGTTGTAACT | |
| HEK_site3_ | GGCCCAGACT | 308 | TCTGCCATCAAAGCGTGCT | 309 |
| CTTins | GAGCACGTGA | | CAGTCTG | |
| FANCF_site1_ | GGAATCCCTT | 310 | GGAAAAGCGATCAAGGTGC | 311 |
| GtoT | CTGCAGCACC | | TGCAGAAGGGA | |

All nicking gRNAs for PE3 system were of the form
(SEQ ID NO: 145)
5′-NNNNNNNNNNNNNNNNNNNNCGTTTTAGAGCTAGAAATAGCAAGTTAA
AATAAGGCTAGTCCGTTATCAACTTGAAAAAGTGGCACCGAGTCGGTGCTT
TTTTT-3′.

TABLE N
Shown below are the protospacer regions for these
nicking gRNAs (all written 5′ to 3′).

| | | SEQ ID |
|---|---|---|
| target gene/site | PE3 nicking guide RNA | NO: |
| ABE_site 7_CtoG | AGAATGGCAGGCACGTAGTA | 312 |
| ABE_site 8_CtoG | TGCAACAGCTATGAAATAGC | 313 |
| HEK_site 2_CtoG | TTGTTTGCAGCTATTCAGGC | 314 |
| RNF2_site 1_CtoG | TACACGTCTCATATGCCCCT | 315 |
| HEK_site 3 CTTins | GTCAACCAGTATCCCGGTGC | 316 |
| FANCF_site 1_GtoT | GAAGCTCGGAAAAGCGATCA | 317 |

### Cell Culture

STR-authenticated HEK293T (CRL-3216), K562 (CCL-243), HeLa (CCL-2), and U2OS cells (similar match to HTB-96; gain of #8 allele at the D5S818 locus) were used in this study. HEK293T and HeLa cells were grown in Dulbecco's Modified Eagle Medium (DMEM, Gibco) with 10% heat-inactivated fetal bovine serum (FBS, Gibco) supplemented with 1% penicillin-streptomycin (Gibco) antibiotic mix. K562 cells were grown in Roswell Park Memorial Institute (RPMI) 1640 Medium (Gibco) with 10% FBS supplemented with 1% Pen-Strep and 1% GlutaMAX (Gibco). U2OS cells were grown in DMEM with 10% FBS supplemented with 1% Pen-Strep and 1% GlutaMAX. Cells were grown at 37° C. in 5% CO2 incubators and periodically passaged upon reaching around 80% confluency. Cell culture media supernatant was tested for mycoplasma contamination using the MycoAlert mycoplasma detection kit (Lonza) and all tests were negative throughout the experiments.

### Transfections

HEK293T cells were seeded at 1.25×10⁴ cells per well into 96-well flat bottom cell culture plates (Corning) for DNA on-target experiments or at 6.25×10⁴ cells per well into 24-well cell culture plates (Corning) for DNA off-target experiments. 24 hours post-seeding, cells were transfected with 30 ng of control or base/prime editor plasmid and 10 ng of gRNA plasmid (and 3.3 ng nicking gRNA plasmid for PE3) using 0.3 μL of TransIT-X2 (Mirus) lipofection reagent for experiments in 96-well plates, or 150 ng control or base editor plasmid and 50 ng gRNA, and 1.5 μL TransIT-X2 for experiments in 24-well plates. K562 cells were electroporated using the SF Cell Line Nucleofector X Kit (Lonza), according to the manufacturer's protocol with 2×10⁵ cells per nucleofection and 800 ng control or base/prime editor plasmid, 200 ng gRNA or pegRNA plasmid, and 83 ng nicking gRNA plasmid (for PE3). U2OS cells were electroporated using the SE Cell Line Nucleofector X Kit (Lonza) with 2×10⁵ cells and 800 ng control or base/prime editor plasmid, 200 ng gRNA or pegRNA, and 83 ng nicking gRNA (for PE3). HeLa cells were electroporated using the SE Cell Line 4D-Nucleofector X Kit (Lonza) with 5×10⁵ cells and 800 ng control or base/prime editor, 200 ng gRNA or pegRNA, and 83 ng nicking gRNA (for PE3). 72 hours post-transfection, cells were lysed for extraction of genomic DNA (gDNA).

### DNA Extraction

HEK293T cells were washed with 1×PBS (Corning) and lysed overnight by shaking at 55° C. with 43.5 μl of gDNA lysis buffer (100 mM Tris-HCl at pH 8, 200 mM NaCl, 5 mM EDTA, 0.05% SDS) supplemented with 5.25 μl of 20 mg/ml Proteinase K (NEB) and 1.25 μl of 1M DTT (Sigma) per well for experiments in 96-well plates, or with 174 μl DNA lysis buffer, 21 μl Proteinase K, and 5 μL 1M DTT per well for experiments in 24-well plates. K562 cells were centrifuged for 5 min, media removed, and lysed overnight by shaking at 55° C. with 174 μl DNA lysis buffer, 21 μl Proteinase K, and 5 μL 1M DTT per well in 24-well plates. U2OS cells and HeLa cells were washed with 1×PBS and lysed overnight shaking at 55° C. with 174 μl DNA lysis buffer, 21 μl Proteinase K, and 5 μL 1M DTT per well in 24-well plates. Subsequently, gDNA was extracted from lysates using 1-2× paramagnetic beads as previously described⁷ and eluted in 45 μl of 0.1×EB buffer. DNA extraction was performed using a Biomek FX^{P} Laboratory Automation Workstation (Beckman Coulter).

### Targeted Amplicon Sequencing

DNA targeted amplicon sequencing was performed as previously described.⁷ Briefly, extracted gDNA was quantified using the Qubit dsDNA HS Assay Kit (Thermo Fisher). Amplicons were constructed in 2 PCR steps. In the first PCR, regions of interest (170-250 bp) were amplified from 5-20 ng of gDNA with primers containing Illumina forward and reverse adapters on both ends (Supplementary Table 9). PCR products were quantified on a Synergy HT microplate reader (BioTek) at 485/528 nm using a Quantifluor dsDNA quantification system (Promega), pooled and cleaned with 0.7× paramagnetic beads, as previously described.⁷ In a second PCR step (barcoding), unique pairs of Illumina-compatible indexes (equivalent to TruSeq CD indexes, formerly known as TruSeq HT) were added to the amplicons. The amplified products were cleaned up with 0.7× paramagnetic beads, quantified with the Quantifluor or Qubit systems, and pooled before sequencing. The final library was sequenced on an Illumina MiSeq machine using the Miseq Reagent Kit v2 (300 cycles, 2×150 bp, paired-end). Demultiplexed FASTQ files were downloaded from BaseSpace (Illumina).

### Example 1. ABE Induces C-to-G Editing in Human HEK293T Cells

Human HEK293T cells were transfected with plasmids encoding nCas9, ABEmax, miniABEmax-K20/R21A, and miniABEmax-V82G (FIG. 1-2) and gRNAs targeting several genomic sites (e.g. FANCF site 1, HEK site 2 and ABE site 7). After 72 hours, gDNA was extracted and targeted amplicon sequencing was performed to determine the on-target DNA editing of ABE constructs. C-to-G editing was seen on all three sites next to the expectedly robust A-to-G DNA base editing and probably stemmed from deamination of cytosine by the adenosine deaminase TadA, followed by downstream DNA and base excision repair (FIG. 1-4)

### Example 2. Engineering of CGBE1 and its Testing at 25 Genomic Loci

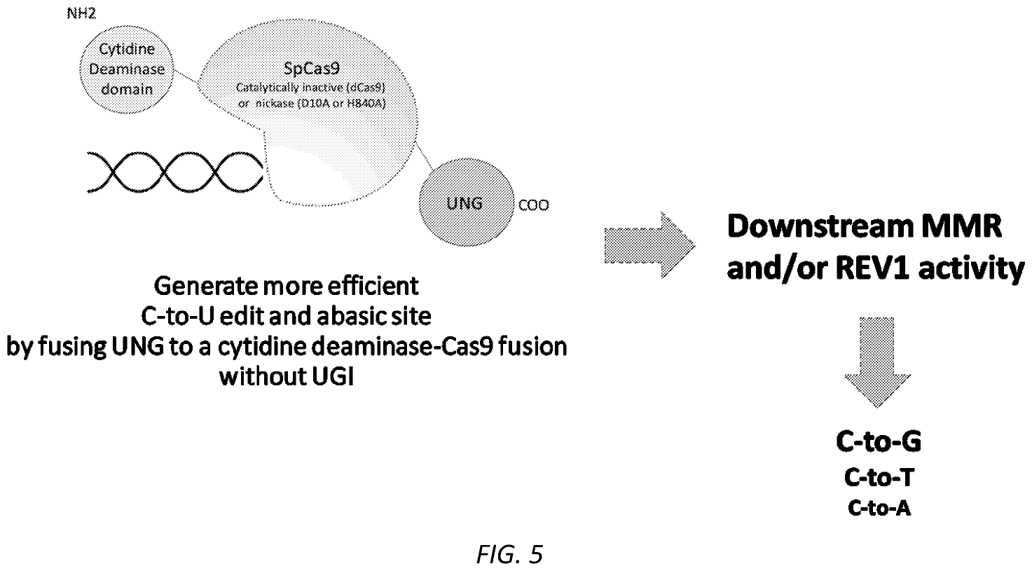

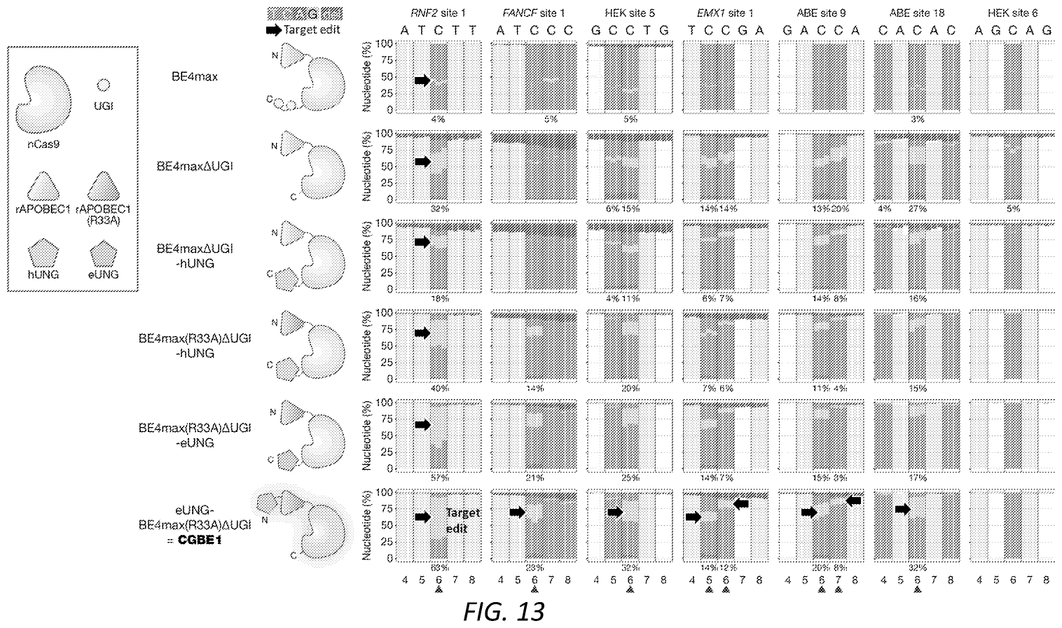

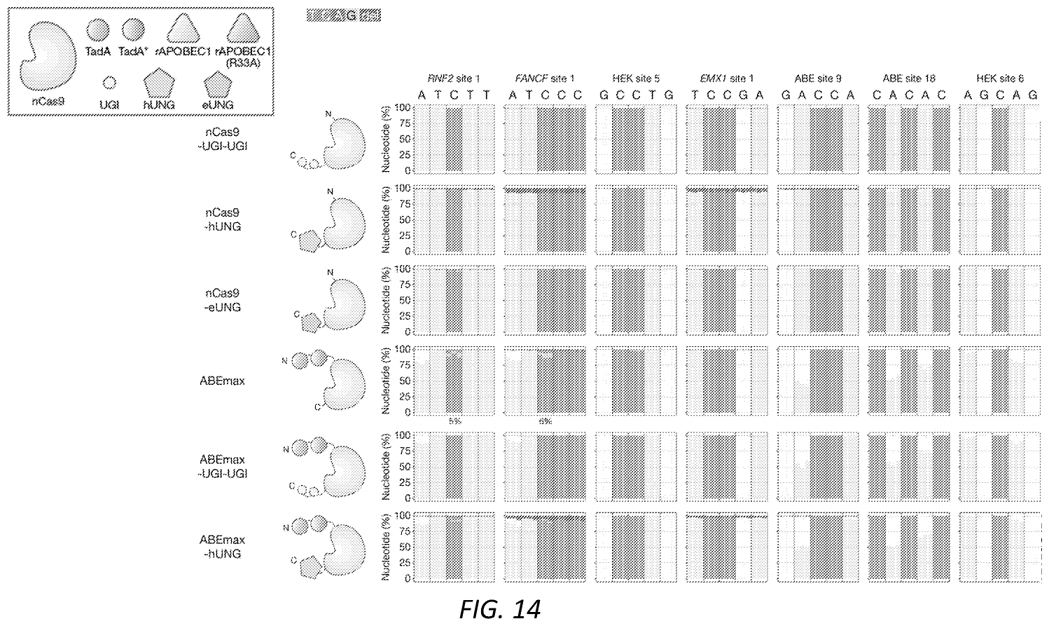

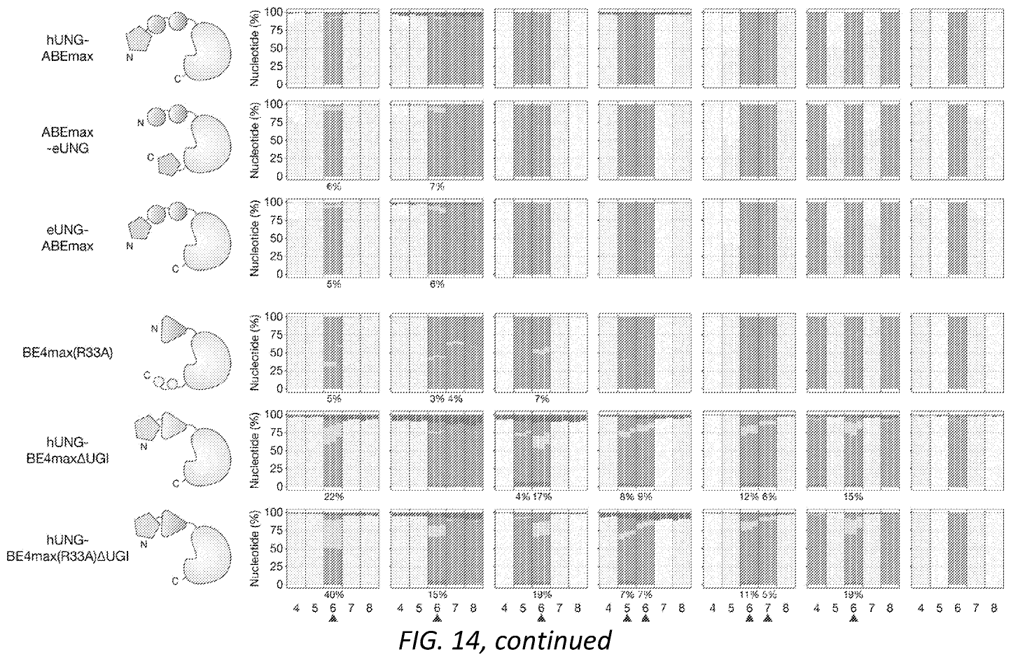

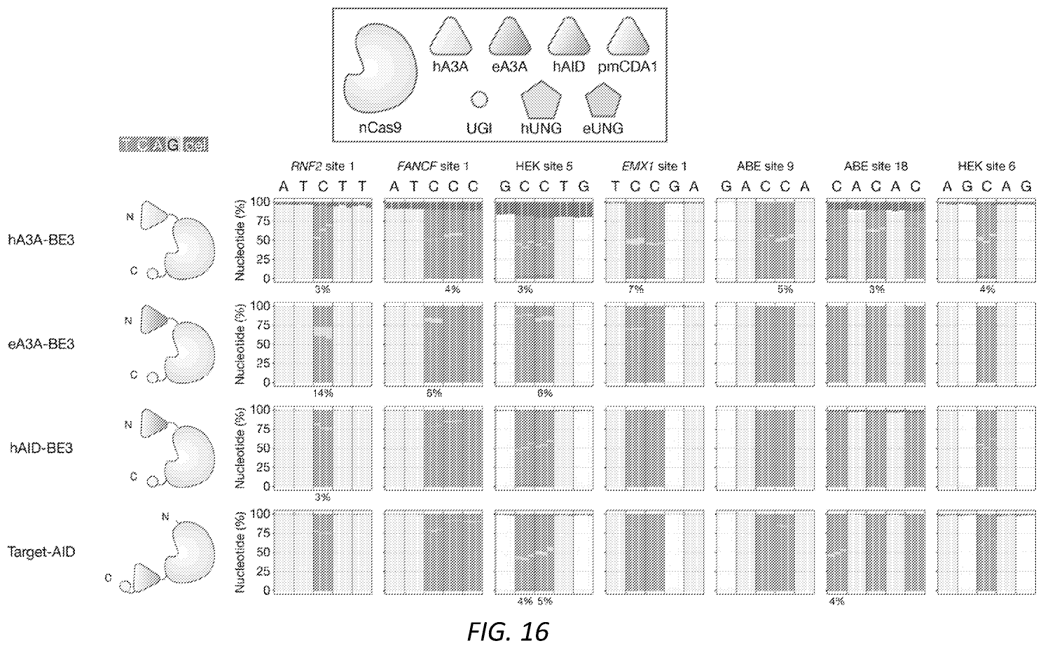

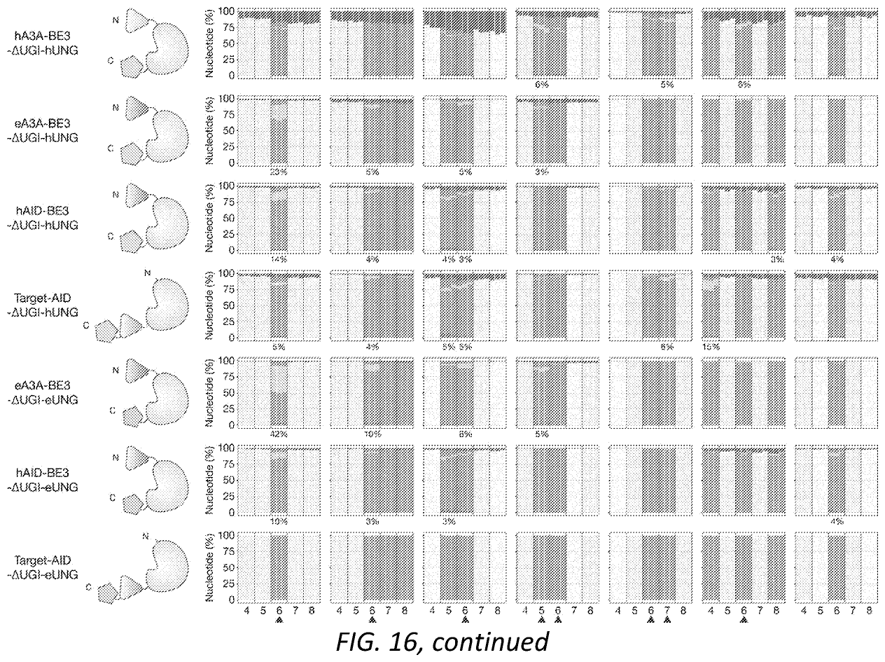

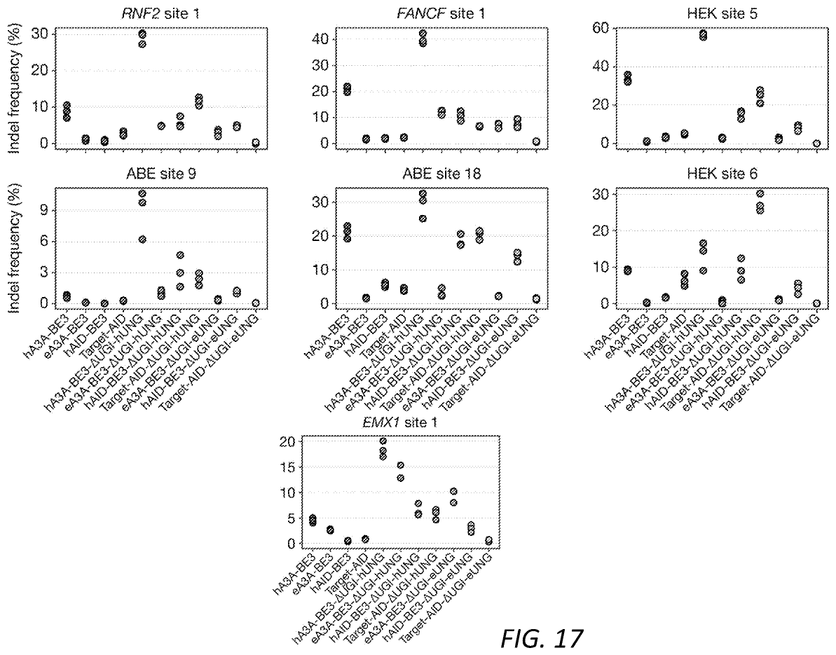

Given the observation outlined in Example 1 on ABE-mediated C-to-G alterations, we wondered whether we could induce these edits more efficiently by modifying the BE4max CBE^{8,15}, which harbors an enzyme actually intended to deaminate cytosines (the rat APOBEC1 cytidine deaminase)(FIG. 5-6). Removal of the two UGIs from BE4max to create BE4maxΔUGI resulted in an increase in C-to-G (and to a lesser degree C.-to-A) edits relative to wild-type BE4max when tested with seven different gRNAs targeted to sites with Cs at protospacer positions 5, 6, and 7 in HEK293T cells (FIG. 13). In general, C-to-G editing was observed with BE4maxΔUGI at Cs that were preceded by A, C, or T, with the most efficient editing generally observed with Cs at protospacer position 6 (FIG. 13). We also observed a substantially higher frequency of indels with BE4maxΔUGI relative to BE4max (FIGS. 13 & 15), consistent with the idea that this fusion is likely more efficient at creating abasic sites.¹ Reasoning that creation of an abasic site is important for increased C-to-G editing, we further hypothesized that adding human UNG (hUNG) enzyme to BE4maxΔUGI might enhance the frequency of desired edits. However, a BE4maxΔUGI-hUNG fusion possessed somewhat decreased C-to-G editing activity and did not induce appreciably changed frequencies of indels with the seven gRNAs tested (although it did show decreased C-to-T editing activity) (FIGS. 13 & 15). Similar results were obtained when hUNG was fused at the N-terminus of BE4maxΔUGI (FIG. 14). Fusion of UNG to ABEmax did not yield enhanced C-to-G editing compared to ABEmax (FIG. 14). We also tested a variety of CBEs that are based on non-APOBEC1 deaminase architectures, such as human A3A and enhanced A3A-BE3¹⁷, human AID-BE3¹⁵, and the Petromyzon marinus CDA1-based Target-AID², as well as variants thereof lacking UGIs and having added UNGs. Among this larger ensemble of variants, none consistently showed higher activity than the BE4maxΔUGI-hUNG editor (FIG. 16).

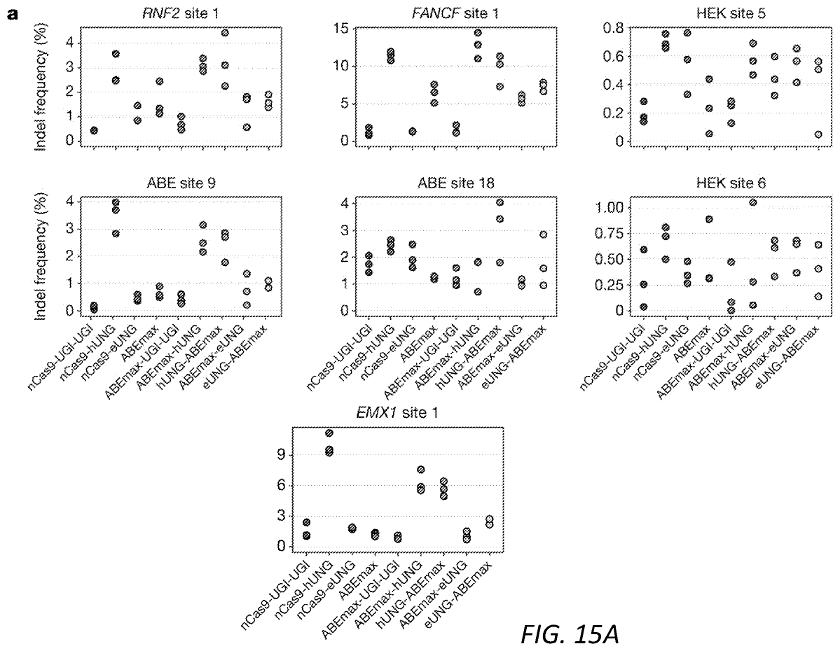

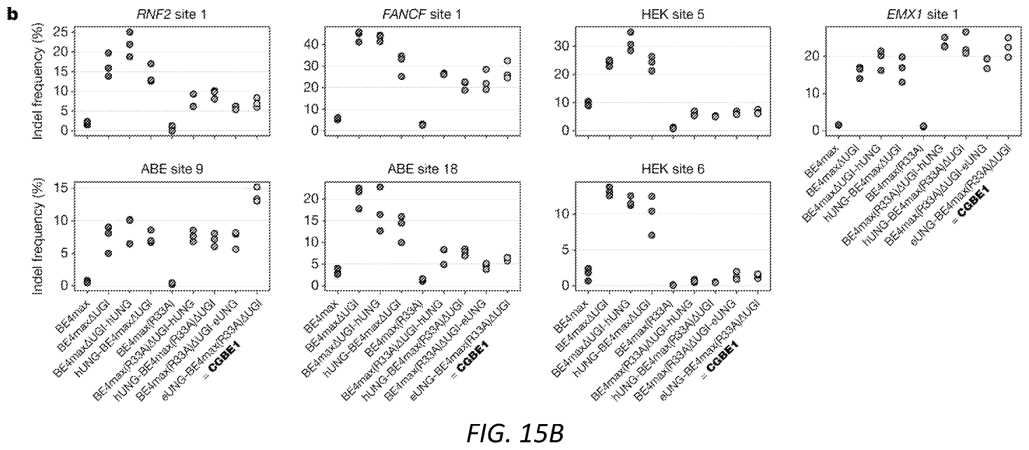

We also investigated whether introducing mutations into the APOBEC1 part of BE4maxΔUGI-hUNG might further increase the frequency of C-to-G editing. Although we do not have a mechanistic understanding of how C-to-G edits are induced, we reasoned that altering the deamination dynamics of APOBEC1 might also influence the editing outcome. We focused on the APOBEC1 R33A mutation, a substitution we previously showed can decrease off-target RNA editing while substantially preserving the efficiency and increasing the precision of on-target DNA editing by CBEs⁵. We found that introduction of R33A into BE4maxΔUGI-hUNG increased C-to-G editing frequencies with three of the seven gRNAs tested in HEK293T cells while leaving editing frequencies essentially unaltered with the other four (FIG. 13). The effect of the R33A variant was most striking with the FANCF site 1 gRNA, which had shown virtually no C-to-G editing with any of the other editors we tested but now showed a mean editing frequency of 14.0% (FIG. 13). Interestingly, BE4max(R33A)ΔUGI-hUNG on average showed lower indel byproducts with 6 out of 7 gRNAs compared to BE4maxΔUGI-hUNG (FIG. 15).

We additionally explored whether replacing the hUNG present in the BE4max(R33A)ΔUGI editor with an orthologous UNG from Escherichia coli (eUNG) might further increase the efficiency of C-to-G edits. We created two additional editors: BE4max(R33A)ΔUGI-eUNG or eUNG-BE4max(R33A)ΔUGI with an eUNG added to the carboxy- or amino-terminal ends, respectively. Testing of these fusions in HEK293T cells revealed that both induced C-to-G edits with higher frequencies than BE4max(R33A)ΔUGI-hUNG for six out of seven gRNAs tested (mean editing frequencies ranging from 3.3-57.0% and 8.5-62.6% for BE4max(R33A)ΔUGI-eUNG and eUNG-BE4max(R33A)ΔUGI, respectively) (FIG. 13). Indel frequencies with both fusions were generally comparable to those observed with BE4max(R33A)ΔUGI-hUNG (FIG. 15). Given its higher C-to-G editing activity, we chose the eUNG-BE4max(R33A)ΔUGI fusion (hereafter referred to as C-to-G Base Editor 1 (CGBE1)) for additional characterization.

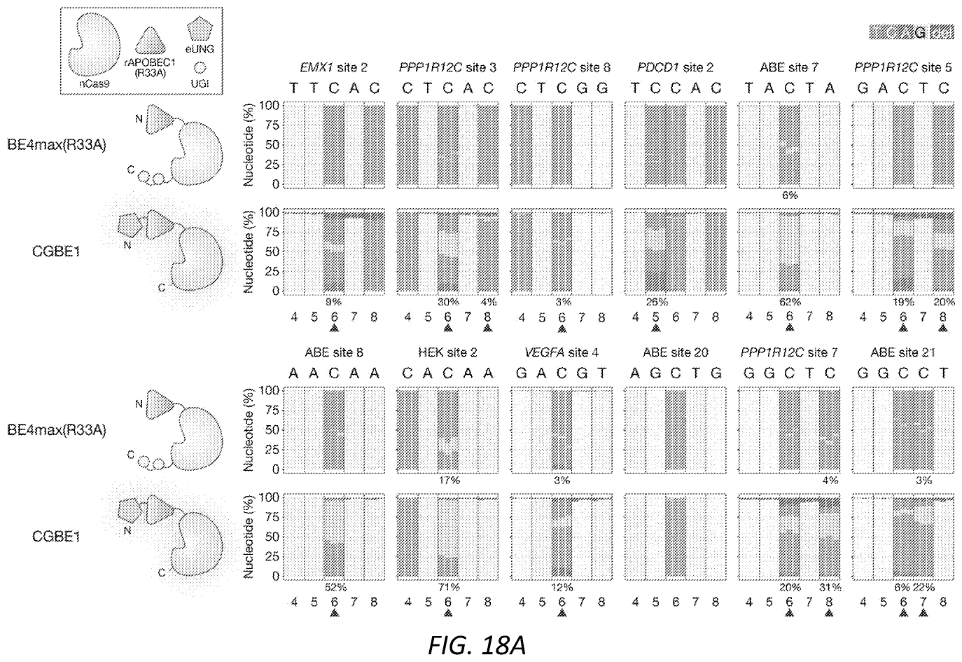

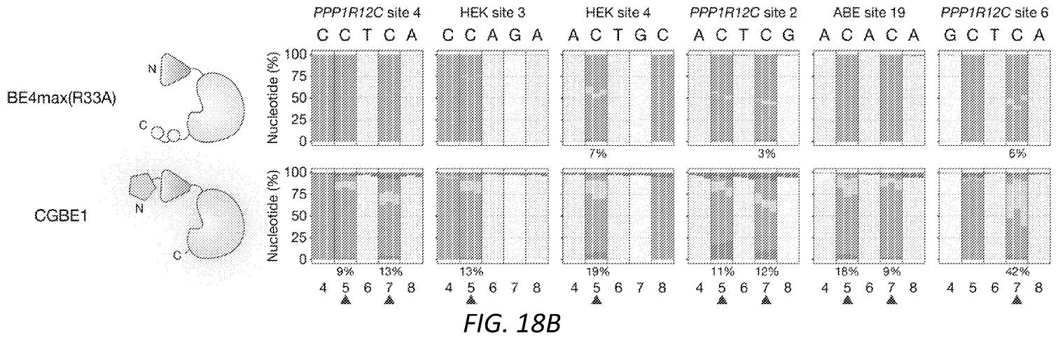

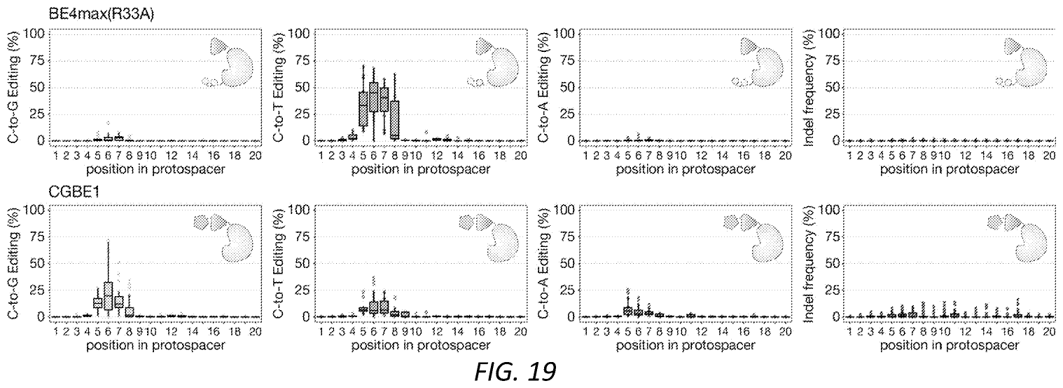

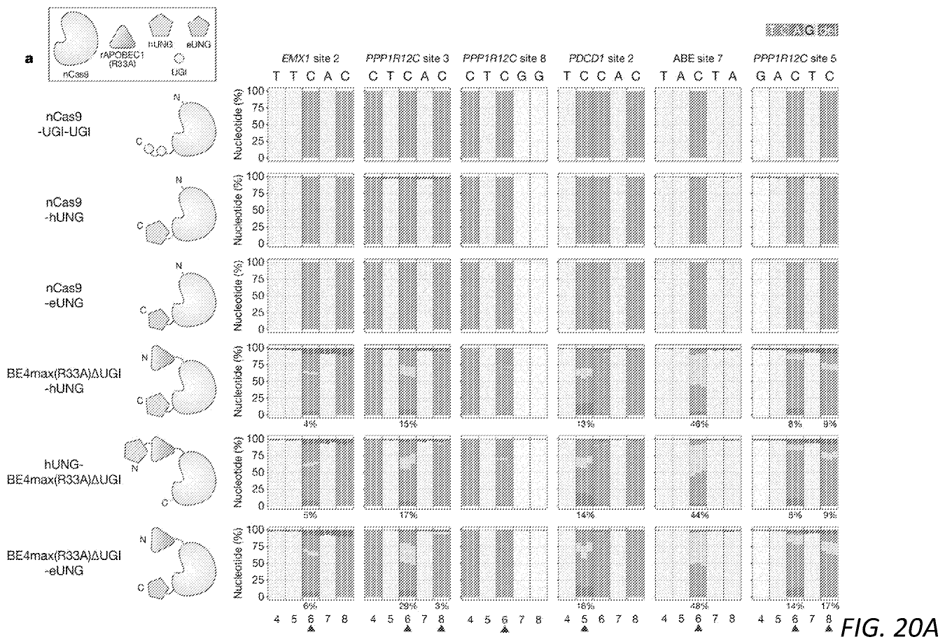

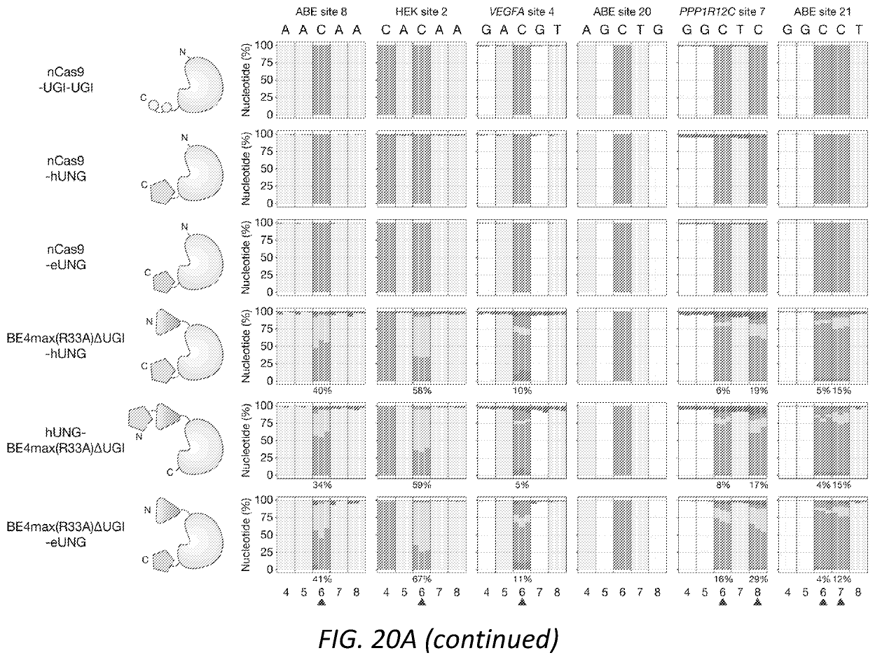

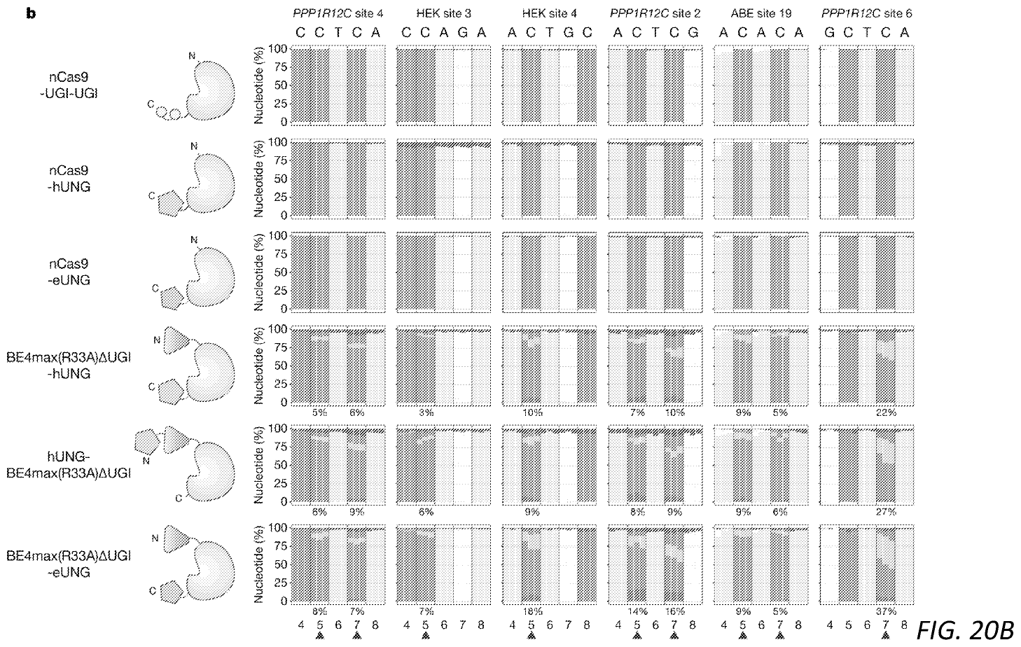

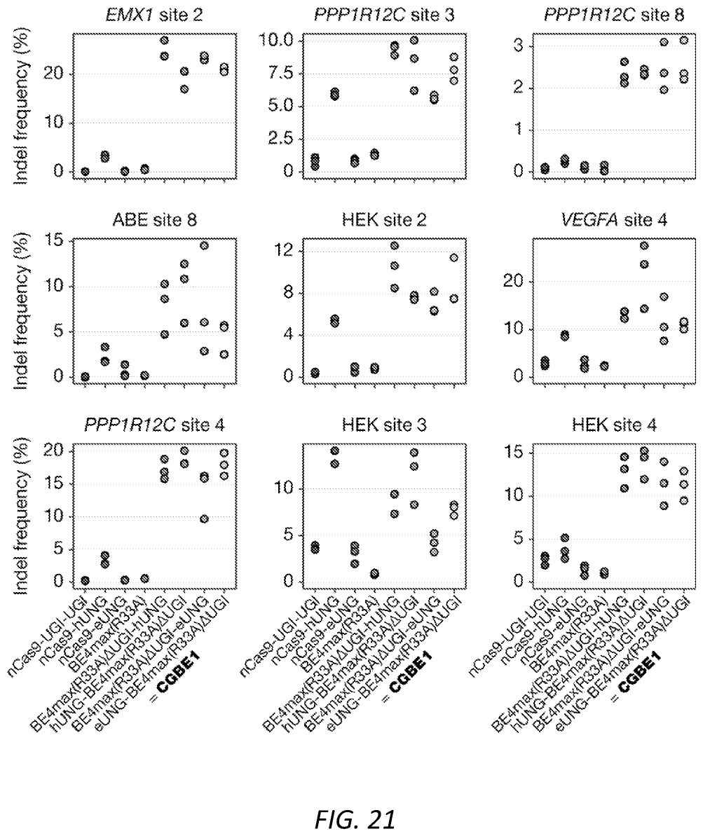

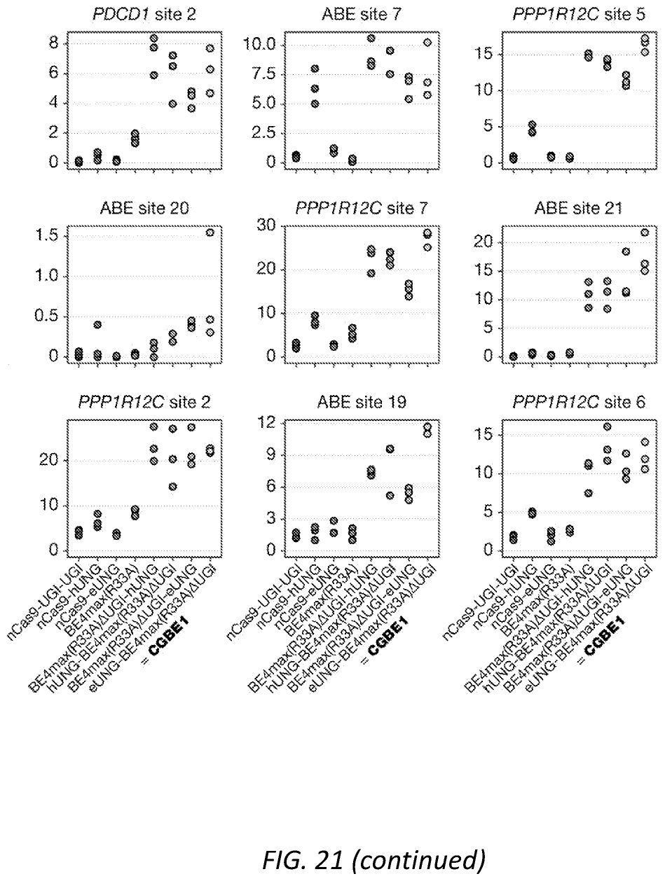

To more comprehensively characterize CGBE1, we tested its activity with 18 additional gRNAs in human HEK293T cells. 12 of the sites targeted by these 18 gRNAs have a C at position 6 (“C6-sites”) (FIGS. 18 & 20) and 6 have a C at positions 4, 5, 7, or 8 (“non-C6-sites”) (FIGS. 18 & 20). For 16 of the 18 sites, CGBE1 induced C-to-G edits with substantially higher frequencies than what was observed with its parental CBE control (BE4max(R33A)) (FIG. 18). Highly efficient C-to-G edits were observed for 4 of the 18 sites (ABE site 7, ABE site 8, HEK site 2, and PPP1R12C site 6), with mean editing frequencies ranging from 41.7 to 71.5% (FIG. 18). C-to-G edits were by far the most efficiently induced edits at these 4 sites with only very low levels of C-to-T or C-to-A byproducts observed (FIG. 18). C-to-G was also the most efficiently induced edit for 6 additional sites albeit at lower frequencies (three C6-sites and three non-C6-sites) (FIG. 18). In total, when combined with the results obtained with the initial seven gRNAs described above (FIG. 13), CGBE1 induced C-to-G editing with mean frequencies of 20% or higher at 14 of the 25 sites tested (FIGS. 13 & 18). Notably, C-to-G editing was most efficient for Cs embedded in an AT-rich sequence context (FIGS. 13 & 18). Analysis of the spatial distribution of editing across all 25 sites tested shows that the mean frequency of C-to-G editing was highest at position 6 and that indels were distributed throughout the length of the protospacer (FIG. 19).

### Example 3. Characterization of miniCGBE1 and its Side by Side Comparison with CGBE1

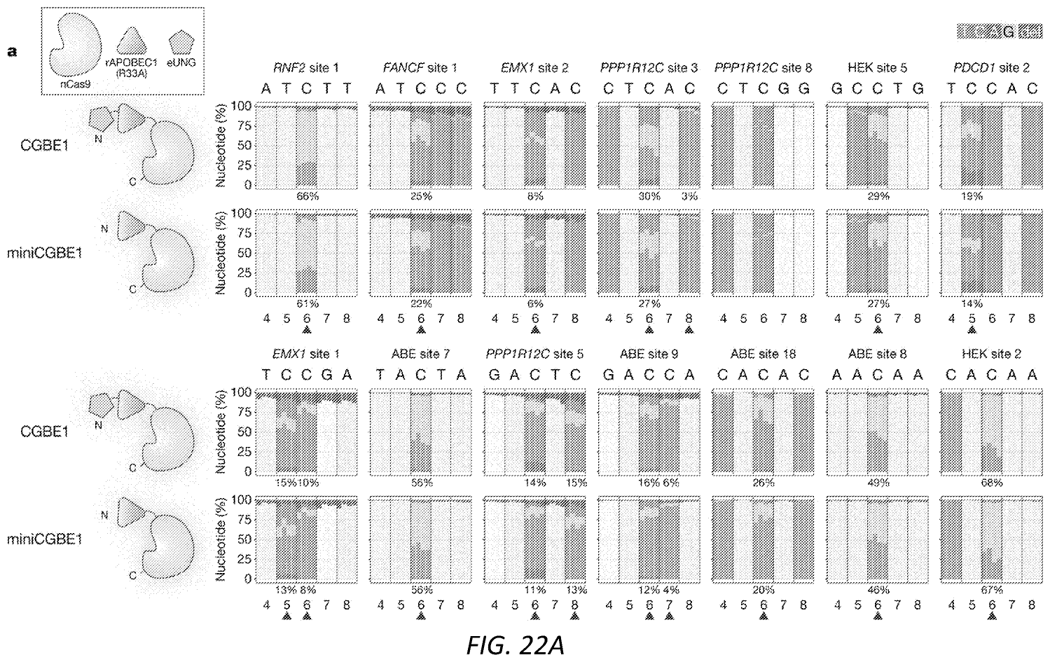

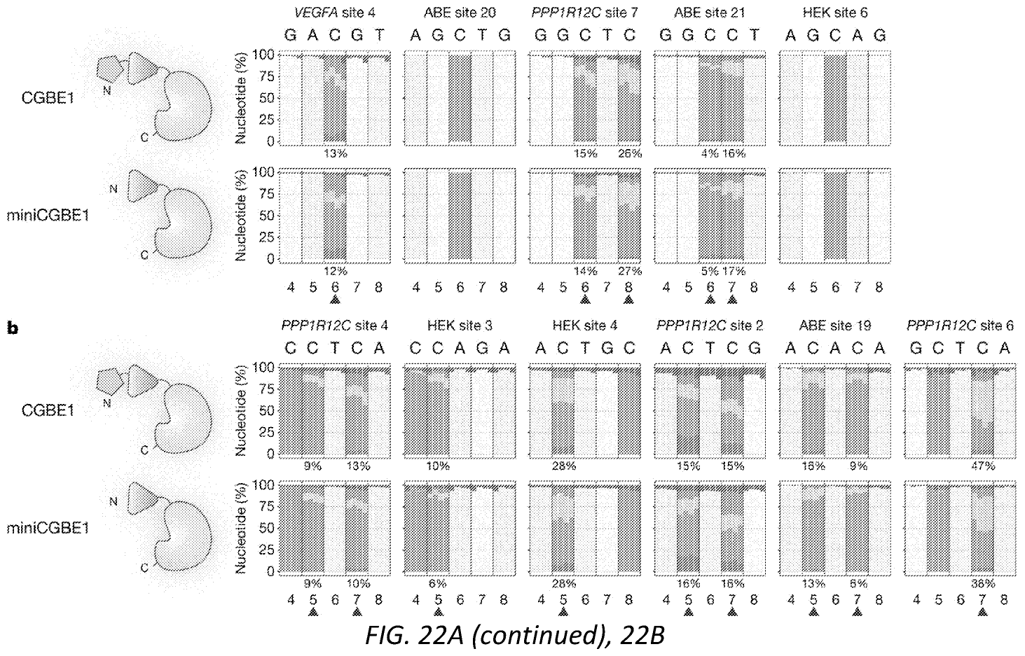

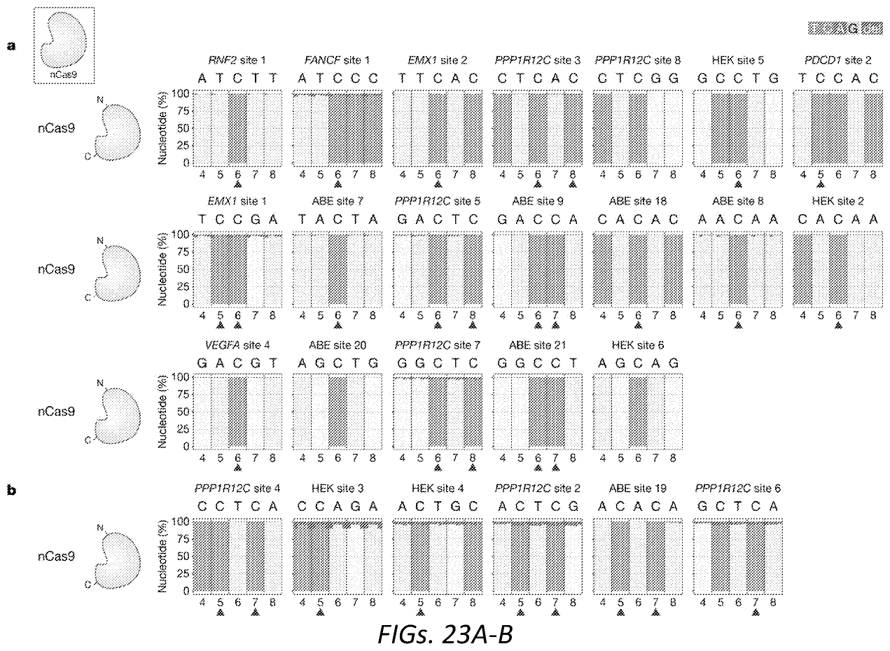

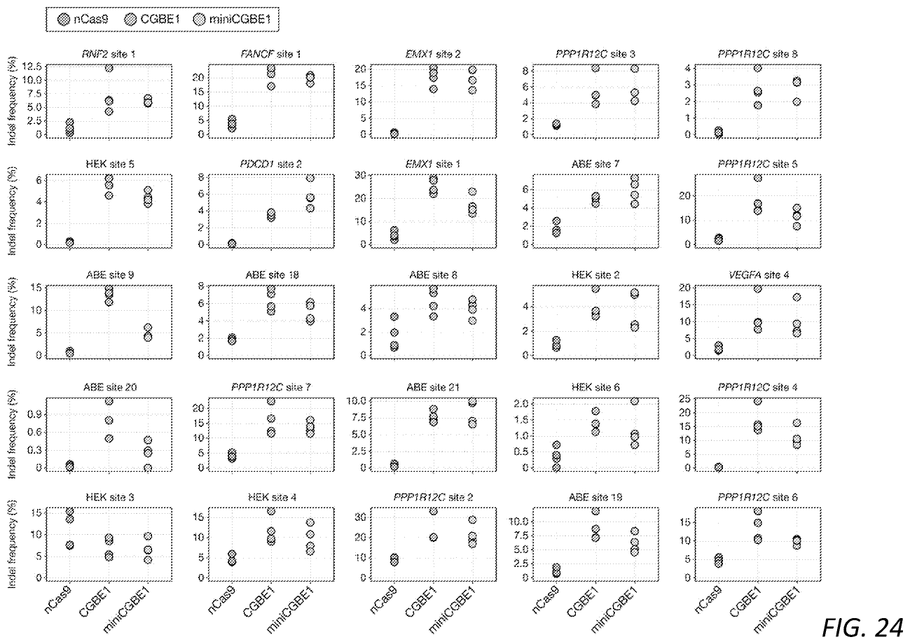

We explored the impact of deleting the eUNG domain from the CGBE1 editor on its activity. This particular editor architecture, which we named miniCGBE1 (FIG. 22), had not been made or tested over the course of the stepwise progression from BE4max to CGBE1 and also has the added advantage of being smaller in size. Side-by-side comparisons of miniCGBE1 with CGBE1 at the same 25 sites we had previously tested showed that the frequencies of editing observed with miniCGBE1 were comparable but moderately lower at 6 out of 25 sites tested (mean editing frequencies across all 25 sites of 14.4% and 13% with CGBE1 and miniCGBE1, respectively), whereas the indel frequencies induced by miniCGBE1 were lower at 15 out of 25 sites (mean indel frequencies of 10.4% and 8.5% for CGBE1 and miniCGBE1, respectively; FIG. 22-24).

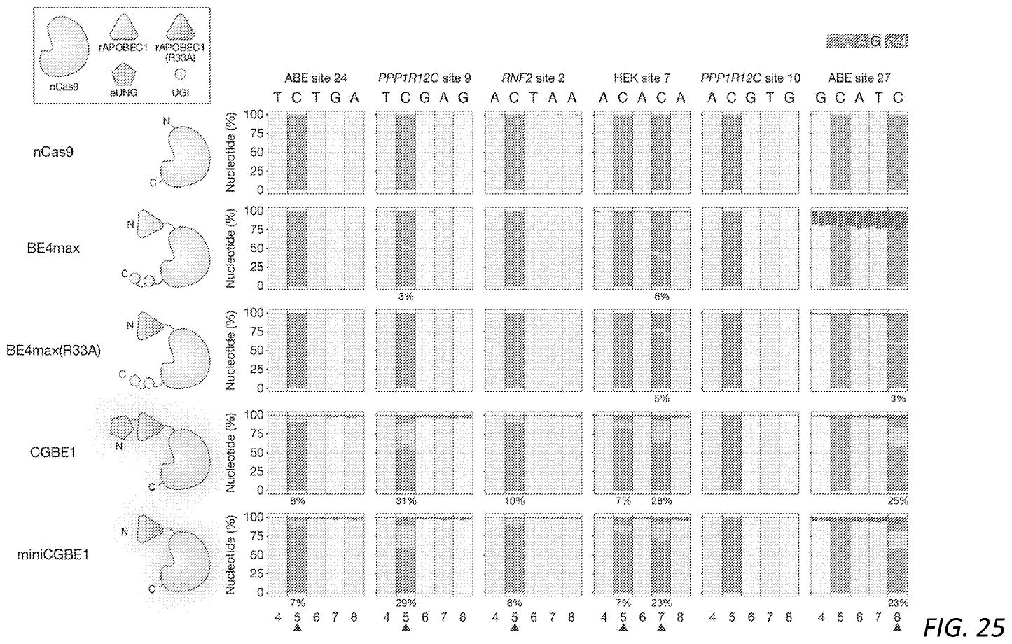

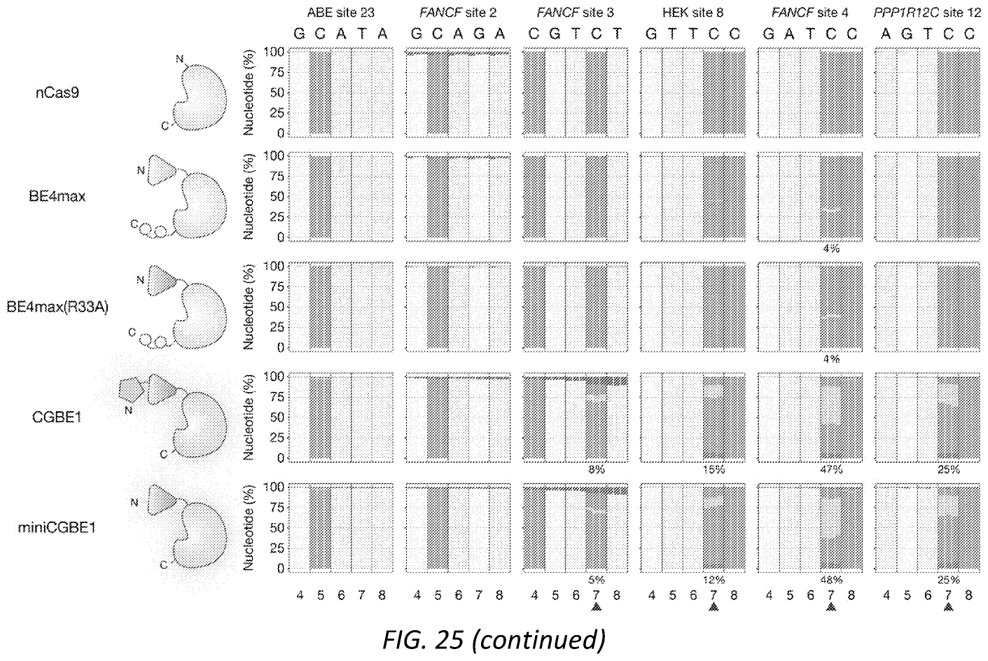

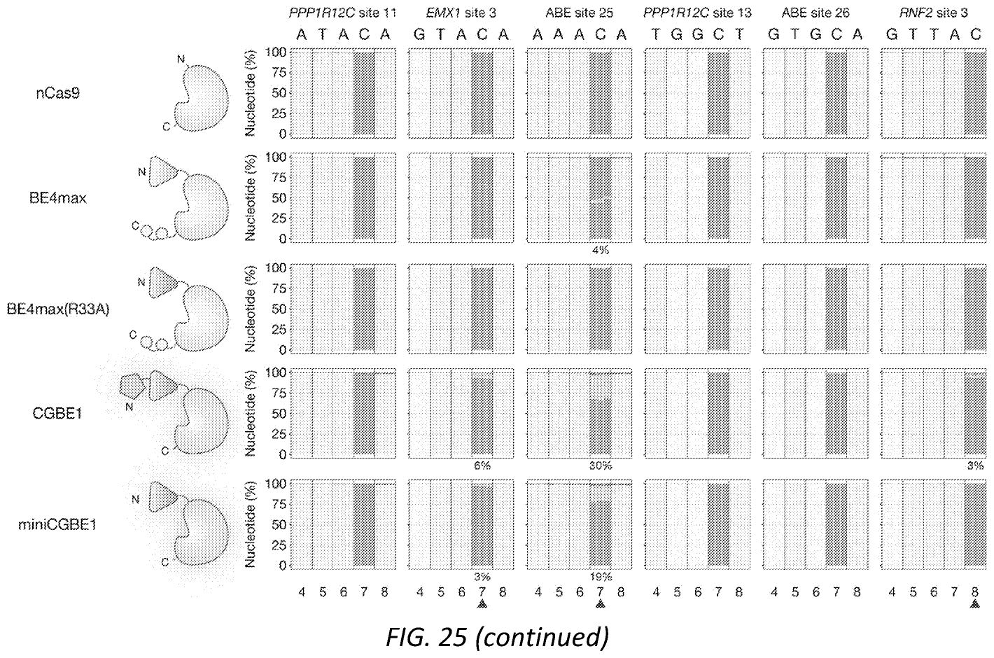

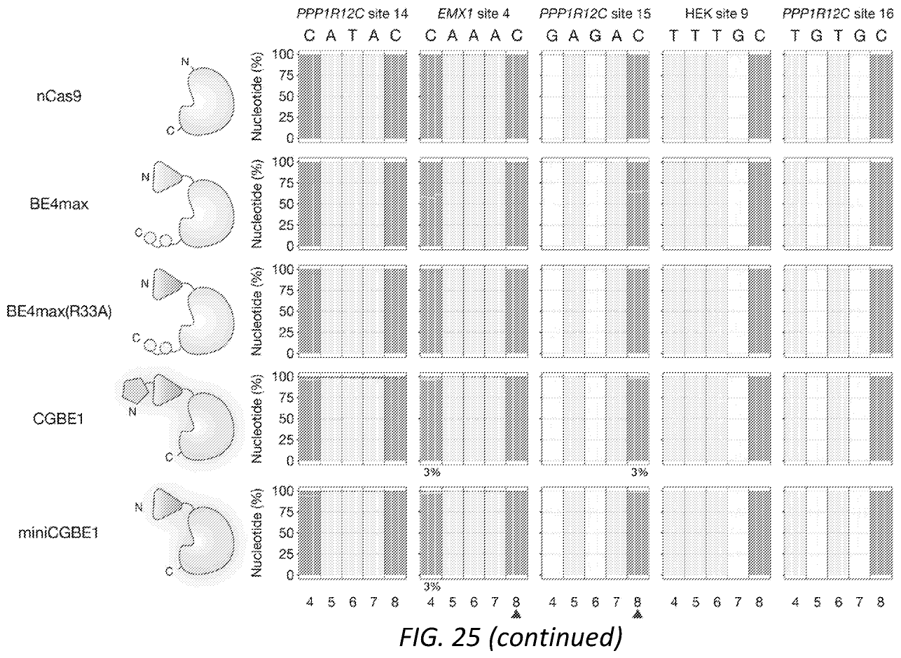

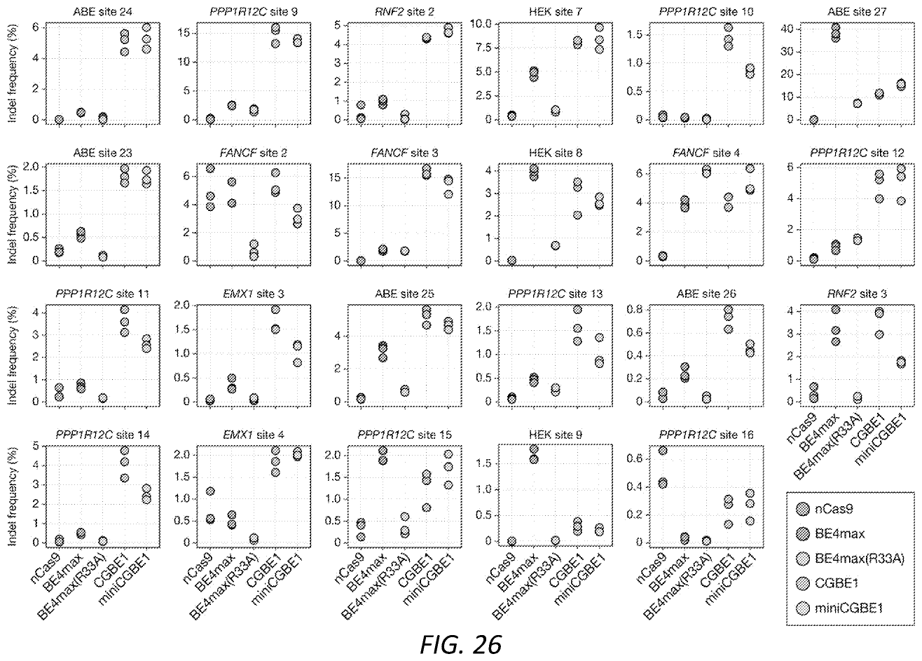

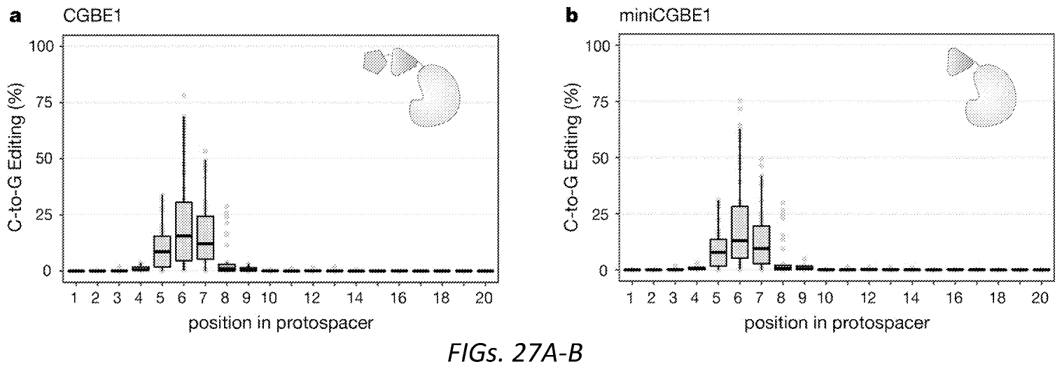

To more fully characterize the positional preferences within the editing windows of CGBE1 and miniCBGE1, we tested these two editors side-by-side with BE4max and BE4max(R33A) using 23 additional gRNAs that target sites with cytosines at protospacer positions 4, 5, 7, and 8 (FIG. 25). The targets of these 23 gRNAs included six sites with a C5, five with a C7, four with a C8, and eight with two Cs at various positions (C4 and C7, C4 and C8, C5 and C7, C5 and C8, and C7 and C8). Mean editing frequencies induced by miniCGBE1 were comparable to those of CGBE1: 1.7% and 1.5% at C4, 7.3% and 6.7% at C5, 16.0% and 13.5% at C7 and 3.4% and 2.9% at C8 for CGBE1 and miniCGBE1, respectively (FIG. 25). In addition, indel frequencies induced by CGBE1 and miniCGBE1 were comparable at 10 sites, lower with CGBE1 at five sites, and lower with miniCGBE1 at eight sites (FIG. 26). Collectively, our testing of CGBE1 and miniCGBE1 with 48 different gRNAs demonstrates that both have an optimal editing window for cytosines at positions 5-7 in the protospacer with those at position 6 being edited most efficiently (FIG. 27). This finding is consistent with our previously published studies showing that a CBE with the APOBEC1-R33A variant edits optimally on positions 5-7 of the protospacer and more weakly on positions 4 and 87.

### Example 4. Evaluation of DNA Off-Target Editing Induced by CGBE

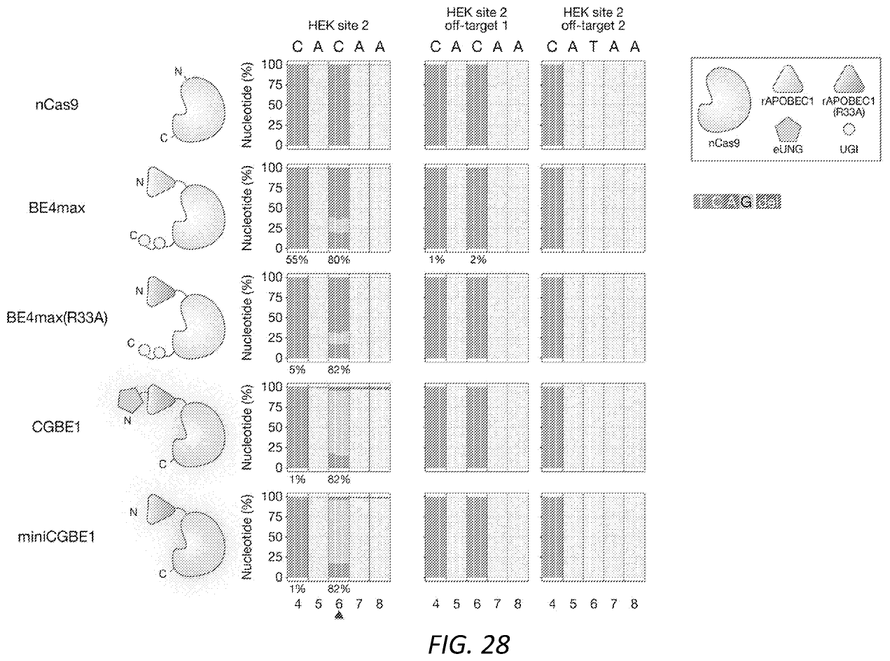

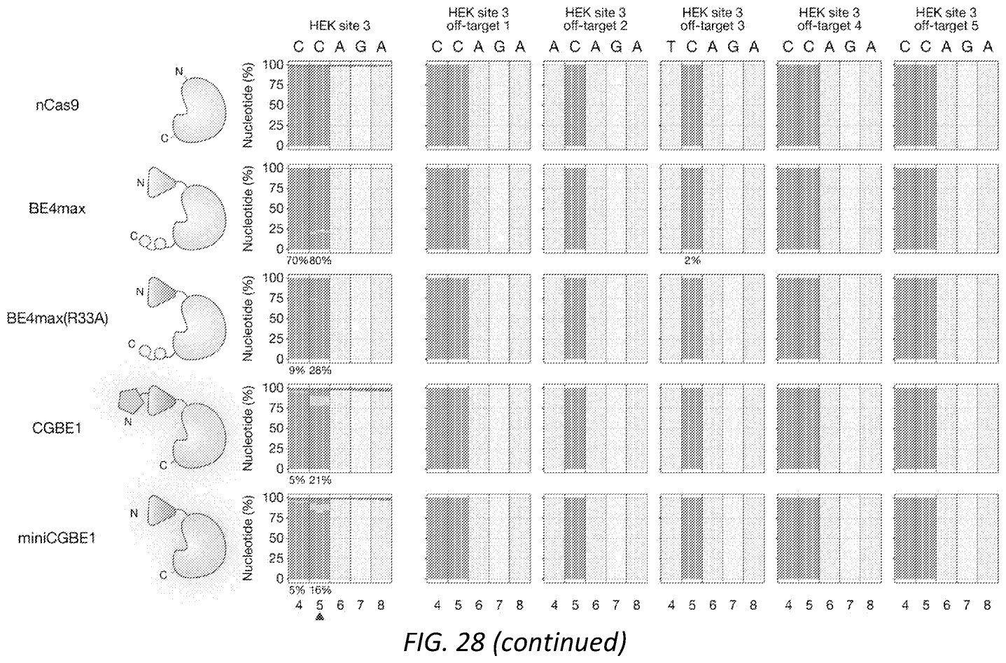

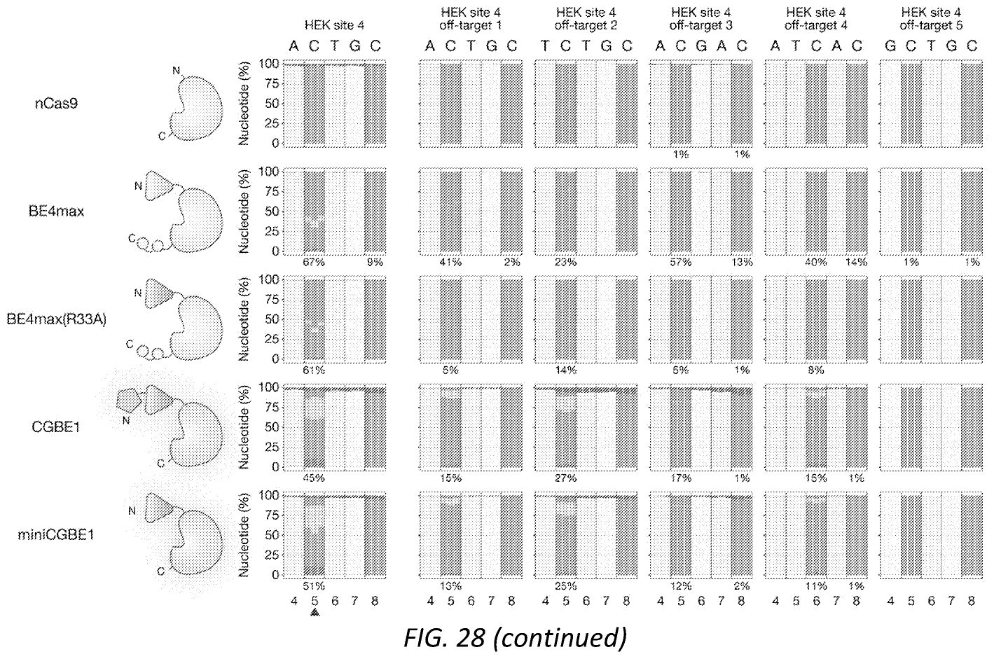

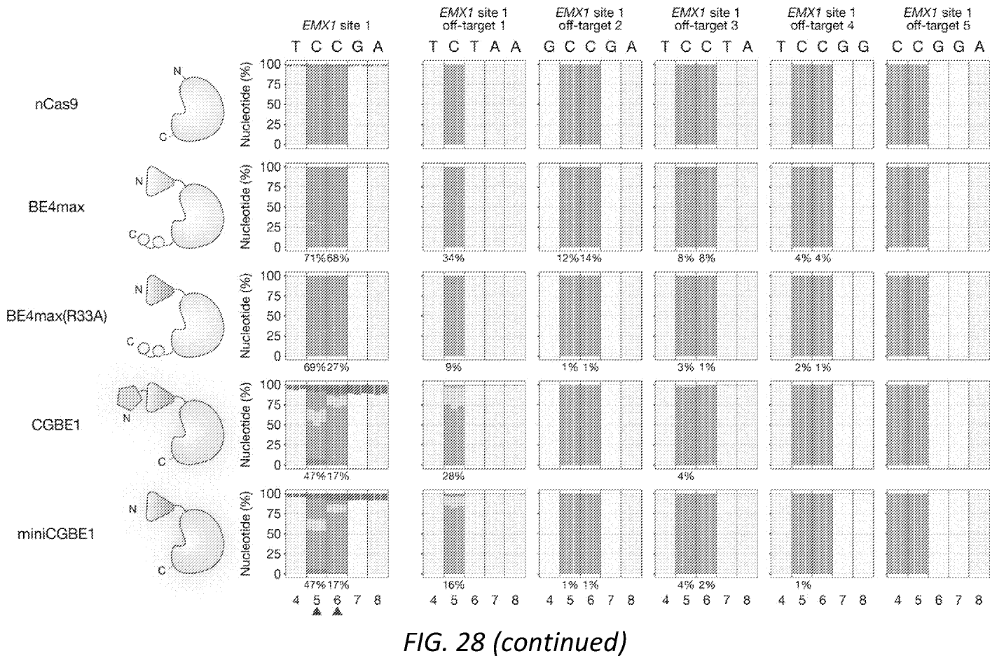

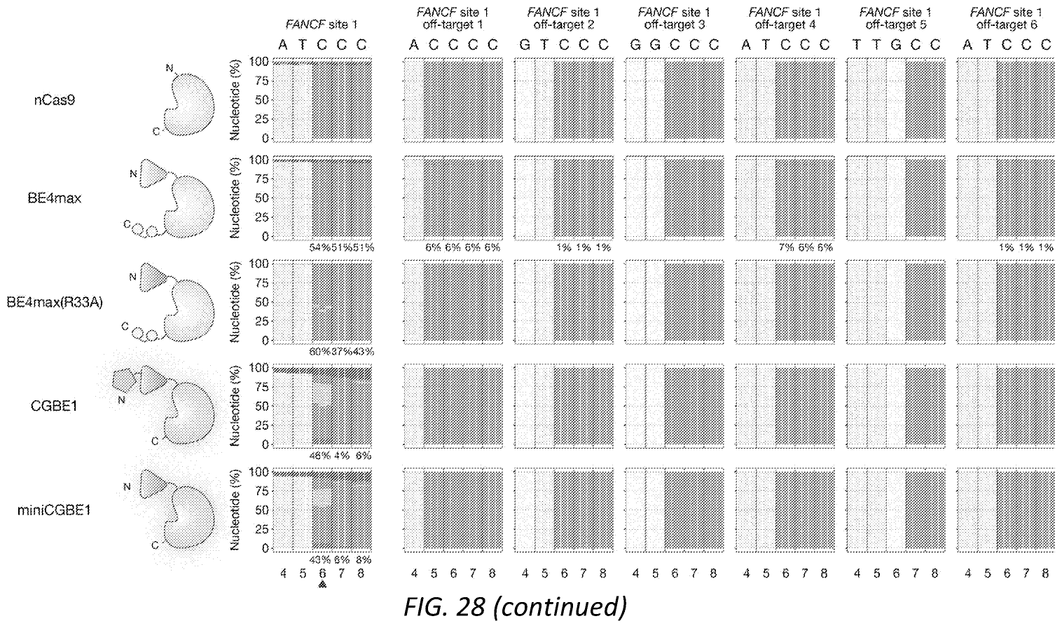

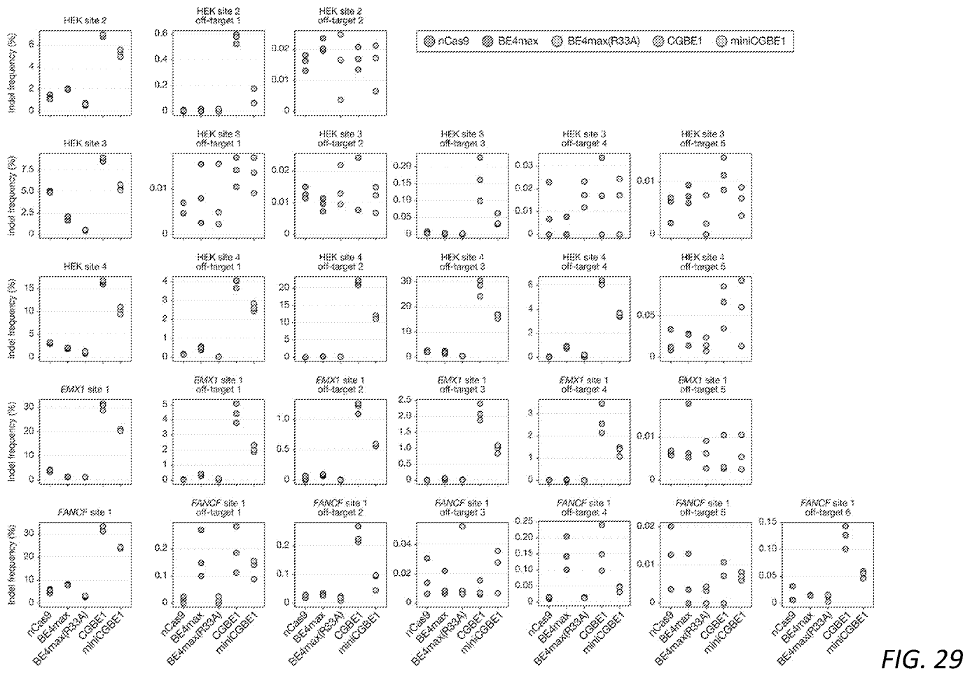

Cas9-dependent DNA off-target profiles of CGBEs was assessed by transfecting HEK 293T cells with nCas9 control, BE4max, BE4max(R33A), CGBE1, and miniCGBE1 using HEK site 2, HEK site 3, HEK site 4, EMX1 site 1, and FANCF site 1 gRNAs. 23 genomic sites that have previously been described as known off-target sites for said gRNAs (Tsai et al, NBT 2014) were sequenced with NGS to detect potential off-target base editing of CGBE constructs. BE4max induced C-to-D (D=A, G, or T) edits at 15 of the 23 off-target sites with BE4max-R33A inducing edits less efficiently at all 15 sites, consistent with previously published observations that introduction of R33A reduces Cas9-dependent DNA off-target edits by the BE3 CBE (FIG. 28). Similarly, both CGBE1 and miniCGBE1 showed lower C-to-D off-target editing at 14 out of the 15 off-target sites that were edited by BE4max (FIG. 28). As expected, off-target indel frequencies were higher with CGBE1 and miniCGBE1 relative to BE4max at 18 out of 23 sites, although miniCGBE1 again showed reduced activity compared with CGBE1 at 14 out of these 18 sites (FIG. 28). Overall, this assessment of Cas9/gRNA-dependent DNA off-target editing shows that CGBE1 and miniCGBE1 induce fewer off-target DNA edits than BE4max, that CGBE-induced indels can occur at off-target sites, and that indels are reduced with miniCGBE1 relative to CGBE1.

### Example 5. CGBEs with SpCas9-NG and SpCas9-VRQR Variants of are Functional

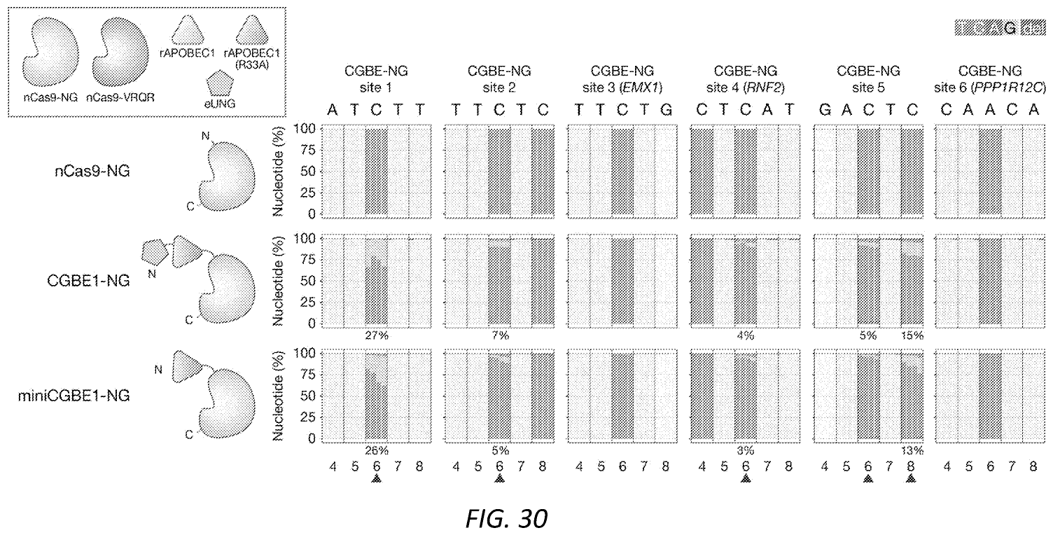

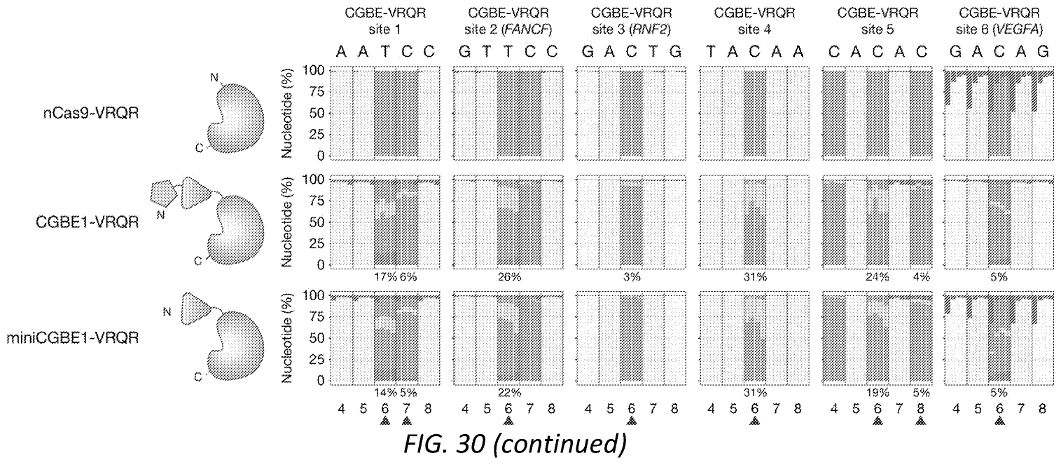

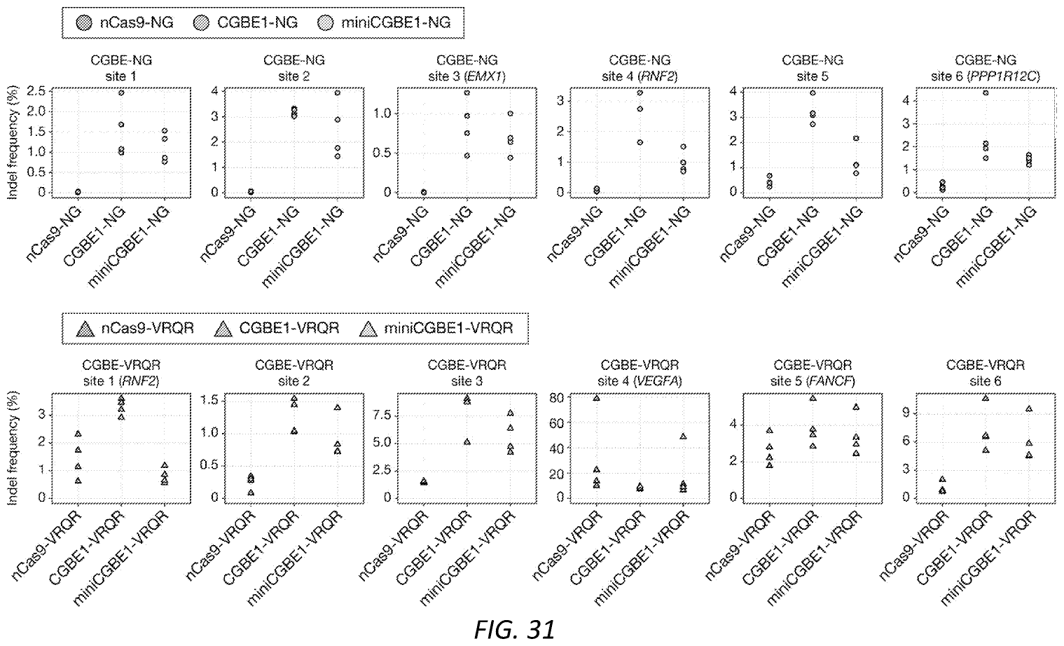

We tested whether we could improve the somewhat more restricted targeting range of CGBEs by using previously described SpCas9-NG and SpCas9-VRQR variants that recognize shorter NG¹⁹ and alternative NGA²⁰ PAMs, respectively. We targeted six sites with NGT PAMs using modified CGBE1-NG and miniCGBE1-NG variants and six sites with NGAG PAMs using CGBE1-VRQR and miniCGBE1-VRQR variants. Each of these 12 new sites have a cytosine at position 6 embedded within an AT-rich sequence context to provide an optimal target for C-to-G editing (FIG. 30). On these target sites, CGBE1-NG and miniCGBE1-NG induced C-to-G edits with frequencies as high as 27% and 26%, respectively, and CGBE1-VRQR and miniCGBE1-VRQR induced C-to-G edits with frequencies of up to 31% (FIG. 30). These results show that the targeting range of CGBE constructs can be expanded by using Cas9 variants with altered or relaxed PAM recognition specificities.

### Example 6. Comparison of CGBEs with Prime Editor Technologies

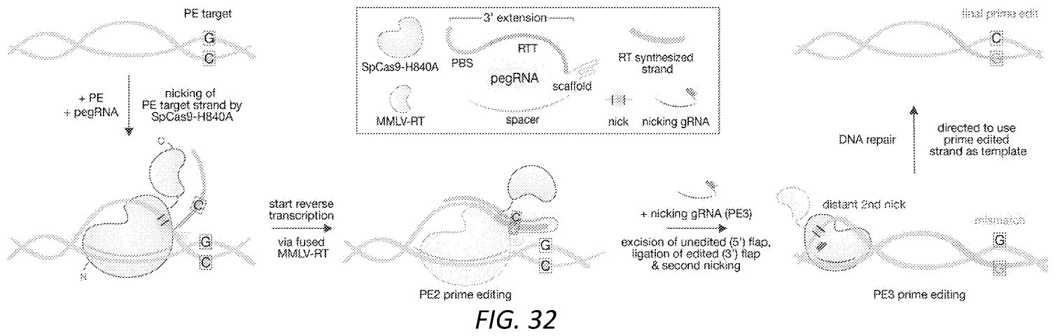

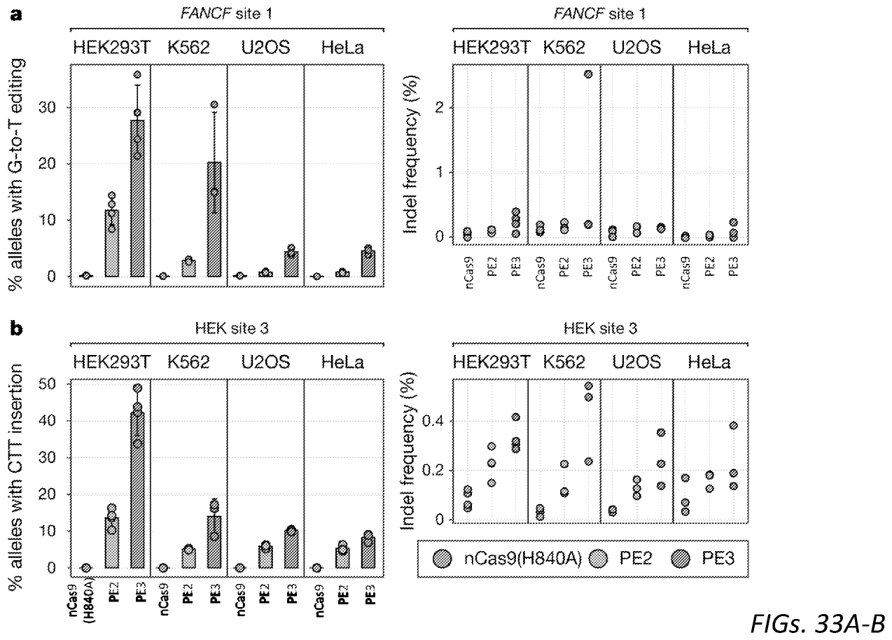

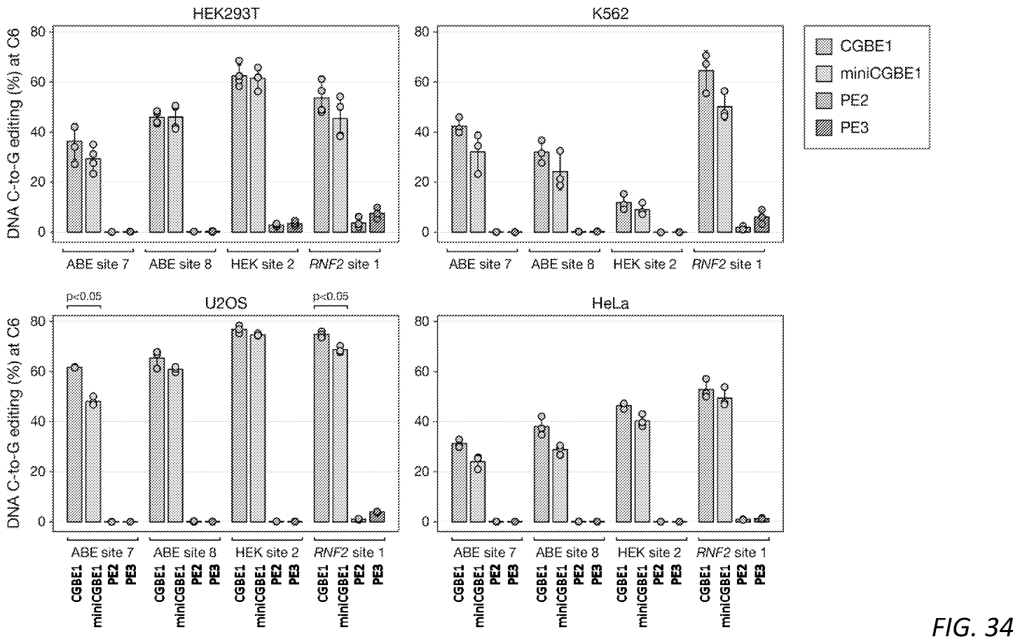

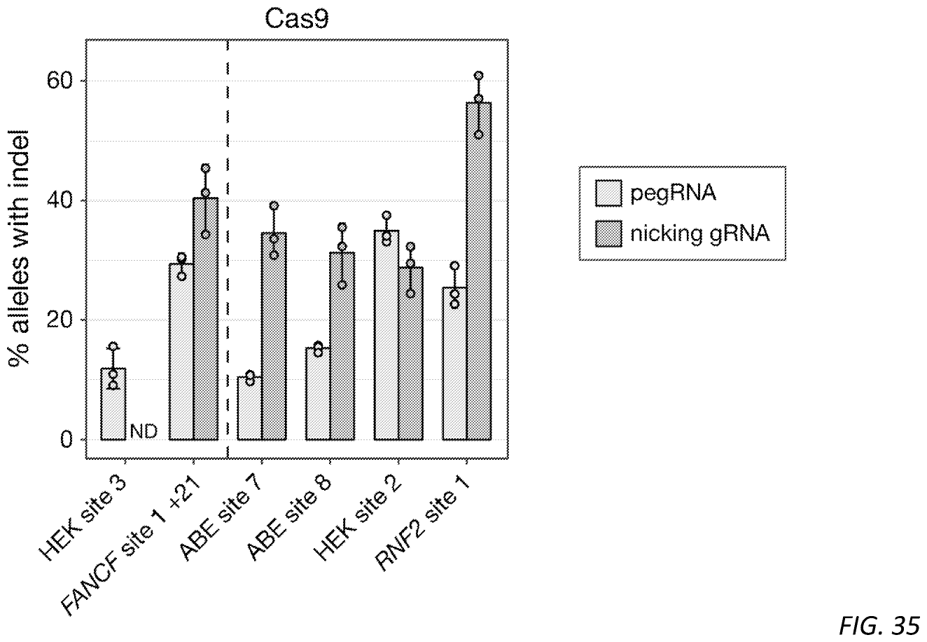

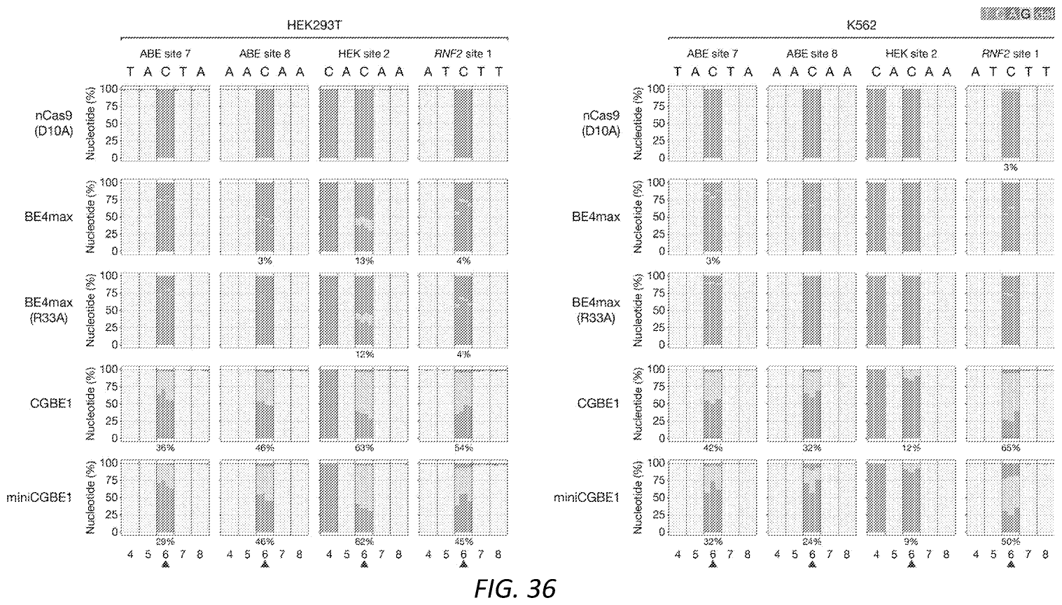

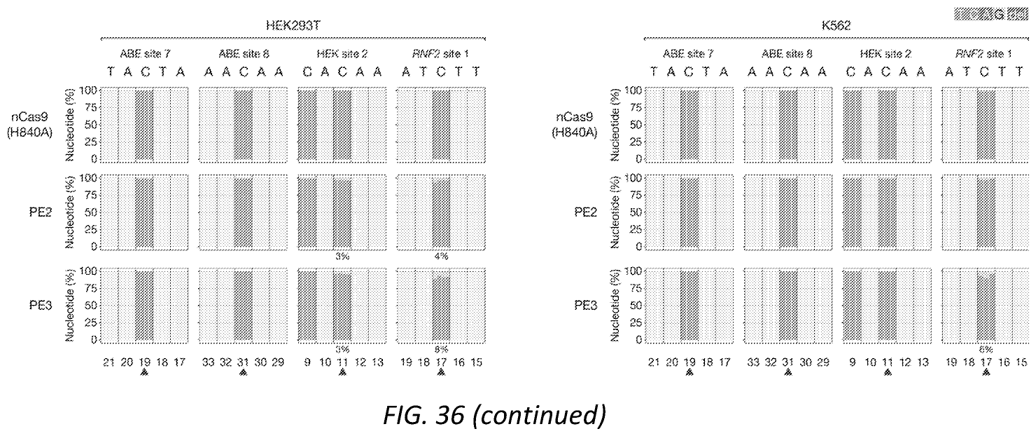

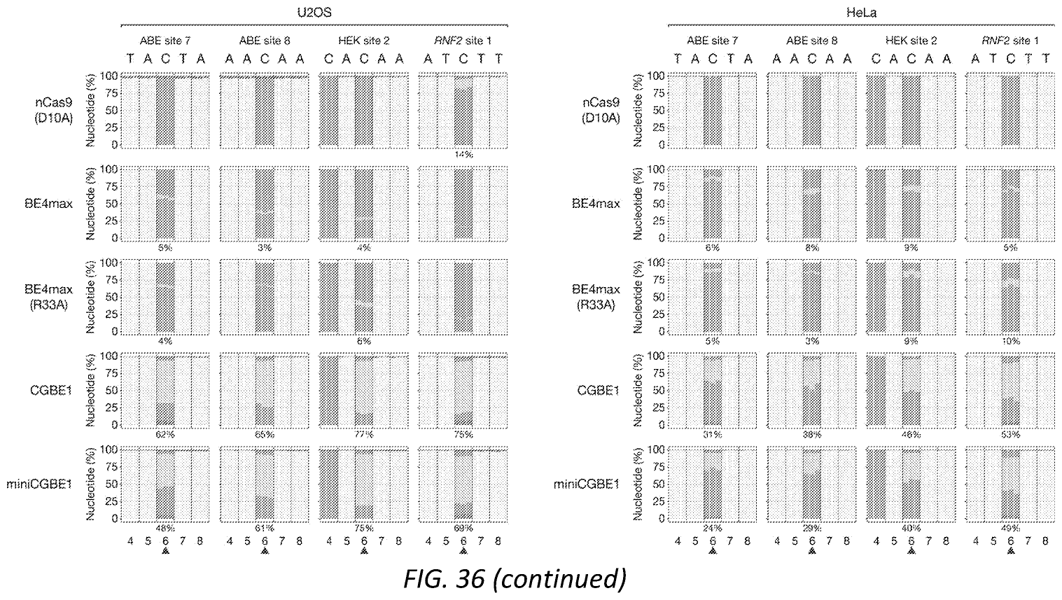

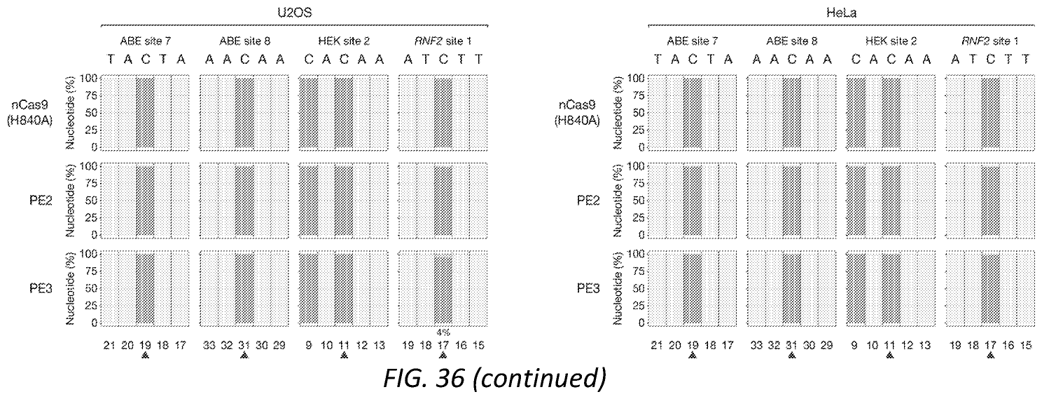

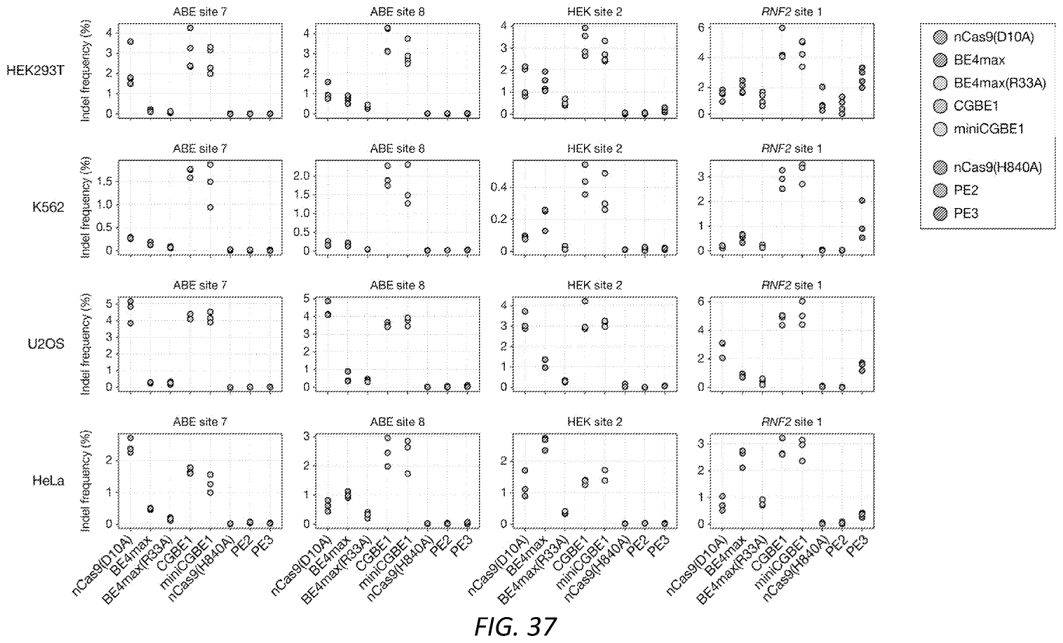

We compared our CGBEs with Prime Editing (PE) methods that can introduce a diverse range of different edits and that were published15 while we were completing this project. The PE2 system uses two components: (1) a Prime Editor fusion protein and (2) a prime editing gRNA (pegRNA) (FIG. 32).²¹A more efficient PE3 system adds a secondary “nicking gRNA” (ngRNA) that directs a nick to the DNA strand opposite the edited one, thereby increasing editing efficiency (FIG. 32).²¹ We performed side-by-side comparisons of our CGBEs with PE2 and PE3 systems for making four different C-to-G edits, assessing frequencies of these alterations across four different human cell lines (HEK293T, K562, U205, and HeLa cells). Positive control experiments we performed in all four cell lines re-confirmed that two other previously described pegRNAs could induce a G-to-T transversion in FANCF site 1 and a CTT insertion in HEK site 3 (CTT-insertion), that PE3 is more efficient than PE2, and that the highest prime edit frequencies were observed in HEK293T cells (FIG. 33). For all four C-to-G edits (which we had already established could be efficiently induced by our CGBEs in HEK293T cells), we found that both PE2 and PE3 were substantially less efficient than CGBE and miniCGBE1 across all four cell lines (FIGS. 34 & 36). Importantly, these data also establish that our CGBEs can function robustly and efficiently across multiple human cancer cell lines. In addition, we found that the frequencies of unwanted indels were lower with prime editors compared to the CGBEs in all four cell lines (FIG. 37). To rule out that the pegRNAs and ngRNAs we designed were inactive or unable to interact with Cas9, we tested their abilities to induce Cas9-mediated indels at their intended target sites in HEK293T cells (note that we could not assess the activity of the HEK site 3 ngRNA due to its overlap with a required PCR primer). The indel frequencies we observed with these pegRNAs and ngRNAs were comparable to those used with the two positive control target sites (FIG. 35).

### Example 7. mRNA and RNP Production of CGBEs and Testing in Primary Human CD34+ and T Cells

CGBE architectures described in FIGS. 6-9 will be tested in primary human CD34+ and T cells by electroporating CGBE mRNAs (produced via IVT or by TriLink). CGBE constructs will be subcloned into pET vectors with an N-terminal 6×His-tag and codon-optimized for expression in E. coli to enable protein purification. RNPs will be electroporated with a Lonza device into HEK293T and primary human T cells to determine if CGBE RNP delivery yields efficient ex vivo DNA transversion base editing.

### Example 8. Evaluation of RNA Off-Target Editing Induced by CGBE

Unbiased detection of RNA off-target editing with the help of RNA-seq will be assessed. Cells will be transfected with two different gRNAs and CGBE constructs that are co-translationally expressed with P2A-EGFP in 15 cm dishes and trypsinized 36 hours post-transfection. Subsequently, GFP+ cells will be sorted on a BD FACSAria II and lysed to harvest both DNA and RNA. After efficient on-target editing is confirmed via targeted amplicon sequencing, RNA-seq will be performed using a TruSeq stranded total RNA library prep and sequencing on a NextSeq 500 machine at the MGH or a NovaSeq at the Broad Institute.

### Example 9. Evaluation of UNG Recruitment Strategy Using Peptide Aptamers

Next generation CGBE constructs fused with the candidate peptide aptamers will be assessed by transfection experiments, for example, those using lipofection and nucleofection techniques into human cells such as HEK 293T, U2OS and K562 cell lines. The transfections will be carried out with gRNA constructs with spacer sequences targeting human genomic loci having cytosines in the editing windows that is generated by our CGBE constructs. 72 hours post-transfection, genomic DNA (gDNA) will be harvested, and target loci will be PCR amplified. PCR amplicons will be subjected to targeted next generation sequencing (NGS) to quantify on-target editing efficiencies. The DNA off-target activities of the next generation CGBE constructs will be assessed by analyzing the top in-silico predicted candidate off-target sites using targeted amplicon sequencing (NGS) using the treated gDNAs. In order to assess the potential RNA off-target activities of our next generation CGBE constructs, we will be harvesting total RNA in parallel in the treated cells in order to conduct stranded libraries for transcriptome-wide analysis via RNA sequencing (RNA-seq).

### Example 10. Evaluation of UNG Recruitment Strategy Using RNA Aptamers

The next generation CGBE constructs will be analyzed using RNA aptamers fused to the gRNA in a series of transfection experiments (using, for example, lipofection and nucleofection techniques) in human cells such as HEK 293T, U2OS and K562 cell lines. The transfections will be carried out with fusion gRNA constructs with spacer sequences targeting human genomic loci having cytosines in the editing windows generated by our CGBE constructs. 72 hours post-transfection, genomic DNA (gDNA) will be harvested, and target loci will be PCR amplified. PCR amplicons will be subjected to targeted next generation sequencing (NGS) to quantify on-target editing efficiencies. In order to test the potential DNA off-target activities of our next generation CGBE constructs, the top in-silico predicted candidate off-target sites will be analyzed with targeted amplicon sequencing (NGS) using the treated gDNAs. In order to assess the potential RNA off-target activities of our next generation CGBE constructs, we will be harvesting total RNAs in parallel in the treated cells in order to conduct transcriptome-wide analysis via RNA sequencing (RNA-seq).

### Example 11. Evaluation of UNG Recruitment Strategy Using Fabs, scFVs or sdABs

Next generation CGBE constructs fused with the candidate Fab, scFv, or sdAb, will be assessed in a series of transfection experiments (e.g., using lipofection or nucleofection techniques) in human cells such as HEK 293T, U2OS and K562 cell lines. The transfections will be carried out with gRNA constructs with spacer sequences targeting human genomic loci having cytosines in the editing windows generated by CGBE constructs. 72 hours post-transfection, genomic DNA (gDNA) will be harvested, and target loci will be PCR amplified. PCR amplicons will be subjected to targeted next generation sequencing (NGS) to quantify on-target editing efficiencies. DNA off-target activities of the next generation CGBE constructs will be assessed by analyzing the top in silico predicted candidate off target sites using targeted amplicon sequencing (NGS). In order to assess the potential RNA off-target activities of our next generation CGBE constructs, we will be harvesting total RNA in parallel in the treated cells in order to conduct transcriptome-wide analysis via RNA sequencing (RNA-seq).

### REFERENCES

1. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
2. Nishida, K. et al. Targeted nucleotide editing using hybrid prokaryotic and vertebrate adaptive immune systems. Science (80-). (2016). doi:10.1126/science.aaf8729
3. Gaudelli, N. M. et al. Programmable base editing of AT to GC in genomic DNA without DNA cleavage. Nature 551, 464-471 (2017).
4. Rees, H. A. & Liu, D. R. Base editing: precision chemistry on the genome and transcriptome of living cells. Nat. Rev. Genet. (2018). doi:10.1038/s41576-018-0059-1
5. Grünewald, J. et al. Transcriptome-wide off-target RNA editing induced by CRISPR-guided DNA base editors. Nature (2019). doi:10.1038/s41586-019-1161-z
6. Zhou, C. et al. Off-target RNA mutation induced by DNA base editing and its elimination by mutagenesis. Nature 571, 275-278 (2019). Rees, H. A., Wilson, C., Doman, J. L. & Liu, D. R. Analysis and minimization of cellular RNA editing by DNA adenine base editors. Sci. Adv. 5, eaax5717 (2019).
8. Koblan, L. W. et al. Improving cytidine and adenine base editors by expression optimization and ancestral reconstruction. Nat. Biotechnol. (2018). doi:10.1038/nbt.4172
9. Thuronyi, B. W. et al. Continuous evolution of base editors with expanded target compatibility and improved activity. Nat. Biotechnol. (2019). doi:10.1038/s41587-019-0193-0
10. Shinmura, K. et al. Aberrant Expression and Mutation-Inducing Activity of AID in

Human Lung Cancer. Ann. Surg. Oncol. 18, 2084-2092 (2011).
11. Gannon, H. S. et al. Identification of ADAR1 adenosine deaminase dependency in a subset of cancer cells. Nat. Commun. 9, 5450 (2018).
12. Weeks, L. D., Fu, P. & Gerson, S. L. Uracil-DNA glycosylase expression determines human lung cancer cell sensitivity to pemetrexed. Mol. Cancer Ther. 12, 2248-60 (2013).
13. Xin, H., Wan, T. & Ping, Y. Off-Targeting of Base Editors: BE3 but not ABE induces substantial off-target single nucleotide variants. Signal Transduct. Target. Ther. 4, 9 (2019).
14. Grünewald, J. et al. Transcriptome-wide off-target RNA editing induced by CRISPR-guided DNA base editors. Nature 569, 433-437 (2019).
15. Komor, A. C. et al. Improved base excision repair inhibition and bacteriophage Mu Gam protein yields C: G-to-T: A base editors with higher efficiency and product purity. Sci. Adv. 1-10 (2017).
16. Kim, Y. B. B. et al. Increasing the genome-targeting scope and precision of base editing with engineered Cas9-cytidine deaminase fusions. Nat. Biotechnol. 35, 371-376 (2017).
17. Gehrke, J. M. et al. An APOBEC3A-Cas9 base editor with minimized bystander and off-target activities. Nat. Biotechnol. (2018). doi:10.1038/nbt.4199
18. Wang, X. et al. Efficient base editing in methylated regions with a human APOBEC3A-Cas9 fusion. Nat. Biotechnol. 36, (2018).
19. Nishimasu, H. et al. Engineered CRISPR-Cas9 nuclease with expanded targeting space. Science (80-). 361, 1259-1262 (2018).
20. Kleinstiver, B. P. et al. High-fidelity CRISPR-Cas9 nucleases with no detectable genome-wide off-target effects. Nature 529, 490-495 (2016).
21. Anzalone, A. V. et al. Search-and-replace genome editing without double-strand breaks or donor DNA. Nature 576, 149-157 (2019).

EXEMPLARY SEQUENCES

SEQ ID NO: 1
>tr|G3U0R4|G3U0R4_LOXAF Apolipoprotein B mRNA editing enzyme catalytic subunit 1
OS = Loxodonta africana = african elephant OX = 9785 GN = APOBEC1 PE = 4 SV = 1
FRRRIKPWEFEIFFDPRQLRKETCLLYEIKWGTSHKVWRNSGQNTTKHVEVNFIEKFTSERK
LCPSISCSITWFLSWSPCWECSKAIREFLRQHPNVTLVIYVARLFHHMDQRNRQGLKDLILS
GITVQIMRVSEYHHCWRNFVSYSPGEETYWPRYPPLWMMMYALELHCIILSLPPCLKISRR
CQHQLTLFSLTPQKCHYQMIPPYILLATGLIEPPMTWR
SEQ ID NO: 2
>tr|A0A0M3N0G8|A0A0M3N0G8_PROAN APOBEC-1 OS = Protopterus annectens OX = 7888
PE = 2 SV = 1
MVQKRTSASKTRMTKKVLLSEYQKFYYSPRTCIGYVIQYDEDNVIFQNWICNKRTTHAELQC
IYEIKQNSLIKRFTPCTLKWYMSWTPCSECANEIIRFLNKFCQVKLEICAARIYFHKK
KDNRRALRNLVKAGVKLTTMRWKDYKSMWRRFGTGEEIKKYEFFEKSSDHKSVNWRWTL
KKILKEKDRDSDLENALSLLKI
SEQ ID NO: 3
>tr|A0A151P6M4|A0A151P6M4_ALLMI C->U-editing enzyme APOBEC-1 OS = Alligator
mississippiensis OX = 8496 GN = APOBEC1A PE = 4 SV = 1
MAVEEEKGLLGTSQGWKIELKDFQENYMPSTWPKVTHLLYEIRWGKGSKVWRNWCSNTL
TQHAEVNCLENAFGKLQFNPPVPCHITWFLSWSPCCQCCRRILQFLRAHSHITLVIKAAQLF
KHMDERNRQGLRDLVQSGVHVQVMDLPDYRYCWRTFVSHPHEGEGDFWPWFFPLWITF
YTLELQHILLQQHALSYNL
SEQ ID NO: 4
>tr|F1CGT0|F1CGT0_ANOCA Apolipoprotein B mRNA-editing enzyme 1a isoform
(Fragment) OS = Anolis carolinensis OX = 28377 PE = 2 SV = 1
KAAILLSNLFFRWQMEPEAFQRNFDPREFPECTLLLYEIHWDNNTSRNWCTNKPGLHAEEN
FLQIFNEKIDIKQDTPCSITWFLSWSPCYPCSQAIIKFLEAHPNVSLEIKAARLYMHQI
DCNKEGLRNLGRNRVSIMNLPDYRHCWTTFVVPRGANEDYWPQDFLPAITNYSRELDSILQ
D
SEQ ID NO: 5
>tr|A0A091EQ78|A0A091EQ78_CORBR C->U-editing enzyme APOBEC-1 (Fragment)
OS = Corvus brachyrhynchos OX = 85066 GN = N302_10757 PE = 4 SV = 1
RWKIEPGDFQINYSPSQHRRGVYLLYEIRWRRGSIWRNWCSNTHRQHAEVNFLENCFKDR
PQVPCSITWFLSASPCGKCSKRILEFLKSRPYVTLKIYAAKLFRHHDIRNREGLCNLGMHGV
TIHIMNLEDYSYCWRNFVVY
SEQ ID NO: 6
>tr|A0A091IIG0|A0A091IIG0_CALAN C->U-editing enzyme APOBEC-1 (Fragment)
OS = Calypte anna OX = 9244 GN = N300_12023 PE = 4 SV = 1
RWKIQPNDFKRNYQPGRRPNWYLLYEIRWRRGTIWRNWCSNEFPQHAEDNFFQNRFNA
VPSVSCSITWFLSTTPCGRCSKRILEFLRLHPNVTLKIYAARLFRHLDNRNRQGLRKLASNG
VIIQIMGLPDYSYSWKKFVAY
SEQ ID NO: 7
>tr|A0A2U4ALA1|A0A2U4ALA1_TURTR C->U-editing enzyme APOBEC-1 isoform X1
OS = Tursiops truncatus OX = 9739 GN = APOBEC1 PE = 4 SV = 1
MIICWSTGPSAGDATLRRRIEPWEFEVSFDPRELSKETRLLYEIKWGKSQRIWRHSGKNTT
KHVERNFIEQITSERRFHRSVSCCIIWFLSWSPCWECSEAIREFLKQHPRVTLLIYVARLFQH
MDPRNRQGLRDLTHSGVTIQIMGPTEYDYCWRYFVNYAPGKEAHWPRYPPLLMKLYALEL
HCIILGLPPCLNISRYQNQLTLFRPILRNCHYQMIPPHILLHTGLIQLPLTWR
SEQ ID NO: 8
>tr|A0A093FY71|A0A093FY71_TYTAL C->U-editing enzyme APOBEC-1 (Fragment)
OS = Tyto alba 0X = 56313 GN = N341_11998 PE = 4 SV = 1
RWKIQPNDFKRNFLPGQHPKVVYLMYEIRWIRGTAWRSWCSNNSKQDAEVNLLENCFKA
MPSVFCSVTWVLFTTPCGKCFRRILEFLRVHSNVALERYAAQLFRHLDICNWQGIRSLAMN
GVIIHIMNLADYSYCWKRFVAY
SEQ ID NO: 9
>tr|L5KGJ8|L5KGJ8_PTEAL C->U-editing enzyme APOBEC-1 OS = Pteropus alecto
OX = 9402 GN = PAL_GLEAN10015600 PE = 4 SV = 1
MWVLFDILISWSTGPSTGDPTLRRRIEPWEFEVFFDPRELRKEACLLYEIQWGTSHKIWRNS
GKNTTKHVELNFIEKFTSERHFCSSVSCSIIWFLSWSPCWECSKAIREFLSQRPTVTLVIFVS
RLFQHMDQQNRQGLRDLINSGVTIQIMRASEYDHCWRNFVNYPPGKEAHWPRYPPLWMK
LYALELHCIILSLPPCVMISRRCQKQLTLFTLILKKCHYQMIPAHILLATGLIQVPVTWR
SEQ ID NO: 10
>tr|A0A2K6KS69|A0A2K6KS69_RHIBE CMP/dCMP-type deaminase domain-containing
protein OS = Rhinopithecus bieti OX = 61621 GN = APOBEC1 PE = 4 SV = 1
MTSEKGPSTGDPTLRRRIEPWEFDIFYDPRELRKEACLLYEIKWGMSWKIWRSSGKNTTNH
VEVNFIEKFTSERRFHSSISCSITWFLSWSPCWDCSQAIRKFLSQHPGVTLVIYVARLFWHT
DQQNRQGLRDLVNSGVTIQMMTASEYYHCWRNFVNYPPGEEAHWPRYPPLWMMLYALE
LHCIILSLPPCLKISRRWQNHLTFFRLHLQNCHYQTIPPHILLATGLIQPSVTWR
SEQ ID NO: 11
>tr|A0A2Y9NGP5|A0A2Y9NGP5_DELLE C->U-editing enzyme APOBEC-1 isoform X1
OS = Delphinapterus leucas OX = 9749 GN = APOBEC1 PE = 4 SV = 1
MIICWSTGPSAGDATSRRRIEPWEFEVSFDPRELCKETRLLYEIKWGKSQHVWRHSDKNTT
KHVECKFIEKITSERHFHPSVSCCIIWFLSWSPCWECSKAIREFLNQHPRVTLFIYVARLFQH
MDPQNRQGLRDLIHSGVTIHVMGPTEYDYCWRNFVNYPPGKEAHWPRYPPMLMKLYALE
LHCIILGLPPCLNISRYQNQLTLFRLIPQNCHYRMIPPHILLHRGLIRLPLTWR
SEQ ID NO: 12
>tr|A0A218ULD2|A0A218ULD2_9PASE C->U-editing enzyme APOBEC-1 OS = Lonchura
striata domestica OX = 299123 GN = APOBEC1 PE = 4 SV = 1
MYRRKMRGMYISKRALRKHFDPRNYPRETYLLCELQWRGSHKSWQHWLRNDDSKDCHA
EKYFLEEIFEPRSYNICDMTWYLSWSPCGECCDIIQDFLEEQPNVNINIRIARLYYADRASNR
RGLMELANSPGVSIEIMDADDYNDCWETFIQPGVYYRFSPENFESAIRRNCSQLEDILQGLH
L
SEQ ID NO: 13
>tr|A0A0Q3WRD0|A0A0Q3WRD0_AMAAE C->U-editing enzyme APOBEC-1 OS = Amazona
aestiva OX = 12930 GN = AAES_27783 PE = 4 SV = 1
MLPAPAPVPLVLPLQGGGVVVVTVGVXPTALLQPSGAPEVARTFVGAVIAFVIAEYVDTSVS
EDTTICGMYIPKEALKYHFDPREVXRDTYLLCILRWGETGTPWSHWVKNYRYHAEVYFLEKI
FQTRKSSKNINCSITWYLSWSPCAKCCRKILNFLKKHSYVSIKIHVARLFRIDDKETXQNLKN
LGSLVGVTVSVMEXEDYTNCWKTFIRGHADGDSWIDDLKSEIRKNRLKFQGIFKDLPHQTE
DVDFWLILAANPGPAWFSFSGYTGWAVASKAPSLLSPLSCLTRLLTP
SEQ ID NO: 14
>tr|A0A2K6U925|A0A2K6U925_SAIBB Apolipoprotein B mRNA editing enzyme catalytic
subunit 1 OS = Saimiri boliviensis boliviensis OX = 39432 GN = APOBEC1 PE = 4
SV = 1
MTSERRRIEPWEFSISYDPRELCKETCLLYEIKWGMSWKIWRSSGKNTTNHVEVNFIEKFTS
ERHFHSSVSCSITWFLSWSPCWECSQAIREFLSQHPGVTLVIYVARLFQHMDQQNRQGLR
ELVNSGVTIQIMTASEYYHCWRNFVNYPPGEEAHWPRHPPLWMMLYALELHCIIL
SEQ ID NO: 15
>tr|A0A2R9A0R0|A0A2R9A0R0_PANPA CMP/dCMP-type deaminase domain-containing
protein OS = Pan paniscus OX = 9597 GN = APOBEC1 PE = 4 SV = 1
ISWSTGPSTGDPTLRRRIEPWEFDVFYDPRELRKEACLLYEIKWGMSRKIWRSSERDFHPSI
SCSITWFLSWSPCWECSQAIREFLSQHPGVTLVIYVARLFWHMDQQNRQGLRDLVNSGVTI
QIMTASEYYHCWRNFVNYPPGDEAHWPQYPPLWMMLYALELHCIILSLPPCLKISRRWQN
HLTFFSLHLQNCHYQTIPPHILLATGLIHPSVAWR
SEQ ID NO: 16
>sp|Q694B3|ABEC1_PONPY C->U-editing enzyme APOBEC-1 OS = Pongo I
OX = 9600 GN = APOBEC1 PE = 3 SV = 2
MTSEKGPSTGDPTLRRRIESWEFDVFYDPRELRKETCLLYEIKWGMSRKIWRSSGKNTTNH
VEVNFIKKFTSERRFHSSISCSITWFLSWSPCWECSQAIREFLSQHPGVTLVIYVARLFWHM
DQRNRQGLRDLVNSGVTIQIMRASEYYHCWRNFVNYPPGDEAHWPQYPPLWMMLYALEL
HCIILSLPPCLKISRRWQNHLAFFRLHLQNCHYQTIPPHILLATGLIHPSVTWR
SEQ ID NO: 17
>tr|E1BP99|E1BP99_BOVIN Apolipoprotein B mRNA editing enzyme catalytic subunit 1
OS = Bos taurus OX = 9913 GN = APOBEC1 PE = 4 SV = 1
MASDRGPPAGDPTLRRRIEPWEFEFSFDPRKFCKEACLLYEIQWGNNRDVWRHSGKNTTK
HVERNFIEKIASERYFCPSIRCFIFWYLSWSPCWECSKAIREFLNQHPNVTLVIYIARLFQHM
DPQNRQGLKDLVQSGVTIQVMRAPEYEYCWRNFVNYPRGKEAHWPRYPPLWMNLYALEL
YCIILGLPPCLHISRRYQNQLIVFRLTLQNCHYQMIPPYILLATGMVQLPMTWR
SEQ ID NO: 18
>tr|S7PYX0|S7PYX0_MYOBR C->U-editing enzyme APOBEC-1 OS = Myotis brandtii
OX = 109478 GN = D623_10002956 PE = 4 SV = 1
MDEQNRQGLRDLIKSGVTVQIMTTPEYDYCWRNFVNYPPGKDTHCPMYPPLWMKLYALEL
HCIILSLPPCLMISRRCQKQLTWYRLNLQNCHYQQIPHHILLATVWI
SEQ ID NO: 19
>tr|M3WB96|M3WB96_FELCA Apolipoprotein B mRNA editing enzyme catalytic subunit 1
OS = Felis catus OX = 9685 GN = APOBEC1 PE = 4 SV = 2
MASDKGPSAGDATLRRRIEPREFEVFFDPRELRKEACLLYEIKWGTSHRIWRNSGRNTANH
VELNFIEKFTSERHFCPSVSCSITWFLSWSPCWECSKAIRGFLSQHPSVTLVIYVSRLFWHL
DQQNRQGLRDLVNSGVTVQIMRVPEYDHCWRNFVNYPPGEEDHWPRYPVVWMKLYALE
LHCIILSLPPCLKILRRCQNQLTLFRLTLQNCHYQMIPPHILLATGLIQLPVTWR
SEQ ID NO: 20
>tr|A0A2K5PZC0|A0A2K5PZC0_CEBCA CMP/dCMP-type deaminase domain-containing
protein OS = Cebus capucinus imitator OX = 1737458 GN = APOBEC1 PE = 4 SV = 1
MTSERGPSTGDPTLRRRIEPWEFYISYDPKELCKETCLLYEIKWGMSWKIWRSSGKNTTNH
VEVNFIEKFTSERRFHSSISCSITWFLSWSPCWECSQAIREFLSQHPGVTLVIYVARLFQHM
DQQNRQGLRDLVNSGVTIQIMRASEYYYCWRNFVNYPPGEEAHWPRHPPLWMMLYALEL
HCIILGLPPCLKISRRRQNRLTFFRLHLQNCHYQMIPPHILLAAGLIQPSVTWR
SEQ ID NO: 21
>tr|H2Q5C6|H2Q5C6_PANTR Apolipoprotein B mRNA editing enzyme catalytic subunit 1
OS = Pan troglodytes OX = 9598 GN = APOBEC1 PE = 4 SV = 1
MTSEKGPSTGDPTLRRRIEPWEFDVFYDPRELRKEACLLYEIKWGMSRKIWRSSGKNTTN
HVEVNFIKKFTSERHFHPSISCSITWFLSWSPCWECSQAIREFLSQHPGVTLVIYVARLFWH
MDQQNRQGLRDLVNSGVTIQIMRASEYYHCWRNFVNYPPGDEAHWPQYPPLWMMLYAL
ELHCIILSLPPCLKISRRWQNHLTFFSLHLQNCHYQTIPPHILLATGLIHPSVAWR
SEQ ID NO: 22
>tr|A0A1U7S7K7|A0A1U7S7K7_ALLSI C->U-editing enzyme APOBEC-1-like OS = Alligator
sinensis OX = 38654 GN = LOC102373005 PE = 4 SV = 1
MGEHWQYAGSGEYIPQDQFEENFDPSVLLAETHLLSELTWGGRPYKHWYENTEHCHAEIH
FLENFSSKNRSCTITWYLSWSPCAECSARIADFMQENTNVKLNIHVARLYLHDDEHTRQGL
RYLMKMKRVTIQVMTIPDYTYCWNTFLEDDGEDESDDYGGYAGVHEDEDESDDDDYLPTH
FAPWIMLYSLELSCILQGFAPCLKIIQGNHMSPTFQLHVQDQEQKRLLEPANPWGAD
SEQ ID NO: 23
>tr|G3HS7|G3HS7_CRIGR C->U-editing enzyme APOBEC-1 OS = Cricetulus griseus
OX = 10029 GN = I79_017346 PE = 4 SV = 1
MTEQEYCYCWRNFVNYPPSNEVYWPRYPNVWMRMYALELYCIVLGLPPCLKIIRRHQHPL
TFFTLHLQSCHYQRIPPHILWATGLV
SEQ ID NO: 24
>tr|A0A094MFH1|A0A094MFH1_ANTCR C->U-editing enzyme APOBEC-1 (Fragment)
OS = Antrostomus carolinensis OX = 279965 GN = N321_09417 PE = 4 SV = 1
RWKMQPNDFKRNYLPVQYPNMVYLLYEIRWSTGTIWRNWCSNNSTQHAEVNFLENRFNS
RPSVSCSITWVLSTTPCGKCSTKILEFLRLHPNVTLKIYAAKLFKHLDIRNRQGLRNLAMNGV
IIRIMNLADYSYCWKTFVAY
SEQ ID NO: 25
>tr|A0A2K6EVT9|A0A2K6EVT9_PROCO CMP/dCMP-type deaminase domain-containing
protein OS = Propithecus I OX = 379532 GN = APOBEC1 PE = 4 SV = 1
MTSEKRRIEPWEFEAFFDPRELRKEACLLYEIKWGASHKIWRNTGKSTTRHVEVNFIEKFTS
ERRSDSLISCSITWFLSWSPCWECSKAIREFLSQHPNVTLVIYVARLFWHMNQQNRQGLRD
LINSGVTVQIMGVSEYCHCWRNFVNYPPGKEASCPTYPPLWMTLYALELHCIILSLPPCLKIS
RRCQNQLTFFRLTPQNCHYQTIPPHILLATGLIQPSVTWR
SEQ ID NO: 26
>tr|G8F4P7|G8F4P7_MACFA C->U-editing enzyme APOBEC-1 (Fragment) OS = Macaca
fascicularis OX = 9541 GN = EGM_20518 PE = 4 SV = 1
GPSTGDPTLRRRIEPWEFDIFYDPRELRKEACLLYEIKWGMSPKIWRSSGKNTTNHVEVNFI
EKLTSERRFHSSISCSITWFLSWSPCWECSQAIREFLSQHPGVTLVIYVARLFWHTDQQNR
QGLRDLVNSGVTIQIMRASEYYHCWRNFVNYPPGEEAHWPRYPPLWMMLYALELHCIILSL
PPCLKISRRWQNHLTFFRLHLQNCHYQMIPPHILLATGLIQPSVTWR
SEQ ID NO: 27
>tr|A0A091V7F8|A0A091V7F8_NIPNI C->U-editing enzyme APOBEC-1 (Fragment)
OS = Nipponia nippon OX = 128390 GN = Y956_13652 PE = 4 SV = 1
RWKIQPNDFRSNYLPCQHPRVVYLLYEIRWSRGTIWRNWCSNNSTQHAEVNFLENCFKAM
PSVPCSITWVLSTTPCGKCSRRILEFLRVHPNVTLEIYAAKLFKHLDIRNRQGLRNLAKNGVV
IRIMKLADYSYWWKRFVAY
SEQ ID NO: 28
>tr|A0A091SSF0|A0A091SSF0_PELCR C->U-editing enzyme APOBEC-1 (Fragment)
OS = Pelecanus crispus OX = 36300 GN = N334_11718 PE = 4 SV = 1
RWKLQPEDFKRNYLPGQHPKVVYLLYEIRWSRGTIWRSWCSNNSKQHAEVNFLENCFKAR
PSVSCSITWVLSTTPCGKCSRRILEFLRVHPNVTLEIYAAKLFKHLDIRNQQGLRNLAMNGVII
RIMNLADYSYCWKRFVAH
SEQ ID NO: 29
>tr|A0A091CVE5|A0A091CVE5_FUKDA C->U-editing enzyme APOBEC-1 OS = Fukomys
damarensis OX = 885580 GN = H920_16562 PE = 4 SV = 1
MSDPEFCHCWRNFVNYPPGQEARWPRFPPVWTMLYTLELCCVLLNLPPCLKISRRCHNQL
AFFQLNLQNCHYRAIPPAVLFAVGLIHPFVAWA
SEQ ID NO: 30
>tr|L5LUG3|L5LUG3_MYODS C->U-editing enzyme APOBEC-1 OS = Myotis davidii
OX = 225400 GN = MDA_GLEAN10003736 PE = 4 SV = 1
MASDAGKMDRGPVSFIVLKSVETLCVRRIEPWEFEAIFDPRELRKEACLLYEIKWGTGHKIW
RHSGKNTTRHVEVNFIEKITSERQFCSSTSCSIIWFLSWSPCWECSKAITEFLRQRPGVTLVI
YVARLYHHMDEQNRQGLRDLVKSGVTVQIMTTPEYDYCWRNFVNYPPGKDTHCPIYPPLL
MKLYALELHCIILSLPPCLMISRRCQKQLTWYRLNLQNCHYQQIPHHILLATAWI
SEQ ID NO: 31
>tr|F1PUJ5|F1PUJ5_CANLF Apolipoprotein B mRNA editing enzyme catalytic subunit 1
OS = Canis lupus familiaris OX = 9615 GN = APOBEC1 PE = 4 SV = 2
MASDKGPSAGDATLRRRIEPWEFEGFFDPRELRKETCLLYEIQWGTSHKTWRNSGKNTTN
HVEINFMEKFAAERQYCPSIRCSITWFLSWSPCWECSNAIRGFLSQHPSVTLVIYVARLFWH
TDPQNRQGLRDLINSGVTIQIMTVPEYDHCWRNFVNYPPGKEDHWPRYPVLWMKLYALEL
HCIILNLPPCLKISRRNQHQLTLFRLTLQDCHYQTIPPPILLDMGLIQPLVTWR
SEQ ID NO: 32
>tr|A0A093GVH6|A0A093GVH6_DRYPU C->U-editing enzyme APOBEC-1 (Fragment)
OS = Dryobates pubescens OX = 118200 GN = N307_04563 PE = 4 SV = 1
RWKIHPDEFKLNYVPVGRPRWYLLYEIRWSRGSIWRNWCSNSSTQHAEVNFLENCFKAM
PSVSCSITWFLSTTPCGNCSRRILEFLRAHPKVTLAIHAAKLFKHLDVRNRHGLKALATDGVV
LHIMSIADYRYCWTKFVAY
SEQ ID NO: 33
>tr|A0A2K5Z8Y4|A0A2K5Z8Y4_MANLE CMP/dCMP-type deaminase domain-containing
protein OS = Mandrillus leucophaeus OX = 9568 GN = APOBEC1 PE = 4 SV = 1
MTSEKGPSTGDPTLRRRIEPWEFDIFYDPRELRKEACLLYEIKWGMSPKIWRSSGKNTTNH
VEVNFIEKLTSERRFHSSISCSITWFLSWSPCWECSQAIREFLSQHPGVTLVIYVARLFWHT
DQQNRQGLRDLVNSGVTIQIMRASEYYHCWRNFVNYPPGEEAHWPRYPPLWMMLYALEL
HCIILSLPPCLKISRRQQNHLTFFRLHLQNCHYQTIPPHILLATGLIQPSVTWR
SEQ ID NO: 34
>tr|A0A087VMP5|A0A087VMP5_BALRE C->U-editing enzyme APOBEC-1 (Fragment)
OS = Balearica regulorum gibbericeps OX = 100784 GN = N312_10691 PE = 4 SV = 1
RWKIQPDDFKRNYLPGKHPRWYLLYEIRWSRGTIWRSWCSNNATQHAEINFLETCFLART
SVSCSITWVLSTTPCGKCSRRILEFLNAYPNVTLEIYAAKLFRHLDNRNRQGLRNLAMKGVR
IHIMNLADYSYFWKIFVAY
SEQ ID NO: 35
>tr|A0A087QNJ5|A0A087QNJ5_APTFO C->U-editing enzyme APOBEC-1 (Fragment)
OS = Aptenodytes forsteri OX = 9233 GN = AS27_08049 PE = 4 SV = 1
RWKIRPNDFKRNYLPGQHPKVVYLLYEIRWSRGTIWRNWCSNNSTQHAEVNFLENCFKAM
PSVSCSITWVLSTTPCGKCSRRILEFLRVHPNVTLEIYAAKLFKHLDIRNRQGLRNLAMNGVII
RIMNLADYSYGWKRFVAY
SEQ ID NO: 36
>tr|A0A2Y9IYV0|A0A2Y9IYV0_ENHLU C->U-editing enzyme APOBEC-1 OS = Enhydra lutris
kenyoni OX = 391180 GN = LOC111142361 PE = 4SV = 1
MASDKGPSAGDATLRRRIEPWEFEVFFDPRELRKEACLLYEIQWGTSHKMWRNTGKNTAN
HVELNFIEKFTSERRYCPSTHCSITWFLSWSPCWECCKAIRGFLSQHPSVTLVIYVTRLFWH
MDPQNRQGLRDLLKSGVTVQIMRAPEYDHCWKNFVNYPPGKEDHWPRYPELWMKLYELE
LYCIILSLPPCLKISRRNQNQLTLFRLTLQNCHYQIIPPHILLDTGLIQLPVIWR
SEQ ID NO: 37
>tr|B2NIW5|B2NIW5_MUSPF Apolipoprotein B mRNA editing protein OS = Mustela putorius
furo OX = 9669 GN = APOBEC1 PE = 2 SV = 1
MASDKGPSAGDATLRRRIEPWEFEVFFDPRELRKEACLLYEIQWGTSHKMWRNTGKNTAN
HVELNFIEKFTSERRYCPSTHCSITWFLSWSPCWECSKAIRGFLSQCPSVTLVIYVTRLFWH
MDPQNRQGLRDLLKSGVTVRIMRAPEYDHCWKNFVNYPPGKEDHWPRYPELWMKLYELE
LYCIILSLPPCLKISRRNQKQLTLFRLTLQNCHYQIIPPHILLDTGLIQLPVIWR
SEQ ID NO: 38
>tr|A0A2Y9E587|A0A2Y9E587_TRIMA C->U-editing enzyme APOBEC-1 OS = Trichechus
manatus latirostris OX = 127582 GN = LOC101361717 PE = 4 SV = 1
MTSEEADQRHSTMTSEKGPSTGDGTLRRRITPWEFEIFFDPRELRKETCLLYEIKWGTSHRI
WRNSGQNTTKHAEVNFIEKFTSERNFCPSVSCSITWFLSWSPCWECSKAIREFLSQHPNVI
LVIYVARLFHHMDQQNREGLRDLVLSGVTVQIMSVSEYGHCWRNFVNYPPGEEARWPRYP
PLWMMLYALELHCIILGLPPCLKISRRRQSQLTLFSLTPQNCHYQMIPPHILLATGLIQPYVTW
R
SEQ ID NO: 39
>tr|G1LKL4|G1LKL4_AILME CMP/dCMP-type deaminase domain-containing protein
OS = Ailuropoda melanoleuca OX = 9646 GN = APOBEC1 PE = 4 SV = 1
ISWSTGPSGGDATSRRRIEPWEFEVFFDPRQLRKEACLLYEIQWGTSRKIWRNSGKNTTNH
VEINFIEKFTLERQYCPSIHCSVTWFLSWSPCWECSKAIRAFLSQHPSVTLVIYVARLFWHM
EPQNRQGLRDLINSGVTIQIMSVPEYDHCWRNFVNYPPGKDHWPGYPVLWMKLYALELHC
IILSLPPCLKISRRNQNQLTLFRLTLQNCHYQTIPPHVLLATGLIQLPVTWR
SEQ ID NO: 40
>tr|A0A093PWR2|A0A093PWR2_9PASS C->U-editing enzyme APOBEC-1 (Fragment)
OS = Manacus vitellinus OX = 328815 GN = N305_14278 PE = 4 SV = 1
RWKIQPKDFKRNYLPGQHPQWYLLYEIRWRNGSIWRNWFSNNRNQHAEVNFLENCFSDV
PPAPCSITWFLSTSPCGKCSRRILEFLRTHRNVTLEIYAAKLFRHQDIRNRQGLCNLVMNGV
TIHIMNLADYSYCWKRFVAY
SEQ ID NO: 41
>sp|Q9EQP0|ABEC1_MESAU C->U-editing enzyme APOBEC-1 OS = Mesocricetus auratus
OX = 10036 GN = APOBEC1 PE = 2 SV = 1
MSSETGPVVVDPTLRRRIEPHEFDAFFDQGELRKETCLLYEIRWGGRHNIWRHTGQNTSR
HVEINFIEKFTSERYFYPSTRCSIVWFLSWSPCGECSKAITEFLSGHPNVTLFIYAARLYHHT
DQRNRQGLRDLISRGVTIRIMTEQEYCYCWRNFVNYPPSNEVYWPRYPNLWMRLYALELY
CIHLGLPPCLKIKRRHQYPLTFFRLNLQSCHYQRIPPHILWATGFI
SEQ ID NO: 42
>tr|A0A2K6PRF3|A0A2K6PRF3_RHIRO CMP/dCMP-type deaminase domain-containing
protein OS = Rhinopithecus roxellana OX = 61622 GN = APOBEC1 PE = 4 SV = 1
MSWKIWRSSGKNTTNHVEVNFIEKFTSERRFHSSISCSITWFLSWSPCWDCSQAIRKFLSQ
HPGVTLVIYVARLFWHTDQQNRQGLRDLVNSGVTIQMMTASEYYHCWRNFVNYPPGEEA
HWPRYPPLWMMLYALELHCIILSLPPCLKISRRWQNHLTFFRLRLQNCHYQTIPPHILLATGL
IQPSVTWR
SEQ ID NO: 43
>tr|A0A0D9RBS4|A0A0D9RBS4_CHLSB Apolipoprotein B mRNA editing enzyme catalytic
subunit 1 OS = Chlorocebus sabaeus OX = 60711 GN = APOBEC1 PE = 4 SV = 1
MSRKIWRSSGKNTTNHVEVNFIEKLTSERRFHSSVSCSVTWFLSWSPCWECSQAIREFLS
QHPGVTLVIYVARLFWHTDQQNRQGLRDLVNSGVTIQIMRASEYYHCWRNFVNYPPGEEA
HWPRYPPLWMMLYALELHCIILSLPPCLKISRRWQNHLTFFRLHLQNCHYQTIPPHILLATGL
IQPPVTWR
SEQ ID NO: 44
>tr|A0A286XNR2|A0A286XNR2_CAVPO CMP/dCMP-type deaminase domain-containing
protein OS = Cavia porcellus OX = 10141 GN = APOBEC1 PE = 4 SV = 1
MASGTGPSTGDATLRRRIEPWQFEAYFDPRQLRKEACMLSEVRWGASPRTWRESSLNTT
SHVEINFIEKFTSGRSLRPAVRCSMTWFLSWSPCWECARAIREFLHQHPNVSLVIYVARLY
WHVDEQNRQGLRDLVTSGVRVQIMSDSEYRHCWRNFVNFPPGQEAGWPRFPPMWTTLY
ALELSCILLSLPPCLKISRRRQYRLIVFQLILQTCHYRAIPPQVLSAAELMHPLVAWC
SEQ ID NO: 45
>tr|A0A2Y9HAT6|A0A2Y9HAT6_NEOSC C->U-editing enzyme APOBEC-1
OS = Neomonachus schauinslandi OX = 29088 GN = APOBEC1 PE = 4 SV = 1
MASDKGPSAGDATLRRRIKPWEFEVFFDPRELRKETCLLYEIQWGTSHKIWRNSGKNTAN
HVEINFIEKFTSERQYCPSIRCSITWFLSWSPCWECSKAIRGFLSQHPSVTLVIYVARLFWH
MDPQNRQGLRDLINSGVTIQIMRVPEYDHCWRNFVNYLPGKEDHWPRYPVLWMKLYALEL
HCIILSLPPCLRISRRQNQLTLFTLTLQNCHYQMIPPHILLATGLIQVPVTWK
SEQ ID NO: 46
>tr|A0A091XJL0|A0A091XJL0_OPIHO C->U-editing enzyme APOBEC-1 (Fragment)
OS = Opisthocomus hoazin OX = 30419 GN = N306_09750 PE = 4 SV = 1
RWKVQPNDFKRNYLPGQHPKVVYILYEIRWSRGTIWRNWCTNNSTQHAEVNFLENCFKAM
PSVSCSITWVLSTTPCGKCSKRIQDFLRIYPNVTLEIHAAKLFKHLDTRNREGLRNLAKDGVII
HIMNLADYSYWWKRFVAY
SEQ ID NO: 47
>tr|F6WR88|F6WR88_HORSE Apolipoprotein B mRNA editing enzyme catalytic subunit 1
OS = Equus caballus OX = 9796 GN = APOBEC1 PE = 4 SV = 2
MSHNIWRYSGKNTTKHVEINFIEKFTSERHLRPSISCSIVWFLSWSPCWECSKAIREFLS
QHPNVTLVIYVARLFQHMDRLNRQGLRDLINSGVTIQIMRTSEYDHCWRNFVNYPPGKEAH
WPRYPLLWMKLYALELHCIILSLPPCLMISRRCQNQLTFFRLTLQNCHYQMIPPHILLATGLV
QLPVTWR
SEQ ID NO: 48
>sp|P41238|ABEC1_HUMAN C->U-editing enzyme APOBEC-1 OS = Homo sapiens
OX = 9606 GN = APOBEC1 PE = 1 SV = 3
MTSEKGPSTGDPTLRRRIEPWEFDVFYDPRELRKEACLLYEIKWGMSRKIWRSSGKNTTN
HVEVNFIKKFTSERDFHPSMSCSITWFLSWSPCWECSQAIREFLSRHPGVTLVIYVARLFW
HMDQQNRQGLRDLVNSGVTIQIMRASEYYHCWRNFVNYPPGDEAHWPQYPPLWMMLYA
LELHCIILSLPPCLKISRRWQNHLTFFRLHLQNCHYQTIPPHILLATGLIHPSVAWR
SEQ ID NO: 49
>tr|A0A091RU17|A0A091RU17_NESNO C->U-editing enzyme APOBEC-1 (Fragment)
OS = Nestor notabilis OX = 176057 GN = N333_10787 PE = 4 SV = 1
RWKIQPNDFKRNYLPYQHPKVVCLLYEIRWNRGTIWRSWCSNNSTQHAEVNFLENCFKAK
PSVSCSITWVLSTTPCGECSRRILDFLSVYPNVTLKIYAAKLFKHLDNRNRQGLWNLANNRV
IIRIMNLEDYNYYWKRFVAY
SEQ ID NO: 50
>tr|A0A091IWL9|A0A091IWL9_EGRGA C->U-editing enzyme APOBEC-1 (Fragment)
OS = Egretta garzetta OX = 188379 GN = Z169_O8812 PE = 4 SV = 1
RWKIQPNDFKRNYLPGQHPKVVYLLYEIRWSRGTIWRNWCSNNSTQHAEVNFLENCFKAM
PSVSCSITWVLSTTPCGKCSRRILEFLRVHPSVTLEIYAAKLFKHLDIRNRQGLRNLAMNGVII
HIMNLADYSYWWKIFVAY
SEQ ID NO: 51
>tr|A0A2K5DG70|A0A2K5DG70_AOTNA CMP/dCMP-type deaminase domain-containing
protein OS = Aotus nancymaae OX = 37293 GN = APOBEC1 PE = 4 SV = 1
MTPEEEVQRQSTMTSERGPSTGDPTLRRRIEPWEFCISYDPKELCKETCLLYEIKWGTSWK
IWRSSGKNTTNHVEVNFIEKFMSERHFHSSISCSITWFLSWSPCWECSQAIREFLSRHPGV
TLVIYVARLFQHMDRQNRQGLRDLVNSGVTIQIMRASEYYHCWRNFVNYPPGEEAHWPRY
PPLWMMLYALELHCIILGLPPCLKISRRWQNRLTFFRLHLQNCHYQMIPQHILFATGLIQPPV
TWR
SEQ ID NO: 52
>sp|P51908|ABEC1_MOUSE C->U-editing enzyme APOBEC-1 OS = Mus I
OX = 10090GN = Apobec1 PE = 1 SV = 1
MSSETGPVAVDPTLRRRIEPHEFEVFFDPRELRKETCLLYEINWGGRHSVWRHTSQNTSN
HVEVNFLEKFTTERYFRPNTRCSITWFLSWSPCGECSRAITEFLSRHPYVTLFIYIARLYHHT
DQRNRQGLRDLISSGVTIQIMTEQEYCYCWRNFVNYPPSNEAYWPRYPHLWVKLYVLELY
CIILGLPPCLKILRRKQPQLTFFTITLQTCHYQRIPPHLLWATGLK
SEQ ID NO: 53
>tr|G5BPM8|G5BPM8_HETGA C->U-editing enzyme APOBEC-1 (Fragment)
OS = Heterocephalus glaber OX = 10181 GN = GW7_17308 PE = 4 SV = 1
RRRIEPWQFEASFDPRQLRRETCLLSEVRWGTSPRAWRGCSLNTARHAEVSFMDRLTSE
GRLRGPVRCSITWFLSWSPCGACAQAIGEFLRQHPNVSLVIYIARLFWHVDEQNRQGLRDL
VTRGVRMQVMSDPEFAHCWRNFVNYSPGQEARWPQVPPWVTWLYSLELHCILLNLPPCL
KISRRHHNQLTFFQLILQNCHYQAIPSPVLLASGLIHPFVTW
SEQ ID NO: 54
>tr|A0A091QEK6|A0A091QEK6_MERNU C->U-editing enzyme APOBEC-1 (Fragment)
OS = Merops nubicus OX = 57421 GN = N331_01832 PE = 4 SV = 1
RWKIEPDEFKTNYSPDHRPRVVYLLYEIRWRRGTIWRNWCSNNIDQHAEVNFLENCFKAK
PSVSCSITWFLSTAPCAKCSRRILKFLTAHPKVTLEIYAAKLFRHLEIRNRQGLMDLAVN
GVILRIMNLADYSYCWKQFVAY
SEQ ID NO: 55
>tr|A0A093LP85|A0A093LP85_FULGA C->U-editing enzyme APOBEC-1 (Fragment)
OS = Fulmarus glacialis OX = 30455 GN = N327_13724 PE = 4 SV = 1
RWKIQPNDFKRNFLPSKYPKVVYLLYEIRWSSGTIWRSWCSNNSTQHAEVNFLENCFKAM
PSVSCSITWVLPITPCGKCSKKILEFLSVHPNVTLEIYAAKLFRHLDIRNQQGLRNLAMN
GVIIRIMNLADYSYSWKRFVAY
SEQ ID NO: 56
>tr|G1QZV0|G1QZV0_NOMLE CMP/dCMP-type deaminase domain-containing protein
OS = Nomascus leucogenys OX = 61853 GN = APOBEC1 PE = 4 SV = 1
MTSEKGPSTGDPTLRRRIEPWEFDVFYDPRELRKEACLLYEIKWGMSQKIWRSSGKNTTN
HVEVNFIKKFTSEGRFQSSISCSITWFLSWSPCWECSQAIREFLSQHPGVTLVIYVARLF
WHMDQQNRQGLRDLVNSGVTIQIMRASEYYHCWRNFVNYPPGDEAHWPRYPPLWMMLY
AL
ELHCIILSLPPCLKISRRWQNHLTFFRLHLQNCHYQTIPPHILLATGLIHPSVTWR
SEQ ID NO: 57
>tr|A0A096MWB4|A0A096MWB4_PAPAN CMP/dCMP-type deaminase domain-containing
protein OS = Papio anubis OX = 9555 GN = APOBEC1 PE = 4 SV = 2
MTSEKGPSTGDPTLRRRIEPWEFDIFYDPRELRKEACLLYEIKWGMSPKIWRSSGKNTTN
HVEVNFIEKLTSERRFHSSISCSITWFLSWSPCWECSQAIREFLSQHPGVTLVIYVARLF
WHTDQQNRQGLRDLVNSGVTIQIMTASEYYHCWRNFVNYPPGEEAHWPRYPPLWMMLY
AL
ELHCIILSLPPCLKISRRWQNHLTFFRLHLQNCHYQTIPPHILLATGLIQPSVTWR
SEQ ID NO: 58
>sp|Q9TUI7|ABEC1_MONDO C->U-editing enzyme APOBEC-1 OS = Monodelphis
domestica OX = 13616 GN = APOBEC1 PE = 1 SV = 1
MNSKTGPSVGDATLRRRIKPWEFVAFFNPQELRKETCLLYEIKWGNQNIWRHSNQNTSQH
AEINFMEKFTAERHFNSSVRCSITWFLSWSPCWECSKAIRKFLDHYPNVTLAIFISRLYW
HMDQQHRQGLKELVHSGVTIQIMSYSEYHYCWRNFVDYPQGEEDYWPKYPYLWIMLYVLE
LHCIILGLPPCLKISGSHSNQLALFSLDLQDCHYQKIPYNVLVATGLVQPFVTWR
SEQ ID NO: 59
>tr|A0A1S3FTE2|A0A1S3FTE2_DIPOR C->U-editing enzyme APOBEC-1 OS = Dipodomys
ordii OX = 10020 GN = Apobec1 PE = 4 SV = 1
MHHSARLPPNCIVSRYANAPWTVLPLPLPPTEAPATGDDTLRRRIEPWEFEAFFNPQELR
REACLLYQITWSSHKVWRETAKNTVDSHVEVNFIQNLTAGRYCRPSTRCSILWFLSWSPC
SSCSKAIRLFLSQHPGVSLVIYVARLFQHMDPQNRQGLRELIHSGVTIQVMRPQEYDYCWK
NFVNYPPGQEEHWPRYPVQCMTLYNLELYCIIHNLPPCVRISKQRQSQLAFFSLGLENVHY
QRIPPPLLLLTGLVFVFPWK
SEQ ID NO: 60
>tr|A0A2U3WPA5|AO0A2U3WPA5_ODORO C->U-editing enzyme APOBEC-1
OS = Odobenus rosmarus divergens OX = 9708 GN = APOBEC1 PE = 4 SV = 1
MASDKGPSAGDATLRRRIEPWEFEVFFDPRELRKEACLLYEIQWGTSHKIWRNSGKNTSN
H VEIN FI EKFTSERQYCPSIHCSITWFLSWSPCWECSEAIRGFLSQHPSVTLVIYVARLFWH
MDPQNRQGLRDLINSGVTIQIMRVPEYDHCWRNFVNYPPGKEDHWPRYPVLWMKLYALEL
HCIILSLPPCLRISRRQNQLTLFRLTLQNCHYQMIPPHILLATGLIQVPVTWK
SEQ ID NO: 61
>tr|A0A1V4JAP2|A0A1V4JAP2_PATFA C->U-editing enzyme APOBEC-1 OS = Patagioenas
fasciata monilis OX = 372326 GN = APOBEC1 PE = 4 SV = 1
MRRKKPSGMYISKRALKDNFDPHKFPHDTYLLCKLQWGDTGRSWIHWIRKDRYHAEVYFL
EKIFKMRRSKNYVNCSITWYLSWSPCVRCCCEILNFLEKHSYVNIDIYVARLYKIQNSEVREG
LKKLVSSKKVTIAVMEIKDYTYCWKNFIQGDADDDSWTVDFQSAITKNRLKLKDVFEFLKSH
PNVTLEIYAAKLFKHLDIRNREGLRNLAKNGVIIHIMNLADYSYWWKIFVTRQHGEDDYLPWS
FALHIFLNCIEFQQILLVSRHLKESLRVKSNEKAQEKEVWRIPAMVLAEMIVGKMNRDLMLHE
QRANRARNCKGLWCYIVPL
SEQ ID NO: 62
>tr|A0A2K5JKV4|A0A2K5JKV4_COLAP CMP/dCMP-type deaminase domain-containing
protein OS = Colobus angolensis palliatus OX = 336983 GN = APOBEC1 PE = 4 SV = 1
PSTGDPTLRRRIEPWEFDIFYDPRELRKEACLLYEIKWGMSQKIWRSSGKNTTNHVEVNFIE
KLTSERRFHSSVSCSITWFLSWSPCWECSQAIREFLSQHPGVTLVIYVARLFWHTDQQNRQ
GLRDLVNSGVTIQMMTASEYYHCWRNFVNYPPGEEAHWPRYPPLWMMLYALELHCIILSL
PPCLKISRRWQNHLTFFRLHLQNCHYQTIPPHILLATGLIQPSVTWR
SEQ ID NO: 63
>tr|A0A1U7U8J6|A0A1U7U8J6_TARSY LOW QUALITY PROTEIN: C->U-editing enzyme
APOBEC-1 OS = Tarsius syrichta OX = 1868482 GN = APOBEC1 PE = 4 SV = 2
MLTALMEEVQDTMRFGRRAFFLSNSVGIWVLFDISISXSTGPSMGDPTLRRRIEPWEFEVLF
DPRELRKEACLLYEIKWGTSCKIWRNSGKNTSNHAEVNFLEKFTSERHFCSSTSYSITWFLS
WSPCWECSRAIREFLSQHPRVTLVIYVARLFWHMEPQNRQGLRDLINSGVTIQIMRDSGKS
NKQIIRIVCERTW
SEQ ID NO: 64
>tr|F1SLW4|F1SLW4_PIG CMP/dCMP-type deaminase domain-containing protein OS = Sus
scrota OX = 9823 GN = APOBEC1 PE = 4 SV = 2
MASDRGPSAGDATSRRRIEPWEFEVFFDPRELRKETCLLYELQWGRSRDTWRHTGKNTT
NHVERNFLAKITSERHFHPSVHCSIVWFLSWSPCWECSEAIREFLDQHPSVTLVIYVARLFQ
HMDPQNRQGLRDLVNHGVTIQIMGAPEYDYCWRNFVNYPPGKEAHWPRFPPVWMTLYAL
ELHCIILGLPPCLKISRRCQNQLTFFRLTLQNCHYQTIPPHILLATGLIQLPVIYR
SEQ ID NO: 65
>tr|A0A2K6BGI5|A0A2K6BGI5_MACNE CMP/dCMP-type deaminase domain-containing
protein OS = Macaca nemestrina OX = 9545 GN = APOBEC1 PE = 4 SV = 1
MTSEKGPSTGDPTLRRRIEPWEFDIFYDPRELRKEACLLYEIKWGMSPKIWRSSGKNTTNH
VEVNFIEKLTSERRFHSSISCSITWFLSWSPCWECSQAIREFLSQHPGVTLVIYVARLFWHT
DQQNRQGLRDLVNSGVTIQIMRASEYYHCWRNFVNYLPGEEAHWPRYPPLWMMLYALEL
HCIILSLPPCLKISRRWQNHLTFFRLHLQNCHYQMIPPHILLATGLIQPSVTWR
SEQ ID NO: 66
>sp|P47855|ABEC1_RABIT C->U-editing enzyme APOBEC-1 OS = Oryctolagus cuniculus
OX = 9986 GN = APOBEC1 PE = 1 SV = 1
MASEKGPSNKDYTLRRRIEPWEFEVFFDPQELRKEACLLYEIKWGASSKTWRSSGKNTTN
HVEVNFLEKLTSEGRLGPSTCCSITWFLSWSPCWECSMAIREFLSQHPGVTLIIFVARLFQH
MDRRNRQGLKDLVTSGVTVRVMSVSEYCYCWENFVNYPPGKAAQWPRYPPRWMLMYAL
ELYCIILGLPPCLKISRRHQKQLTFFSLTPQYCHYKMIPPYILLATGLLQPSVPWR
SEQ ID NO: 67
>sp|P38483|ABEC1_RAT C->U-editing enzyme APOBEC-1 OS = Rattus norvegicus
OX = 10116GN = Apobec1 PE = 1 SV = 1
MSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLK
SEQ ID NO: 68
>tr|A0A091M4D7|A0A091M4D7_CARIC C->U-editing enzyme APOBEC-1 (Fragment)
OS = Cariama cristata OX = 54380 GN = N322_12137 PE = 4 SV = 1
RWKIQPDDFKRNYLPGQHPEVVYLLYEIKWNSGTIWRNWCSNNPTQHAEVNFLENHFNVM
SSVSCSITWGISTTPCGKCSRRILEFLTTHPNVTLEIYAAKLFKHLDIRNRQGLRNLAMNGVVI
CIMNLADYSYFWKTFVAY
SEQ ID NO: 69
>tr|A0A093F3R4|A0A093F3R4_GAVST C->U-editing enzyme APOBEC-1 (Fragment)
OS = Gavia stellata OX = 37040 GN = N328_12441 PE = 4 SV = 1
RWKIQPNDFKRNYLPAQHPKVVYLLYEIRWSRGTIWRNWCSNNSTQHAEVNFLENCFKAM
PSVSCSITWFLSTTPCGKCSRRILTFLREHPNVTLEIYAAKLFKHLDVRNQQGLRNLDRNGVI
IRIMNFADYSYCWKRFVAY
SEQ ID NO: 70
>tr|G7N5W0|G7N5W0_MACMU C->U-editing enzyme APOBEC-1 (Fragment) OS = Macaca
mulatta OX = 9544 GN = EGK_03318 PE = 4 SV = 1
GPSTGDPTLRRRIEPWEFDIFYDPRELRKEACLLYEIKWGMSPKIWRSSGKNTTNHVEVNFI
EKLTSERRFHSSISCSITWFLSWSPCWECSQAIREFLSQHPGVTLVIYVARLFWHTDQQNR
QGLRDLVNSGVTIQIMRASEYYHCWRNFVNYPPGEEAHWPRYPPLWMMLYALELHCIILSL
PPCLKISRRWQNHLTFFRLHLQNCHYQMIPPHILLATGLIQPSVTWR
SEQ ID NO: 71
>tr|A0A091MEP8|A0A091MEP8_9PASS C->U-editing enzyme APOBEC-1 (Fragment)
OS = Acanthisitta chloris OX = 57068 GN = N310_12928 PE = 4 SV = 1
RWKIQPNDFQRNYLPDQHPQAVYLLYEFRWRRGSIWRKWCSNNRAQHAEVNFLENCFNG
IPPVPCSITWFLSTTPCGNCSRRILEFLRLHPNVTLEIYAAKLFRHTDIRNRKGLYNLAMNGVII
RIMNLADYSYCWRNFVAY
SEQ ID NO: 72
>tr|A0A2I0LXZ8|A0A2I0LXZ8_COLLI Apolipoprotein B mRNA editing enzyme, catalytic
polypeptide 1 OS = Columba livia OX = 8932 GN = APOBEC1 PE = 4 SV = 1
MAAVTNRDSACRENNQRWKIQPNDFRRNYLPDKQPRVVYLLYEIRWRRGTIWRNWCSNN
PNQHAEVNFLKNYFNAMPSVSCSITWVLSTTPCGKCSIKIMEFLKLHPNVTLEIYAAKLFKHL
DIRNREGLRNLAKNGVIIHIMNLADYSYWWKIFVTRQHGEEDYLPWSFALHIFLNCIEFQQILL
GLPPLLPNFKY
SEQ ID NO: 73
>tr|W5NVH9|W5NVH9_SHEEP CMP/dCMP-type deaminase domain-containing protein
OS = Ovis aries OX = 9940 GN = APOBEC1 PE = 4 SV = 1
MASDRGPPAGDPTLRRRIEPLEFEFSFDPRNFCKEAYLLYEIQWGNSRDVWRHSGKNTTK
HVERNFIEKIASERHFRPSISCSISWYLSWSPCWECSKAIREFLNQHPNVTLVIYIARLFQHM
DPQNRQGLKDLFHSGVTIQVMRDPEYDYCWRNFVNYPQGKEAHWPRYPPLWMNLYALEL
YCIISGLPPCLQISRRHQNQLRVFRLIPQNCHYQMIPPCILLATGMIQLPVTWRWIE
SEQ ID NO: 74
>tr|H0XVG8|H0XVG8_OTOGA CMP/dCMP-type deaminase domain-containing protein
OS = Otolemur garnettii OX = 30611 GN = APOBEC1 PE = 4 SV = 1
ISWSTGISTGDPTLRRRIEPWEFEVFFDPRELRKETCLLYEIKWGTSHKIWRSTARNTTS
HAEMNFIEKFTSERCSDAPISCSITWFLSWSPCWECSKAIREFVSRHPSVTLVIYVARLY
WHMDQQNRQGLRDLISSGVTVQIMRVSEYCHCWRNFVNYLPGKEAHCPRCPPLWMTLYA
LELHCIILSLPPCLKISRGHQNQLTLFRLTLQNCHYQTIPPHVLLATGLIQPYVTWR
SEQ ID NO: 75
>tr|A0A2B4RXQ3|A0A2B4RXQ3_STYPI C->U-editing enzyme APOBEC-1 OS = Stylophora
pistillata OX = 50429 GN = APOBEC1 PE = 4 SV = 1
MASVTELRTPDDFLAELLWTGVTGRTWPNRTFLIVSIKAKDGKPIFGKRFKNRYPEHAEI
IMLRNSNFSDVVEKNHDIDITLTLNYSPCSSCACILKEFYVNNSNIKCFTIQFSFIYYKE
DMKNKTGLQNLEEAGVTLQAMNAESWREVGIDLESFTPEDKEKINKRDKDTANDLNEVLSS
KQDQDASVDELSSQLNAKLRAKET
SEQ ID NO: 76
>tr|A0A2K5L2J6|A0A2K5L2J6_CERAT CMP/dCMP-type deaminase domain-containing
protein OS = Cercocebus atys OX = 9531 GN = APOBEC1 PE = 4 SV = 1
MTPEEEVQRQSTMTSEKGPSTGDPTLRRRIEPWEFDIFYDPRELRKEACLLYEIKWGMSPK
IWRSSGKNTTNHVEVNFIEKLTSERRFHSSISCSITWFLSWSPCWECSQAIREFLSQHPGVT
LVIYVARLFWHTDQQNRQGLRDLVNSGVTIQIMRASEYYHCWRNFVNYPPGEEAHWPRYP
PLWMMLYALELHCIILSLPPCLKISRRQQNHLTFFRLHLQNCHYQTIPPHILLATGLIQPSVTW
R
SEQ ID NO: 77
>tr|A0A2Y9T649|A0A2Y9T649_PHYMC C->U-editing enzyme APOBEC-1 isoform X1
OS = Physeter macrocephalus OX = 9755 GN = APOBEC1 PE = 4 SV = 1
MIICWSTGPSAGDATSRRRIEPWEFEVSFDPREFCKEARLLYEIKWGKSQDVWRHSGKNT
TKHVECNFIEKMTSERHFHPSISCCIIWFLSWSPCWECSKAIREFLNQHPSVTLVIYIARLFQ
HTDPQNRQGLRDLIHSGVTLQIMGPPEYDYCWRNFVNYPPGKEAHWPRYPPLWMKLYAL
ELHCIILGLPPCLKISRRCQNQLTWFRLILQNCHYQMIPPHILLGTGLIQLPVAWR
SEQ ID NO: 78
>tr|H2NGDO|H2NGDO_PONAB Apolipoprotein B mRNA editing enzyme catalytic subunit 1
OS = Pongo abelii OX = 9601 GN = APOBEC1 PE = 4 SV = 1
MTPEEEVQRQSTMTSEKGPSTGDPTLRRRIESWEFDVFYDPRELRKETCLLYEIKWGMSR
KIWRSSGKNTTNHVEVNFIKKFTSERRFHSSISCSITWFLSWSPCWECSQAIREFLSQHPGV
TLVIYVARLFWHMDQRNRQGLRDLVNSGVTIQIMRASEYYHCWRNFVNYPPGDEAHWPQ
YPPLWMMLYALELHCIILSLPPCLKISRRWQNHLAFFRLHLQNCHYQTIPPHILLATGLIHPSV
TWK
SEQ ID NO: 79
>tr|A0A093JI54|A0A093JI54_EURHL C->U-editing enzyme APOBEC-1 (Fragment)
OS = Eurypyga helias OX = 54383 GN = N326_10046 PE = 4 SV = 1
RWKIQPNDFKRNYMPSQYPKVVYLLYEIRWSRGTVWRNWCSNSFTQHAEVNFLENYFKP
MPSVSCSITWVLSTTPCGKCSRRILEFLRVHPNVTLEIYAAKLFKHLDIRNRQGLRDLAMNG
VTIRIMNLADYSFCWKRFVAY
SEQ ID NO: 80
>tr|G3W4H|G3W4H_SARHA Apolipoprotein B mRNA editing enzyme catalytic subunit 1
OS = Sarcophilus harrisii OX = 9305 GN = APOBEC1 PE = 4 SV = 1
MGDATLRRRIKSWEFEAFFNPQELRKETCLLYEIKWGASHNIWRSSNQNTTQHAEINFMEK
FTSERNFKPSVKCSITWFLSWSPCWRCSKAIREFLNQYPNVTLVIFVSRLYWHMEQQHRQ
ELKELVCSGVTIQIMNYSEYRHCWRNFVDYLPEEEDHWPKYPTLWIMLYVLELHCIILGLPP
CLKISVRHSDQLVLFSLDLQDCHYQKIPYHVLVATGIIRPFVTWR
SEQ ID NO: 81
>tr|A0A2U3Y3M5|A0A2U3Y3M5_LEPWE c->U-editing enzyme APOBEC-1
OS = Leptonychotes weddellii OX = 9713 GN = APOBEC1 PE = 4 SV = 1
MASDKGPSAGDATLRRRIKPWEFEVFFDPRELRKETCLLYEIQWGTSHKIWRNSGKNTAN
HVEINFIEKFTSERQYCPSIRCSITWFLSWSPCWECSKAIRGFLSQHPSVTLVIYVARLFWH
MDPQNRQGLRDLINSGVTIQIMRVPEYDHCWRNFVNYLPGKEDHWPRYPVLWMKLYALEL
HCIILPIEMPGKIRDAPNNMEIFSLFVGRYIPKTKFHVTCLLSDVRNDDSHLDKTAPKWIRFDS
LQPVASDPSAEHWKMKLPGRDDKTAVVVGTVTEDVACAQGAKLYLCALRVHGHAQRHFL
KGRDEILALDQLALDSPQGLWRQPDLRSHPLKG
SEQ ID NO: 82
>tr|A0A1S3AN78|A0A1S3AN78_ERIEU C->U-editing enzyme APOBEC-1-like
OS = Erinaceus europaeus OX = 9365 GN = LOC103126721 PE = 4 SV = 1
RRIEPWEFEDFFDPRQFRPETCLLYEVRWGSSRNAWRSTARNTTRHAEVNFLERFAAERH
FDKPVSCSITWFLSWSPCWECSQAIGAFLSQHPQVTLAIHVTRLFHHEDEQNRQGLRDLLA
RGVTLQVMGDSEYAHCWRTFVNSPPGAEGHYPRYPSDFTRLYALELHCIILGLPPCLEILRR
YQNQFTLFRLVPQNCHYQMIPHLNFFVVRHYFF
SEQ ID NO: 83
>tr|A0A091PSV3|A0A091PSV3_HALAL C->U-editing enzyme APOBEC-1 (Fragment)
OS = Haliaeetus albicilla OX = 8969 GN = N329_07103 PE = 4 SV = 1
RWKLQPNDFKRNYLPGQHPKVVYLLYEIRWSRGTIWRNWCSNNSTQHAEVNFLENCFKAT
PSVSCSITWVLSTTPCGKCSRRILEFLRVHPNVTLEIYAAKLFKHLDIRNRKGLRDLAMNGVII
RIMNLSDYSYCWKTFVAY
SEQ ID NO: 84
>tr|F7F6M6|F7F6M6_CALJA Apolipoprotein B mRNA editing enzyme catalytic subunit 1
OS = Callithrix jacchus OX = 9483 GN = APOBEC1 PE = 4 SV = 2
RRIEPWEFYISYDPKELCKETCLLYEIKWGMSWKIWRSSGKNTTNHVEINFIEKFTSERH
FHLSVSCSITWFLSWSPCWECSQAIREFLSQHPGVTLVIYVARLFQHMDQQNRQGLRDLV
NSGVTIQMMTVSEYYHCWRNFVNYPPGEEAHWPRHPPLWLMLYALELHCIILGLPPCLKIS
RRRQNRLTFFRLHLQNCHYQMIPRHILLATGLIQPSVTWR
SEQ ID NO: 85
>tr|L8IDZ0|L8IDZ0_9CETA C->U-editing enzyme APOBEC-1 OS = Bos mutus OX = 72004
GN = M91_02456 PE = 4 SV = 1
MIISWSTGPPAGDPTLRRRIEPWEFEFSFDPRKFCKEACLLYEIQWGNNRDVWRHSGKNTT
KHVERNFIEKIASERYFCPSIRCFIFWYLSWSPCWECSKAIREFLNQHPNVTLVIYIARLFQH
MDPQNRQGLKDLVQSGVTIQVMRAPEYEYCWRNFVNYPRGKEAHWPRYPPLWMNLYAL
ELYCIILGLPPCLHISRRYQNQLIVFRLTLQNCHYQMIPPYILLATGMVQIPMTWR
SEQ ID NO: 86
>tr|A0A093CIQ8|A0A093CIQ8_9AVES C->U-editing enzyme APOBEC-1 (Fragment)
OS = Pterocles gutturalis OX = 240206 GN = N339_03265 PE = 4 SV = 1
RWKIQPNYFKINNLPGQHPRVVCLLYAIRWSRSTLWKSWCSNNSTQHAEVNFLENCFKGN
PSVFCFMTWPFFHTTPHGKCCRRTPEFLGVHPNVTLKIRAAKLFKHLDRYNQQGLRNVAM
NGVVIRIINL
SEQ ID NO: 87
>sp|Q9GZX7|AICDA_HUMAN Single-stranded DNA cytosine deaminase OS = Homo sapiens
OX = 9606 GN = AICDA PE = 1 SV = 1
MDSLLMNRRKFLYQFKNVRWAKGRRETYLCYVVKRRDSATSFSLDFGYLRNKNGCHVELL
FLRYISDWDLDPGRCYRVTWFTSWSPCYDCARHVADFLRGNPNLSLRIFTARLYFCEDRKA
EPEGLRRLHRAGVQIAIMTFKDYFYCWNTFVENHERTFKAWEGLHENSVRLSRQLRRILLP
LYEVDDLRDAFRTLGL
SEQ ID NO: 88
>sp|Q9Y235|ABEC2_HUMAN C->U-editing enzyme APOBEC-2 OS = Homo sapiens
OX = 9606 GN = APOBEC2 PE = 1 SV = 1
MAQKEEAAVATEAASQNGEDLENLDDPEKLKELIELPPFEIVTGERLPANFFKFQFRNVEYS
SGRNKTFLCYVVEAQGKGGQVQASRGYLEDEHAAAHAEEAFFNTILPAFDPALRYNVTWY
VSSSPCAACADRIIKTLSKTKNLRLLILVGRLFMWEEPEIQAALKKLKEAGCKLRIMKPQDFE
YVWQNFVEQEEGESKAFQPWEDIQENFLYYEEKLADILK
SEQ ID NO: 89
>sp|P31941|ABC3A_HUMAN DNA dC->dll-editing enzyme APOBEC-3A OS = Homo sapiens
OX = 9606 GN = APOBEC3A PE = 1 SV = 3
MEASPASGPRHLMDPHIFTSNFNNGIGRHKTYLCYEVERLDNGTSVKMDQHRGFLHNQAK
NLLCGFYGRHAELRFLDLVPSLQLDPAQIYRVTWFISWSPCFSWGCAGEVRAFLQENTHVR
LRIFAARIYDYDPLYKEALQMLRDAGAQVSIMTYDEFKHCWDTFVDHQGCPFQPWDGLDE
HSQALSGRLRAILQNQGN
SEQ ID NO: 90
>sp|Q9UH17|ABC3B_HUMAN DNA dC->dU-editing enzyme APOBEC-3B OS = Homo
sapiens OX = 9606 GN = APOBEC3B PE = 1 SV = 1
MNPQIRNPMERMYRDTFYDNFENEPILYGRSYTWLCYEVKIKRGRSNLLWDTGVFRGQVY
FKPQYHAEMCFLSWFCGNQLPAYKCFQITWFVSWTPCPDCVAKLAEFLSEHPNVTLTISAA
RLYYYWERDYRRALCRLSQAGARVTIMDYEEFAYCWENFVYNEGQQFMPWYKFDENYAF
LHRTLKEILRYLMDPDTFTFNFNNDPLVLRRRQTYLCYEVERLDNGTWVLMDQHMGFLCNE
AKNLLCGFYGRHAELRFLDLVPSLQLDPAQIYRVTWFISWSPCFSWGCAGEVRAFLQENTH
VRLRIFAARIYDYDPLYKEALQMLRDAGAQVSIMTYDEFEYCWDTFVYRQGCPFQPWDGLE
EHSQALSGRLRAILQNQGN
SEQ ID NO: 91
>sp|Q9NRW3|ABC3C_HUMAN DNA dC->dU-editing enzyme APOBEC-3C OS = Homo
sapiens OX = 9606 GN = APOBEC3C PE = 1 SV = 2
MNPQIRNPMKAMYPGTFYFQFKNLWEANDRNETWLCFTVEGIKRRSVVSWKTGVFRNQV
DSETHCHAERCFLSWFCDDILSPNTKYQVTWYTSWSPCPDCAGEVAEFLARHSNVNLTIFT
ARLYYFQYPCYQEGLRSLSQEGVAVEIMDYEDFKYCWENFVYNDNEPFKPWKGLKTNFRL
LKRRLRESLQ
SEQ ID NO: 92
>sp|Q96AK3|ABC3D_HUMAN DNA dC->dU-editing enzyme APOBEC-3D OS = Homo
sapiens OX = 9606 GN = APOBEC3D PE = 1 SV = 1
MNPQIRNPMERMYRDTFYDNFENEPILYGRSYTWLCYEVKIKRGRSNLLWDTGVFRGPVL
PKRQSNHRQEVYFRFENHAEMCFLSWFCGNRLPANRRFQITWFVSWNPCLPCVVKVTKF
LAEHPNVTLTISAARLYYYRDRDWRWVLLRLHKAGARVKIMDYEDFAYCWENFVCNEGQP
FMPWYKFDDNYASLHRTLKEILRNPMEAMYPHIFYFHFKNLLKACGRNESWLCFTMEVTKH
HSAVFRKRGVFRNQVDPETHCHAERCFLSWFCDDILSPNTNYEVTWYTSWSPCPECAGE
VAEFLARHSNVNLTIFTARLCYFWDTDYQEGLCSLSQEGASVKIMGYKDFVSCWKNFVYSD
DEPFKPWKGLQTNFRLLKRRLREILQ
SEQ ID NO: 93
>sp|Q8IUX4|ABC3F_HUMAN DNA dC->dU-editing enzyme APOBEC-3F OS = Homo sapiens
OX = 9606 GN = APOBEC3F PE = 1 SV = 3
MKPHFRNTVERMYRDTFSYNFYNRPILSRRNTVWLCYEVKTKGPSRPRLDAKIFRGQVYS
QPEHHAEMCFLSWFCGNQLPAYKCFQITWFVSWTPCPDCVAKLAEFLAEHPNVTLTISAAR
LYYYWERDYRRALCRLSQAGARVKIMDDEEFAYCWENFVYSEGQPFMPWYKFDDNYAFL
HRTLKEILRNPMEAMYPHIFYFHFKNLRKAYGRNESWLCFTMEWKHHSPVSWKRGVFRN
QVDPETHCHAERCFLSWFCDDILSPNTNYEVTWYTSWSPCPECAGEVAEFLARHSNVNLTI
FTARLYYFWDTDYQEGLRSLSQEGASVEIMGYKDFKYCWENFVYNDDEPFKPWKGLKYNF
LFLDSKLQEILE
SEQ ID NO: 94
>sp|Q9HC16|ABC3G_HUMAN DNA dC->dU-editing enzyme APOBEC-3G OS = Homo
sapiens OX = 9606 GN = APOBEC3G PE = 1 SV = 1
MKPHFRNTVERMYRDTFSYNFYNRPILSRRNTVWLCYEVKTKGPSRPPLDAKIFRGQVYSE
LKYHPEMRFFHWFSKWRKLHRDQEYEVTWYISWSPCTKCTRDMATFLAEDPKVTLTIFVA
RLYYFWDPDYQEALRSLCQKRDGPRATMKIMNYDEFQHCWSKFVYSQRELFEPWNNLPK
YYILLHIMLGEILRHSMDPPTFTFNFNNEPWVRGRHETYLCYEVERMHNDTWVLLNQRRGF
LCNQAPHKHGFLEGRHAELCFLDVIPFWKLDLDQDYRVTCFTSWSPCFSCAQEMAKFISKN
KHVSLCIFTARIYDDQGRCQEGLRTLAEAGAKISIMTYSEFKHCWDTFVDHQGCPFQPWDG
LDEHSQDLSGRLRAILQNQEN
SEQ ID NO: 95
>sp|Q6NTF7|ABC3H_HUMAN DNA dC->dU-editing enzyme APOBEC-3H OS = Homo
sapiens OX = 9606 GN = APOBEC3H PE = 1 SV = 4
MALLTAETFRLQFNNKRRLRRPYYPRKALLCYQLTPQNGSTPTRGYFENKKKCHAEICFINE
IKSMGLDETQCYQVTCYLTWSPCSSCAWELVDFIKAHDHLNLGIFASRLYYHWCKPQQKGL
RLLCGSQVPVEVMGFPEFADCWENFVDHEKPLSFNPYKMLEELDKNSRAIKRRLERIKIPG
VRAQGRYMDILCDAEV
Petromyzon marinus cytosine deaminase (pmCDA1), Genbank ABO15149.1
SEQ ID NO: 96
MTDAEYVRIHEKLDIYTFKKQFFNNKKSVSHRCYVLFELKRRGERRACFWGYAVNKPQSGT
ERGIHAEIFSIRKVEEYLRDNPGQFTINWYSSWSPCADCAEKILEWYNQELRGNGHTLKIWA
CKLYYEKNARNQIGLWNLRDNGVGLNVMVSEHYQCCRKIFIQSSHNQLNENRWLEKTLKR
AEKRRSELSIMIQVKILHTTKSPAV
Petromyzon marinus cytosine deaminase (pmCDA1) R187W, as used in Target-AID,
SEQ ID NO: 97
MTDAEYVRIHEKLDIYTFKKQFFNNKKSVSHRCYVLFELKRRGERRACFWGYAVNKPQSGT
ERGIHAEIFSIRKVEEYLRDNPGQFTINWYSSWSPCADCAEKILEWYNQELRGNGHTLKIWA
CKLYYEKNARNQIGLWNLRDNGVGLNVMVSEHYQCCRKIFIQSSHNQLNENRWLEKTLKR
AEKWRSELSIMIQVKILHTTKSPAV
E. coli TadA, SEQ ID NO: 98
MSEVEFSHEYWMRHALTLAKRAWDEREVPVGAVLVHNNRVIGEGWNRPIGRHDPTAHAEI
MALRQGGLVMQNYRLIDATLYVTLEPCVMCAGAMIHSRIGRVVFGARDAKTGAAGSLMDVL
HHPGMNHRVEITEGILADECAALLSDFFRMRRQEIKAQKKAQSSTD
S. aureus TadA, SEQ ID NO: 99
MTNDIYFMTLAIEEAKKAAQLGEVPIGAIITKDDEVIARAHNLRETLQQPTAHAEHIAIERAAKV
LGSWRLEGCTLYVTLEPCVMCAGTIVMSRIPRVVYGADDPKGGCSGSLMNLLQQSNFNHR
AIVDKGVLKEACSTLLTTFFKNLRANKKSTN
S. pyogenes TadA, SEQ ID NO: 100
MPYSLEEQTYFMQEALKEAEKSLQKAEIPIGCVIVKDGEIIGRGHNAREESNQAIMHAEMMAI
NEANAHEGNWRLLDTTLFVTIEPCVMCSGAIGLARIPHVIYGASNQKFGGADSLYQILTDER
LNHRVQVERGLLAADCANIMQTFFRQGRERKKIAKHLIKEQSDPFD
S. typhi TadA, SEQ ID NO: 101
MSDVELDHEYWMRHALTLAKRAWDEREVPVGAVLVHNHRVIGEGWNRPIGRHDPTAHAEI
MALRQGGLVLQNYRLLDTTLYVTLEPCVMCAGAMVHSRIGRWFGARDAKTGAAGSLIDVL
HHPGMNHRVEIIEGVLRDECATLLSDFFRMRRQEIKALKKADRAEGAGPAV
A. aeolicus TadA, SEQ ID NO: 102
MGKEYFLKVALREAKRAFEKGEVPVGAIIVKEGEIISKAHNSVEELKDPTAHAEMLAIKEACR
RLNTKYLEGCELYVTLEPCIMCSYALVLSRIEKVIFSALDKKHGGVVSVFNILDEPTLNHRVK
WEYYPLEEASELLSEFFKKLRNNII
S. pombe TAD2, SEQ ID NO: 103
MAGDSVKSAIIGIAGGPFSGKTQLCEQLLERLKSSAPSTFSKLIHLTSFLYPNSVDRYALSSY
DIEAFKKVLSLISQGAEKICLPDGSCIKLPVDQNRIILIEGYYLLLPELLPYYTSKIFVYEDADTR
LERCVLQRVKAEKGDLTKVLNDFVTLSKPAYDSSIHPTRENADIILPQKENIDTALLFVSQHL
QDILAEMNKTSSSNTVKYDTQHETYMKLAHEILNLGPYFVIQPRSPGSCVFVYKGEVIGRGF
NETNCSLSGIRHAELIAIEKILEHYPASVFKETTLYVTVEPCLMCAAALKQLHIKAVYFGCGND
RFGGCGSVFSINKDQSIDPSYPVYPGLFYSEAVMLMREFYVQENVKAPVPQSKKQRVLKR
EVKSLDLSRFK
S. cerevisiae TAD1, SEQ ID NO: 104
MVSCQGTRPCIVNLLTMPSEDKLGEEISTRVINEYSKLKSACRPIIRPSGIREWTILAGVAAIN
RDGGANKIEILSIATGVKALPDSELQRSEGKILHDCHAEILALRGANTVLLNRIQNYNPSSGD
KFIQHNDEIPARFNLKENWELALYISRLPCGDASMSFLNDNCKNDDFIKIEDSDEFQYVDRS
VKTILRGRLNFNRRNVVRTKPGRYDSNITLSKSCSDKLLMKQRSSVLNCLNYELFEKPVFLK
YIVIPNLEDETKHHLEQSFHTRLPNLDNEIKFLNCLKPFYDDKLDEEDVPGLMCSVKLFMDDF
STEEAILNGVRNGFYTKSSKPLRKHCQSQVSRFAQWELFKKIRPEYEGISYLEFKSRQKKRS
QLIIAIKNILSPDGWIPTRTDDVK
S. cerevisiae TAD2, SEQ ID NO: 105
MQHIKHMRTAVRLARYALDHDETPVACIFVHTPTGQVMAYGMNDTNKSLTGVAHAEFMGI
DQIKAMLGSRGVVDVFKDITLYVTVEPCIMCASALKQLDIGKVVFGCGNERFGGNGTVLSVN
HDTCTLVPKNNSAAGYESIPGILRKEAIMLLRYFYVRQNERAPKPRSKSDRVLDKNTFPPME
WSKYLNEEAFIETFGDDYRTCFANKVDLSSNSVDWDLIDSHQDNIIQELEEQCKMFKFNVH
KKSKV
A. thaliana TAD2, SEQ ID NO: 106
MEEDHCEDSHNYMGFALHQAKLALEALEVPVGCVFLEDGKVIASGRNRTNETRNATRHAE
MEAIDQLVGQWQKDGLSPSQVAEKFSKCVLYVTCEPCIMCASALSFLGIKEVYYGCPNDKF
GGCGSILSLHLGSEEAQRGKGYKCRGGIMAEEAVSLFKCFYEQGNPNAPKPHRPVVQRER
T
X. laevis ADAT2, SEQ ID NO: 107
MEPLQITEEIQNWMHKAFQMAQDALNNGEVPVGCLMVYGNQWGKGRNEVNETKNATQH
AEMVAIDQVLDWCEMNSKKSTDVFENIVLYVTVEPCIMCAGALRLLKIPLWYGCRNERFGG
CGSVLNVSGDDIPDTGTKFKCIGGYQAEKAIELLKTFYKQENPNAPKSKVRKKE
X. tropicalis ADAT2, SEQ ID NO: 108
MTEEIQNWMHKAFQMAQDALNNGEVPVGCLMVYDNQVVGKGRNEVNETKNATRHAEMV
AIDQVLDWCEKNSKKSRDVFENIVLYVTVEPCIMCAGALRLLKIPLWYGCRNERFGGCGSV
LNVAGDNIPDTGTEFKYIGGYQAEKAVELLKTFYKQENPNAPRSKVRKKE
D. rerio ADAT2, SEQ ID NO: 109
MQEVGVDPEKNDFLQPSDSEVQTWMAKAFDMAVEALENGEVPVGCLMVYNNEIIGKGRN
EVNETKNATRHAEMVALDQVLDWCRLREKDCKEVCEQTVLYVTVEPCIMCAAALRLLRIPF
VVYGCKNERFGGCGSVLDVSSDHLPHTGTSFKCIAGYRAEEAVEMLKTFYKQENPNAPKP
KVRKDSINPQDGAAVIQVMRGPPDEETETIAHLS
B. Taurus ADAT2, SEQ ID NO: 110
MEAKAGPTAATDGAYSVSAEETEKWMEQAMQMAKDALDNTEVPVGCLMVYNNEVVGKG
RNEVNQTKNATRHAEMVAIDQALDWCRRRGRSPSEVFEHTVLYVTVEPCIMCAAALRLMRI
PLVVYGCQNERFGGCGSVLDIASADLPSTGKPFQCTPGYRAEEAVEMLKTFYKQENPNAP
KSKVRKKECHKS
M. musculus ADAT2, SEQ ID NO: 111
MEEKVESTTTPDGPCVVSVQETEKWMEEAMRMAKEALENIEVPVGCLMVYNNEVVGKGR
NEVNQTKNATRHAEMVAIDQVLDWCHQHGQSPSTVFEHTVLYVTVEPCIMCAAALRLMKIP
LVVYGCQNERFGGCGSVLNIASADLPNTGRPFQCIPGYRAEEAVELLKTFYKQENPNAPKS
KVRKKDCQKS
H. sapiens ADAT2 SEQ ID NO: 112
MEAKAAPKPAASGACSVSAEETEKWMEEAMHMAKEALENTEVPVGCLMVYNNEVVGKGR
NEVNQTKNATRHAEMVAIDQVLDWCRQSGKSPSEVFEHTVLYVTVEPCIMCAAALRLMKIP
LWYGCQNERFGGCGSVLNIASADLPNTGRPFQCIPGYRAEEAVEMLKTFYKQENPNAPKS
KVRKKECQKS
BE1 for Mammalian expression (rAPOBEC1-XTEN-dCas9-NLS)
SEQ ID NO: 113
MSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGSETPGTSESATPESDKK
YSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTFR
IPYYVGPLARGNSRFAWMTRKSEETITPWNFEEWDKGASAQSFIERMTNFDKNLPNEKVL
PKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEER
LKTYAHLFDDKVMKQLKRRRYTGWGRLSRKLINGIRDKQSGKTILDFLKSDGFANRNFMQLI
HDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGR
DMYVDQELDINRLSDYDVDAIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSPKKKRKV
BE2 (rAPOBEC1-XTEN-dCas9-UGI-NLS) SEQ ID NO: 114
MSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGSETPGTSESATPESDKK
YSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTFR
IPYYVGPLARGNSRFAWMTRKSEETITPWNFEEWDKGASAQSFIERMTNFDKNLPNEKVL
PKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEER
LKTYAHLFDDKVMKQLKRRRYTGWGRLSRKLINGIRDKQSGKTILDFLKSDGFANRNFMQLI
HDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGR
DMYVDQELDINRLSDYDVDAIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSTNLSDIIEKETG
KQLVIQESILMLPEEVEEVIGNKPESDILVHTAYDESTDENVMLLTSDAPEYKPWALVIQDSN
GENKIKMLSGGSPKKKRKV
BE3 (rAPOBEC1-XTEN-Cas9n-UGI-NLS) SEQ ID NO: 115
MSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGSETPGTSESATPESDKK
YSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTFR
IPYYVGPLARGNSRFAWMTRKSEETITPWNFEEVVDKGASAQSFIERMTNFDKNLPNEKVL
PKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEER
LKTYAHLFDDKVMKQLKRRRYTGWGRLSRKLINGIRDKQSGKTILDFLKSDGFANRNFMQLI
HDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGR
DMYVDQELDINRLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSTNLSDIIEKETG
KQLVIQESILMLPEEVEEVIGNKPESDILVHTAYDESTDENVMLLTSDAPEYKPWALVIQDSN
GENKIKMLSGGSPKKKRKV
CDA1-BE3: SEQ ID NO: 116
MTDAEYVRIHEKLDIYTFKKQFFNNKKSVSHRCYVLFELKRRGERRACFWGYAVNKPQSGT
ERGIHAEIFSIRKVEEYLRDNPGQFTINWYSSWSPCADCAEKILEWYNQELRGNGHTLKIWA
CKLYYEKNARNQIGLWNLRDNGVGLNVMVSEHYQCCRKIFIQSSHNQLNENRWLEKTLKR
AEKRRSELSIMIQVKILHTTKSPAVSGSETPGTSESATPESDKKYSIGLAIGTNSVGWAVITD
EYKVPSKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPL
IETNGETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDSGGSTNLS
DIIEKETGKQLVIQESILMLPEEVEEVIGNKPESDILVHTAYDESTDENVMLLTSDAPEYKPWA
LVIQDSNGENKIKMLSGGSPKKKRKV
AID-BE3: SEQ ID NO: 117
MDSLLMNRRKFLYQFKNVRWAKGRRETYLCYVVKRRDSATSFSLDFGYLRNKNGCHVELL
FLRYISDWDLDPGRCYRVTWFTSWSPCYDCARHVADFLRGNPNLSLRIFTARLYFCEDRKA
EPEGLRRLHRAGVQIAIMTFKDYFYCWNTFVENHERTFKAWEGLHENSVRLSRQLRRILLP
LYEVDDLRDAFRTLGLSGSETPGTSESATPESDKKYSIGLAIGTNSVGWAVITDEYKVPSKK
FKVLGNTDRHSIKKNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAEDAKLQLSKDTYDDD
LDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYFDTTIDRKRYTSTK
EVLDATLIHQSITGLYETRIDLSQLGGDSGGSTNLSDIIEKETGKQLVIQESILMLPEEVEEVIG
NKPESDILVHTAYDESTDENVMLLTSDAPEYKPWALVIQDSNGENKIKMLSGGSPKKKRKV
BE3-Gam: SEQ ID NO: 118
MAKPAKRIKSAAAAYVPQNRDAVITDIKRIGDLQREASRLETEMNDAIAEITEKFAARIAPIKT
DIETLSKGVQGWCEANRDELTNGGKVKTANLVTGDVSWRVRPPSVSIRGMDAVMETLERL
GLQRFIRTKQEINKEAILLEPKAVAGVAGITVKSGIEDFSIIPFEQEAGISGSETPGTSESATPE
SSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGSETPGTSESATPESDKK
YSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTFR
IPYYVGPLARGNSRFAWMTRKSEETITPWNFEEVVDKGASAQSFIERMTNFDKNLPNEKVL
PKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEER
LKTYAHLFDDKVMKQLKRRRYTGWGRLSRKLINGIRDKQSGKTILDFLKSDGFANRNFMQLI
HDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGR
DMYVDQELDINRLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSTNLSDIIEKETG
KQLVIQESILMLPEEVEEVIGNKPESDILVHTAYDESTDENVMLLTSDAPEYKPWALVIQDSN
GENKIKMLSGGSPKKKRKV
SaBE3-Gam: SEQ ID NO: 119
MAKPAKRIKSAAAAYVPQNRDAVITDIKRIGDLQREASRLETEMNDAIAEITEKFAARIAPIKT
DIETLSKGVQGWCEANRDELTNGGKVKTANLVTGDVSWRVRPPSVSIRGMDAVMETLERL
GLQRFIRTKQEINKEAILLEPKAVAGVAGITVKSGIEDFSIIPFEQEAGISGSETPGTSESATPE
SSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGSETPGTSESATPESGKR
NYILGLAIGITSVGYGIIDYETRDVIDAGVRLFKEANVENNEGRRSKRGARRLKRRRRHRIQR
VKKLLFDYNLLTDHSELSGINPYEARVKGLSQKLSEEEFSAALLHLAKRRGVHNVNEVEEDT
GNELSTKEQISRNSKALEEKYVAELQLERLKKDGEVRGSINRFKTSDYVKEAKQLLKVQKAY
HQLDQSFIDTYIDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAKEILVNEEDIKGYRV
TSTGKPEFTNLKVYHDIKDITARKEIIENAELLDQIAKILTIYQSSEDIQEELTNLNSELTQEEIE
QISNLKGYTGTHNLSLKAINLILDELWHTNDNQIAIFNRLKLVPKKVDLSQQKEIPTTLVDDFIL
SPVVKRSFIQSIKVINAIIKKYGLPNDIIIELAREKNSKDAQKMINEMQKRNRQTNERIEEIIRTT
GKENAKYLIEKIKLHDMQEGKCLYSLEAIPLEDLLNNPFNYEVDHIIPRSVSFDNSFNNKVLVK
QEENSKKGNRTPFQYLSSSDSKISYETFKKHILNLAKGKGRISKTKKEYLLEERDINRFSVQK
DFINRNLVDTRYATRGLMNLLRSYFRVNNLDVKVKSINGGFTSFLRRKWKFKKERNKGYKH
HAEDALHANADFIFKEWKKLDKAKKVMENQMFEEKQAESMPEIETEQEYKEIFITPHQIKHIK
DFKDYKYSHRVDKKPNRELINDTLYSTRKDDKGNTLIVNNLNGLYDKDNDKLKKLINKSPEK
LLMYHHDPQTYQKLKLIMEQYGDEKNPLYKYYEETGNYLTKYSKKDNGPVIKKIKYYGNKLN
AHLDITDDYPNSRNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDITYREYLENMNDKRPP
RIIKTIASKTQSIKKYSTDILGNLYEVKSKKHPQIIKKGGSPKKKRKVSSDYKDHDGDYKDHDI
DYKDDDDKSGGSTNLSDIIEKETGKQLVIQESILMLPEEVEEVIGNKPESDILVHTAYDESTD
ENVMLLT SDAPEYKPWALVIQDSNGENKIKMLSGGSPKKKRKV
BE4: SEQ ID NO: 120
MSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGGSSGGSSGSETPGTSE
SATPESSGGSSGGSDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAILRRQE
DFYPFLKDNREKIEKILTFRIPYYVGPL
ARGNSRFAWMTRKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVDQE
LDINRLSDYDVDHIVPQSFLKDDSIDNKVLTRSDKNRGKSDNVPSEEVVKKMKNYWRQLLN
AKLITQRKFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGAPAAFKYFD
TTIDRKRYTSTKEVLDATLIHQSITGLYETRIDLSQLGGDSGGSGGSGGSTNLSDIIEKETGK
QLVIQESILMLPEEVEEVIGNKPESDILVHTAYDESTDENVMLLTSDAPEYKPWALVIQDSNG
ENKIKMLSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSPKKKRK
BE4-Gam: SEQ ID NO: 121
MAKPAKRIKSAAAAYVPQNRDAVITDIKRIGDLQREASRLETEMNDAIAEITEKFAARIAPIKT
DIETLSKGVQGWCEANRDELTNGGKVKTANLVTGDVSWRVRPPSVSIRGMDAVMETLERL
GLQRFIRTKQEINKEAILLEPKAVAGVAGITVKSGIEDFSIIPFEQEAGISGSETPGTSESATPE
SSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGGSSGGSSGSETPGTSE
SATPESSGGSSGGSDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGK
TILDFLKSDGFANRNFMQLIH
DDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHVAQILDSRMNTKYD
ENDKLIREVKVITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDL
HKLPKYSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSGGSGGSGGSTNLSDII
EKETGKQLVIQESILMLPEEVEEVIGNKPESDILVHTAYDESTDENVMLLTSDAPEYKPWALV
IQDSNGENKIKMLSGGSGGSGGSTNLSDIIEKETGKQLVIQESILMLPEEVEEVIGNKPESDIL
VHTAYDESTDENVMLLTSDAPEYKPWALVIQDSNGENKIKMLSGGSPKKKRK
SaBE4: SEQ ID NO: 122
MSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGGSSGGSSGSETPGTSE
SATPESSGGSSGGSGKRNYILGLAIGITSVGYGIIDYETRDVIDAGVRLFKEANVENNEGRRS
KRGARRLKRRRRHRIQRVKKLLFDYNLLTDHSELSGINPYEARVKGLSQKLSEEEFSAALLH
LAKRRGVHNVNEVEEDTGNELSTKEQISRNSKALEEKYVAELQLERLKKDGEVRGSINRFK
TSDYVKEAKQLLKVQKAYHQLDQSFIDTYIDLLETRRTYYEGPGEGSPFGWKDIKEWYEML
MGHCTYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIYQSSE
DIQEELTNLNSELTQEEIEQISNLKGYTGTHNLSLKAINLILDELWHTNDNQIAIFNRLKLVPKK
VDLSQQKEIPTTLVDDFILSPVVKRSFIQSIKVINAIIKKYGLPNDIIIELAREKNSKDAQKMINE
MQKRNRQTNERIEEIIRTTGKENAKYLIEKIKLHDMQEGKCLYSLEAIPLEDLLNNPFNYEVD
HIIPRSVSFDNSFNNKVLVKQEENSKKGNRTPFQYLSSSDSKISYETFKKHILNLAKGKGRIS
KTKKEYLLEERDINRFSVQKDFINRNLVDTRYATRGLMNLLRSYFRVNNLDVKVKSINGGFT
SFLRRKWKFKKERNKGYKHHAEDALIIANADFIFKEWKKLDKAKKVMENQMFEEKQAESMP
EIETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKN
LDVIKKENYYEVNSKCYEEAKKLKKISNQAEFIASFYNNDLIKINGELYRVIGVNNDLLNRIEV
NMIDITYREYLENMNDKRPPRIIKTIASKTQSIKKYSTDILGNLYEVKSKKHPQIIKKGGSPKKK
RKVSSDYKDHDGDYKDHDIDYKDDDDKSGGSGGSGGSTNLSDIIEKETGKQLVIQESILMLP
EEVEEVIGNKPESDILVHTAYDESTDENVMLLTSDAPEYKPWALVIQDSNGEN
KIKMLSGGSGGSGGSTNLSDIIEKETGKQLVIQESILMLPEEVEEVIGNKPESDILVHTAYDES
TDENVMLL TSDAPEYKPWALVIQDSNGENKIKMLSGGSPKKKRKV
SaBE4-Gam: SEQ ID NO: 123
MAKPAKRIKSAAAAYVPQNRDAVITDIKRIGDLQREASRLETEMNDAIAEITEKFAARIAPIKT
DIETLSKGVQGWCEANRDELTNGGKVKTANLVTGDVSWRVRPPSVSIRGMDAVMETLERL
GLQRFIRTKQEINKEAILLEPKAVAGVAGITVKSGIEDFSIIPFEQEAGISGSETPGTSESATPE
SSSETGPVAVDPTLRRRIEPHEFEVFFDPRELRKETCLLYEINWGGRHSIWRHTSQNTNKH
VEVNFIEKFTTERYFCPNTRCSITWFLSWSPCGECSRAITEFLSRYPHVTLFIYIARLYHHAD
PRNRQGLRDLISSGVTIQIMTEQESGYCWRNFVNYSPSNEAHWPRYPHLWVRLYVLELYCI
ILGLPPCLNILRRKQPQLTFFTIALQSCHYQRLPPHILWATGLKSGGSSGGSSGSETPGTSE
SATPESSGGSSGGSGKRNYILGLAIGITSVGYGIIDYETRDVIDAGVRLFKEANVENNEGRRS
KRGARRLKRRRRHRIQRVKKLLFDYNLLTDHSELSGINPYEARVKGLSQKLSEEEFSAALLH
LAKRRGVHNVNEVEEDTGNELSTKEQISRNSKALEEKYVAELQLERLKKDGEVRGSINRFK
TSDYVKEAKQLLKVQKAYHQLDQSFIDTYIDLLETRRTYYEGPGEGSPFGWKDIKEWYEML
MGHCTYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIYQSSE
DIQEELTNLNSELTQEEIEQISNLKGYTGTHNLSLKAINLILDELWHTNDNQIAIFNRLKLVPKK
VDLSQQKEIPTTLVDDFILSPVVKRSFIQSIKVINAIIKKYGLPNDIIIELAREKNSKDAQKMINE
MQKRNRQTNERIEEIIRTTGKENAKYLIEKIKLHDMQEGKCLYSLEAIPLEDLLNNPFNYEVD
HIIPRSVSFDNSFNNKVLVKQEENSKKGNRTPFQYLSSSDSKISYETFKKHILNLAKGKGRIS
KTKKEYLLEERDINRFSVQKDFINRNLVDTRYATRGLMNLLRSYFRVNNLDVKVKSINGGFT
SFLRRKWKFKKERNKGYKHHAEDALIIANADFIFKEWKKLDKAKKVMENQMFEEKQAESMP
EIETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKN
LDVIKKENYYEVNSKCYEEAKKLKKISNQAEFIASFYNNDLIKINGELYRVIGVNNDLLNRIEV
NMIDITYREYLENMNDKRPPRIIKTIASKTQSIKKYSTDILGNLYEVKSKKHPQIIKKGGSPKKK
RKVSSDYKDHDGDYKDHDIDYKDDDDKSGGSGGSGGSTNLSDIIEKETGKQLVIQESILMLP
EEVEEVIGNKPESDILVHTAYDESTDENVMLLTSDAPEYKPWALVIQDSNGENKIKMLSGGS
GGSGGSTNLSDIIEKETGKQLVIQESILMLPEEVEEVIGNKPESDILVHTAYDESTDENVMLLT
SDAPEYKPWALVIQDSNGENKIKMLSGGSPKKKRKV
BE4max and AncBE4max, SEQ ID NO: 124
MKRTADGSEFESPKKKRKV[APOBEC or ancestral APOBEC, sequences see
below]SGGSSGGSSGSETPGTSESATPESSGGSSGGSDKKYSIGLAIGTNSVGWAVITDEY
KVPSKKFKVLGNTDRHSIKKNLIGALLFDSGETAEATRLKRTARRRYTRRKNRICYLQEIFSN
EMAKVDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWM
TRKSEETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVMKQLKR
RRYTGWGRLSRKLINGIRDKQSGKTILDFLKSDGFANRNFMQLIHDDSLTFKEDIQKAQVSG
QGDSLHEHIANLAGSPAIKKGILQTVKVVDELVKVMGRHKPENIVIEMARENQTTQKGQKNS
RERMKRIEEGIKELGSQILKEHPVENTQLQNEKLYLYYLQNGRDMYVDQELDINRLSDYDVD
HIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLV
SDFRKDFQFYKVREINNYHHAHDAYLNAWGTALIKKYPKLESEFVYGDYKVYDVRKMIAKS
EQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYFDTTIDRKRYTSTK
EVLDATLIHQSITGLYETRIDLSQLGGDSGGSGGSGGSTNLSDIIEKETGKQLVIQESILMLPE
EVEEVIGNKPESDILVHTAYDESTDENVMLLTSDAPEYKPWALVIQDSNGENKIKML_SGGS
GGSGGS_TNLSDIIEKETGKQLVIQESILMLPEEVEEVIGNKPESDILVHTAYDESTDENVMLL
TSDAPEYKPWALVIQDSNGENKIKMLSGGSKRTADGSEFEPKKKRKV
Rat APOBEC1, SEQ ID NO: 125
SSETGPVAVDPTLRRRIEPHEFEVFFDPRELRKETCLLYEINWGGRHSIWRHTSQNTNKHV
EVNFIEKFTTERYFCPNTRCSITWFLSWSPCGECSRAITEFLSRYPHVTLFIYIARLYHHADP
RNRQGLRDLISSGVTIQIMTEQESGYCWRNFVNYSPSNEAHWPRYPHLWVRLYVLELYCIIL
GLPPCLNILRRKQPQLTFFTIALQSCHYQRLPPHILWATGLK
Anc689 APOBEC, SEQ ID NO: 126
SSETGPVAVDPTLRRRIEPHEFEVFFDPRELRKETCLLYEIKWGTSHKIWRHSSKNTTKHVE
VNFIEKFTSERHFCPSTSCSITWFLSWSPCGECSKAITEFLSQHPNVTLVIYVARLYHHMDQ
QNRQGLRDLVNSGVTIQIMTAPEYDYCWRNFVNYPPGKEAHWPRYPPLWMKLYALELHA
GILGLPPCLNILRRKQPQLTFFTIALQSCHYQRLPPHILWAT GLK
Anc687 APOBEC, SEQ ID NO: 127
SSETGPVAVDPTLRRRIEPHEFEVFFDPRELRKEACLLYEIKWGTSHKIWRNSGKNTTKHVE
VNFIEKFTSERHFCPSISCSITWFLSWSPCWECSKAIREFLSQHPNVTLVIYVARLFQHMDQ
QNRQGLRDLVNSGVTIQIMTASEYDHCWRNFVNYPPGKEAHWPRYPPLWMKLYALELHA
GILGLPPCLNILRRKQPQLTFFTIALQSCHYQRLPPHILWATGLK
Anc686 APOBEC, SEQ ID NO: 128
SSETGPVAVDPTLRRRIEPEFFNRNYDPRELRKETYLLYEIKWGKESKIWRHTSNNRTQHA
EVNFLENFFNELYFNPSTHCSITWFLSWSPCGECSKAIVEFLKEHPNVNLEIYVARLYLCED
ERNRQGLRDLVNSGVTIRIMNLPDYNYCWRTFVSHQGGDEDYWPRHFAPWVRLYVLELY
CIILGLPPCLNILRRKQPQLTFFTIALQSCHYQRLPPHILWAT GLK
Anc655 APOBEC, SEQ ID NO: 129
SSETGPVAVDPTLRRRIEPFYFQFNNDPRACRRKTYLCYELKQDGSTWVWKRTLHNKGRH
AEICFLEKISSLEKLDPAQHYRITWYMSWSPCSNCAQKIVDFLKEHPHVNLRIYVARLYYHEE
ERYQEGLRNLRRSGVSIRVMDLPDFEHCWETFVDNGGGPFQPWPGLEELNSKQLSRRLQ
AGILGLPPCLNILRRKQPQLTFFTIALQSCHYQRLPPHIL WATGLK
Anc733 APOBEC, SEQ ID NO: 130
SSETGPVAVDPTLRRRIEPFHFQFNNDPRAYRRKTYLCYELKQDGSTWVLDRTLRNKGRH
AEICFLDKINSWERLDPAQHYRVTWYMSWSPCSNCAQQVVDFLKEHPHVNLRIFAARLYYH
EQRRYQEGLRSLRGSGVPVAVMTLPDFEHCWETFVDHGGRPFQPWDGLEELNSRSLSRR
LQAGILGLPPCLNILRRKQPQLTFFTIALQSCHYQRLPPHI LWATGLK
APOBEC ancestor #686, SEQ ID NO: 131
EFFNRNYDPRELRKETYLLYEIKWGKESKIWRHWCTSNNRTQHAEVNFLENFFNELYFNPS
THCSITWFLSWSPCGECSKAIVEFLKEHPNVNLEIYVARLYLCEDERNRQGLRDLVNSGVTI
RIMNLPDYNYCWRTFVSHQGGDEDYWPRHFAPWVRL
APOBEC ancestor #733, SEQ ID NO: 132
FHFQFNNDPRAYRRKTYLCYELKQDGSTWVLDRGCTLRNKGRHAEICFLDKINSWERLDP
AQHYRVTWYMSWSPCSNCAQQVVDFLKEHPHVNLRIFAARLYYHEQRRYQEGLRSLRGS
GVPVAVMTLPDFEHCWETFVDHGGRPFQPWDGLEELNSRSLSRRLQAG
APOBEC ancestor #656_FERNY, SEQ ID NO: 133
FERNYDPRELRKETYLLYEIKWGKSGKLWRHWCQNNRTQHAEVYFLENIFNARRFNPSTH
CSITWYLSWSPCAECSQKIVDFLKEHPNVNLEIYVARLYYHEDERNRQGLRDLVNSGVTIRI
MDLPDYNYCWKTFVSDQGGDEDYWPGHFAPWIKQYSLKL
APOBEC ancestor #655, SEQ ID NO: 134
FYFQFNNDPRACRRKTYLCYELKQDGSTWVWKRGCTLHNKGRHAEICFLEKISSLEKLDPA
QHYRITWYMSWSPCSNCAQKIVDFLKEHPHVNLRIYVARLYYHEEERYQEGLRNLRRSGV
SIRVMDLPDFEHCWETFVDNGGGPFQPWPGLEENSKQLSRRLQAG
APOBEC ancestor #649, SEQ ID NO: 135
FYEEFNNTLKSCRHKTLLCFSLKQDENTTLWKWGYAHNNGRHAEILVLREIENYEKLDPAA
KYRITLYMSYSPCNDCADKIVDFLKKHPNVNLNIKVSRLYYHEDEKYQEGLRNLKQPGVSLK
VMDRSDFEECFDLFVDPGGGEFQPWPGLEEKSKQYSATLQAG
ABE6.3, SED ID: 136
MSEVEFSHEYWMRHALTLAKRAWDEREVPVGAVLVHNNRVIGEGWNRPIGRHDPTAHAEI
MALRQGGLVMQNYRLIDATYVTLEPCVMCAGAMIHSRIGRVVFGARDAKTGAAGSLMDVL
HHPGMNHRVEITEGILADECAALLSDFFRMRRQEIKAQKKAQSSTDSGGSSGGSSGSETP
GTSESATPESSGGSSGGSSEVEFSHEYWMRHALTLAKRAWDEREVPVGAVLVLNNRVIGE
GWNRSIGLHDPTAHAEIMALRQGGLVMQNYRLIDATLYVTFEPCVMCAGAMIHSRIGRVVF
GVRNAKTGAAGSLMDVLHYPGMNHRVEITEGILADECAALLCYFFRMRRQVFNAQKKAQS
STDSGGSSGGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGDSGGSPKKKRKV
ABE7.8, SED ID: 137
MSEVEFSHEYWMRHALTLAKRAWDEREVPVGAVLVHNNRVIGEGWNRPIGRHDPTAHAEI
MALRQGGLVMQNYRLIDATLYVTLEPCVMCAGAMIHSRIGRVVFGARDAKTGAAGSLMDVL
HHPGMNHRVEITEGILADECAALLSDFFRMRRQEIKAQKKAQSSTDSGGSSGGSSGSETP
GTSESATPESSGGSSGGSSEVEFSHEYWMRHALTLAKRALDEREVPVGAVLVLNNRVIGE
GWNRAIGLHDPTAHAEIMALRQGGLVMQNYRLIDATLYVTFEPCVMCAGAMIHSRIGRVVF
GVRNAKTGAAGSLMDVLHYPGMNHRVEITEGILADECNALLCYFFRMRRQVFNAQKKAQS
STDSGGSSGGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGDSGGSPKKKRKV
ABE7.9, SED ID: 138
MSEVEFSHEYWMRHALTLAKRAWDEREVPVGAVLVHNNRVIGEGWNRPIGRHDPTAHAEI
MALRQGGLVMQNYRLIDATLYVTLEPCVMCAGAMIHSRIGRVVFGARDAKTGAAGSLMDVL
HHPGMNHRVEITEGILADECAALLSDFFRMRRQEIKAQKKAQSSTDSGGSSGGSSGSETP
GTSESATPESSGGSSGGSSEVEFSHEYWMRHALTLAKRALDEREVPVGAVLVLNNRVIGE
GWNRAIGLHDPTAHAEIMALRQGGLVMQNYRLIDATLYVTFEPCVMCAGAMIHSRIGRVVF
GVRNAKTGAAGSLMDVLHYPGMNHRVEITEGILADECNALLCYFFRMPRQVFNAQKKAQS
STDSGGSSGGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDFR
KDFQFYKVREINNYHHAHDAYLNAWGTALIKKYPKLESEFVYGDYKVYDVRKMIAKSEQEI
GKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGDSGGSPKKKRKV
ABE7.10, SED ID: 139
MSEVEFSHEYWMRHALTLAKRAWDEREVPVGAVLVHNNRVIGEGWNRPIGRHDPTAHAEI
MALRQGGLVMQNYRLIDATLYVTLEPCVMCAGAMIHSRIGRVVFGARDAKTGAAGSLMDVL
HHPGMNHRVEITEGILADECAALLSDFFRMRRQEIKAQKKAQSSTDSGGSSGGSSGSETP
GTSESATPESSGGSSGGSSEVEFSHEYWMRHALTLAKRARDEREVPVGAVLVLNNRVIGE
GWNRAIGLHDPTAHAEIMALRQGGLVMQNYRLIDATLYVTFEPCVMCAGAMIHSRIGRVVF
GVRNAKTGAAGSLMDVLHYPGMNHRVEITEGILADECAALLCYFFRMPRQVFNAQKKAQS
STDSGGSSGGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGDSGGSPKKKRKV
ABEmax, SEQ ID NO: 140
MKRTADGSEFESPKKKRKVSEVEFSHEYWMRHALTLAKRAWDEREVPVGAVLVHNNRVIG
EGWNRPIGRHDPTAHAEIMALRQGGLVMQNYRLIDATLYVTLEPCVMCAGAMIHSRIGRW
FGARDAKTGAAGSLMDVLHHPGMNHRVEITEGILADECAALLSDFFRMRRQEIKAQKKAQS
STDSGGSSGGSSGSETPGTSESATPESSGGSSGGSSEVEFSHEYWMRHALTLAKRARDE
REVPVGAVLVLNNRVIGEGWNRAIGLHDPTAHAEIMALRQGGLVMQNYRLIDATLYVTFEP
CVMCAGAMIHSRIGRVVFGVRNAKTGAAGSLMDVLHYPGMNHRVEITEGILADECAALLCY
FFRMPRQVFNAQKKAQSSTDSGGSSGGSSGSETPGTSESATPESSGGSSGGSDKKYSIG
LAIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIKR
YDEHHQDLTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTNLGAPAA
FKYFDTTIDRKRYTSTKEVLDATLIHQSITGLYETRIDLSQLGGDSGGSKRTADGSEFEPKKK
RKV
SpACE, SEQ ID NO: 140
MKRTADGSEFESPKKKRKVSEVEFSHEYWMRHALTLAKRARDEREVPVGAVLVLNNRVIG
EGWNRAIGLHDPTAHAEIMALRQGGLVMQNYRLIDATLYGTFEPCVMCAGAMIHSRIGRVV
FGVRNAKTGAAGSLMDVLHYPGMNHRVEITEGILADECAALLCYFFRMPRQVFNAQKKAQ
SSTDSGGSSGGSSGSETPGTSESATPESSGGSSGGSDKKYSIGLAIGTNSVGWAVITDEYK
VPSKKFKVLGNTDRHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQGD
SLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQ
VNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVL
DATLIHQSITGLYETRIDLSQLGGDSGGSKRTADGSEFEPKKKRKVSGGSSGGSSGSETPG
TSESATPESSGGSSGGSTDAEYVRIHEKLDIYTFKKQFFNNKKSVSHRCYVLFELKRRGER
RACFWGYAVNKPQSGTERGIHAEIFSIRKVEEYLRDNPGQFTINWYSSWSPCADCAEKILE
WYNQELRGNGHTLKIWACKLYYEKNARNQIGLWNLRDNGVGLNVMVSEHYQCCRKIFIQS
SHNQLNENRWLEKTLKRAEKWRSELSIMIQVKILHTTKSPAVSGGSGGSGGSTNLSDIIEKE
TGKQLVIQESILMLPEEVEEVIGNKPESDILVHTAYDESTDENVMLLTSDAPEYKPWALVIQD
SNGENKIKMLSGGSGGSGGSTNLSDIIEKETGKQLVIQESILMLPEEVEEVIGNKPESDILVH
TAYDESTDENVMLLTSDAPEYKPWALVIQDSNGENKIKMLGSGATNFSLLKQAGDVEENPG
PMVSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDTL
VNRIELKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITLGMDELYKSGGSP
KKKRK
SPACEΔUGI, SEQ ID NO: 141
MKRTADGSEFESPKKKRKVSEVEFSHEYWMRHALTLAKRARDEREVPVGAVLVLNNRVIG
EGWNRAIGLHDPTAHAEIMALRQGGLVMQNYRLIDATLYGTFEPCVMCAGAMIHSRIGRVV
FGVRNAKTGAAGSLMDVLHYPGMNHRVEITEGILADECAALLCYFFRMPRQVFNAQKKAQ
SSTDSGGSSGGSSGSETPGTSESATPESSGGSSGGSDKKYSIGLAIGTNSVGWAVITDEYK
VPSKKFKVLGNTDRHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQGD
SLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQ
VNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVL
DATLIHQSITGLYETRIDLSQLGGDSGGSKRTADGSEFEPKKKRKVSGGSSGGSSGSETPG
TSESATPESSGGSSGGSTDAEYVRIHEKLDIYTFKKQFFNNKKSVSHRCYVLFELKRRGER
RACFWGYAVN KPQSGTERGIHAEIFSIRKVEEYLRDNPGQFTINWYSSWSPCADCAEKILE
WYNQELRGNGHTLKIWACKLYYEKNARNQIGLWNLRDNGVGLNVMVSEHYQCCRKIFIQS
SHNQLNENRWLEKTLKRAEKWRSELSIMIQVKILHTTKSPAVGSGATNFSLLKQAGDVEEN
PGPMVSKGEELFTGWPILVELDGDVNGHKFSVSGEGEGDATYGKLTLKFICTTGKLPVPW
PTLVTTLTYGVQCFSRYPDHMKQHDFFKSAMPEGYVQERTIFFKDDGNYKTRAEVKFEGD
TLVNRIELKGIDFKEDGNILGHKLEYNYNSHNVYIMADKQKNGIKVNFKIRHNIEDGSVQLAD
HYQQNTPIGDGPVLLPDNHYLSTQSALSKDPNEKRDHMVLLEFVTAAGITLGMDELYKSGG
SPKKKRKV
SPACE-NG, SEQ ID NO: 142
MKRTADGSEFESPKKKRKVSEVEFSHEYWMRHALTLAKRARDEREVPVGAVLVLNNRVIG
EGWNRAIGLHDPTAHAEIMALRQGGLVMQNYRLIDATLYGTFEPCVMCAGAMIHSRIGRVV
FGVRNAKTGAAGSLMDVLHYPGMNHRVEITEGILADECAALLCYFFRMPRQVFNAQKKAQ
SSTDSGGSSGGSSGSETPGTSESATPESSGGSSGGSDKKYSIGLAIGTNSVGWAVITDEYK
VPSKKFKVLGNTDRHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQGD
SLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQ
VNIVKKTEVQTGGFSKESIRPKRNSDKLIARKKDWDPKKYGGFVSPTVAYSVLVVAKVEKG
KSKKLKSVKELLGITIMERSSFEKNPIDFLEAKGYKEVKKDLIIKLPKYSLFELENGRKRMLAS
ARFLQKGNELALPSKYVNFLYLASHYEKLKGSPEDNEQKQLFVEQHKHYLDEIIEQISEFSK
RVILADANLDKVLSAYNKHRDKPIREQAENIIHLFTLTNLGAPRAFKYFDTTIDRKVYRSTKEV
LDATLIHQSITGLYETRIDLSQLGGDSGGSKRTADGSEFEPKKKRKVSGGSSGGSSGSETP
GTSESATPESSGGSSGGSTDAEYVRIHEKLDIYTFKKQFFNNKKSVSHRCYVLFELKRRGE
RRACFWGYAVNKPQSGTERGIHAEIFSIRKVEEYLRDNPGQFTINWYSSWSPCADCAEKIL
EWYNQELRGNGHTLKIWACKLYYEKNARNQIGLWNLRDNGVGLNVMVSEHYQCCRKIFIQ
SSHNQLNENRWLEKTLKRAEKWRSELSIMIQVKILHTTKSPAVSGGSGGSGGSTNLSDIIEK
ETGKQLVIQESILMLPEEVEEVIGNKPESDILVHTAYDESTDENVMLLTSDAPEYKPWALVIQ
DSNGENKIKMLSGGSGGSGGSTNLSDIIEKETGKQLVIQESILMLPEEVEEVIGNKPESDILV
HTAYDESTDENVMLLTSDAPEYKPWALVIQDSNGENKIKMLGSGATNFSLLKQAGDVEENP
GPMVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGDTL
VNRIELKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITLGMDELYKSGGSP
KKKRKV
SPACE-VRQR, SEQ ID NO: 143
MKRTADGSEFESPKKKRKVSEVEFSHEYWMRHALTLAKRARDEREVPVGAVLVLNNRVIG
EGWNRAIGLHDPTAHAEIMALRQGGLVMQNYRLIDATLYGTFEPCVMCAGAMIHSRIGRVV
FGVRNAKTGAAGSLMDVLHYPGMNHRVEITEGILADECAALLCYFFRMPRQVFNAQKKAQ
SSTDSGGSSGGSSGSETPGTSESATPESSGGSSGGSDKKYSIGLAIGTNSVGWAVITDEYK
VPSKKFKVLGNTDRHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQGD
SLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQ
VNIVKKTEVQTGGFSKESILPKRNSDKLIARKKDWDPKKYGGFVSPTVAYSVLVVAKVEKGK
SKKLKSVKELLGITIMERSSFEKNPIDFLEAKGYKEVKKDLIIKLPKYSLFELENGRKRMLASA
RELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKQYRSTKEVL
DATLIHQSITGLYETRIDLSQLGGDSGGSKRTADGSEFEPKKKRKVSGGSSGGSSGSETPG
TSESATPESSGGSSGGSTDAEYVRIHEKLDIYTFKKQFFNNKKSVSHRCYVLFELKRRGER
RACFWGYAVNKPQSGTERGIHAEIFSIRKVEEYLRDNPGQFTINWYSSWSPCADCAEKILE
WYNQELRGNGHTLKIWACKLYYEKNARNQIGLWNLRDNGVGLNVMVSEHYQCCRKIFIQS
SHNQLNENRWLEKTLKRAEKWRSELSIMIQVKILHTTKSPAVSGGSGGSGGSTNLSDIIEKE
TGKQLVIQESILMLPEEVEEVIGNKPESDILVHTAYDESTDENVMLLTSDAPEYKPWALVIQD
SNGENKIKMLSGGSGGSGGSTNLSDIIEKETGKQLVIQESILMLPEEVEEVIGNKPESDILVH
TAYDESTDENVMLLTSDAPEYKPWALVIQDSNGENKIKMLGSGATNFSLLKQAGDVEENPG
PMVSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDTL
VNRIELKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITLGMDELYKSGGSP
KKKRKV
SPACE-NAA, SEQ ID NO: 144
MKRTADGSEFESPKKKRKVSEVEFSHEYWMRHALTLAKRARDEREVPVGAVLVLNNRVIG
EGWNRAIGLHDPTAHAEIMALRQGGLVMQNYRLIDATLYGTFEPCVMCAGAMIHSRIGRVV
FGVRNAKTGAAGSLMDVLHYPGMNHRVEITEGILADECAALLCYFFRMPRQVFNAQKKAQ
SSTDSGGSSGGSSGSETPGTSESATPESSGGSSGGSDKKYSIGLAIGTNSVGWAVITDEYK
VPSKKFKVLGNTDRHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRK
SEETITPWNFEEWDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQGD
SLHEHIANLAGSPAIKKGILQTVKVVDELVKVMGRHKPENMEMARENQTTQKGQKNSRER
MKRIEEGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQ
VNIVKKTEIQTVGQNGGLFDDNPKSPLEVTPSKLVPLKKELNPKKYGGYQKPTTAYPVLLITD
TKQLIPISVMNKKQFEQNPVKFLRDRGYQQVGKNDFIKLPKYTLVDIGDGIKRLWASSKEIHK
GNQLVVSKKSQILLYHAHHLDSDLSNDYLQNHNQQFDVLFNEIISFSKKCKLGKEHIQKIENV
YSNKKNSASIEELAESFIKLLGFTQLGATSPFNFLGVKLNQKQYKGKKDYILPCTEGTLIRQSI
TGLYETRVDLSKIGEDSGGSKRTADGSEFEPKKKRKVSGGSSGGSSGSETPGTSESATPE
SSGGSSGGSTDAEYVRIHEKLDIYTFKKQFFNNKKSVSHRCYVLFELKRRGERRACFWGYA
VNKPQSGTERGIHAEIFSIRKVEEYLRDNPGQFTINWYSSWSPCADCAEKILEWYNQELRG
NGHTLKIWACKLYYEKNARNQIGLWNLRDNGVGLNVMVSEHYQCCRKIFIQSSHNQLNEN
RWLEKTLKRAEKWRSELSIMIQVKILHTTKSPAVSGGSGGSGGSTNLSDIIEKETGKQLVIQE
SILMLPEEVEEVIGNKPESDILVHTAYDESTDENVMLLTSDAPEYKPWALVIQDSNGENKIKM
LSGGSGGSGGSTNLSDIIEKETGKQLVIQESILMLPEEVEEVIGNKPESDILVHTAYDESTDE
NVMLLTSDAPEYKPWALVIQDSNGENKIKMLGSGATNFSLLKQAGDVEENPGPMVSKGEE
LFTGVVPILVELDGDVNGHKFSVSGEGEGDATYGKLTLKFICTTGKLPVPWPTLVTTLTYGV
QCFSRYPDHMKQHDFFKSAMPEGYVQERTIFFKDDGNYKTRAEVKFEGDTLVNRIELKGID
FKEDGNILGHKLEYNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYKSGGSPKKKRKV
BE4max-ΔUG1-eUNG, SEQ ID NO: 147
MKRTADGSEFESPKKKRKVSSETGPVAVDPTLRRRIEPHEFEVFFDPRELRKETCLLYEIN
WGGRHSIWRHTSQNTNKHVEVNFIEKFTTERYFCPNTRCSITWFLSWSPCGECSRAITEFL
SRYPHVTLFIYIARLYHHADPRNRQGLRDLISSGVTIQIMTEQESGYCWRNFVNYSPSNEAH
WPRYPHLWVRLYVLELYCIILGLPPCLNILRRKQPQLTFFTIALQSCHYQRLPPHILWATGLK
SGGSSGGSSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSARLSKS
RRLENLIAQLPGEKKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYVTEG
MRKPAFLSGEQKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVMKQLKRRRYTGW
GRLSRKLINGIRDKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQKGQKNSRERMKR
IEEGIKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKEVLDATL
IHQSITGLYETRIDLSQLGGDSGGSGGSGGSANELTWHDVLAEEKQQPYFLNTLQTVASER
QSGVTIYPPQKDVFNAFRFTELGDVKVVILGQDPYHGPGQAHGLAFSVRPGIAIPPSLLNMY
KELENTIPGFTRPNHGYLESWARQGVLLLNTVLTVRAGQAHSHASLGWETFTDKVISLINQH
REGVVFLLWGSHAQKKGAIIDKQRHHVLKAPHPSPLSAHRGFFGCNHFVLANQWLEQRGE
TPIDWMPVLPAESESGGSKRTADGSEFEPKKKRKVGGGGSGATNFSLLKQAGDVEENPG
PMVSKGEELFTGWPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDTL
VNRIELKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITLGMDELYK
BE4max(R33A)-ΔUGI-eUNG, SEQ ID NO: 148
MKRTADGSEFESPKKKRKVSSETGPVAVDPTLRRRIEPHEFEVFFDPRELAKETCLL
YEINWGGRHSIWRHTSQNTNKHVEVNFIEKFTTERYFCPNTRCSITWFLSWSPCGE
CSRAITEFLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLTFFTIALQ
SCHYQRLPPHILWATGLKSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLWAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSGGSGGSANELTWHDVL
AEEKQQPYFLNTLQTVASERQSGVTIYPPQKDVFNAFRFTELGDVKVVILGQDPYH
GPGQAHGLAFSVRPGIAIPPSLLNMYKELENTIPGFTRPNHGYLESWARQGVLLLNT
VLTVRAGQAHSHASLGWETFTDKVISLINQHREGVVFLLWGSHAQKKGAIIDKQRH
HVLKAPHPSPLSAHRGFFGCNHFVLANQWLEQRGETPIDWMPVLPAESESGGSKR
TADGSEFEPKKKRKVGGGGSGATNFSLLKQAGDVEENPGPMVSKGEELFTGVVPI
LVELDGDVNGHKFSVSGEGEGDATYGKLTLKFICTTGKLPVPWPTLVTTLTYGVQC
FSRYPDHMKQHDFFKSAMPEGYVQERTIFFKDDGNYKTRAEVKFEGDTLVNRIELK
GIDFKEDGNILGHKLEYNYNSHNVYIMADKQKNGIKVNFKIRHNIEDGSVQLADHYQ
QNTPIGDGPVLLPDNHYLSTQSALSKDPNEKRDHMVLLEFVTAAGITLGMDELYK
BE4max(R33A/K34A)-ΔUGI-eUNG, SEQ ID NO: 149
MKRTADGSEFESPKKKRKVSSETGPVAVDPTLRRRIEPHEFEVFFDPRELAAETCLL
YEINWGGRHSIWRHTSQNTNKHVEVNFIEKFTTERYFCPNTRCSITWFLSWSPCGE
CSRAITEFLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLTFFTIALQ
SCHYQRLPPHILWATGLKSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLWAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSGGSGGSANELTWHDVL
AEEKQQPYFLNTLQTVASERQSGVTIYPPQKDVFNAFRFTELGDVKVVILGQDPYH
GPGQAHGLAFSVRPGIAIPPSLLNMYKELENTIPGFTRPNHGYLESWARQGVLLLNT
VLTVRAGQAHSHASLGWETFTDKVISLINQHREGVVFLLWGSHAQKKGAIIDKQRH
HVLKAPHPSPLSAHRGFFGCNHFVLANQWLEQRGETPIDWMPVLPAESESGGSKR
TADGSEFEPKKKRKVGGGGSGATNFSLLKQAGDVEENPGPMVSKGEELFTGVVPI
LVELDGDVNGHKFSVSGEGEGDATYGKLTLKFICTTGKLPVPWPTLVTTLTYGVQC
FSRYPDHMKQHDFFKSAMPEGYVQERTIFFKDDGNYKTRAEVKFEGDTLVNRIELK
GIDFKEDGNILGHKLEYNYNSHNVYIMADKQKNGIKVNFKIRHNIEDGSVQLADHYQ
QNTPIGDGPVLLPDNHYLSTQSALSKDPNEKRDHMVLLEFVTAAGITLGMDELYK
nCas9-eUNG for BE4max, SEQ ID NO: 150
MKRTADGSEFESPKKKRKVSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRK
SEETITPWNFEEWDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYFD
TTIDRKRYTSTKEVLDATLIHQSITGLYETRIDLSQLGGDSGGSGGSGGSANELTWH
DVLAEEKQQPYFLNTLQTVASERQSGVTIYPPQKDVFNAFRFTELGDVKVVILGQDP
YHGPGQAHGLAFSVRPGIAIPPSLLNMYKELENTIPGFTRPNHGYLESWARQGVLLL
NTVLTVRAGQAHSHASLGWETFTDKVISLINQHREGVVFLLWGSHAQKKGAIIDKQR
HHVLKAPHPSPLSAHRGFFGCNHFVLANQWLEQRGETPIDWMPVLPAESESGGSK
RTADGSEFEPKKKRKVGGGGSGATNFSLLKQAGDVEENPGPMVSKGEELFTGVVP
ILVELDGDVNGHKFSVSGEGEGDATYGKLTLKFICTTGKLPVPWPTLVTTLTYGVQC
FSRYPDHMKQHDFFKSAMPEGYVQERTIFFKDDGNYKTRAEVKFEGDTLVNRIELK
GIDFKEDGNILGHKLEYNYNSHNVYIMADKQKNGIKVNFKIRHNIEDGSVQLADHYQ
QNTPIGDGPVLLPDNHYLSTQSALSKDPNEKRDHMVLLEFVTAAGITLGMDELYK
eUNG-BE4max-ΔUGI, SEQ ID NO: 151
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSSETGPVAVDPTLRRRIEPHEFE
VFFDPRELRKETCLLYEINWGGRHSIWRHTSQNTNKHVEVNFIEKFTTERYFCPNTR
CSITWFLSWSPCGECSRAITEFLSRYPHVTLFIYIARLYHHADPRNRQGLRDLISSGV
TIQIMTEQESGYCWRNFVNYSPSNEAHWPRYPHLWVRLYVLELYCIILGLPPCLNIL
RRKQPQLTFFTIALQSCHYQRLPPHILWATGLKSGGSSGGSSGSETPGTSESATPE
SSGGSSGGSDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDL
LRKQRTFDNGSIPHQIHLGELHAILRRQEDFYPFLKDNREKIEKILTFRIPYYVGPLAR
GNSRFAWMTRKSEETITPWNFEEWDKGASAQSFIERMTNFDKNLPNEKVLPKHSL
LYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSV
LVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSGGSKRTAD
GSEFEPKKKRKVGGGGSGATNFSLLKQAGDVEENPGPMVSKGEELFTGWPILVEL
DGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTP
IGDGPVLLPDNHYLSTQSALSKDPNEKRDHMVLLEFVTAAGITLGMDELYK
eUNG-BE4max(R33A)-ΔUGI = CGBE1, SEQ ID NO: 152
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSSETGPVAVDPTLRRRIEPHEFE
VFFDPRELAKETCLLYEINWGGRHSIWRHTSQNTNKHVEVNFIEKFTTERYFCPNTR
CSITWFLSWSPCGECSRAITEFLSRYPHVTLFIYIARLYHHADPRNRQGLRDLISSGV
TIQIMTEQESGYCWRNFVNYSPSNEAHWPRYPHLWVRLYVLELYCIILGLPPCLNIL
RRKQPQLTFFTIALQSCHYQRLPPHILWATGLKSGGSSGGSSGSETPGTSESATPE
SSGGSSGGSDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDL
LRKQRTFDNGSIPHQIHLGELHAILRRQEDFYPFLKDNREKIEKILTFRIPYYVGPLAR
GNSRFAWMTRKSEETITPWNFEEWDKGASAQSFIERMTNFDKNLPNEKVLPKHSL
LYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSV
LVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSGGSKRTAD
GSEFEPKKKRKVGGGGSGATNFSLLKQAGDVEENPGPMVSKGEELFTGWPILVEL
DGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTP
IGDGPVLLPDNHYLSTQSALSKDPNEKRDHMVLLEFVTAAGITLGMDELYK
eUNG-BE4max(R33A/K34A)-ΔUGI, SEQ ID NO: 153
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSSETGPVAVDPTLRRRIEPHEFE
VFFDPRELAAETCLLYEINWGGRHSIWRHTSQNTNKHVEVNFIEKFTTERYFCPNTR
CSITWFLSWSPCGECSRAITEFLSRYPHVTLFIYIARLYHHADPRNRQGLRDLISSGV
TIQIMTEQESGYCWRNFVNYSPSNEAHWPRYPHLWVRLYVLELYCIILGLPPCLNIL
RRKQPQLTFFTIALQSCHYQRLPPHILWATGLKSGGSSGGSSGSETPGTSESATPE
SSGGSSGGSDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDL
LRKQRTFDNGSIPHQIHLGELHAILRRQEDFYPFLKDNREKIEKILTFRIPYYVGPLAR
GNSRFAWMTRKSEETITPWNFEEWDKGASAQSFIERMTNFDKNLPNEKVLPKHSL
LYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSV
LVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSGGSKRTAD
GSEFEPKKKRKVGGGGSGATNFSLLKQAGDVEENPGPMVSKGEELFTGWPILVEL
DGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTP
IGDGPVLLPDNHYLSTQSALSKDPNEKRDHMVLLEFVTAAGITLGMDELYK
eUNG-nCas9 for BE4max, SEQ ID NO: 154
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSGGSSGGSSGSETPGTSESATP
ESSGGSSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRL
ENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFE
DREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTV
KWDELVKVMGRHKPENIVIEMARENQTTQKGQKNSRERMKRIEEGIKELGSQILKE
HPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKL
PKYSLFELENGRKRMLASAGELQKGNELALPSKYVNFLYLASHYEKLKGSPEDNEQ
KQLFVEQHKHYLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSGGS
KRTADGSEFEPKKKRKVGGGGSGATNFSLLKQAGDVEENPGPMVSKGEELFTGVV
PILVELDGDVNGHKFSVSGEGEGDATYGKLTLKFICTTGKLPVPWPTLVTTLTYGVQ
CFSRYPDHMKQHDFFKSAMPEGYVQERTIFFKDDGNYKTRAEVKFEGDTLVNRIEL
KGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITLGMDELYK
BE4max-ΔUGI-hUNG, SEQ ID NO: 155
MKRTADGSEFESPKKKRKVSSETGPVAVDPTLRRRIEPHEFEVFFDPRELRKETCL
LYEINWGGRHSIWRHTSQNTNKHVEVNFIEKFTTERYFCPNTRCSITWFLSWSPCG
ECSRAITEFLSRYPHVTLFIYIARLYHHADPRNRQGLRDLISSGVTIQIMTEQESGYC
WRNFVNYSPSNEAHWPRYPHLWVRLYVLELYCIILGLPPCLNILRRKQPQLTFFTIAL
QSCHYQRLPPHILWATGLKSGGSSGGSSGSETPGTSESATPESSGGSSGGSDKKY
SIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDSGGSGGSGGSIGQKTLYSF
FSPSPARKRHAPSPEPAVQGTGVAGVPEESGDAAAIPAKKAPAGQEEPGTPPSSP
LSAEQLDRIQRNKAAALLRLAARNVPVGFGESWKKHLSGEFGKPYFIKLMGFVAEE
RKHYTVYPPPHQVFTWTQMCDIKDVKVVILGQDPYHGPNQAHGLCFSVQRPVPPP
PSLENIYKELSTDIEDFVHPGHGDLSGWAKQGVLLLNAVLTVRAHQANSHKERGWE
QFTDAVVSWLNQNSNGLVFLLWGSYAQKKGSAIDRKRHHVLQTAHPSPLSVYRGF
FGCRHFSKTNELLQKSGKKPIDWKELSGGSKRTADGSEFEPKKKRKVGGGGSGAT
NFSLLKQAGDVEENPGPMVSKGEELFTGVVPILVELDGDVNGHKFSVSGEGEGDA
TYGKLTLKFICTTGKLPVPWPTLVTTLTYGVQCFSRYPDHMKQHDFFKSAMPEGYV
QERTIFFKDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHYLSTQSAL
SKDPNEKRDHMVLLEFVTAAGITLGMDELYK
BE4max(R33A)-ΔUGI-hUNG, SEQ ID NO: 156
MKRTADGSEFESPKKKRKVSSETGPVAVDPTLRRRIEPHEFEVFFDPRELAKETCLL
YEINWGGRHSIWRHTSQNTNKHVEVNFIEKFTTERYFCPNTRCSITWFLSWSPCGE
CSRAITEFLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLTFFTIALQ
SCHYQRLPPHILWATGLKSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSGGSGGSIGQKTLYSFFS
PSPARKRHAPSPEPAVQGTGVAGVPEESGDAAAIPAKKAPAGQEEPGTPPSSPLS
AEQLDRIQRNKAAALLRLAARNVPVGFGESWKKHLSGEFGKPYFIKLMGFVAEERK
HYTVYPPPHQVFTWTQMCDIKDVKVVILGQDPYHGPNQAHGLCFSVQRPVPPPPS
LENIYKELSTDIEDFVHPGHGDLSGWAKQGVLLLNAVLTVRAHQANSHKERGWEQF
TDAVVSWLNQNSNGLVFLLWGSYAQKKGSAIDRKRHHVLQTAHPSPLSVYRGFFG
CRHFSKTNELLQKSGKKPIDWKELSGGSKRTADGSEFEPKKKRKVGGGGSGATNF
SLLKQAGDVEENPGPMVSKGEELFTGVVPILVELDGDVNGHKFSVSGEGEGDATY
GKLTLKFICTTGKLPVPWPTLVTTLTYGVQCFSRYPDHMKQHDFFKSAMPEGYVQE
RTIFFKDDGNYKTRAEVKFEGDTLVNRIELKGIDFKEDGNILGHKLEYNYNSHNVYIM
ADKQKNGIKVNFKIRHNIEDGSVQLADHYQQNTPIGDGPVLLPDNHYLSTQSALSKD
PNEKRDHMVLLEFVTAAGITLGMDELYK
BE4max(R33A/K34A)-ΔUGI-hUNG, SEQ ID NO: 157
MKRTADGSEFESPKKKRKVSSETGPVAVDPTLRRRIEPHEFEVFFDPRELAAETCLL
YEINWGGRHSIWRHTSQNTNKHVEVNFIEKFTTERYFCPNTRCSITWFLSWSPCGE
CSRAITEFLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLTFFTIALQ
SCHYQRLPPHILWATGLKSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSGGSGGSIGQKTLYSFFS
PSPARKRHAPSPEPAVQGTGVAGVPEESGDAAAIPAKKAPAGQEEPGTPPSSPLS
AEQLDRIQRNKAAALLRLAARNVPVGFGESWKKHLSGEFGKPYFIKLMGFVAEERK
HYTVYPPPHQVFTWTQMCDIKDVKVVILGQDPYHGPNQAHGLCFSVQRPVPPPPS
LENIYKELSTDIEDFVHPGHGDLSGWAKQGVLLLNAVLTVRAHQANSHKERGWEQF
TDAVVSWLNQNSNGLVFLLWGSYAQKKGSAIDRKRHHVLQTAHPSPLSVYRGFFG
CRHFSKTNELLQKSGKKPIDWKELSGGSKRTADGSEFEPKKKRKVGGGGSGATNF
SLLKQAGDVEENPGPMVSKGEELFTGVVPILVELDGDVNGHKFSVSGEGEGDATY
GKLTLKFICTTGKLPVPWPTLVTTLTYGVQCFSRYPDHMKQHDFFKSAMPEGYVQE
RTIFFKDDGNYKTRAEVKFEGDTLVNRIELKGIDFKEDGNILGHKLEYNYNSHNVYIM
ADKQKNGIKVNFKIRHNIEDGSVQLADHYQQNTPIGDGPVLLPDNHYLSTQSALSKD
PNEKRDHMVLLEFVTAAGITLGMDELYK
nCas9-hUNG for BE4max, SEQ ID NO: 158
MKRTADGSEFESPKKKRKVSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYFD
TTIDRKRYTSTKEVLDATLIHQSITGLYETRIDLSQLGGDSGGSGGSGGSIGQKTLYS
FFSPSPARKRHAPSPEPAVQGTGVAGVPEESGDAAAIPAKKAPAGQEEPGTPPSS
PLSAEQLDRIQRNKAAALLRLAARNVPVGFGESWKKHLSGEFGKPYFIKLMGFVAE
ERKHYTVYPPPHQVFTWTQMCDIKDVKVVILGQDPYHGPNQAHGLCFSVQRPVPP
PPSLENIYKELSTDIEDFVHPGHGDLSGWAKQGVLLLNAVLTVRAHQANSHKERGW
EQFTDAVVSWLNQNSNGLVFLLWGSYAQKKGSAIDRKRHHVLQTAHPSPLSVYRG
FFGCRHFSKTNELLQKSGKKPIDWKELSGGSKRTADGSEFEPKKKRKVGGGGSGA
TNFSLLKQAGDVEENPGPMVSKGEELFTGVVPILVELDGDVNGHKFSVSGEGEGD
ATYGKLTLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNHYLSTQSA
LSKDPNEKRDHMVLLEFVTAAGITLGMDELYK
hUNG-BE4max-ΔUGI, SEQ ID NO: 159
MKRTADGSEFESPKKKRKVIGQKTLYSFFSPSPARKRHAPSPEPAVQGTGVAGVP
EESGDAAAIPAKKAPAGQEEPGTPPSSPLSAEQLDRIQRNKAAALLRLAARNVPVG
FGESWKKHLSGEFGKPYFIKLMGFVAEERKHYTVYPPPHQVFTWTQMCDIKDVKV
VILGQDPYHGPNQAHGLCFSVQRPVPPPPSLENIYKELSTDIEDFVHPGHGDLSGW
AKQGVLLLNAVLTVRAHQANSHKERGWEQFTDAVVSWLNQNSNGLVFLLWGSYA
QKKGSAIDRKRHHVLQTAHPSPLSVYRGFFGCRHFSKTNELLQKSGKKPIDWKELS
GGSGGSGGSSSETGPVAVDPTLRRRIEPHEFEVFFDPRELRKETCLLYEINWGGRH
SIWRHTSQNTNKHVEVNFIEKFTTERYFCPNTRCSITWFLSWSPCGECSRAITEFLS
RYPHVTLFIYIARLYHHADPRNRQGLRDLISSGVTIQIMTEQESGYCWRNFVNYSPS
NEAHWPRYPHLWVRLYVLELYCIILGLPPCLNILRRKQPQLTFFTIALQSCHYQRLPP
HILWATGLKSGGSSGGSSGSETPGTSESATPESSGGSSGGSDKKYSIGLAIGTNSV
GWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSKRTADGSEFEPKKKRKVGGGGSGAT
NFSLLKQAGDVEENPGPMVSKGEELFTGVVPILVELDGDVNGHKFSVSGEGEGDA
TYGKLTLKFICTTGKLPVPWPTLVTTLTYGVQCFSRYPDHMKQHDFFKSAMPEGYV
QERTIFFKDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHYLSTQSAL
SKDPNEKRDHMVLLEFVTAAGITLGMDELYK
hUNG-BE4max(R33A)-ΔUGI, SEQ ID NO: 160
MKRTADGSEFESPKKKRKVIGQKTLYSFFSPSPARKRHAPSPEPAVQGTGVAGVP
EESGDAAAIPAKKAPAGQEEPGTPPSSPLSAEQLDRIQRNKAAALLRLAARNVPVG
FGESWKKHLSGEFGKPYFIKLMGFVAEERKHYTVYPPPHQVFTWTQMCDIKDVKV
VILGQDPYHGPNQAHGLCFSVQRPVPPPPSLENIYKELSTDIEDFVHPGHGDLSGW
AKQGVLLLNAVLTVRAHQANSHKERGWEQFTDAVVSWLNQNSNGLVFLLWGSYA
QKKGSAIDRKRHHVLQTAHPSPLSVYRGFFGCRHFSKTNELLQKSGKKPIDWKELS
GGSGGSGGSSSETGPVAVDPTLRRRIEPHEFEVFFDPRELAKETCLLYEINWGGRH
SIWRHTSQNTNKHVEVNFIEKFTTERYFCPNTRCSITWFLSWSPCGECSRAITEFLS
RYPHVTLFIYIARLYHHADPRNRQGLRDLISSGVTIQIMTEQESGYCWRNFVNYSPS
NEAHWPRYPHLWVRLYVLELYCIILGLPPCLNILRRKQPQLTFFTIALQSCHYQRLPP
HILWATGLKSGGSSGGSSGSETPGTSESATPESSGGSSGGSDKKYSIGLAIGTNSV
GWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSKRTADGSEFEPKKKRKVGGGGSGAT
NFSLLKQAGDVEENPGPMVSKGEELFTGVVPILVELDGDVNGHKFSVSGEGEGDA
TYGKLTLKFICTTGKLPVPWPTLVTTLTYGVQCFSRYPDHMKQHDFFKSAMPEGYV
QERTIFFKDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHYLSTQSAL
SKDPNEKRDHMVLLEFVTAAGITLGMDELYK
hUNG-BE4max(R33A/K34A)-ΔUGI, SEQ ID NO: 161
MKRTADGSEFESPKKKRKVIGQKTLYSFFSPSPARKRHAPSPEPAVQGTGVAGVP
EESGDAAAIPAKKAPAGQEEPGTPPSSPLSAEQLDRIQRNKAAALLRLAARNVPVG
FGESWKKHLSGEFGKPYFIKLMGFVAEERKHYTVYPPPHQVFTWTQMCDIKDVKV
VILGQDPYHGPNQAHGLCFSVQRPVPPPPSLENIYKELSTDIEDFVHPGHGDLSGW
AKQGVLLLNAVLTVRAHQANSHKERGWEQFTDAVVSWLNQNSNGLVFLLWGSYA
QKKGSAIDRKRHHVLQTAHPSPLSVYRGFFGCRHFSKTNELLQKSGKKPIDWKELS
GGSGGSGGSSSETGPVAVDPTLRRRIEPHEFEVFFDPRELAAETCLLYEINWGGRH
SIWRHTSQNTNKHVEVNFIEKFTTERYFCPNTRCSITWFLSWSPCGECSRAITEFLS
RYPHVTLFIYIARLYHHADPRNRQGLRDLISSGVTIQIMTEQESGYCWRNFVNYSPS
NEAHWPRYPHLWRLYVLELYCIILGLPPCLNILRRKQPQLTFFTIALQSCHYQRLPP
HILWATGLKSGGSSGGSSGSETPGTSESATPESSGGSSGGSDKKYSIGLAIGTNSV
GWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSKRTADGSEFEPKKKRKVGGGGSGAT
NFSLLKQAGDVEENPGPMVSKGEELFTGVVPILVELDGDVNGHKFSVSGEGEGDA
TYGKLTLKFICTTGKLPVPWPTLVTTLTYGVQCFSRYPDHMKQHDFFKSAMPEGYV
QERTIFFKDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHYLSTQSAL
SKDPNEKRDHMVLLEFVTAAGITLGMDELYK
hUNG-nCas9 for BE4max, SEQ ID NO: 162
MKRTADGSEFESPKKKRKVIGQKTLYSFFSPSPARKRHAPSPEPAVQGTGVAGVP
EESGDAAAIPAKKAPAGQEEPGTPPSSPLSAEQLDRIQRNKAAALLRLAARNVPVG
FGESWKKHLSGEFGKPYFIKLMGFVAEERKHYTVYPPPHQVFTWTQMCDIKDVKV
VILGQDPYHGPNQAHGLCFSVQRPVPPPPSLENIYKELSTDIEDFVHPGHGDLSGW
AKQGVLLLNAVLTVRAHQANSHKERGWEQFTDAVVSWLNQNSNGLVFLLWGSYA
QKKGSAIDRKRHHVLQTAHPSPLSVYRGFFGCRHFSKTNELLQKSGKKPIDWKELS
GGSGGSGGSSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYI
DGGASQEEFYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSKRTADGSEFEPKKKRKVGGGGSGAT
NFSLLKQAGDVEENPGPMVSKGEELFTGVVPILVELDGDVNGHKFSVSGEGEGDA
TYGKLTLKFICTTGKLPVPWPTLVTTLTYGVQCFSRYPDHMKQHDFFKSAMPEGYV
QERTIFFKDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHYLSTQSAL
SKDPNEKRDHMVLLEFVTAAGITLGMDELYK
BE4max w/o UGI, SEQ ID NO: 163
MKRTADGSEFESPKKKRKVSSETGPVAVDPTLRRRIEPHEFEVFFDPRELRKETCL
LYEINWGGRHSIWRHTSQNTNKHVEVNFIEKFTTERYFCPNTRCSITWFLSWSPCG
ECSRAITEFLSRYPHVTLFIYIARLYHHADPRNRQGLRDLISSGVTIQIMTEQESGYC
WRNFVNYSPSNEAHWPRYPHLWVRLYVLELYCIILGLPPCLNILRRKQPQLTFFTIAL
QSCHYQRLPPHILWATGLKSGGSSGGSSGSETPGTSESATPESSGGSSGGSDKKY
SIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDSGGSKRTADGSEFEPKKKR
KVGGGGSGATNFSLLKQAGDVEENPGPMVSKGEELFTGVVPILVELDGDVNGHKF
SVSGEGEGDATYGKLTLKFICTTGKLPVPWPTLVTTLTYGVQCFSRYPDHMKQHDF
FKSAMPEGYVQERTIFFKDDGNYKTRAEVKFEGDTLVNRIELKGIDFKEDGNILGHK
LEYNYNSHNVYIMADKQKNGIKVNFKIRHNIEDGSVQLADHYQQNTPIGDGPVLLPD
NHYLSTQSALSKDPNEKRDHMVLLEFVTAAGITLGMDELYK
nCas9 for BE4max-ΔUGI, SEQ ID NO: 164
MKRTADGSEFESPKKKRKVSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRK
SEETITPWNFEEWDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYFD
TTIDRKRYTSTKEVLDATLIHQSITGLYETRIDLSQLGGDSGGSKRTADGSEFEPKKK
RKVGGGGSGATNFSLLKQAGDVEENPGPMVSKGEELFTGVVPILVELDGDVNGHK
FSVSGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLP
DNHYLSTQSALSKDPNEKRDHMVLLEFVTAAGITLGMDELYK
hA3A-BE3-ΔUGI-eUNG, SEQ ID NO: 165
MEASPASGPRHLMDPHIFTSNFNNGIGRHKTYLCYEVERLDNGTSVKMDQHRGFL
HNQAKNLLCGFYGRHAELRFLDLVPSLQLDPAQIYRVTWFISWSPCFSWGCAGEV
RAFLQENTHVRLRIFAARIYDYDPLYKEALQMLRDAGAQVSIMTYDEFKHCWDTFV
DHQGCPFQPWDGLDEHSQALSGRLRAILQNQGNSGSETPGTSESATPESDKKYSI
GLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSANELTWHDVLAEEKQQP
YFLNTLQTVASERQSGVTIYPPQKDVFNAFRFTELGDVKVVILGQDPYHGPGQAHG
LAFSVRPGIAIPPSLLNMYKELENTIPGFTRPNHGYLESWARQGVLLLNTVLTVRAG
QAHSHASLGWETFTDKVISLINQHREGVVFLLWGSHAQKKGAIIDKQRHHVLKAPH
PSPLSAHRGFFGCNHFVLANQWLEQRGETPIDWMPVLPAESESGGSPKKKRKVG
GGGSGATNFSLLKQAGDVEENPGPMVSKGEELFTGVVPILVELDGDVNGHKFSVS
GEGEGDATYGKLTLKFICTTGKLPVPWPTLVTTLTYGVQCFSRYPDHMKQHDFFKS
AMPEGYVQERTIFFKDDGNYKTRAEVKFEGDTLVNRIELKGIDFKEDGNILGHKLEY
NYNSHNVYIMADKQKNGIKVNFKIRHNIEDGSVQLADHYQQNTPIGDGPVLLPDNHY
LSTQSALSKDPNEKRDHMVLLEFVTAAGITLGMDELYK
eA3A-BE3-ΔUGI-eUNG, SEQ ID NO: 166
MEASPASGPRHLMDPHIFTSNFNNGIGRHKTYLCYEVERLDNGTSVKMDQHRGFL
HGQAKNLLCGFYGRHAELRFLDLVPSLQLDPAQIYRVTWFISWSPCFSWGCAGEV
RAFLQENTHVRLRIFAARIYDYDPLYKEALQMLRDAGAQVSIMTYDEFKHCWDTFV
DHQGCPFQPWDGLDEHSQALSGRLRAILQNQGNSGSETPGTSESATPESDKKYSI
GLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSANELTWHDVLAEEKQQP
YFLNTLQTVASERQSGVTIYPPQKDVFNAFRFTELGDVKVVILGQDPYHGPGQAHG
LAFSVRPGIAIPPSLLNMYKELENTIPGFTRPNHGYLESWARQGVLLLNTVLTVRAG
QAHSHASLGWETFTDKVISLINQHREGVVFLLWGSHAQKKGAIIDKQRHHVLKAPH
PSPLSAHRGFFGCNHFVLANQWLEQRGETPIDWMPVLPAESESGGSPKKKRKVG
GGGSGATNFSLLKQAGDVEENPGPMVSKGEELFTGVVPILVELDGDVNGHKFSVS
GEGEGDATYGKLTLKFICTTGKLPVPWPTLVTTLTYGVQCFSRYPDHMKQHDFFKS
AMPEGYVQERTIFFKDDGNYKTRAEVKFEGDTLVNRIELKGIDFKEDGNILGHKLEY
NYNSHNVYIMADKQKNGIKVNFKIRHNIEDGSVQLADHYQQNTPIGDGPVLLPDNHY
LSTQSALSKDPNEKRDHMVLLEFVTAAGITLGMDELYK
hAID-BE3-ΔUGI-eUNG, SEQ ID NO: 167
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTA
RLYFCEDRKAEPEGLRRLHRAGVQIAIMTFKDYFYCWNTFVENHERTFKAWEGLHE
NSVRLSRQLRRILLPLYEVDDLRDAFRTLGLSGSETPGTSESATPESDKKYSIGLAIG
TNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLG
ELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTS
TKEVLDATLIHQSITGLYETRIDLSQLGGDSGGSANELTWHDVLAEEKQQPYFLNTL
QTVASERQSGVTIYPPQKDVFNAFRFTELGDVKVVILGQDPYHGPGQAHGLAFSVR
PGIAIPPSLLNMYKELENTIPGFTRPNHGYLESWARQGVLLLNTVLTVRAGQAHSHA
SLGWETFTDKVISLINQHREGVVFLLWGSHAQKKGAIIDKQRHHVLKAPHPSPLSAH
RGFFGCNHFVLANQWLEQRGETPIDWMPVLPAESESGGSPKKKRKVGGGGSGAT
NFSLLKQAGDVEENPGPMVSKGEELFTGVVPILVELDGDVNGHKFSVSGEGEGDA
TYGKLTLKFICTTGKLPVPWPTLVTTLTYGVQCFSRYPDHMKQHDFFKSAMPEGYV
QERTIFFKDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHYLSTQSAL
SKDPNEKRDHMVLLEFVTAAGITLGMDELYK
nCas9-eUNG for BE3, SEQ ID NO: 168
MDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGFIKRQLV
ETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGDSGGSANELTWHDVLAE
EKQQPYFLNTLQTVASERQSGVTIYPPQKDVFNAFRFTELGDVKVVILGQDPYHGP
GQAHGLAFSVRPGIAIPPSLLNMYKELENTIPGFTRPNHGYLESWARQGVLLLNTVL
TVRAGQAHSHASLGWETFTDKVISLINQHREGVVFLLWGSHAQKKGAIIDKQRHHV
LKAPHPSPLSAHRGFFGCNHFVLANQWLEQRGETPIDWMPVLPAESESGGSPKKK
RKVGGGGSGATNFSLLKQAGDVEENPGPMVSKGEELFTGVVPILVELDGDVNGHK
FSVSGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLP
DNHYLSTQSALSKDPNEKRDHMVLLEFVTAAGITLGMDELYK
Target-AID-ΔUGI-eUNG, SEQ ID NO: 169
MAPKKKRKVGIHGVPAAMDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIP
YYVGPLARGNSRFAWMTRKSEETITPWNFEEWDKGASAQSFIERMTNFDKNLPN
EKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKK
GILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDS
RADPKKKRKVGGGGTGGGGSAEYVRALFDFNGNDEEDLPFKKGDILRIRDKPEEQ
WWNAEDSEGKRGMILVPYVEKYSGDYKDHDGDYKDHDIDYKDDDDKSGMTDAEY
VRIHEKLDIYTFKKQFFNNKKSVSHRCYVLFELKRRGERRACFWGYAVNKPQSGTE
RGIHAEIFSIRKVEEYLRDNPGQFTINWYSSWSPCADCAEKILEWYNQELRGNGHTL
KIWACKLYYEKNARNQIGLWNLRDNGVGLNVMVSEHYQCCRKIFIQSSHNQLNENR
WLEKTLKRAEKWRSELSIMIQVKILHTTKSPAVGPKKKRKVGTANELTWHDVLAEEK
QQPYFLNTLQTVASERQSGVTIYPPQKDVFNAFRFTELGDVKVVILGQDPYHGPGQ
AHGLAFSVRPGIAIPPSLLNMYKELENTIPGFTRPNHGYLESWARQGVLLLNTVLTV
RAGQAHSHASLGWETFTDKVISLINQHREGVVFLLWGSHAQKKGAIIDKQRHHVLK
APHPSPLSAHRGFFGCNHFVLANQWLEQRGETPIDWMPVLPAESEGGGGSGATN
FSLLKQAGDVEENPGPMVSKGEELFTGVVPILVELDGDVNGHKFSVSGEGEGDAT
YGKLTLKFICTTGKLPVPWPTLVTTLTYGVQCFSRYPDHMKQHDFFKSAMPEGYVQ
ERTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSALSK
DPNEKRDHMVLLEFVTAAGITLGMDELYK
nCas9-eUNG for Target-AID, SEQ ID NO: 170
MAPKKKRKVGIHGVPAAMDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIP
YYVGPLARGNSRFAWMTRKSEETITPWNFEEWDKGASAQSFIERMTNFDKNLPN
EKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKK
GILQTVKWDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDS
RADPKKKRKVGGGGTGGGGSAEYVRALFDFNGNDEEDLPFKKGDILRIRDKPEEQ
WWNAEDSEGKRGMILVPYVEKYSGDYKDHDGDYKDHDIDYKDDDDKSGGPKKKR
KVGTANELTWHDVLAEEKQQPYFLNTLQTVASERQSGVTIYPPQKDVFNAFRFTEL
GDVKVVILGQDPYHGPGQAHGLAFSVRPGIAIPPSLLNMYKELENTIPGFTRPNHGY
LESWARQGVLLLNTVLTVRAGQAHSHASLGWETFTDKVISLINQHREGVVFLLWGS
HAQKKGAIIDKQRHHVLKAPHPSPLSAHRGFFGCNHFVLANQWLEQRGETPIDWM
PVLPAESEGGGGSGATNFSLLKQAGDVEENPGPMVSKGEELFTGVVPILVELDGDV
NGHKFSVSGEGEGDATYGKLTLKFICTTGKLPVPWPTLVTTLTYGVQCFSRYPDHM
KQHDFFKSAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYK
hA3A-BE3-ΔUGI-hUNG, SEQ ID NO: 171
MEASPASGPRHLMDPHIFTSNFNNGIGRHKTYLCYEVERLDNGTSVKMDQHRGFL
HNQAKNLLCGFYGRHAELRFLDLVPSLQLDPAQIYRVTWFISWSPCFSWGCAGEV
RAFLQENTHVRLRIFAARIYDYDPLYKEALQMLRDAGAQVSIMTYDEFKHCWDTFV
DHQGCPFQPWDGLDEHSQALSGRLRAILQNQGNSGSETPGTSESATPESDKKYSI
GLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSIGQKTLYSFFSPSPARKR
HAPSPEPAVQGTGVAGVPEESGDAAAIPAKKAPAGQEEPGTPPSSPLSAEQLDRIQ
RNKAAALLRLAARNVPVGFGESWKKHLSGEFGKPYFIKLMGFVAEERKHYTVYPPP
HQVFTWTQMCDIKDVKWILGQDPYHGPNQAHGLCFSVQRPVPPPPSLENIYKELS
TDIEDFVHPGHGDLSGWAKQGVLLLNAVLTVRAHQANSHKERGWEQFTDAVVSWL
NQNSNGLVFLLWGSYAQKKGSAIDRKRHHVLQTAHPSPLSVYRGFFGCRHFSKTN
ELLQKSGKKPIDWKELSGGSPKKKRKVGGGGSGATNFSLLKQAGDVEENPGPMVS
KGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFE
GDTLVNRIELKGIDFKEDGNILGHKLEYNYNSHNVYIMADKQKNGIKVNFKIRHNIED
GSVQLADHYQQNTPIGDGPVLLPDNHYLSTQSALSKDPNEKRDHMVLLEFVTAAGI
TLGMDELYK
eA3A-BE3-ΔUGI-hUNG, SEQ ID NO: 172
MEASPASGPRHLMDPHIFTSNFNNGIGRHKTYLCYEVERLDNGTSVKMDQHRGFL
HGQAKNLLCGFYGRHAELRFLDLVPSLQLDPAQIYRVTWFISWSPCFSWGCAGEV
RAFLQENTHVRLRIFAARIYDYDPLYKEALQMLRDAGAQVSIMTYDEFKHCWDTFV
DHQGCPFQPWDGLDEHSQALSGRLRAILQNQGNSGSETPGTSESATPESDKKYSI
GLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLWAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSIGQKTLYSFFSPSPARKR
HAPSPEPAVQGTGVAGVPEESGDAAAIPAKKAPAGQEEPGTPPSSPLSAEQLDRIQ
RNKAAALLRLAARNVPVGFGESWKKHLSGEFGKPYFIKLMGFVAEERKHYTVYPPP
HQVFTWTQMCDIKDVKVVILGQDPYHGPNQAHGLCFSVQRPVPPPPSLENIYKELS
TDIEDFVHPGHGDLSGWAKQGVLLLNAVLTVRAHQANSHKERGWEQFTDAVVSWL
NQNSNGLVFLLWGSYAQKKGSAIDRKRHHVLQTAHPSPLSVYRGFFGCRHFSKTN
ELLQKSGKKPIDWKELSGGSPKKKRKVGGGGSGATNFSLLKQAGDVEENPGPMVS
KGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFE
GDTLVNRIELKGIDFKEDGNILGHKLEYNYNSHNVYIMADKQKNGIKVNFKIRHNIED
GSVQLADHYQQNTPIGDGPVLLPDNHYLSTQSALSKDPNEKRDHMVLLEFVTAAGI
TLGMDELYK
hAID-BE3-ΔUGI-hUNG, SEQ ID NO: 173
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTA
RLYFCEDRKAEPEGLRRLHRAGVQIAIMTFKDYFYCWNTFVENHERTFKAWEGLHE
NSVRLSRQLRRILLPLYEVDDLRDAFRTLGLSGSETPGTSESATPESDKKYSIGLAIG
TNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLG
ELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTS
TKEVLDATLIHQSITGLYETRIDLSQLGGDSGGSIGQKTLYSFFSPSPARKRHAPSPE
PAVQGTGVAGVPEESGDAAAIPAKKAPAGQEEPGTPPSSPLSAEQLDRIQRNKAAA
LLRLAARNVPVGFGESWKKHLSGEFGKPYFIKLMGFVAEERKHYTVYPPPHQVFT
WTQMCDIKDVKVVILGQDPYHGPNQAHGLCFSVQRPVPPPPSLENIYKELSTDIEDF
VHPGHGDLSGWAKQGVLLLNAVLTVRAHQANSHKERGWEQFTDAVVSWLNQNSN
GLVFLLWGSYAQKKGSAIDRKRHHVLQTAHPSPLSVYRGFFGCRHFSKTNELLQKS
GKKPIDWKELSGGSPKKKRKVGGGGSGATNFSLLKQAGDVEENPGPMVSKGEELF
TGVVPILVELDGDVNGHKFSVSGEGEGDATYGKLTLKFICTTGKLPVPWPTLVTTLT
YGVQCFSRYPDHMKQHDFFKSAMPEGYVQERTIFFKDDGNYKTRAEVKFEGDTLV
NRIELKGIDFKEDGNILGHKLEYNYNSHNVYIMADKQKNGIKVNFKIRHNIEDGSVQL
ADHYQQNTPIGDGPVLLPDNHYLSTQSALSKDPNEKRDHMVLLEFVTAAGITLGMD
ELYK
nCas9-hUNG for BE3, SEQ ID NO: 174
MDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGFIKRQLV
ETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGDSGGSIGQKTLYSFFSPS
PARKRHAPSPEPAVQGTGVAGVPEESGDAAAIPAKKAPAGQEEPGTPPSSPLSAE
QLDRIQRNKAAALLRLAARNVPVGFGESWKKHLSGEFGKPYFIKLMGFVAEERKHY
TVYPPPHQVFTWTQMCDIKDVKVVILGQDPYHGPNQAHGLCFSVQRPVPPPPSLE
NIYKELSTDIEDFVHPGHGDLSGWAKQGVLLLNAVLTVRAHQANSHKERGWEQFT
DAVVSWLNQNSNGLVFLLWGSYAQKKGSAIDRKRHHVLQTAHPSPLSVYRGFFGC
RHFSKTNELLQKSGKKPIDWKELSGGSPKKKRKVGGGGSGATNFSLLKQAGDVEE
NPGPMVSKGEELFTGVVPILVELDGDVNGHKFSVSGEGEGDATYGKLTLKFICTTG
KLPVPWPTLVTTLTYGVQCFSRYPDHMKQHDFFKSAMPEGYVQERTIFFKDDGNY
KTRAEVKFEGDTLVNRIELKGIDFKEDGNILGHKLEYNYNSHNVYIMADKQKNGIKVN
FKIRHNIEDGSVQLADHYQQNTPIGDGPVLLPDNHYLSTQSALSKDPNEKRDHMVLL
EFVTAAGITLGMDELYK
Target-AID-ΔUGI-hUNG, SEQ ID NO: 175
MAPKKKRKVGIHGVPAAMDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKK
GILQTVKWDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDS
RADPKKKRKVGGGGTGGGGSAEYVRALFDFNGNDEEDLPFKKGDILRIRDKPEEQ
WWNAEDSEGKRGMILVPYVEKYSGDYKDHDGDYKDHDIDYKDDDDKSGMTDAEY
VRIHEKLDIYTFKKQFFNNKKSVSHRCYVLFELKRRGERRACFWGYAVNKPQSGTE
RGIHAEIFSIRKVEEYLRDNPGQFTINWYSSWSPCADCAEKILEWYNQELRGNGHTL
KIWACKLYYEKNARNQIGLWNLRDNGVGLNVMVSEHYQCCRKIFIQSSHNQLNENR
WLEKTLKRAEKWRSELSIMIQVKILHTTKSPAVGPKKKRKVGTIGQKTLYSFFSPSPA
RKRHAPSPEPAVQGTGVAGVPEESGDAAAIPAKKAPAGQEEPGTPPSSPLSAEQL
DRIQRNKAAALLRLAARNVPVGFGESWKKHLSGEFGKPYFIKLMGFVAEERKHYTV
YPPPHQVFTWTQMCDIKDVKVVILGQDPYHGPNQAHGLCFSVQRPVPPPPSLENIY
KELSTDIEDFVHPGHGDLSGWAKQGVLLLNAVLTVRAHQANSHKERGWEQFTDAV
VSWLNQNSNGLVFLLWGSYAQKKGSAIDRKRHHVLQTAHPSPLSVYRGFFGCRHF
SKTNELLQKSGKKPIDWKELGGGGSGATNFSLLKQAGDVEENPGPMVSKGEELFT
GVVPILVELDGDVNGHKFSVSGEGEGDATYGKLTLKFICTTGKLPVPWPTLVTTLTY
GVQCFSRYPDHMKQHDFFKSAMPEGYVQERTIFFKDDGNYKTRAEVKFEGDTLVN
RIELKGIDFKEDGNILGHKLEYNYNSHNVYIMADKQKNGIKVNFKIRHNIEDGSVQLA
DHYQQNTPIGDGPVLLPDNHYLSTQSALSKDPNEKRDHMVLLEFVTAAGITLGMDE
LYK
nCas9-hUNG for Target-AID, SEQ ID NO: 176
MAPKKKRKVGIHGVPAAMDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKK
GILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDS
RADPKKKRKVGGGGTGGGGSAEYVRALFDFNGNDEEDLPFKKGDILRIRDKPEEQ
WWNAEDSEGKRGMILVPYVEKYSGDYKDHDGDYKDHDIDYKDDDDKSGGPKKKR
KVGTIGQKTLYSFFSPSPARKRHAPSPEPAVQGTGVAGVPEESGDAAAIPAKKAPA
GQEEPGTPPSSPLSAEQLDRIQRNKAAALLRLAARNVPVGFGESWKKHLSGEFGK
PYFIKLMGFVAEERKHYTVYPPPHQVFTWTQMCDIKDVKVVILGQDPYHGPNQAHG
LCFSVQRPVPPPPSLENIYKELSTDIEDFVHPGHGDLSGWAKQGVLLLNAVLTVRAH
QANSHKERGWEQFTDAVVSWLNQNSNGLVFLLWGSYAQKKGSAIDRKRHHVLQT
AHPSPLSVYRGFFGCRHFSKTNELLQKSGKKPIDWKELGGGGSGATNFSLLKQAG
DVEENPGPMVSKGEELFTGVVPILVELDGDVNGHKFSVSGEGEGDATYGKLTLKFI
CTTGKLPVPWPTLVTTLTYGVQCFSRYPDHMKQHDFFKSAMPEGYVQERTIFFKD
DGNYKTRAEVKFEGDTLVNRIELKGIDFKEDGNILGHKLEYNYNSHNVYIMADKQKN
GIKVNFKIRHNIEDGSVQLADHYQQNTPIGDGPVLLPDNHYLSTQSALSKDPNEKRD
HMVLLEFVTAAGITLGMDELYK
hA3A-BE3 w/o UGI, SEQ ID NO: 177
MEASPASGPRHLMDPHIFTSNFNNGIGRHKTYLCYEVERLDNGTSVKMDQHRGFL
HNQAKNLLCGFYGRHAELRFLDLVPSLQLDPAQIYRVTWFISWSPCFSWGCAGEV
RAFLQENTHVRLRIFAARIYDYDPLYKEALQMLRDAGAQVSIMTYDEFKHCWDTFV
DHQGCPFQPWDGLDEHSQALSGRLRAILQNQGNSGSETPGTSESATPESDKKYSI
GLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLWAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSPKKKRKVGGGGSGATNF
SLLKQAGDVEENPGPMVSKGEELFTGVVPILVELDGDVNGHKFSVSGEGEGDATY
GKLTLKFICTTGKLPVPWPTLVTTLTYGVQCFSRYPDHMKQHDFFKSAMPEGYVQE
RTIFFKDDGNYKTRAEVKFEGDTLVNRIELKGIDFKEDGNILGHKLEYNYNSHNVYIM
ADKQKNGIKVNFKIRHNIEDGSVQLADHYQQNTPIGDGPVLLPDNHYLSTQSALSKD
PNEKRDHMVLLEFVTAAGITLGMDELYK
eA3A-BE3 w/o UGI, SEQ ID NO: 178
MEASPASGPRHLMDPHIFTSNFNNGIGRHKTYLCYEVERLDNGTSVKMDQHRGFL
HGQAKNLLCGFYGRHAELRFLDLVPSLQLDPAQIYRVTWFISWSPCFSWGCAGEV
RAFLQENTHVRLRIFAARIYDYDPLYKEALQMLRDAGAQVSIMTYDEFKHCWDTFV
DHQGCPFQPWDGLDEHSQALSGRLRAILQNQGNSGSETPGTSESATPESDKKYSI
GLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSPKKKRKVGGGGSGATNF
SLLKQAGDVEENPGPMVSKGEELFTGVVPILVELDGDVNGHKFSVSGEGEGDATY
GKLTLKFICTTGKLPVPWPTLVTTLTYGVQCFSRYPDHMKQHDFFKSAMPEGYVQE
RTIFFKDDGNYKTRAEVKFEGDTLVNRIELKGIDFKEDGNILGHKLEYNYNSHNVYIM
ADKQKNGIKVNFKIRHNIEDGSVQLADHYQQNTPIGDGPVLLPDNHYLSTQSALSKD
PNEKRDHMVLLEFVTAAGITLGMDELYK
hAID-BE3 w/o UGI, SEQ ID NO: 179
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTA
RLYFCEDRKAEPEGLRRLHRAGVQIAIMTFKDYFYCWNTFVENHERTFKAWEGLHE
NSVRLSRQLRRILLPLYEVDDLRDAFRTLGLSGSETPGTSESATPESDKKYSIGLAIG
TNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLG
ELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTS
TKEVLDATLIHQSITGLYETRIDLSQLGGDSGGSPKKKRKVGGGGSGATNFSLLKQA
GDVEENPGPMVSKGEELFTGVVPILVELDGDVNGHKFSVSGEGEGDATYGKLTLKF
ICTTGKLPVPWPTLVTTLTYGVQCFSRYPDHMKQHDFFKSAMPEGYVQERTIFFKD
DGNYKTRAEVKFEGDTLVNRIELKGIDFKEDGNILGHKLEYNYNSHNVYIMADKQKN
GIKVNFKIRHNIEDGSVQLADHYQQNTPIGDGPVLLPDNHYLSTQSALSKDPNEKRD
HMVLLEFVTAAGITLGMDELYK
nCas9 for BE3 w/o UGI, SEQ ID NO: 180
MDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGFIKRQLV
ETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGDSGGSPKKKRKVGGGG
SGATNFSLLKQAGDVEENPGPMVSKGEELFTGVVPILVELDGDVNGHKFSVSGEG
EGDATYGKLTLKFICTTGKLPVPWPTLVTTLTYGVQCFSRYPDHMKQHDFFKSAMP
EGYVQERTIFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLLPDNHYLST
QSALSKDPNEKRDHMVLLEFVTAAGITLGMDELYK
Target-AID w/o UGI, SEQ ID NO: 181
MAPKKKRKVGIHGVPAAMDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKK
GILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDS
RADPKKKRKVGGGGTGGGGSAEYVRALFDFNGNDEEDLPFKKGDILRIRDKPEEQ
WWNAEDSEGKRGMILVPYVEKYSGDYKDHDGDYKDHDIDYKDDDDKSGMTDAEY
VRIHEKLDIYTFKKQFFNNKKSVSHRCYVLFELKRRGERRACFWGYAVNKPQSGTE
RGIHAEIFSIRKVEEYLRDNPGQFTINWYSSWSPCADCAEKILEWYNQELRGNGHTL
KIWACKLYYEKNARNQIGLWNLRDNGVGLNVMVSEHYQCCRKIFIQSSHNQLNENR
WLEKTLKRAEKWRSELSIMIQVKILHTTKSPAVGPKKKRKVGGGGSGATNFSLLKQ
AGDVEENPGPMVSKGEELFTGVVPILVELDGDVNGHKFSVSGEGEGDATYGKLTL
KFICTTGKLPVPWPTLVTTLTYGVQCFSRYPDHMKQHDFFKSAMPEGYVQERTIFF
KDDGNYKTRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSALSKDPNEK
RDHMVLLEFVTAAGITLGMDELYK
nCas9 for Target-AID w/o UGI, SEQ ID NO: 182
MAPKKKRKVGIHGVPAAMDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKK
GILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDS
RADPKKKRKVGGGGTGGGGSAEYVRALFDFNGNDEEDLPFKKGDILRIRDKPEEQ
WWNAEDSEGKRGMILVPYVEKYSGDYKDHDGDYKDHDIDYKDDDDKSGGPKKKR
KVGGGGSGATNFSLLKQAGDVEENPGPMVSKGEELFTGVVPILVELDGDVNGHKF
SVSGEGEGDATYGKLTLKFICTTGKLPVPWPTLVTTLTYGVQCFSRYPDHMKQHDF
FKSAMPEGYVQERTIFFKDDGNYKTRAEVKFEGDTLVNRIELKGIDFKEDGNILGHK
LEYNYNSHNVYIMADKQKNGIKVNFKIRHNIEDGSVQLADHYQQNTPIGDGPVLLPD
NHYLSTQSALSKDPNEKRDHMVLLEFVTAAGITLGMDE
SPACE-ΔUGI-eUNG, SEQ ID NO: 183
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYGTFEPCVMCAGA
MIHSRIGRVVFGVRNAKTGAAGSLMDVLHYPGMNHRVEITEGILADECAALLCYFFR
MPRQVFNAQKKAQSSTDSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSKRTADGSEFEPKKKRKV
SGGSSGGSSGSETPGTSESATPESSGGSSGGSTDAEYVRIHEKLDIYTFKKQFFNN
KKSVSHRCYVLFELKRRGERRACFWGYAVNKPQSGTERGIHAEIFSIRKVEEYLRD
NPGQFTINWYSSWSPCADCAEKILEWYNQELRGNGHTLKIWACKLYYEKNARNQI
GLWNLRDNGVGLNVMVSEHYQCCRKIFIQSSHNQLNENRWLEKTLKRAEKWRSEL
SIMIQVKILHTTKSPAVSGGSGGSGGSANELTWHDVLAEEKQQPYFLNTLQTVASE
RQSGVTIYPPQKDVFNAFRFTELGDVKVVILGQDPYHGPGQAHGLAFSVRPGIAIPP
SLLNMYKELENTIPGFTRPNHGYLESWARQGVLLLNTVLTVRAGQAHSHASLGWET
FTDKVISLINQHREGVVFLLWGSHAQKKGAIIDKQRHHVLKAPHPSPLSAHRGFFGC
NHFVLANQWLEQRGETPIDWMPVLPAESEGSGATNFSLLKQAGDVEENPGPMVSK
GEELFTGVVPILVELDGDVNGHKFSVSGEGEGDATYGKLTLKFICTTGKLPVPWPTL
VTTLTYGVQCFSRYPDHMKQHDFFKSAMPEGYVQERTIFFKDDGNYKTRAEVKFE
GDTLVNRIELKGIDFKEDGNILGHKLEYNYNSHNVYIMADKQKNGIKVNFKIRHNIED
GSVQLADHYQQNTPIGDGPVLLPDNHYLSTQSALSKDPNEKRDHMVLLEFVTAAGI
TLGMDELYKSGGSPKKKRKV
SPACE-ΔUGI-hUNG, SEQ ID NO: 184
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYGTFEPCVMCAGA
MIHSRIGRVVFGVRNAKTGAAGSLMDVLHYPGMNHRVEITEGILADECAALLCYFFR
MPRQVFNAQKKAQSSTDSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSKRTADGSEFEPKKKRKV
SGGSSGGSSGSETPGTSESATPESSGGSSGGSTDAEYVRIHEKLDIYTFKKQFFNN
KKSVSHRCYVLFELKRRGERRACFWGYAVNKPQSGTERGIHAEIFSIRKVEEYLRD
NPGQFTINWYSSWSPCADCAEKILEWYNQELRGNGHTLKIWACKLYYEKNARNQI
GLWNLRDNGVGLNVMVSEHYQCCRKIFIQSSHNQLNENRWLEKTLKRAEKWRSEL
SIMIQVKILHTTKSPAVSGGSGGSGGSIGQKTLYSFFSPSPARKRHAPSPEPAVQGT
GVAGVPEESGDAAAIPAKKAPAGQEEPGTPPSSPLSAEQLDRIQRNKAAALLRLAA
RNVPVGFGESWKKHLSGEFGKPYFIKLMGFVAEERKHYTVYPPPHQVFTWTQMCD
IKDVKVVILGQDPYHGPNQAHGLCFSVQRPVPPPPSLENIYKELSTDIEDFVHPGHG
DLSGWAKQGVLLLNAVLTVRAHQANSHKERGWEQFTDAVVSWLNQNSNGLVFLL
WGSYAQKKGSAIDRKRHHVLQTAHPSPLSVYRGFFGCRHFSKTNELLQKSGKKPID
WKELGSGATNFSLLKQAGDVEENPGPMVSKGEELFTGVVPILVELDGDVNGHKFS
VSGEGEGDATYGKLTLKFICTTGKLPVPWPTLVTTLTYGVQCFSRYPDHMKQHDFF
KSAMPEGYVQERTIFFKDDGNYKTRAEVKFEGDTLVNRIELKGIDFKEDGNILGHKL
EYNYNSHNVYIMADKQKNGIKVNFKIRHNIEDGSVQLADHYQQNTPIGDGPVLLPDN
HYLSTQSALSKDPNEKRDHMVLLEFVTAAGITLGMDELYKSGGSPKKKRKV
ABEmax-eUNG, SEQ ID NO: 185
MKRTADGSEFESPKKKRKVSEVEFSHEYWMRHALTLAKRAWDEREVPVGAVLVH
NNRVIGEGWNRPIGRHDPTAHAEIMALRQGGLVMQNYRLIDATLYVTLEPCVMCAG
AMIHSRIGRVVFGARDAKTGAAGSLMDVLHHPGMNHRVEITEGILADECAALLSDFF
RMRRQEIKAQKKAQSSTDSGGSSGGSSGSETPGTSESATPESSGGSSGGSSEVE
FSHEYWMRHALTLAKRARDEREVPVGAVLVLNNRVIGEGWNRAIGLHDPTAHAEIM
ALRQGGLVMQNYRLIDATLYVTFEPCVMCAGAMIHSRIGRVVFGVRNAKTGAAGSL
MDVLHYPGMNHRVEITEGILADECAALLCYFFRMPRQVFNAQKKAQSSTDSGGSS
GGSSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIFSN
EMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDA
KLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPI
LEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENMEMARENQTTQKGQKNSRER
MKRIEEGIKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYE
TRIDLSQLGGDSGGSGGSGGSANELTWHDVLAEEKQQPYFLNTLQTVASERQSGV
TIYPPQKDVFNAFRFTELGDVKVVILGQDPYHGPGQAHGLAFSVRPGIAIPPSLLNM
YKELENTIPGFTRPNHGYLESWARQGVLLLNTVLTVRAGQAHSHASLGWETFTDKV
ISLINQHREGVVFLLWGSHAQKKGAIIDKQRHHVLKAPHPSPLSAHRGFFGCNHFVL
ANQWLEQRGETPIDWMPVLPAESESGGSKRTADGSEFEPKKKRKVGSGATNFSLL
KQAGDVEENPGPMVSKGEELFTGVVPILVELDGDVNGHKFSVSGEGEGDATYGKL
TLKFICTTGKLPVPWPTLVTTLTYGVQCFSRYPDHMKQHDFFKSAMPEGYVQERTI
FFKDDGNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALSKDPN
EKRDHMVLLEFVTAAGITLGMDELYKSGGSPKKKRKV
miniABEmax(V82G)-eUNG, SEQ ID NO: 186
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYGTFEPCVMCAGA
MIHSRIGRWFGVRNAKTGAAGSLMDVLHYPGMNHRVEITEGILADECAALLCYFFR
MPRQVFNAQKKAQSSTDSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSGGSGGSANELTWHDVL
AEEKQQPYFLNTLQTVASERQSGVTIYPPQKDVFNAFRFTELGDVKVVILGQDPYH
GPGQAHGLAFSVRPGIAIPPSLLNMYKELENTIPGFTRPNHGYLESWARQGVLLLNT
VLTVRAGQAHSHASLGWETFTDKVISLINQHREGVVFLLWGSHAQKKGAIIDKQRH
HVLKAPHPSPLSAHRGFFGCNHFVLANQWLEQRGETPIDWMPVLPAESESGGSKR
TADGSEFEPKKKRKVGSGATNFSLLKQAGDVEENPGPMVSKGEELFTGVVPILVEL
DGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTP
IGDGPVLLPDNHYLSTQSALSKDPNEKRDHMVLLEFVTAAGITLGMDELYKSGGSP
KKKRKV
nCas9-eUNG for ABEmax, SEQ ID NO: 187
MKRTADGSEFESPKKKRKVSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRK
SEETITPWNFEEWDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYFD
TTIDRKRYTSTKEVLDATLIHQSITGLYETRIDLSQLGGDSGGSGGSGGSANELTWH
DVLAEEKQQPYFLNTLQTVASERQSGVTIYPPQKDVFNAFRFTELGDVKVVILGQDP
YHGPGQAHGLAFSVRPGIAIPPSLLNMYKELENTIPGFTRPNHGYLESWARQGVLLL
NTVLTVRAGQAHSHASLGWETFTDKVISLINQHREGVVFLLWGSHAQKKGAIIDKQR
HHVLKAPHPSPLSAHRGFFGCNHFVLANQWLEQRGETPIDWMPVLPAESESGGSK
RTADGSEFEPKKKRKVGSGATNFSLLKQAGDVEENPGPMVSKGEELFTGVVPILVE
LDGDVNGHKFSVSGEGEGDATYGKLTLKFICTTGKLPVPWPTLVTTLTYGVQCFSR
YPDHMKQHDFFKSAMPEGYVQERTIFFKDDGNYKTRAEVKFEGDTLVNRIELKGID
FKEDGNILGHKLEYNYNSHNVYIMADKQKNGIKVNFKIRHNIEDGSVQLADHYQQNT
PIGDGPVLLPDNHYLSTQSALSKDPNEKRDHMVLLEFVTAAGITLGMDELYKSGGS
PKKKRKV
eUNG-ABEmax, SEQ ID NO: 188
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSEVEFSHEYWMRHALTLAKRA
WDEREVPVGAVLVHNNRVIGEGWNRPIGRHDPTAHAEIMALRQGGLVMQNYRLID
ATLYVTLEPCVMCAGAMIHSRIGRVVFGARDAKTGAAGSLMDVLHHPGMNHRVEIT
EGILADECAALLSDFFRMRRQEIKAQKKAQSSTDSGGSSGGSSGSETPGTSESATP
ESSGGSSGGSSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRV
VFGVRNAKTGAAGSLMDVLHYPGMNHRVEITEGILADECAALLCYFFRMPRQVFNA
QKKAQSSTDSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYI
DGGASQEEFYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSKRTADGSEFEPKKKRKVGSGATNFSL
LKQAGDVEENPGPMVSKGEELFTGVVPILVELDGDVNGHKFSVSGEGEGDATYGK
LTLKFICTTGKLPVPWPTLVTTLTYGVQCFSRYPDHMKQHDFFKSAMPEGYVQERTI
FFKDDGNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALSKDPN
EKRDHMVLLEFVTAAGITLGMDELYKSGGSPKKKRKV
eUNG-miniABEmax(V82G), SEQ ID NO: 189
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSEVEFSHEYWMRHALTLAKRAR
DEREVPVGAVLVLNNRVIGEGWNRAIGLHDPTAHAEIMALRQGGLVMQNYRLIDAT
LYGTFEPCVMCAGAMIHSRIGRWFGVRNAKTGAAGSLMDVLHYPGMNHRVEITE
GILADECAALLCYFFRMPRQVFNAQKKAQSSTDSGGSSGGSSGSETPGTSESATP
ESSGGSSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRL
ENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFE
DREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTV
KWDELVKVMGRHKPENIVIEMARENQTTQKGQKNSRERMKRIEEGIKELGSQILKE
HPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKL
PKYSLFELENGRKRMLASAGELQKGNELALPSKYVNFLYLASHYEKLKGSPEDNEQ
KQLFVEQHKHYLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSGGS
KRTADGSEFEPKKKRKVGSGATNFSLLKQAGDVEENPGPMVSKGEELFTGVVPILV
ELDGDVNGHKFSVSGEGEGDATYGKLTLKFICTTGKLPVPWPTLVTTLTYGVQCFS
RYPDHMKQHDFFKSAMPEGYVQERTIFFKDDGNYKTRAEVKFEGDTLVNRIELKGI
DFKEDGNILGHKLEYNYNSHNVYIMADKQKNGIKVNFKIRHNIEDGSVQLADHYQQN
TPIGDGPVLLPDNHYLSTQSALSKDPNEKRDHMVLLEFVTAAGITLGMDELYKSGGS
PKKKRKV
eUNG-nCas9 for ABEmax, SEQ ID NO: 190
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSGGSSGGSSGSETPGTSESATP
ESSGGSSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRL
ENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFE
DREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKE
HPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKL
PKYSLFELENGRKRMLASAGELQKGNELALPSKYVNFLYLASHYEKLKGSPEDNEQ
KQLFVEQHKHYLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSGGS
KRTADGSEFEPKKKRKVGSGATNFSLLKQAGDVEENPGPMVSKGEELFTGVVPILV
ELDGDVNGHKFSVSGEGEGDATYGKLTLKFICTTGKLPVPWPTLVTTLTYGVQCFS
RYPDHMKQHDFFKSAMPEGYVQERTIFFKDDGNYKTRAEVKFEGDTLVNRIELKGI
DFKEDGNILGHKLEYNYNSHNVYIMADKQKNGIKVNFKIRHNIEDGSVQLADHYQQN
TPIGDGPVLLPDNHYLSTQSALSKDPNEKRDHMVLLEFVTAAGITLGMDELYKSGGS
PKKKRKV
ABEmax-hUNG, SEQ ID NO: 191
MKRTADGSEFESPKKKRKVSEVEFSHEYWMRHALTLAKRAWDEREVPVGAVLVH
NNRVIGEGWNRPIGRHDPTAHAEIMALRQGGLVMQNYRLIDATLYVTLEPCVMCAG
AMIHSRIGRVVFGARDAKTGAAGSLMDVLHHPGMNHRVEITEGILADECAALLSDFF
RMRRQEIKAQKKAQSSTDSGGSSGGSSGSETPGTSESATPESSGGSSGGSSEVE
FSHEYWMRHALTLAKRARDEREVPVGAVLVLNNRVIGEGWNRAIGLHDPTAHAEIM
ALRQGGLVMQNYRLIDATLYVTFEPCVMCAGAMIHSRIGRVVFGVRNAKTGAAGSL
MDVLHYPGMNHRVEITEGILADECAALLCYFFRMPRQVFNAQKKAQSSTDSGGSS
GGSSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIFSN
EMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDA
KLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPI
LEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYE
TRIDLSQLGGDSGGSGGSGGSIGQKTLYSFFSPSPARKRHAPSPEPAVQGTGVAG
VPEESGDAAAIPAKKAPAGQEEPGTPPSSPLSAEQLDRIQRNKAAALLRLAARNVPV
GFGESWKKHLSGEFGKPYFIKLMGFVAEERKHYTVYPPPHQVFTWTQMCDIKDVK
VVILGQDPYHGPNQAHGLCFSVQRPVPPPPSLENIYKELSTDIEDFVHPGHGDLSG
WAKQGVLLLNAVLTVRAHQANSHKERGWEQFTDAVVSWLNQNSNGLVFLLWGSY
AQKKGSAIDRKRHHVLQTAHPSPLSVYRGFFGCRHFSKTNELLQKSGKKPIDWKEL
SGGSKRTADGSEFEPKKKRKVGSGATNFSLLKQAGDVEENPGPMVSKGEELFTGV
VPILVELDGDVNGHKFSVSGEGEGDATYGKLTLKFICTTGKLPVPWPTLVTTLTYGV
QCFSRYPDHMKQHDFFKSAMPEGYVQERTIFFKDDGNYKTRAEVKFEGDTLVNRIE
LKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITLGMDELYK
SGGSPKKKRKV
miniABEmax(V82G)-hUNG, SEQ ID NO: 192
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYGTFEPCVMCAGA
MIHSRIGRVVFGVRNAKTGAAGSLMDVLHYPGMNHRVEITEGILADECAALLCYFFR
MPRQVFNAQKKAQSSTDSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSGGSGGSIGQKTLYSFFS
PSPARKRHAPSPEPAVQGTGVAGVPEESGDAAAIPAKKAPAGQEEPGTPPSSPLS
AEQLDRIQRNKAAALLRLAARNVPVGFGESWKKHLSGEFGKPYFIKLMGFVAEERK
HYTVYPPPHQVFTWTQMCDIKDVKVVILGQDPYHGPNQAHGLCFSVQRPVPPPPS
LENIYKELSTDIEDFVHPGHGDLSGWAKQGVLLLNAVLTVRAHQANSHKERGWEQF
TDAVVSWLNQNSNGLVFLLWGSYAQKKGSAIDRKRHHVLQTAHPSPLSVYRGFFG
CRHFSKTNELLQKSGKKPIDWKELSGGSKRTADGSEFEPKKKRKVGSGATNFSLLK
QAGDVEENPGPMVSKGEELFTGWPILVELDGDVNGHKFSVSGEGEGDATYGKLT
LKFICTTGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHNVYIMADK
QKNGIKVNFKIRHNIEDGSVQLADHYQQNTPIGDGPVLLPDNHYLSTQSALSKDPNE
KRDHMVLLEFVTAAGITLGMDELYKSGGSPKKKRKV
nCas9-hUNG for ABEmax, SEQ ID NO: 193
MKRTADGSEFESPKKKRKVSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYFD
TTIDRKRYTSTKEVLDATLIHQSITGLYETRIDLSQLGGDSGGSGGSGGSIGQKTLYS
FFSPSPARKRHAPSPEPAVQGTGVAGVPEESGDAAAIPAKKAPAGQEEPGTPPSS
PLSAEQLDRIQRNKAAALLRLAARNVPVGFGESWKKHLSGEFGKPYFIKLMGFVAE
ERKHYTVYPPPHQVFTWTQMCDIKDVKVVILGQDPYHGPNQAHGLCFSVQRPVPP
PPSLENIYKELSTDIEDFVHPGHGDLSGWAKQGVLLLNAVLTVRAHQANSHKERGW
EQFTDAVVSWLNQNSNGLVFLLWGSYAQKKGSAIDRKRHHVLQTAHPSPLSVYRG
FFGCRHFSKTNELLQKSGKKPIDWKELSGGSKRTADGSEFEPKKKRKVGSGATNF
SLLKQAGDVEENPGPMVSKGEELFTGVVPILVELDGDVNGHKFSVSGEGEGDATY
GKLTLKFICTTGKLPVPWPTLVTTLTYGVQCFSRYPDHMKQHDFFKSAMPEGYVQE
RTIFFKDDGNYKTRAEVKFEGDTLVNRIELKGIDFKEDGNILGHKLEYNYNSHNVYIM
ADKQKNGIKVNFKIRHNIEDGSVQLADHYQQNTPIGDGPVLLPDNHYLSTQSALSKD
PNEKRDHMVLLEFVTAAGITLGMDELYKSGGSPKKKRKV
hUNG-ABEmax, SEQ ID NO: 194
MKRTADGSEFESPKKKRKVIGQKTLYSFFSPSPARKRHAPSPEPAVQGTGVAGVP
EESGDAAAIPAKKAPAGQEEPGTPPSSPLSAEQLDRIQRNKAAALLRLAARNVPVG
FGESWKKHLSGEFGKPYFIKLMGFVAEERKHYTVYPPPHQVFTWTQMCDIKDVKV
VILGQDPYHGPNQAHGLCFSVQRPVPPPPSLENIYKELSTDIEDFVHPGHGDLSGW
AKQGVLLLNAVLTVRAHQANSHKERGWEQFTDAVVSWLNQNSNGLVFLLWGSYA
QKKGSAIDRKRHHVLQTAHPSPLSVYRGFFGCRHFSKTNELLQKSGKKPIDWKELS
GGSGGSGGSSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRV
VFGARDAKTGAAGSLMDVLHHPGMNHRVEITEGILADECAALLSDFFRMRRQEIKA
QKKAQSSTDSGGSSGGSSGSETPGTSESATPESSGGSSGGSSEVEFSHEYWMRH
ALTLAKRARDEREVPVGAVLVLNNRVIGEGWNRAIGLHDPTAHAEIMALRQGGLVM
QNYRLIDATLYVTFEPCVMCAGAMIHSRIGRVVFGVRNAKTGAAGSLMDVLHYPGM
NHRVEITEGILADECAALLCYFFRMPRQVFNAQKKAQSSTDSGGSSGGSSGSETPG
TSESATPESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIP
YYVGPLARGNSRFAWMTRKSEETITPWNFEEWDKGASAQSFIERMTNFDKNLPN
EKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKK
GILQTVKWDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDS
GGSKRTADGSEFEPKKKRKVGSGATNFSLLKQAGDVEENPGPMVSKGEELFTGVV
PILVELDGDVNGHKFSVSGEGEGDATYGKLTLKFICTTGKLPVPWPTLVTTLTYGVQ
CFSRYPDHMKQHDFFKSAMPEGYVQERTIFFKDDGNYKTRAEVKFEGDTLVNRIEL
KGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITLGMDELYK
SGGSPKKKRKV
hUNG-miniABEmax(V82G), SEQ ID NO: 195
MKRTADGSEFESPKKKRKVIGQKTLYSFFSPSPARKRHAPSPEPAVQGTGVAGVP
EESGDAAAIPAKKAPAGQEEPGTPPSSPLSAEQLDRIQRNKAAALLRLAARNVPVG
FGESWKKHLSGEFGKPYFIKLMGFVAEERKHYTVYPPPHQVFTWTQMCDIKDVKV
VILGQDPYHGPNQAHGLCFSVQRPVPPPPSLENIYKELSTDIEDFVHPGHGDLSGW
AKQGVLLLNAVLTVRAHQANSHKERGWEQFTDAVVSWLNQNSNGLVFLLWGSYA
QKKGSAIDRKRHHVLQTAHPSPLSVYRGFFGCRHFSKTNELLQKSGKKPIDWKELS
GGSGGSGGSSEVEFSHEYWMRHALTLAKRARDEREVPVGAVLVLNNRVIGEGWN
RAIGLHDPTAHAEIMALRQGGLVMQNYRLIDATLYGTFEPCVMCAGAMIHSRIGRVV
FGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSV
GWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSKRTADGSEFEPKKKRKVGSGATNFSL
LKQAGDVEENPGPMVSKGEELFTGVVPILVELDGDVNGHKFSVSGEGEGDATYGK
LTLKFICTTGKLPVPWPTLVTTLTYGVQCFSRYPDHMKQHDFFKSAMPEGYVQERTI
FFKDDGNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALSKDPN
EKRDHMVLLEFVTAAGITLGMDELYKSGGSPKKKRKV
hUNG-nCas9 for ABEmax, SEQ ID NO: 196
MKRTADGSEFESPKKKRKVIGQKTLYSFFSPSPARKRHAPSPEPAVQGTGVAGVP
EESGDAAAIPAKKAPAGQEEPGTPPSSPLSAEQLDRIQRNKAAALLRLAARNVPVG
FGESWKKHLSGEFGKPYFIKLMGFVAEERKHYTVYPPPHQVFTWTQMCDIKDVKV
VILGQDPYHGPNQAHGLCFSVQRPVPPPPSLENIYKELSTDIEDFVHPGHGDLSGW
AKQGVLLLNAVLTVRAHQANSHKERGWEQFTDAVVSWLNQNSNGLVFLLWGSYA
QKKGSAIDRKRHHVLQTAHPSPLSVYRGFFGCRHFSKTNELLQKSGKKPIDWKELS
GGSGGSGGSSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYI
DGGASQEEFYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKE
VLDATLIHQSITGLYETRIDLSQLGGDSGGSKRTADGSEFEPKKKRKVGSGATNFSL
LKQAGDVEENPGPMVSKGEELFTGVVPILVELDGDVNGHKFSVSGEGEGDATYGK
LTLKFICTTGKLPVPWPTLVTTLTYGVQCFSRYPDHMKQHDFFKSAMPEGYVQERTI
FFKDDGNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALSKDPN
EKRDHMVLLEFVTAAGITLGMDELYKSGGSPKKKRKV
ABEmax-UGI, SEQ ID NO: 197
MKRTADGSEFESPKKKRKVSEVEFSHEYWMRHALTLAKRAWDEREVPVGAVLVH
NNRVIGEGWNRPIGRHDPTAHAEIMALRQGGLVMQNYRLIDATLYVTLEPCVMCAG
AMIHSRIGRVVFGARDAKTGAAGSLMDVLHHPGMNHRVEITEGILADECAALLSDFF
RMRRQEIKAQKKAQSSTDSGGSSGGSSGSETPGTSESATPESSGGSSGGSSEVE
FSHEYWMRHALTLAKRARDEREVPVGAVLVLNNRVIGEGWNRAIGLHDPTAHAEIM
ALRQGGLVMQNYRLIDATLYVTFEPCVMCAGAMIHSRIGRVVFGVRNAKTGAAGSL
MDVLHYPGMNHRVEITEGILADECAALLCYFFRMPRQVFNAQKKAQSSTDSGGSS
GGSSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIFSN
EMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDA
KLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPI
LEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHI
ANLAGSPAIKKGILQTVKVVDELVKVMGRHKPENMEMARENQTTQKGQKNSRER
MKRIEEGIKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYE
TRIDLSQLGGDSGGSGGSGGSTNLSDIIEKETGKQLVIQESILMLPEEVEEVIGNKPE
SDILVHTAYDESTDENVMLLTSDAPEYKPWALVIQDSNGENKIKMLSGGSGGSGGS
TNLSDIIEKETGKQLVIQESILMLPEEVEEVIGNKPESDILVHTAYDESTDENVMLLTS
DAPEYKPWALVIQDSNGENKIKMLSGGSKRTADGSEFEPKKKRKVGSGATNFSLLK
QAGDVEENPGPMVSKGEELFTGVVPILVELDGDVNGHKFSVSGEGEGDATYGKLT
LKFICTTGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHNVYIMADK
QKNGIKVNFKIRHNIEDGSVQLADHYQQNTPIGDGPVLLPDNHYLSTQSALSKDPNE
KRDHMVLLEFVTAAGITLGMDELYKSGGSPKKKRKV
miniABEmax(V82G)-UGI, SEQ ID NO: 198
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYGTFEPCVMCAGA
MIHSRIGRVVFGVRNAKTGAAGSLMDVLHYPGMNHRVEITEGILADECAALLCYFFR
MPRQVFNAQKKAQSSTDSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLWAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSGGSGGSTNLSDIIEKETG
KQLVIQESILMLPEEVEEVIGNKPESDILVHTAYDESTDENVMLLTSDAPEYKPWALV
IQDSNGENKIKMLSGGSGGSGGSTNLSDIIEKETGKQLVIQESILMLPEEVEEVIGNK
PESDILVHTAYDESTDENVMLLTSDAPEYKPWALVIQDSNGENKIKMLSGGSKRTAD
GSEFEPKKKRKVGSGATNFSLLKQAGDVEENPGPMVSKGEELFTGVVPILVELDGD
VNGHKFSVSGEGEGDATYGKLTLKFICTTGKLPVPWPTLVTTLTYGVQCFSRYPDH
MKQHDFFKSAMPEGYVQERTIFFKDDGNYKTRAEVKFEGDTLVNRIELKGIDFKED
GNILGHKLEYNYNSHNVYIMADKQKNGIKVNFKIRHNIEDGSVQLADHYQQNTPIGD
GPVLLPDNHYLSTQSALSKDPNEKRDHMVLLEFVTAAGITLGMDELYKSGGSPKKK
RKV
nCas9-UGI for ABEmax, SEQ ID NO: 199
MKRTADGSEFESPKKKRKVSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRK
SEETITPWNFEEWDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYFD
TTIDRKRYTSTKEVLDATLIHQSITGLYETRIDLSQLGGDSGGSGGSGGSTNLSDIIEK
ETGKQLVIQESILMLPEEVEEVIGNKPESDILVHTAYDESTDENVMLLTSDAPEYKPW
ALVIQDSNGENKIKMLSGGSGGSGGSTNLSDIIEKETGKQLVIQESILMLPEEVEEVI
GNKPESDILVHTAYDESTDENVMLLTSDAPEYKPWALVIQDSNGENKIKMLSGGSK
RTADGSEFEPKKKRKVGSGATNFSLLKQAGDVEENPGPMVSKGEELFTGVVPILVE
LDGDVNGHKFSVSGEGEGDATYGKLTLKFICTTGKLPVPWPTLVTTLTYGVQCFSR
YPDHMKQHDFFKSAMPEGYVQERTIFFKDDGNYKTRAEVKFEGDTLVNRIELKGID
FKEDGNILGHKLEYNYNSHNVYIMADKQKNGIKVNFKIRHNIEDGSVQLADHYQQNT
PIGDGPVLLPDNHYLSTQSALSKDPNEKRDHMVLLEFVTAAGITLGMDELYKSGGS
PKKKRKV
REV1 (human) amino acid sequence, SEQ ID NO: 200
MRRGGWRKRAENDGWETWGGYMAAKVQKLEEQFRSDAAMQKDGTSSTIFSGVA
IYVNGYTDPSAEELRKLMMLHGGQYHVYYSRSKTTHIIATNLPNAKIKELKGEKVIRP
EWIVESIKAGRLLSYIPYQLYTKQSSVQKGLSFNPVCRPEDPLPGPSNIAKQLNNRV
NHIVKKIETENEVKVNGMNSWNEEDENNDFSFVDLEQTSPGRKQNGIPHPRGSTAI
FNGHTPSSNGALKTQDCLVPMVNSVASRLSPAFSQEEDKAEKSSTDFRDCTLQQL
QQSTRNTDALRNPHRTNSFSLSPLHSNTKINGAHHSTVQGPSSTKSTSSVSTFSKA
APSVPSKPSDCNFISNFYSHSRLHHISMWKCELTEFVNTLQRQSNGIFPGREKLKK
MKTGRSALVVTDTGDMSVLNSPRHQSCIMHVDMDCFFVSVGIRNRPDLKGKPVAV
TSNRGTGRAPLRPGANPQLEWQYYQNKILKGKAADIPDSSLWENPDSAQANGIDS
VLSRAEIASCSYEARQLGIKNGMFFGHAKQLCPNLQAVPYDFHAYKEVAQTLYETLA
SYTHNIEAVSCDEALVDITEILAETKLTPDEFANAVRMEIKDQTKCAASVGIGSNILLA
RMATRKAKPDGQYHLKPEEVDDFIRGQLVTNLPGVGHSMESKLASLGIKTCGDLQY
MTMAKLQKEFGPKTGQMLYRFCRGLDDRPVRTEKERKSVSAEINYGIRFTQPKEAE
AFLLSLSEEIQRRLEATGMKGKRLTLKIMVRKPGAPVETAKFGGHGICDNIARTVTLD
QATDNAKIIGKAMLNMFHTMKLNISDMRGVGIHVNQLVPTNLNPSTCPSRPSVQSS
HFPSGSYSVRDVFQVQKAKKSTEEEHKEVFRAAVDLEISSASRTCTFLPPFPAHLPT
SPDTNKAESSGKWNGLHTPVSVQSRLNLSIEVPSPSQLDQSVLEALPPDLREQVEQ
VCAVQQAESHGDKKKEPVNGCNTGILPQPVGTVLLQIPEPQESNSDAGINLIALPAF
SQVDPEVFAALPAELQRELKAAYDQRQRQGENSTHQQSASASVPKNPLLHLKAAV
KEKKRNKKKKTIGSPKRIQSPLNNKLLNSPAKTLPGACGSPQKLIDGFLKHEGPPAE
KPLEELSASTSGVPGLSSLQSDPAGCVRPPAPNLAGAVEFNDVKTLLREWITTISDP
MEEDILQVVKYCTDLIEEKDLEKLDLVIKYMKRLMQQSVESVWNMAFDFILDNVQVV
LQQTYGSTLKVT
BE4max-REV1, SEQ ID NO: 201
MKRTADGSEFESPKKKRKVSSETGPVAVDPTLRRRIEPHEFEVFFDPRELRKETCL
LYEINWGGRHSIWRHTSQNTNKHVEVNFIEKFTTERYFCPNTRCSITWFLSWSPCG
ECSRAITEFLSRYPHVTLFIYIARLYHHADPRNRQGLRDLISSGVTIQIMTEQESGYC
WRNFVNYSPSNEAHWPRYPHLWVRLYVLELYCIILGLPPCLNILRRKQPQLTFFTIAL
QSCHYQRLPPHILWATGLKSGGSSGGSSGSETPGTSESATPESSGGSSGGSDKKY
SIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDSGGSRRGGWRKRAENDG
WETWGGYMAAKVQKLEEQFRSDAAMQKDGTSSTIFSGVAIYVNGYTDPSAEELRK
LMMLHGGQYHVYYSRSKTTHIIATNLPNAKIKELKGEKVIRPEWIVESIKAGRLLSYIP
YQLYTKQSSVQKGLSFNPVCRPEDPLPGPSNIAKQLNNRVNHIVKKIETENEVKVNG
MNSWNEEDENNDFSFVDLEQTSPGRKQNGIPHPRGSTAIFNGHTPSSNGALKTQD
CLVPMVNSVASRLSPAFSQEEDKAEKSSTDFRDCTLQQLQQSTRNTDALRNPHRT
NSFSLSPLHSNTKINGAHHSTVQGPSSTKSTSSVSTFSKAAPSVPSKPSDCNFISNF
YSHSRLHHISMWKCELTEFVNTLQRQSNGIFPGREKLKKMKTGRSALVVTDTGDMS
VLNSPRHQSCIMHVDMDCFFVSVGIRNRPDLKGKPVAVTSNRGTGRAPLRPGANP
QLEWQYYQNKILKGKAADIPDSSLWENPDSAQANGIDSVLSRAEIASCSYEARQLGI
KNGMFFGHAKQLCPNLQAVPYDFHAYKEVAQTLYETLASYTHNIEAVSCDEALVDIT
EILAETKLTPDEFANAVRMEIKDQTKCAASVGIGSNILLARMATRKAKPDGQYHLKPE
EVDDFIRGQLVTNLPGVGHSMESKLASLGIKTCGDLQYMTMAKLQKEFGPKTGQML
YRFCRGLDDRPVRTEKERKSVSAEINYGIRFTQPKEAEAFLLSLSEEIQRRLEATGM
KGKRLTLKIMVRKPGAPVETAKFGGHGICDNIARTVTLDQATDNAKIIGKAMLNMFH
TMKLNISDMRGVGIHVNQLVPTNLNPSTCPSRPSVQSSHFPSGSYSVRDVFQVQKA
KKSTEEEHKEVFRAAVDLEISSASRTCTFLPPFPAHLPTSPDTNKAESSGKWNGLHT
PVSVQSRLNLSIEVPSPSQLDQSVLEALPPDLREQVEQVCAVQQAESHGDKKKEPV
NGCNTGILPQPVGTVLLQIPEPQESNSDAGINLIALPAFSQVDPEVFAALPAELQREL
KAAYDQRQRQGENSTHQQSASASVPKNPLLHLKAAVKEKKRNKKKKTIGSPKRIQS
PLNNKLLNSPAKTLPGACGSPQKLIDGFLKHEGPPAEKPLEELSASTSGVPGLSSLQ
SDPAGCVRPPAPNLAGAVEFNDVKTLLREWITTISDPMEEDILQVVKYCTDLIEEKDL
EKLDLVIKYMKRLMQQSVESVWNMAFDFILDNVQVVLQQTYGSTLKVTSGGSKRTA
DGSEFEPKKKRKVGGGGSGATNFSLLKQAGDVEENPGPMVSKGEELFTGVVPILV
ELDGDVNGHKFSVSGEGEGDATYGKLTLKFICTTGKLPVPWPTLVTTLTYGVQCFS
RYPDHMKQHDFFKSAMPEGYVQERTIFFKDDGNYKTRAEVKFEGDTLVNRIELKGI
DFKEDGNILGHKLEYNYNSHNVYIMADKQKNGIKVNFKIRHNIEDGSVQLADHYQQN
TPIGDGPVLLPDNHYLSTQSALSKDPNEKRDHMVLLEFVTAAGITLGMDELYK
BE4max-REV1-eUNG, SEQ ID NO: 202
MKRTADGSEFESPKKKRKVSSETGPVAVDPTLRRRIEPHEFEVFFDPRELRKETCL
LYEINWGGRHSIWRHTSQNTNKHVEVNFIEKFTTERYFCPNTRCSITWFLSWSPCG
ECSRAITEFLSRYPHVTLFIYIARLYHHADPRNRQGLRDLISSGVTIQIMTEQESGYC
WRNFVNYSPSNEAHWPRYPHLWVRLYVLELYCIILGLPPCLNILRRKQPQLTFFTIAL
QSCHYQRLPPHILWATGLKSGGSSGGSSGSETPGTSESATPESSGGSSGGSDKKY
SIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDSGGSRRGGWRKRAENDG
WETWGGYMAAKVQKLEEQFRSDAAMQKDGTSSTIFSGVAIYVNGYTDPSAEELRK
LMMLHGGQYHVYYSRSKTTHIIATNLPNAKIKELKGEKVIRPEWIVESIKAGRLLSYIP
YQLYTKQSSVQKGLSFNPVCRPEDPLPGPSNIAKQLNNRVNHIVKKIETENEVKVNG
MNSWNEEDENNDFSFVDLEQTSPGRKQNGIPHPRGSTAIFNGHTPSSNGALKTQD
CLVPMVNSVASRLSPAFSQEEDKAEKSSTDFRDCTLQQLQQSTRNTDALRNPHRT
NSFSLSPLHSNTKINGAHHSTVQGPSSTKSTSSVSTFSKAAPSVPSKPSDCNFISNF
YSHSRLHHISMWKCELTEFVNTLQRQSNGIFPGREKLKKMKTGRSALVVTDTGDMS
VLNSPRHQSCIMHVDMDCFFVSVGIRNRPDLKGKPVAVTSNRGTGRAPLRPGANP
QLEWQYYQNKILKGKAADIPDSSLWENPDSAQANGIDSVLSRAEIASCSYEARQLGI
KNGMFFGHAKQLCPNLQAVPYDFHAYKEVAQTLYETLASYTHNIEAVSCDEALVDIT
EILAETKLTPDEFANAVRMEIKDQTKCAASVGIGSNILLARMATRKAKPDGQYHLKPE
EVDDFIRGQLVTNLPGVGHSMESKLASLGIKTCGDLQYMTMAKLQKEFGPKTGQML
YRFCRGLDDRPVRTEKERKSVSAEINYGIRFTQPKEAEAFLLSLSEEIQRRLEATGM
KGKRLTLKIMVRKPGAPVETAKFGGHGICDNIARTVTLDQATDNAKIIGKAMLNMFH
TMKLNISDMRGVGIHVNQLVPTNLNPSTCPSRPSVQSSHFPSGSYSVRDVFQVQKA
KKSTEEEHKEVFRAAVDLEISSASRTCTFLPPFPAHLPTSPDTNKAESSGKWNGLHT
PVSVQSRLNLSIEVPSPSQLDQSVLEALPPDLREQVEQVCAVQQAESHGDKKKEPV
NGCNTGILPQPVGTVLLQIPEPQESNSDAGINLIALPAFSQVDPEVFAALPAELQREL
KAAYDQRQRQGENSTHQQSASASVPKNPLLHLKAAVKEKKRNKKKKTIGSPKRIQS
PLNNKLLNSPAKTLPGACGSPQKLIDGFLKHEGPPAEKPLEELSASTSGVPGLSSLQ
SDPAGCVRPPAPNLAGAVEFNDVKTLLREWITTISDPMEEDILQVVKYCTDLIEEKDL
EKLDLVIKYMKRLMQQSVESVWNMAFDFILDNVQVVLQQTYGSTLKVTSGGSGGS
GGSANELTWHDVLAEEKQQPYFLNTLQTVASERQSGVTIYPPQKDVFNAFRFTELG
DVKVVILGQDPYHGPGQAHGLAFSVRPGIAIPPSLLNMYKELENTIPGFTRPNHGYL
ESWARQGVLLLNTVLTVRAGQAHSHASLGWETFTDKVISLINQHREGVVFLLWGSH
AQKKGAIIDKQRHHVLKAPHPSPLSAHRGFFGCNHFVLANQWLEQRGETPIDWMP
VLPAESESGGSKRTADGSEFEPKKKRKVGGGGSGATNFSLLKQAGDVEENPGPM
VSKGEELFTGVVPILVELDGDVNGHKFSVSGEGEGDATYGKLTLKFICTTGKLPVPW
PTLVTTLTYGVQCFSRYPDHMKQHDFFKSAMPEGYVQERTIFFKDDGNYKTRAEVK
FEGDTLVNRIELKGIDFKEDGNILGHKLEYNYNSHNVYIMADKQKNGIKVNFKIRHNIE
DGSVQLADHYQQNTPIGDGPVLLPDNHYLSTQSALSKDPNEKRDHMVLLEFVTAAG
ITLGMDELYK
BE4max-REV1-hUNG, SEQ ID NO: 203
MKRTADGSEFESPKKKRKVSSETGPVAVDPTLRRRIEPHEFEVFFDPRELRKETCL
LYEINWGGRHSIWRHTSQNTNKHVEVNFIEKFTTERYFCPNTRCSITWFLSWSPCG
ECSRAITEFLSRYPHVTLFIYIARLYHHADPRNRQGLRDLISSGVTIQIMTEQESGYC
WRNFVNYSPSNEAHWPRYPHLWVRLYVLELYCIILGLPPCLNILRRKQPQLTFFTIAL
QSCHYQRLPPHILWATGLKSGGSSGGSSGSETPGTSESATPESSGGSSGGSDKKY
SIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEI
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDSGGSRRGGWRKRAENDG
WETWGGYMAAKVQKLEEQFRSDAAMQKDGTSSTIFSGVAIYVNGYTDPSAEELRK
LMMLHGGQYHVYYSRSKTTHIIATNLPNAKIKELKGEKVIRPEWIVESIKAGRLLSYIP
YQLYTKQSSVQKGLSFNPVCRPEDPLPGPSNIAKQLNNRVNHIVKKIETENEVKVNG
MNSWNEEDENNDFSFVDLEQTSPGRKQNGIPHPRGSTAIFNGHTPSSNGALKTQD
CLVPMVNSVASRLSPAFSQEEDKAEKSSTDFRDCTLQQLQQSTRNTDALRNPHRT
NSFSLSPLHSNTKINGAHHSTVQGPSSTKSTSSVSTFSKAAPSVPSKPSDCNFISNF
YSHSRLHHISMWKCELTEFVNTLQRQSNGIFPGREKLKKMKTGRSALVVTDTGDMS
VLNSPRHQSCIMHVDMDCFFVSVGIRNRPDLKGKPVAVTSNRGTGRAPLRPGANP
QLEWQYYQNKILKGKAADIPDSSLWENPDSAQANGIDSVLSRAEIASCSYEARQLGI
KNGMFFGHAKQLCPNLQAVPYDFHAYKEVAQTLYETLASYTHNIEAVSCDEALVDIT
EILAETKLTPDEFANAVRMEIKDQTKCAASVGIGSNILLARMATRKAKPDGQYHLKPE
EVDDFIRGQLVTNLPGVGHSMESKLASLGIKTCGDLQYMTMAKLQKEFGPKTGQML
YRFCRGLDDRPVRTEKERKSVSAEINYGIRFTQPKEAEAFLLSLSEEIQRRLEATGM
KGKRLTLKIMVRKPGAPVETAKFGGHGICDNIARTVTLDQATDNAKIIGKAMLNMFH
TMKLNISDMRGVGIHVNQLVPTNLNPSTCPSRPSVQSSHFPSGSYSVRDVFQVQKA
KKSTEEEHKEVFRAAVDLEISSASRTCTFLPPFPAHLPTSPDTNKAESSGKWNGLHT
PVSVQSRLNLSIEVPSPSQLDQSVLEALPPDLREQVEQVCAVQQAESHGDKKKEPV
NGCNTGILPQPVGTVLLQIPEPQESNSDAGINLIALPAFSQVDPEVFAALPAELQREL
KAAYDQRQRQGENSTHQQSASASVPKNPLLHLKAAVKEKKRNKKKKTIGSPKRIQS
PLNNKLLNSPAKTLPGACGSPQKLIDGFLKHEGPPAEKPLEELSASTSGVPGLSSLQ
SDPAGCVRPPAPNLAGAVEFNDVKTLLREWITTISDPMEEDILQVVKYCTDLIEEKDL
EKLDLVIKYMKRLMQQSVESVWNMAFDFILDNVQVVLQQTYGSTLKVTSGGSGGS
GGSIGQKTLYSFFSPSPARKRHAPSPEPAVQGTGVAGVPEESGDAAAIPAKKAPAG
QEEPGTPPSSPLSAEQLDRIQRNKAAALLRLAARNVPVGFGESWKKHLSGEFGKPY
FIKLMGFVAEERKHYTVYPPPHQVFTWTQMCDIKDVKVVILGQDPYHGPNQAHGLC
FSVQRPVPPPPSLENIYKELSTDIEDFVHPGHGDLSGWAKQGVLLLNAVLTVRAHQ
ANSHKERGWEQFTDAVVSWLNQNSNGLVFLLWGSYAQKKGSAIDRKRHHVLQTA
HPSPLSVYRGFFGCRHFSKTNELLQKSGKKPIDWKELSGGSKRTADGSEFEPKKK
RKVGGGGSGATNFSLLKQAGDVEENPGPMVSKGEELFTGVVPILVELDGDVNGHK
FSVSGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLP
DNHYLSTQSALSKDPNEKRDHMVLLEFVTAAGITLGMDELYK
evoFERNY-APOBEC1, SEQ ID NO: 204
MSFERNYDPRELRKETYLLYEIKWGKSGKLWRHWCQNNRTQHAEVYFLENIFNAR
RFNPSTHCSITWYLSWSPCAECSQKIVDFLKEHPNVNLEIYVARLYYPENERNRQG
LRDLVNSGVTIRIMDLPDYNYCWKTFVSDQGGDEDYWPGHFAPWIKQYSLKL
evoAPOBEC1, SEQ ID NO: 205
MSSKTGPVAVDPTLRRRIEPHEFEVFFDPRELRKETCLLYEINWGGRHSIWRHTSQ
NTNKHVEVNFIEKFTTERYFCPNTRCSITWFLSWSPCGECSRAITEFLSRYPNVTLFI
YIARLYHLANPRNRQGLRDLISSGVTIQIMTEQESGYCWHNFVNYSPSNESHWPRY
PHLWVRLYVLELYCIILGLPPCLNILRRKQSQLTSFTIALQSCHYQRLPPH1LWATGLK
BE4max(R33A) w/o UGI = miniCGBE1, SEQ ID NO: 206
MKRTADGSEFESPKKKRKVSSETGPVAVDPTLRRRIEPHEFEVFFDPRELAKETCLL
YEINWGGRHSIWRHTSQNTNKHVEVNFIEKFTTERYFCPNTRCSITWFLSWSPCGE
CSRAITEFLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLTFFTIALQ
SCHYQRLPPHILWATGLKSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLWAKVEKGKSKKL
KSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTID
RKRYTSTKEVLDATLIHQSITGLYETRIDLSQLGGDSGGSKRTADGSEFEPKKKRKV
GGGGSGATNFSLLKQAGDVEENPGPMVSKGEELFTGVVPILVELDGDVNGHKFSV
SGEGEGDATYGKLTLKFICTTGKLPVPWPTLVTTLTYGVQCFSRYPDHMKQHDFFK
SAMPEGYVQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGPVLLPDNH
YLSTQSALSKDPNEKRDHMVLLEFVTAAGITLGMDELYK
nCas9_NG, SEQ ID NO: 207
MKRTADGSEFESPKKKRKVSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESIRPKRNSDKLIARKKDWDPKKYGGFVSPTVAYSVLVVAKVEKGK
SKKLKSVKELLGITIMERSSFEKNPIDFLEAKGYKEVKKDLIIKLPKYSLFELENGRKR
MLASARFLQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPRAFKYFD
TTIDRKVYRSTKEVLDATLIHQSITGLYETRIDLSQLGGDSGGSKRTADGSEFEPKKK
RKVGSGATNFSLLKQAGDVEENPGPMVSKGEELFTGVVPILVELDGDVNGHKFSVS
GEGEGDATYGKLTLKFICTTGKLPVPWPTLVTTLTYGVQCFSRYPDHMKQHDFFKS
AMPEGYVQERTIFFKDDGNYKTRAEVKFEGDTLVNRIELKGIDFKEDGNILGHKLEY
NYNSHNVYIMADKQKNGIKVNFKIRHNIEDGSVQLADHYQQNTPIGDGPVLLPDNHY
LSTQSALSKDPNEKRDHMVLLEFVTAAGITLGMDELYKSGGSPKKKRKV
CGBE1_NG, SEQ ID NO: 208
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSSETGPVAVDPTLRRRIEPHEFE
VFFDPRELAKETCLLYEINWGGRHSIWRHTSQNTNKHVEVNFIEKFTTERYFCPNTR
CSITWFLSWSPCGECSRAITEFLSRYPHVTLFIYIARLYHHADPRNRQGLRDLISSGV
TIQIMTEQESGYCWRNFVNYSPSNEAHWPRYPHLWVRLYVLELYCIILGLPPCLNIL
RRKQPQLTFFTIALQSCHYQRLPPHILWATGLKSGGSSGGSSGSETPGTSESATPE
SSGGSSGGSDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDL
LRKQRTFDNGSIPHQIHLGELHAILRRQEDFYPFLKDNREKIEKILTFRIPYYVGPLAR
GNSRFAWMTRKSEETITPWNFEEWDKGASAQSFIERMTNFDKNLPNEKVLPKHSL
LYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESIRPKRNSDKLIARKKDWDPKKYGGFVSPTVAYSV
LVVAKVEKGKSKKLKSVKELLGITIMERSSFEKNPIDFLEAKGYKEVKKDLIIKLPKYSL
FELENGRKRMLASARFLQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLG
APRAFKYFDTTIDRKVYRSTKEVLDATLIHQSITGLYETRIDLSQLGGDSGGSKRTAD
GSEFEPKKKRKVGGGGSGATNFSLLKQAGDVEENPGPMVSKGEELFTGVVPILVEL
DGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTP
IGDGPVLLPDNHYLSTQSALSKDPNEKRDHMVLLEFVTAAGITLGMDELYK
miniCGBE1_NG, SEQ ID NO: 209
MKRTADGSEFESPKKKRKVSSETGPVAVDPTLRRRIEPHEFEVFFDPRELAKETCLL
YEINWGGRHSIWRHTSQNTNKHVEVNFIEKFTTERYFCPNTRCSITWFLSWSPCGE
CSRAITEFLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLTFFTIALQ
SCHYQRLPPHILWATGLKSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESIRPKRNSDKLIARKKDWDPKKYGGFVSPTVAYSVLVVAKVEKGKSKKL
KSVKELLGITIMERSSFEKNPIDFLEAKGYKEVKKDLIIKLPKYSLFELENGRKRMLAS
ARFLQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPRAFKYFDTTIDR
KVYRSTKEVLDATLIHQSITGLYETRIDLSQLGGDSGGSKRTADGSEFEPKKKRKVG
GGGSGATNFSLLKQAGDVEENPGPMVSKGEELFTGVVPILVELDGDVNGHKFSVS
GEGEGDATYGKLTLKFICTTGKLPVPWPTLVTTLTYGVQCFSRYPDHMKQHDFFKS
AMPEGYVQERTIFFKDDGNYKTRAEVKFEGDTLVNRIELKGIDFKEDGNILGHKLEY
NYNSHNVYIMADKQKNGIKVNFKIRHNIEDGSVQLADHYQQNTPIGDGPVLLPDNHY
LSTQSALSKDPNEKRDHMVLLEFVTAAGITLGMDELYK
nCas9_VRQR, SEQ ID NO: 210
MKRTADGSEFESPKKKRKVSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLY
LYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFVSPTVAYSVLVVAKVEKGK
SKKLKSVKELLGITIMERSSFEKNPIDFLEAKGYKEVKKDLIIKLPKYSLFELENGRKR
MLASARELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYFD
TTIDRKQYRSTKEVLDATLIHQSITGLYETRIDLSQLGGDSGGSKRTADGSEFEPKKK
RKVGSGATNFSLLKQAGDVEENPGPMVSKGEELFTGVVPILVELDGDVNGHKFSVS
GEGEGDATYGKLTLKFICTTGKLPVPWPTLVTTLTYGVQCFSRYPDHMKQHDFFKS
AMPEGYVQERTIFFKDDGNYKTRAEVKFEGDTLVNRIELKGIDFKEDGNILGHKLEY
NYNSHNVYIMADKQKNGIKVNFKIRHNIEDGSVQLADHYQQNTPIGDGPVLLPDNHY
LSTQSALSKDPNEKRDHMVLLEFVTAAGITLGMDELYKSGGSPKKKRKV
CGBE1_VRQR, SEQ ID NO: 211
MKRTADGSEFESPKKKRKVANELTWHDVLAEEKQQPYFLNTLQTVASERQSGVTIY
PPQKDVFNAFRFTELGDVKVVILGQDPYHGPGQAHGLAFSVRPGIAIPPSLLNMYKE
LENTIPGFTRPNHGYLESWARQGVLLLNTVLTVRAGQAHSHASLGWETFTDKVISLI
NQHREGVVFLLWGSHAQKKGAIIDKQRHHVLKAPHPSPLSAHRGFFGCNHFVLAN
QWLEQRGETPIDWMPVLPAESESGGSGGSGGSSSETGPVAVDPTLRRRIEPHEFE
VFFDPRELAKETCLLYEINWGGRHSIWRHTSQNTNKHVEVNFIEKFTTERYFCPNTR
CSITWFLSWSPCGECSRAITEFLSRYPHVTLFIYIARLYHHADPRNRQGLRDLISSGV
TIQIMTEQESGYCWRNFVNYSPSNEAHWPRYPHLWVRLYVLELYCIILGLPPCLNIL
RRKQPQLTFFTIALQSCHYQRLPPHILWATGLKSGGSSGGSSGSETPGTSESATPE
SSGGSSGGSDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDL
LRKQRTFDNGSIPHQIHLGELHAILRRQEDFYPFLKDNREKIEKILTFRIPYYVGPLAR
GNSRFAWMTRKSEETITPWNFEEWDKGASAQSFIERMTNFDKNLPNEKVLPKHSL
LYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFVSPTVAYSVL
VVAKVEKGKSKKLKSVKELLGITIMERSSFEKNPIDFLEAKGYKEVKKDLIIKLPKYSL
FELENGRKRMLASARELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLG
APAAFKYFDTTIDRKQYRSTKEVLDATLIHQSITGLYETRIDLSQLGGDSGGSKRTAD
GSEFEPKKKRKVGGGGSGATNFSLLKQAGDVEENPGPMVSKGEELFTGVVPILVEL
DGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTP
IGDGPVLLPDNHYLSTQSALSKDPNEKRDHMVLLEFVTAAGITLGMDELYK
miniCGBE1_VRQR, SEQ ID NO: 212
MKRTADGSEFESPKKKRKVSSETGPVAVDPTLRRRIEPHEFEVFFDPRELAKETCLL
YEINWGGRHSIWRHTSQNTNKHVEVNFIEKFTTERYFCPNTRCSITWFLSWSPCGE
CSRAITEFLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLTFFTIALQ
SCHYQRLPPHILWATGLKSGGSSGGSSGSETPGTSESATPESSGGSSGGSDKKYS
IGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIM
NFFKTEITLANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEKGKSKKLK
SVKELLGITIMERSSFEKNPIDFLEAKGYKEVKKDLIIKLPKYSLFELENGRKRMLASA
RELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDR
KQYRSTKEVLDATLIHQSITGLYETRIDLSQLGGDSGGSKRTADGSEFEPKKKRKVG
GGGSGATNFSLLKQAGDVEENPGPMVSKGEELFTGVVPILVELDGDVNGHKFSVS
GEGEGDATYGKLTLKFICTTGKLPVPWPTLVTTLTYGVQCFSRYPDHMKQHDFFKS
AMPEGYVQERTIFFKDDGNYKTRAEVKFEGDTLVNRIELKGIDFKEDGNILGHKLEY
NYNSHNVYIMADKQKNGIKVNFKIRHNIEDGSVQLADHYQQNTPIGDGPVLLPDNHY
LSTQSALSKDPNEKRDHMVLLEFVTAAGITLGMDELYK
nCas9(H840A) for PE2, SEQ ID NO: 213
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIY
LALAHMIKFRGHFLIEGDLNPDNSDVDKLFIQLVQTYNQLFEENPINASGVDAKAILS
ARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDAIVPQSFLK
DDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
SGGSSGGSSGSETPGTSESATPESSGGSSGGSSSGGSKRTADGSEFEPKKKRKV
GSGATNFSLLKQAGDVEENPGPMVSKGEELFTGVVPILVELDGDVNGHKFSVSGE
GEGDATYGKLTLKFICTTGKLPVPWPTLVTTLTYGVQCFSRYPDHMKQHDFFKSAM
PEGYVQERTIFFKDDGNYKTRAEVKFEGDTLVNRIELKGIDFKEDGNILGHKLEYNY
NSHNVYIMADKQKNGIKVNFKIRHNIEDGSVQLADHYQQNTPIGDGPVLLPDNHYLS
TQSALSKDPNEKRDHMVLLEFVTAAGITLGMDELYK
PE2, SEQ ID NO: 214
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIY
LALAHMIKFRGHFLIEGDLNPDNSDVDKLFIQLVQTYNQLFEENPINASGVDAKAILS
ARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDAIVPQSFLK
DDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
SGGSSGGSSGSETPGTSESATPESSGGSSGGSSTLNIEDEYRLHETSKEPDVSLG
STWLSDFPQAWAETGGMGLAVRQAPLIIPLKATSTPVSIKQYPMSQEARLGIKPHIQ
RLLDQGILVPCQSPWNTPLLPVKKPGTNDYRPVQDLREVNKRVEDIHPTVPNPYNL
LSGLPPSHQWYTVLDLKDAFFCLRLHPTSQPLFAFEWRDPEMGISGQLTWTRLPQ
GFKNSPTLFNEALHRDLADFRIQHPDLILLQYVDDLLLAATSELDCQQGTRALLQTLG
NLGYRASAKKAQICQKQVKYLGYLLKEGQRWLTEARKETVMGQPTPKTPRQLREF
LGKAGFCRLFIPGFAEMAAPLYPLTKPGTLFNWGPDQQKAYQEIKQALLTAPALGLP
DLTKPFELFVDEKQGYAKGVLTQKLGPWRRPVAYLSKKLDPVAAGWPPCLRMVAAI
AVLTKDAGKLTMGQPLVILAPHAVEALVKQPPDRWLSNARMTHYQALLLDTDRVQF
GPVVALNPATLLPLPEEGLQHNCLDILAEAHGTRPDLTDQPLPDADHTWYTDGSSL
LQEGQRKAGAAVTTETEVIWAKALPAGTSAQRAELIALTQALKMAEGKKLNVYTDS
RYAFATAHIHGEIYRRRGWLTSEGKEIKNKDEILALLKALFLPKRLSIIHCPGHQKGHS
AEARGNRMADQAARKAAITETPDTSTLLIENSSPSGGSKRTADGSEFEPKKKRKVG
SGATNFSLLKQAGDVEENPGPMVSKGEELFTGVVPILVELDGDVNGHKFSVSGEG
EGDATYGKLTLKFICTTGKLPVPWPTLVTTLTYGVQCFSRYPDHMKQHDFFKSAMP
EGYVQERTIFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLLPDNHYLST
QSALSKDPNEKRDHMVLLEFVTAAGITLGMDELYK
Wild type Cas9, SEQ ID NO: 215
MDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGFIKRQLV
ETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGDGSPKKKRKVSSDYKDH
DGDYKDHDIDYKDDDDK
SEQ ID NO: 318
>sp|P97931|UNG_MOUSE Uracil-DNA glycosylase OS = Mus musculus OX = 10090
GN = Ung PE = 1 SV = 3
MIGQKTLYSFFSPTPTGKRTTRSPEPVPGSGVAAEIGGDAVASPAKKARVEQNEQG
SPLSAEQLVRIQRNKAAALLRLAARNVPAGFGESWKQQLCGEFGKPYFVKLMGFV
AEERNHHKVYPPPEQVFTWTQMCDIRDVKVVILGQDPYHGPNQAHGLCFSVQRPV
PPPPSLENIFKELSTDIDGFVHPGHGDLSGWARQGVLLLNAVLTVRAHQANSHKER
GWEQFTDAVVSWLNQNLSGLVFLLWGSYAQKKGSVIDRKRHHVLQTAHPSPLSVH
RGFLGCRHFSKANELLQKSGKKPINWKEL
SEQ ID NO: 319
>tr|Q5BK44|Q5BK44_RAT Uracil-DNA glycosylase OS = Rattus norvegicus
OX = 10116GN = Ung PE = 2 SV = 1
MGILGPRPLKLARSLRAPRGARLRSLTPDPDSWQASPAKKARVEQDEPATPPSSPL
SAEQLVRIQRNKAAALLRLAARNVPAGLGESWKQQLCGEFGKPYFVKLMGFVAEE
RKHHKVYPPPEQVFTWTQMCDIRDVKVVILGQDPYHGPNQAHGLCFSVQRPVPPP
PSLENIFKELSTDIDGFVHPGHGDLSGWARQGVLLLNAVLTVRAHQANSHKERGWE
QFTDAVVSWLNQNLNGLVFLLWGSYAQKKGSAIDRKRHHVLQTAHPSPLSVYRGF
FGCRHFSKANELLQRSGKKPISWKEL
SEQ ID NO: 320
>sp|P12887|UNG_YEAST Uracil-DNA glycosylase OS = Saccharomyces cerevisiae
(strain ATCC 204508/S288c) OX = 559292 GN = UNG1 PE = 1 SV = 1
MWCMRRLPTNSVMTVARKRKQTTIEDFFGTKKSTNEAPNKKGKSGATFMTITNGA
AIKTETKAVAKEANTDKYPANSNAKDVYSKNLSSNLRTLLSLELETIDDSWFPHLMD
EFKKPYFVKLKQFVTKEQADHTVFPPAKDIYSWTRLTPFNKVKVVIIGQDPYHNFNQ
AHGLAFSVKPPTPAPPSLKNIYKELKQEYPDFVEDNKVGDLTHWASQGVLLLNTSLT
VRAHNANSHSKHGWETFTKRVVQLLIQDREADGKSLVFLLWGNNAIKLVESLLGST
SVGSGSKYPNIMVMKSVHPSPLSASRGFFGTNHFKMINDWLYNTRGEKMIDWSVV
PGTSLREVQEANARLESESKDP
SEQ ID NO: 321
>sp|Q9U221|UNG_CAEEL Uracil-DNA glycosylase OS = Caenorhabditis elegans
OX = 6239 GN = ung-1 PE = 1 SV = 1
MSKTVRIPDMFLKASAASKRKSASNTENIPEKVPAGNENQEVKKMKLQAPEPTEILL
KSLLTGESWSKLLEEEFKKGYISKIEKFLNSEVNKGKQVFPPPTQIFTTFNLLPFDEIS
VVIIGQDPYHDDNQAHGLSFSVQKGVKPPPSLKNIYKELESDIEGFKRPDHGNLLGW
TRQGVFMLNATLTVRAHEANSHAKIGWQTFTDTVIRIISRQSEKPIVFLLWGGFAHK
KEELIDTKKHVVIKTAHPSPLSARKWWGCKCFSKCNTELENSGRNPINWADL
SEQ ID NO: 322
>sp|Q9LIH6|UNG_ARATH Uracil-DNA glycosylase, mitochondrial OS = Arabidopsis
thaliana OX = 3702 GN = UNG PE = 1 SV = 1
MASSTPKTLMDFFQPAKRLKASPSSSSFPAVSVAGGSRDLGSVANSPPRVTVTTSV
ADDSSGLTPEQIARAEFNKFVAKSKRNLAVCSERVTKAKSEGNCYVPLSELLVEES
WLKALPGEFHKPYAKSLSDFLEREIITDSKSPLIYPPQHLIFNALNTTPFDRVKTVIIGQ
DPYHGPGQAMGLSFSVPEGEKLPSSLLNIFKELHKDVGCSIPRHGNLQKWAVQGVL
LLNAVLTVRSKQPNSHAKKGWEQFTDAVIQSISQQKEGVVFLLWGRYAQEKSKLID
ATKHHILTAAHPSGLSANRGFFDCRHFSRANQLLEEMGIPPIDWQL
SEQ ID NO: 323
>tr|Q7ZVD1|Q7ZVD1_DANRE Uracil-DNA glycosylase OS = Danio rerio OX = 7955
GN = unga PE = 2 SV = 1
MIGQKSIKSFFSPASKKRNLDEIKTGETRDDVKKQKLESGNEAPLSPEQLERIAKNK
KAA
LERLQSAAPDGIGESWLKALSAEFGKSYFKSLMSFVGEERKKHTIYPPPHAVFTWT
QTCDIKDVKVVILGQDPYHGPNQAHGLCFSVQRPVPPPPSLVNIFKELASDIEGFVQ
PDHGDLTGWANQGVLLLNAVLTVRAHQANSHKDKGWETFTDAVVHWLSSNMQGL
VFILWGSYAQKKGAAINKKQHHVLQAVHPSPLSAHRGFFGCKHFSKANELLKKSGK
KPIDWKAL
SEQ ID NO: 324
>tr|G1SJ42|G1SJ42_RABIT Uracil-DNA glycosylase OS = Oryctolagus cuniculus
OX = 9986 GN = UNG PE = 3 SV = 1
MIGQKTLYSFFSPSPAGKRHTRSPEPAAPGTGVAAATEESRDAEASPAKKARAGKD
EPGTPPSSPLSPEQLVRIQRNKAAALLRLAARNVPVGFGESWKKHLCGEFGKPYFI
KLMGFVAEERKHHTVYPPPHQVFTWTQMCDIRDVKVVILGQDPYHGPSQAHGLCF
SVQRPVPPPPSLENIYKELSTDIEGFVHPGHGDLSGWAKQGVLLLNAVLTVRAHQP
TSHKDRGWEQFTDAVVSWLNHNSSGLVFLLWGSYAQRKGSAIDRKRHHVLQTAH
PSPLSVYRGFFGCRHFSKTNELLRKSGKKPIDWTKL
SEQ ID NO: 325
>tr|A0A452THE0|A0A452THE0_URSMA Uracil-DNA glycosylase OS = Ursus
maritimus OX = 29073 GN = UNG PE = 3 SV = 1
MARIQNLNSNSYTGSHARRTLTENKNCDNERALGVWGKGAGSLRLPVHEPRSPEP
CKHRGPPKKARAVQEDPGTPPSSPLSPEQLVRIQRNKAAALLRLAARNVPVGFGES
WKKPLSAEFGKPYFIKLMGFVAEERKHYTVYPPPHQVFTWTQMCDIRQVKVVILGQ
DPYHGPNQAHGLCFSVQRPVPPPPSLENIYKELSTDIDGFVHPGHGDLSGWAKQG
VLLLNAVLTVRAHQANSHKERGWEQFTDAVVSWLNQNSSGLVFLLWGSYAQKKG
SAIDRKRHHVLQTAHPSPLSVYRGFFGCRHFSKTNELLRKSGKEPINWKDL
SEQ ID NO: 326
>tr|A0A2K6MB33|A0A2K6MB33_RHIBE Uracil-DNA glycosylase OS = Rhinopithecus
bieti OX = 61621 GN = UNG PE = 3 SV = 1
MIGQKTLYSFFSPSPARKRRAPSPEPAVLGTGVAAVPEENGDAAANPAKKAPAAQE
ESGTPSSSPLSAEQLDRIQRNKAAALLRLAARNVPVGFGESWKKHLSGEFGKPYFI
KLMGFVAEERKHYTVYPPPHQVFTWTQMCDIRDVKVVILGQDPYHGPNQAHGLCF
SVQRPVPPPPSLENIYKELSTDIEDFVHPGHGDLSGWAKQGVLLLNAVLTVRAHQA
NSHKERGWEQFTDAVVSWLNQNSNGLVFLLWGSYAQKKGSAIDRKRHHVLQTAH
PSPLSVYRGFFGCRHFSKTNELLQKSGKXVKWEFRGLTAFRAGSPEHRFTHIFINS
KPVISIVLQILN
SEQ ID NO: 327
>tr|A0A4X2KC02|A0A4X2KC02_VOMUR Uracil-DNA glycosylase OS = Vombatus
ursinus OX = 29139 GN = UNG PE = 3 SV = 1
MIGQKTLHSFFSPSAPKKRRSCTETPADPGTEAVVQSEDASVSPVRKRRPEDEPRA
PSSPLSPEQLDRIQRNKAAALLRLASRNVPAGFGESWKRQLSAEFGKPYFIQLMGF
VAEERKRHTVYPPPDQVFTWTQLCEIRDVKVVILGQDPYHGPNQAHGLCFSVQRP
VPPPPSLENIYKELSTDIEGFAPPGHGDLSGWARQGVLLLNAVLTVRAHQANSHKE
RGWEQFTDAVVSWLNENLDGLVFMLWGSYAQKKGLSINRKRHHVLQTAHPSPLSV
HRGFLGCRHFSKTNELLKKSGKKPIDWKAL
SEQ ID NO: 328
>tr|A0A1X2AUJ0|A0A1X2AUJ0_9MYCO Uracil-DNA glycosylase
OS = Mycobacterium riyadhense OX = 486698 GN = ung PE = 3 SV = 1
MTARPLSELVEQGWAAALAPVTEQVAQMGQFLRTEIAAGRRYLPAGSNVLRAFTFP
FDEVRVLIVGQDPYPTPGHAVGLSFSVAPDVRPLPRSLANIFDEYTADLGHPQPSC
GDLSPWAQRGVLLLNRVLTVRPSNPASHRGKGWEAVTECAIRALAARSKPLVAILW
GRDASTLKPMLATGNCVAIESPHPSPLSASRGFFGSRPFSRANELLAGMGGDPVD
WRLP
SEQ ID NO: 329
>tr|A0A498LRM7|A0A498LRM7_LABRO Uracil-DNA glycosylase OS = Labeo rohita
OX = 84645 GN = UNG PE = 3 SV = 1
MQLSEEQLHQIEQNRRAALERLAKRNVPVPVGESWRKKIGTEFTKPYFTKLMSFVT
MERKCFTVYPSPEQVFHCTTLCAIEDVKVVILGQDPYHHPGQAHGLAFSVLRPKPP
PPSLENIFMELKEDIVGFRHPGHGDLTGWAKQGVLLLNSVLTVRAHQPTSHEGQG
WEIFTDAVVLWLSRNLNGLVFLLWGSYAQRKGRVIDRSLEERCQRILQGMEGSLTA
RDRVGIQDFVLLDAYTSETAFMDNLRKRFNENLIYTYIGTLLVSVNPYKELGIYTKKQ
MDIYMGVNFFELPPHIFALADNVYRTMISETNNHFILISGESGAGKTEASKKVLQFYA
VCCPSTRLLDNVRDRLLLSNPVLEAFGNAKTLKNDNSSRFGKYMDIQFDHQGAAVG
GHILSYLLEKSRVVHQNHGERNFHIFYQLVEGGEDELLRWLGLERNCQNYRYLIQG
ECAKVSSINDKSDWKTVQKALTIIEFSEKDIEHLFAIIASVLHLGNVHFEASAMGYAKL
NSNAEVHWLSKLLGIPSNMLQEGLTHRKIEAKAEEVLSPFTAEHAKYARDALAKAIY
GRTFSWLVNKINESLANKWEPVPYFNNKIICDLVEEKHRGIISVLDEECLRPGEATDF
TFLEKLEEKMSGHPHFVTHKLADQKTRKTLERGDFRLLHYAGEVTYSVVGFLDKNN
DLLYRNIKEVMRQSKNSIIQHCFHTIEPDGKKRPETVATQFKSSLAGLTEILMTKEPW
YVRCLKPNHCKQPDRFDDVMVRHQVKYLGLMEHLRVRRAGFAYRRRYEVFLKRR
CFSLLLTCEHLTNLNAYLCRYKPLCPDTWPHWKGTPAEGVQRLIKHLGYKPDEYKM
GRTKIFIRHPRTLFATEDAFEICKHELATRIQAKYKGYRVKGEYQRQREAATKIETCW
RGLQARKERERRAWAVKVIKKFIKGFMNRNQPVSMDNSEYLAFVRQSYLTRLQEN
LPKSVLDKTTWLTPPPIMQEYSVPVIKYDRNGFRPRFRQLIFTQAAAYLVEEAKIKQR
VNYSSLKGVSVSNLSDNFLILHVTCEDTKQKGDLVLQCSYLFEALTKICVVTKNHNLI
KVVQGSVRFDIQPGKEGFVDFKSSSESMVYRAKNGHLMVGDFVDRGYYSLETFTY
LLALKAKWPDRITLLRGNHESRQITQVYGFYDECQTKYGNANAWRYCTKVFDMLTV
AALIDEQILCVHGGLSPDIKTLDQIRTIERNQEIPHKGAFCDLVWSDPEDVDTWAISP
RGAGWLFGAKVTNEFVHINNLKLICRAHQLVHEGYKFMFDEKLVTVWSAPNYCYRC
GNIASIMVFKDVNTREPKLFRAVPDSERVIPPRTTTPYFL
SEQ ID NO: 330
>sp|O74834|UNG_SCHPO Uracil-DNA glycosylase OS = Schizosaccharomyces
pombe (strain 972/ATCC 24843) OX = 284812 GN = ung1 PE = 3 SV = 1
MTVLNTTDKRKADDTVNKLDGKLKQPRLDNFFKTNTSSPALKDTQVLDNKENNSVS
KFNKEKWAENLTPAQRKLLQLEIDTLESSWFDALKDEFLKPYFLNLKEFLMKEWQS
QRVFPPKEDIYSWSHHTPLHKTKVILLGQDPYHNIGQAHGLCFSVRPGIPCPPSLVNI
YKAIKIDYPDFVIPKTGYLVPWADQGILMLNASLTVRAHQAASHSGKGWETFTSAVL
QVALNRNRKGLVILAWGTPAAKRLQGLPLKAHYVLRSVHPSPLSAHRGFFECHHFK
KTNEWLEEQYGPEKCINWSAVSEQKAKIKSSELESSSTE
SEQ ID NO: 331
>tr|A0A3B5KG53|A0A3B5KG53_TAKRU Uracil-DNA glycosylase OS = Takifugu
rubripes OX = 31033 GN = ung PE = 3 SV = 1
MIGQKTINSFFSPVPKKRICKDLSETEEDAKDHIIQKKRKSPEPEPASPPAAPLSSEQ
LERIARNKRAALERLTSAQIPAGIGEGWRDKLSAEFGKPYFKQLTTYVAEERKRRTV
YPPADQVFTWTQMCDIRDVKVVILGQDPYHGHNQAHGLCFSVKRPVPPPPSLENM
YKELVSDIPGFQHPGHGDLTGWAKQGVLLLNAVLTVRAHNANSHKDKGWETFTDA
VVQWLNTNLDGVVFMLWGSYAQKKGAAINRKRHHVLQTVHPSPLSAHRGFFGCA
HFSKANELLKKSGKSPVDWKA
L
SEQ ID NO: 332
>tr|I3M8Q6|I3M8Q6_ICTTR Uracil-DNA glycosylase OS = Ictidomys tridecemlineatus
OX = 43179 GN = UNG PE = 3 SV = 1
MIGQKTLYSFFSPSPARKRSVRSPEPADLGTGVVAVAEENGDAADHPTKKARVGQ
EEPDTPPSSPLSQEQLVRIQRNKAAALLRLAARNVPVGFGESWRKPLGAEFGKPYF
IKLMGFVAEERKRYTVYPPPHQVFTWTQTCDIKDVKVVILGQDPYHGPNQAHGLCF
SVQRPVPPPPSLENIYKELSTDIDGFVHPGHGDLSGWAKQGVLLLNAVLTVRAHQA
NSHKERGWEEFTDAVVSWLNQNLNGLVFLLWGSYAQKKGIAIDRKRHHVLQTAHP
SPLSVYRGFFGCRHFSKANELLQKSGKKPIDWKEL
SEQ ID NO: 333
>tr|A0A3P9H4T8|A0A3P9H4T8_ORYLA Uracil-DNA glycosylase OS = Oryzias latipes
OX = 8090 GN = UNG PE = 3 SV = 1
MLWLRHRSCDKLVGRFLGTGSVIRNKMMKNWGVIGGIAAAVAAGVYVLWGPITVK
KKRKKGMSPGLLNLGNTCFLNALLQGLAACPSFIRWLEKFSGLPSIQSCKDNQLSTT
LLQLLKALSSDEPGEDVLDAGCLLDVLRLYRWHISSFEEQDAHELFHVITSSLEEER
DRQPKVTHLFDVQFLESFPNQDDKALTCISRAPLHPLPGSWKFQHPFHGRLTSNMS
CKRCETQSPVRYDSFESLSLSILLPQWGRPISLDQCLQHFISSETIKEVECENCTKLQ
QHSSINGQLLESQRTTFVKQLRLGKLPQCLCIHLQRLMWSNEGSPIKRQEHVQFSE
YLSMDRYKHDSSTPRTQRVRCAPKTIKAESFDSIEKSMANGTEHHNNNKPFLNGTC
SSMFLGSGVKNPFGFTHHDNSSAEYLFQLVAVLVHLGDMHSGHFVTYRRSPSSSR
SSSNFSSQWLWVSDDSKKLKIAAVDPEPQSSPLSPEQLDKIARNKKAALEKLASGLT
PQGFSESWRGELLSEFSKPYFKDLTKFVSDERKRGTVYPPAEQIFTWTQMCDIRDV
KVVILGQDPYHGPGQAHGLCFSVKRPVSPPPSLENMYKELVSDIEGFKHPGHGDLT
GWAQQGVLLLNAVLTVRAHQANSHKDKGWEVFTDAVVQWLSNNLQGLVFLLWGS
YAQKKGSAINRKHHHVLQAVHPSPLSAHRGFFGCKHFSKANELLKKSGKSPIDWKA
L
SEQ ID NO: 334
>tr|A0A4W4HK79|A0A4W4HK79_ELEEL Uracil-DNA glycosylase OS = Electrophorus
electricus OX = 8005 GN = ung PE = 3 SV = 1
MIGQKSIKSFFSPTSKKRDTDEQTRSEDICNVKKFKTNTSAVLPSPSLSPELLEKIAK
NKKAAQERLAARSAPEGIGKSWQRALGAEFGKTYFKSLMSFVAEERQKQTIYPPPH
QVFTWTRMCEIEDVKVVVLGQDPYHGPNQAHGLCFSVQRPVPPPPSLVNMYKELE
ADIEGFRHPGHGDLTGWAKQGVLLLNAVLTVRAHQANSHKDKGWEILTDAVVNWL
SANLEGLVFMLWGAYAQKKGAAIDRKRHHVLQAVHPSPLSAHRGFFGCKHFSKTN
ELLKKSGKKPIDWKAL
SEQ ID NO: 335
>tr|A0A5G3K4Q6|A0A5G3K4Q6_XENTR Uracil-DNA glycosylase OS = Xenopus
tropicalis OX = 8364 GN = aoc3 PE = 3 SV = 1
MSHPICRPNMSVMFWLLPFPKLPVLSESWRQTSVVCSIRTKQRIGAGVIIPGFSRGA
MIGQRTINSFFGPAAKKRAAPEALGEEGPYKGEITPVKKSRQSGENEIPPAVSPPLS
PEQLERIQRNKAAALQKLAARHVPEGLGQSWKQALLAEFAKPYFVKLSNFVAEERK
KYTVYPPPEEVFTWTQMVDIKDVKVVILGQDPYHGPNQAHGLCFSVKKPVPPPPSL
VNMYKELETDIEGFSHPGHGDLTGWAKQGVLLLNAVLTVRAHNANSHKDCGWEQF
TDSVVSWLNKNMDGLVFMLWGAYAQKKGSNIDRKRHLVLQTVHPSPLSAHRGFFG
CCHFSKTNAYLQGLGKKPIDWKAL
SEQ ID NO: 336
>tr|A0A0F0TTY1|A0A0F0TTY1_ENTCL Uracil-DNA glycosylase OS = Enterobacter
cloacae subsp. cloacae OX = 336306 GN = ung PE = 3 SV = 1
MTTPLTWHDVLAEEKQQPYFINTLSTVAAERQSGQTIYPPQKDVFNAFRYTELSDV
KWILGQDPYHGPGQAHGLAFSVRPGVAIPPSLLNMYKELEGTIPGFTRPNHGYLES
WARQGVLLLNTVLTVRAGQAHSHASLGWETFTDKVISLINEHREGVVFLLWGSHAQ
KKGAIIDRQRHHVLKAPHPSPLSAHRGFFGCNHFVLANEWLEKRGETPIDWMPVLP
AESE
SEQ ID NO: 337
>tr|A0A1V4IJH4|A0A1V4IJH4_9CLOT Uracil-DNA glycosylase OS = Clostridium
oryzae OX = 1450648 GN = ung PE = 3 SV = 1
MTVNIKNDWLELLEDQFEMDYYKDLRHFLISEYKTRTIYPDMYDIFNALNYTAYKDVK
VVILGQDPYHGPNQAHGLSFSVKPGVPAPPSLINIYKELKDDLGCYIPNNGYLKKWT
DEGVLLLNTALTVRAGEANSHRNKGWEIFTDAIISLLNKREKSIVFILWGSNAISKEKLI
TNKAHYIIKSPHPSPLSAHRGFFGSKPFSKANNFLKSIGEKPIDWQIENI
SEQ ID NO: 338
>tr|A0A1C3ZIJ7|A0A1C3ZIJ7_9LACO Uracil-DNA glycosylase OS = Lactobacillus
apis OX = 303541 GN = ung PE = 3 SV = 1
MKKFIGNDWDEVLAPVFESNEYHALHEFLKKEYQTKRIFPDMYHIFTAFKLTPFAKT
KWILGQDPYHNPGQATGMSFAVMPGVKLPPSLQNIYKELYDDVGCVPVQHGYLK
KWADQGVLLLNAVLTVPYGHANGHQGKGWEQVTDAAIKALSDRGQVVFILWGKYA
QNKIALIDQEKNYVIKSAHPSPFSADRGFFGSRPFSRCNEALKKFGEAPIDWQLPQQ
VTESDLA
SEQ ID NO: 339
>tr|A0A519N079|A0A519N079_FLASP Uracil-DNA glycosylase OS = Flavobacterium
sp. OX = 239 GN = ung PE = 3 SV = 1
MKIEESWKKELQSEFEKPYFKELREFISREFDAENGKTCYPPESQIFSAFDHCPFDE
VKVVIIGQDPYHGPGQANGLCFSVADGIPIRPSLRNIFVEIKNDLGKPIPATGNLERW
ANQGVLLLNATLTVRQGEAGSHQKQGWETFTDAVIQHISDDRQNVVFLLWGAFAQ
QKGKNIDKSKHCVLTSGHPSPMSANQGKWFGNKHFSKANEYLKSKGLPEIDW
SEQ ID NO: 340
>tr|A0A1H3TI78|A0A1H3TI78_9BURK Uracil-DNA glycosylase OS = Delftia lacustris
OX = 558537 GN = ung PE = 3 SV = 1
MALQDDAIAPAQADQLQSADPADWPVAPDWQPLVEDFFAGATGQQLLTFLHQRLE
AGAVIFPPQPLRALELTPPDEVRVVILGQDPYHGRGQAEGLSFSVAPGVRMPPSLQ
NIFKEMQRDLGVPFPPFPNPGGSLVKWARNGVLLLNTCLTVEEGQAASHSGKGWE
LLTDAVIRHIAQGTRPVVFMLWGSHAQSKRAFIPGDRGHLVLTSNHPSPLSALRPPV
PFIGNGHFGKARDFRAQHGY
SEQ ID NO: 341
>tr|A0A3D4RH89|A0A3D4RH89_9LACT Uracil-DNA glycosylase OS = Lactococcus
garvieae OX = 1363 GN = ung PE = 3 SV = 1
MKKTDWSGPLRERLPQEYFSDLVDFINEVYAKGNVYPPEDKIFRAIELTALSDVKVIL
VGQDPYPQPGKAQGLSFSYPASFVVNRPDSIVNIRKELQSEGFDKKDSDLTHWAE
QGVLLLNAVLTVPEMKSNAHKGKIWEPLTDEIIKIASDDARPKVFLLWGGDARKKAK
LIDSSKHLVLESAHPSPLSASRGFFGSQPFSKANAFLEKTGQKGIDWSK
SEQ ID NO: 342
>tr|A0A2Z6T8A7|A0A2Z6T8A7_9LACO Uracil-DNA glycosylase OS = Lactobacillus
rodentium OX = 947835 GN = ung PE = 3 SV = 1
MKNLIGNDWDEILAPVFQSENYQELHNFLKEEYQTKTIYPDMYHIFTAFKLTPFAKTK
VVILGQDPYHNPGQATGMSFSVNPGIALPPSLKNIYKELYDDVGAVPVDHGYLKKW
ADQGVLLLNAVLTVPYGKANGHQGKGWEFVTDQAIKRLSERGNVVFILWGRFAQN
KIPLIDQNKNFIIKSSHPSPFSADRGFFGSRPFSRCNDALKQFNEAPIDWQLPAKVNR
TEIV
SEQ ID NO: 343
>sp|Q53HV7|SMUG1_HUMAN Single-strand selective monofunctional uracil DNA
glycosylase OS = Homo sapiens OX = 9606 GN = SMUG1 PE = 1 SV = 2
MPQAFLLGSIHEPAGALMEPQPCPGSLAESFLEEELRLNAELSQLQFSEPVGIIYNP
VEY
AWEPHRNYVTRYCQGPKEVLFLGMNPGPFGMAQTGVPFGEVSMVRDWLGIVGPV
LTPPQEHPKRPVLGLECPQSEVSGARFWGFFRNLCGQPEVFFHHCFVHNLCPLLF
LAPSGRNLTPAELPAKQREQLLGICDAALCRQVQLLGVRLVVGVGRLAEQRARRAL
AGLMPEVQVEGLLHPSPRNPQANKGWEAVAKERLNELGLLPLLLK
SEQ ID NO: 344
>sp|Q811Q1|SMUG1_RAT Single-strand selective monofunctional uracil-DNA
glycosylase OS = Rattus norvegicus OX = 10116 GN = Smug1 PE = 2 SV = 1
MAVSQTFPPGPAHEPASALMEPCARSLAEGFLEEELRLNAELSQLQFPEPVGVIYN
PVDYAWEPHRNYVTRYCQGPKEVLFLGMNPGPFGMAQTGVPFGEVNVVRDWLGI
GGSVLSPPQEHPKRPVLGLECPQSEVSGARFWGFFRTLCGQPQVFFRHCFVHNL
CPLLFLAPSGRNLTPADLPAKHREQLLSICDAALCRQVQLLGVRLVVGVGRLAEQR
ARRALAGLTPEVQVEGLLHPSPRSPQANKGWETAARERLQELGLLPLLTDEGSVRP
TP
SEQ ID NO: 345
>sp|Q6P5C5|SMUG1_MOUSE Single-strand selective monofunctional uracil DNA
glycosylase OS = Mus musculus OX = 10090 GN = Smug1 PE = 1 SV = 1
MAASQTFPLGPTHEPASALMEPLPCTRSLAEGFLEEELRLNAELSQLQFPEPVGVIY
NPVDYAWEPHRNYVTRYCQGPKEVLFLGMNPGPFGMAQTGVPFGEVNVVRDWL
GVGGPVLTPPQEHPKRPVLGLECPQSEVSGARFWGFFRTLCGQPQVFFRHCFVH
NLCPLLFLAPSGRNLTPAELPAKQREQLLSICDAALCRQVQLLGVRLVVGVGRLAEQ
RARRALAGLTPEVQVEGLLHPSPRSAQANKGWEAAARERLQELGLLPLLTDEGSA
RPT
SEQ ID NO: 346
>sp|Q9YGN6|SMUG1_XENLA Single-strand selective monofunctional uracil DNA
glycosylase OS = Xenopus laevis OX = 8355 GN = smug1 PE = 1 SV = 1
MAAEACVPAEFSKDEKNGSILSAFCSDIPDITSSTESPADSFLKVELELNLKLSNLVF
QD
PVQYVYNPLVYAWAPHENYVQTYCKSKKEVLFLGMNPGPFGMAQTGVPFGEVNH
VRDWLQIEGPVSKPEVEHPKRRIRGFECPQSEVSGARFWSLFKSLCGQPETFFKH
CFVHNHCPLIFMNHSGKNLTPTDLPKAQRDTLLEICDEALCQAVRVLGVKLVIGVGR
FSEQRARKALMAEGIDVTVKGIMHPSPRNPQANKGWEGIVRGQLLELGVLSLLTG
SEQ ID NO: 347
>sp|Q59l47|SMUG1_BOVIN Single-strand selective monofunctional uracil DNA
glycosylase OS = Bos taurus OX = 9913 GN = SMUG1 PE = 2 SV = 1
MAVPQPFPSGPHLQPAGALMEPQPSPRSLAEGFLQEELRLNDELRQLQFSELVGIV
YNPVEYAWEPHRSYVTRYCQGPKQVLFLGMNPGPFGMAQTGVPFGEVSVVRDWL
GLGGPVRTPPQEHPKRPVLGLECPQSEVSGARFWGFFRNLCGQPEVFFRHCFVH
NLCPLLLLAPSGRNITPAELPAKQREQLLGVCDAALCRQVQLLGVRLVVGVGRVAE
QRARRALASLMPEVQVEGLLHPSPRSPQANKGWEAVAKERLNELGLLPLLTS

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A C-to-G transversion base editor (CGBE) comprising a cytidine deaminase, a programmable DNA binding domain, and further comprising one or more nuclear localization sequences (NLS), and optionally one or more human or E. coli or other uracil-n-glycosylases (UNGs) or SMUG1, preferably wherein the CGBE does not comprise a uracil-N-glycosylase inhibitor (UGI).

2. The CGBE of claim 1, wherein the cytidine deaminase comprises an active cytidine deaminase domain from an engineered rat APOBEC1 (rAPOBEC1) comprising a mutation at residue R33.

3. (canceled)

4. The CGBE of claim 1, wherein the rAPOBEC1 further comprises one or more mutations at amino acid positions that correspond to residues P29, K34, E181, and/or L182 of rAPOBEC1 (SEQ ID NO:67) or to W90Y, R126E, R132E, W90Y+R126E (double mutant), R126E+R132E (double mutant), W90Y+R132E (double mutant), W90Y+R126E+R132E (triple mutant).

5. (canceled)

6. The CGBE of claim 1, wherein the mutation at amino acid position that correspond to residue R33 is a R33A substitution mutation.

7. The CGBE of claim 1, wherein the CGBE comprises N- or C-terminal fusions of one or more human or E. coli UNG or SMUG1 or other orthologues of UNG or SMUG1.

8. The CGBE of claim 7, wherein the one or more UNGs are from E. coli.

9. The CGBE of claim 1, where the UNG(s) is absent.

10. The CGBE of claim 1, wherein the rAPOBEC1 comprises a R33A mutation and one or more mutations at positions: P29F, P29T, K34A, E181Q and/or L182A of rAPOBEC1 (SEQ ID NO:67).

11. The CGBE of claim 10, further comprising one or more mutations in the rAPOBEC1 at residues corresponding to E24, V25; R118, Y120, H121, R126; W224-K229; P168-1186; L173+L180; R15, R16, R17, to K15-17 & A15-17; Deletion E181-L210; P190+P191; Deletion L210-K229 (C-terminal); and/or Deletion S2-L14 (N-terminal) of SEQ ID NO:67.

12. (canceled)

13. (canceled)

14. (canceled)

15. The CGBE of claim 1, comprising a linker between the cytosine deaminase and/or between the cytosine deaminase or single-chain dimers and the programmable DNA binding domain.

16. The CGBE of claim 1, wherein the programmable DNA binding domain is selected from the group consisting of a Clustered Regularly Interspaced Short Palindromic Repeats (CRISPR) Cas RNA-guided nuclease (RGN), an engineered C2H2 zinc-finger, a transcription activator effector-like effector (TALE), and variants thereof.

17. The CGBE of claim 1, wherein the CRISPR RGN is a ssDNA nickase or a catalytically inactive CRISPR Cas RNA-guided nuclease, optionally a Cas9 or Cas12a that has ssDNA nickase activity or is catalytically inactive.

18. A base editing system comprising:
(i) an CGBE of claim 1, wherein the programmable DNA binding domain is a CRISPR Cas RGN or a variant thereof; and
(ii) at least one guide RNA compatible with the base editor comprising a spacer sequence that directs the base editor to a target sequence, preferably wherein the target sequence comprises a cytosine at position 4-8, 5-7, or position 6 (with 1 being the most PAM-distal position).

19. An isolated nucleic acid encoding a CGBE of claim 1.

20. A vector comprising the isolated nucleic acid of claim 19.

21. An isolated host cell, preferably a mammalian host cell, comprising the nucleic acid of claim 19.

22. The isolated host cell of claim 21, wherein the isolated host cell expresses a CGBE.

23. A composition comprising:
(i) a CGBE of claim 1, wherein the programmable DNA binding domain is a CRISPR Cas RGN or a variant thereof;
(ii) at least one guide RNA compatible with the base editor comprising a spacer sequence that directs the base editor to a target sequence, preferably wherein the target sequence comprises a cytosine at position 4-8, 5-7, or position 6 (with 1 being the most PAM-distal position), and
(iii) a pharmaceutically acceptable carrier.

24. The composition of claim 23, comprising one or more ribonucleoprotein (RNP) complexes.

25. A method of generating a cytosine-to-guanine and guanine-to-cytosine alteration in a nucleic acid, the method comprising contacting the nucleic acid with the CGBE of claim 1.

26. (canceled)

27. (canceled)

28. (canceled)

29. (canceled)

30. (canceled)

31. (canceled)

32. (canceled)

33. (canceled)

34. (canceled)
